(19) United States
(12) Patent Application Publication
(10) Pub. No.: US 2024/0254533 A1
(43) Pub. Date: Aug. 1, 2024

(54) METHODS FOR PRODUCING TRYPTAMINE DERIVATIVES

(71) Applicant: OCTARINE BIO APS, Copenhagen (DK)

(72) Inventors: Nicholas Stuart William MILNE, Copenhagen (DK); Annette Munch NIELSEN, Copenhagen (DK); Camilla Knudsen BADEN, Copenhagen (DK); Nethaji Janeshawari GALLAGE, Copenhagen (DK)

(21) Appl. No.: 18/563,923

(22) PCT Filed: May 25, 2022
(86) PCT No.: PCT/EP2022/064352

§ 371 Date: Nov. 24, 2023

(30) Foreign Application Priority Data
May 27, 2021 (EP) 21176391.7
Jan. 12, 2022 (EP) 22151219.7

(51) Int. Cl. C12P 19/44; C12N 9/02; C12N 9/10; C12N 9/12; C12N 9/88; C12N 15/52

(52) U.S. Cl. CPC C12P 19/44; C12N 9/0061; C12N 9/0071; C12N 9/1007; C12N 9/1029; C12N 9/1051; C12N 9/1077; C12N 9/1205; C12N 9/88; C12N 15/52; C12Y 203/01087; C12Y 203/01152; C12Y 403/03002

(57) ABSTRACT

The present disclosure relate to a method for producing a tryptamine derivative of formula (I), wherein the tryptamine derivative (I) is not tryptophane, 4-hydroxytryptamine, N-acetyl-4-hydroxytryptamine, norbaeocystin, baeocystin; psilocybin, psilocin, aeruginascin, halogenated tryptophan, halogenated tryptamine, halogenated N-methylated tryptamine, halogenated N,N-dimethyltryptamine or halogenated N,N,N-trimethyltryptamine; said method comprising providing an indole acceptor of the formula (II), wherein the tryptamine derivative (I) is not tryptophane, 4-hydroxytryptamine, N-acetyl-4-hydroxytryptamine, norbaeocystin, baeocystin; psilocybin, psilocin, aeruginascin, halogenated tryptophan, halogenated tryptamine, halogenated N-methylated tryptamine, halogenated N,N-dimethyltryptamine or halogenated N,N,N-trimethyltryptamine; said method comprising providing an indole acceptor of the formula (II), wherein one or more of RII, RIV, RV, RVI or RVII is not H and Rill is H or CH2CH2NH2 or CH2CHCOOHNH2; and contacting the indole acceptor with a substituent donor in the presence of one or more enzymes substituting one or more H, OH and/or COOH in the indole acceptor with one or more substituents of the substituent donor.

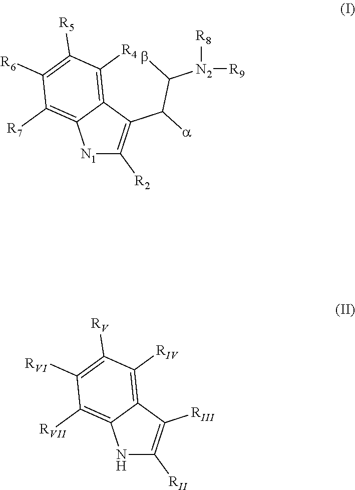

### FIELD

The present invention relates to methods for making tryptamine derivatives and to tryptamine derivatives resulting therefrom.

### BACKGROUND

Tryptamines are well known including serotonin, an important neurotransmitter, and melatonin, a hormone involved in regulating the sleep-wake cycle. Tryptamine alkaloids are found in fungi, plants, and animals; and sometimes used by humans for the neurological or psychotropic effects of the substance. Prominent examples of tryptamine alkaloids include psilocybin (from “Psilocybin mushrooms”) and Dimethyltryptamine (DMT). DMT is obtained from numerous plant sources, like chacruna, and it is often used in ayahuasca brews. Many synthetic tryptamines have also been made, including migraine drugs and psychedelic drugs. Substituted tryptamines, or serotonin analogues, are organic compounds which may be thought of as being derived from tryptamine itself. The molecular structures of all tryptamines contain an indole ring, joined to an amino (NH2) group via an ethyl (—CH2-CH2-) sidechain. In substituted tryptamines, the indole ring, sidechain, and/or amino group are modified by substituting another group for one of the hydrogen (H) atoms.

WO2019173797 relates to microbial cells that contain enzymes involved in a biosynthesis pathway converting anthranilate, indole or tryptophan into a tryptamine. WO2021052989 relates to biotechnological production of psilocybin and to halogenated tryptamines in a cell factory.

Still there is a continuous need for identifying new substituted tryptamines having new and/or enhanced properties with respect to therapeutic effect, administration, formulation and/or production of the substituted tryptamine and/or to devise new and optimized methods for producing such substituted tryptamines.

### SUMMARY

The present invention provides certain improvements offering solutions to drawbacks of known substituted tryptamine derivatives and methods for producing them using known technology. The invention also provides enzymes, which surprisingly acts to catalyze substitutions in tryptamines both in vitro and in vivo and thereby circumvent drawbacks of the known technology and moreover which also integrate and work in genetically modified host cells to produce therein such substituted tryptamine derivatives. The inventors have also found that these substitutions in tryptamines not only produce hitherto unknown substituted tryptamine derivatives, which possesses interesting useful properties, but in vivo expression of substituting enzymes also offers a range of hitherto unknown advantages in processes of producing the substituted tryptamine derivatives in genetically modified cell factories, such as yeast, including but not limited to (i) reducing product inhibition of pathways producing tryptamine by presence of high concentrations of tryptamines or tryptophan; (ii) facilitating export of compounds into the extracellular space; (iii) and reducing the toxicity of tryptamine derived compounds to the microbial host.

Accordingly, in a first aspect the invention provides a method for producing a tryptamine derivative of formula (I):

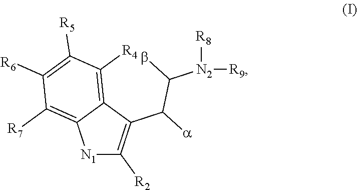

wherein the tryptamine derivative (I) is not tryptophan, 4-hydroxytryptamine, N-acetyl-4-hydroxytryptamine, norbaeocystin, baeocystin; psilocybin, psilocin, aeruginascin, halogenated tryptophan, halogenated tryptamine, halogenated N-methylated tryptamine, halogenated N,N-dimethyltryptamine or halogenated N,N,N-trimethyltryptamine; said method comprising providing an indole acceptor of the formula (II):

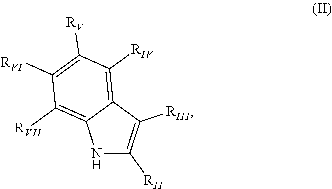

wherein one or more of R_{II}, R_{IV}, R_{V}, R_{VI} or R_{VII} is not H and R_{III} is H or CH₂CH₂NH₂ or CH₂CHCOOHNH₂; and contacting the indole acceptor with a substituent donor in the presence of one or more enzymes substituting one or more H, OH and/or COOH in the indole acceptor with one or more substituents of the substituent donor.

In a further aspect of the invention provides a tryptamine derivative of formula (I):

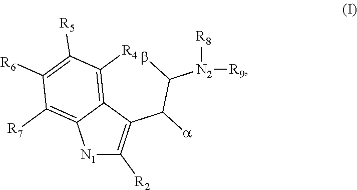

wherein at least one of R₂, R₄ to R₉, α and β is a glycosyl group. Particularly R₂, R₄ to R₇, α and β may be an O-glycosyl group.

In a further aspect the invention provides a microbial host cell genetically modified to perform the method of the invention and produce the tryptamine derivative, wherein the host cell expresses one or more heterologous genes encoding the one or more enzymes, which in the presence of the indole acceptor and one or more substituent donors, transfers one or more substituents to the one or more H, OH and/or COOH of the indole acceptor.

In further aspects the invention provides a cell culture, comprising host cell of the invention and a growth medium; a fermentation liquid comprising the tryptamine derivative (I) comprised in the cell culture of the invention; and a composition comprising the fermentation liquid of the invention and/or the the tryptamine derivative (I) of the invention and one or more agents, additives and/or excipients.

### DESCRIPTION OF DRAWINGS AND FIGURES

FIG. 1, shows a biosynthetic pathway for the production of tryptamine in S. cerevisiae. Overexpressed genes are shown in bold, relevant gene deletions involved in the biosynthetic pathway are indicated with a cross.

FIG. 2 shows a general scheme for integration of gene overexpression cassettes into S. cerevisiae genome. Linear expression cassettes contain overlapping homology to each other while the outermost cassettes (Rec 1 and Rec 5) contain homology to a genomic landing pad in the S. cerevisiae genome. Transformation of linear cassettes results in assembly and integration by homologous recombination.

FIG. 3 shows gene deletion by Ura3 marker replacement. The Ura3 cassette is amplified with primers introducing upstream and downstream homology to the gene to be deleted. Gene deletion occurs by homologous recombination and replacement with the Ura3 marker. The marker can subsequently be looped out in a scarless manner and the Ura3 marker can be reused.

FIG. 4 shows structures of substituted tryptamine glucosides validated by LC-MS/QTOF.

FIG. 5 shows an example of a LC-MS-QTOF chromatogram from in vitro conversion of psilocin to OBT-001 (psilocin-O-β-D-glucoside) by At71C2 (SEQ ID NO's. 193, 194) and further showing the retention time (RT), expected and measured mass of each compound and fragmentation pattern as determined by LC-MS/QTOF analysis.

FIG. 6 shows an example of LC-MS-QTOF chromatogram from in vitro conversion of noribogaine to OBT-002 noribogaine-O-β-D-glucoside by Pt73Y (SEQ ID NO's 203, 204) and further showing the retention time (RT), expected and measured mass of each compound and fragmentation pattern as determined by LC-MS/QTOF analysis.

FIG. 7 shows an example of a LC-MS-QTOF chromatogram from in vitro conversion of bufotenine to OBT-003 (bufotenine-O-β-D-glucoside) by At71C1_At71C2_353 (SEQ ID NO's. 243, 244), further showing the retention time (RT), expected and measured mass of each compound and fragmentation pattern as determined by LC-MS/QTOF analysis.

FIG. 8 shows an example of LC-MS-QTOF chromatogram from in vitro conversion of serotonin to OBT-004 (serotonin-O-β-D-glucoside) by At71C1-Sr71E1_354 (SEQ ID NO's. 199, 200) and further showing the retention time (RT), expected and measured mass of each compound and fragmentation pattern as determined by LC-MS/QTOF analysis.

FIG. 9 show structures of substituted tryptamine xylosides validated by LC-MS/QTOF.

FIG. 10 shows a LC-MS-QTOF chromatogram from in vitro conversion of Psilocin to OBT-005 (psilocin-O-β-D-xyloside) by At71C2 (SEQ ID NO's. 193, 194) and further showing the retention time (RT), expected and measured mass of each compound and fragmentation pattern as determined by LC-MS/QTOF analysis.

FIG. 11 shows a LC-MS-QTOF chromatogram from in vitro conversion of Noribogaine to OBT-006 (noribogaine-O-β-D-xyloside) by Pt73Y (SEQ ID NO's. 203, 204) and further showing the retention time (RT), expected and measured mass of each compound and fragmentation pattern as determined by LC-MS/QTOF analysis.

FIG. 12 shows a LC-MS-QTOF chromatogram from in vitro conversion of Bufotenine to OBT-007 (bufotenine-O-β-D-xyloside) by Pt73Y (SEQ ID NO's. 203, 204) and further showing the retention time (RT), expected and measured mass of each compound and fragmentation pattern as determined by LC-MS/QTOF analysis.

FIG. 13 show the structure of Psilocin di-glucoside produced by combining multiple glycosyltransferases OBT-008 (Psilocin-O-β-D-glucoside-O-β-D-glucoside).

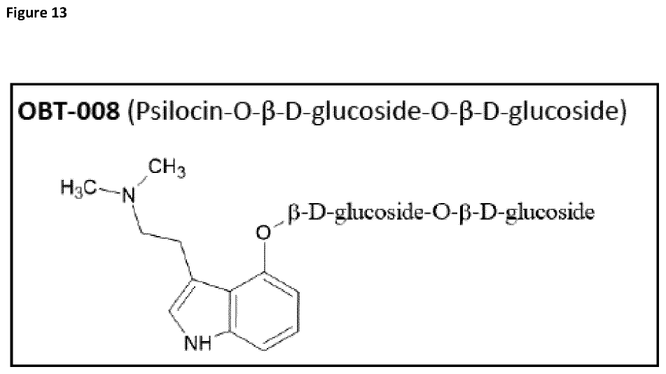

FIG. 14 shows an example of tryptamine production in SC-62, a S. cerevisiae strain engineered to efficiently produce tryptamine. Shown is an HPLC chromatogram of SC-62 after cultivation compared to an authentic tryptamine analytical standard.

FIG. 15 shows an example of serotonin production in SC-75, a S. cerevisiae strain engineered to efficiently produce serotonin. Shown is an HPLC chromatogram of SC-75 after cultivation compared to an authentic serotonin analytical standard.

FIG. 16 shows an example of 4-coumaroylserotonin production in ST-4CS2, a S. cerevisiae strain engineered to efficiently produce 4-coumaroylserotonin. Shown is an HPLC chromatogram of ST-4CS2 after cultivation compared to an authentic 4-coumaroylserotonin analytical standard.

FIG. 17 shows an example of psilocybin production in SC-206 and SC-302. SC-206 is a S. cerevisiae strain engineered to produce psilocybin, SC-302 is the same parental strain but with the DIA3 gene knocked out. Shown is HPLC chromatograms of SC-206 and SC-302 after cultivation compared to an authentic psilocybin analytical standard.

FIG. 18 shows an example of psilocybin production in SC-275 and SC-268. SC-275 is a S. cerevisiae strain engineered to produce psilocybin with a biosynthetic pathway from P. cubensis, SC-268 is a strain with a biosynthetic pathway from P. cyanescens. Shown in HPLC chromatograms of SC-275 and SC268 after cultivation compared to an authentic psilocybin analytical standard. Insert shows a zoomed in view of the psilocybin peaks for clarity.

FIG. 19 shows an example of melatonin production in SC-124, a S. cerevisiae strain engineered to efficiently produce melatonin. Shown is an HPLC chromatogram of SC-124 after cultivation compared to an authentic melatonin analytical standard. Insert shows a zoomed in view of the psilocybin peaks for clarity.

### INCORPORATION BY REFERENCE

All publications, patents, and patent applications referred to herein are incorporated by reference to the same extent as if each individual publication, patent, or patent application was specifically and individually indicated to be incorporated by reference. In the event of a conflict between a term herein and a term in an incorporated reference, the term herein prevails and controls.

### DETAILED DESCRIPTION

### Definitions

Any EC numbers than may be used herein refers to Enzyme Nomenclature 1992 from NC-IUBMB, Academic Press, San Diego, California, including 30 supplements 1-5 published in Eur. J. Bio-chem. 1994, 223, 1-5; Eur. J. Biochem. 1995, 232, 1-6; Eur. J. Biochem. 1996, 237, 1-5; Eur. J. Biochem. 1997, 250, 1-6; and Eur. J. Biochem. 1999, 264, 610-650; respectively. The nomenclature is regularly supplemented and updated; see e.g. http://enzvme.expasv.org/.

The term “fructose-6-phosphate phosphoketolase” as used herein refers to an enzyme catalyzing the reaction of fructose-6-phosphate into Erythrose-4-phosphate and acetyl phosphate.

The term “Phosphotransacetylase” as used herein refers to an enzyme catalyzing the reaction of Acetyl phosphate into Acetyl-CoA.

The term “DAHP synthase” as used herein refers to a 3-deoxy-D-arabino-heptulosonate 7-phosphate synthase enzyme catalyzing the reaction of phosphoenolpyruvate and erythrose-4-phosphate to 3-deoxy-D-arabino-heptulosonate-7-phosphate (DAHP).

The term “Arol” as used herein refers to EPSP synthase catalyzing conversion of DAHP into 5-enolpyruvoyl-shikimate 3-phosphate (EPSP).

The term “Shikimate kinase” as used herein refers to an enzyme catalyzing the reaction of Shikimate into Shikimate-3-phosphate.

The term “Chorismate synthase” as used herein refers to an enzyme catalyzing the reaction of 5-enolpyruvoyl-shikimate 3-phosphate into Chorismate.

The term “Anthranilate synthase” as used herein refers to an enzyme catalyzing the reaction of Chorismate into Anthranilate.

The term “Ribose-phosphate pyrophosphokinase” as used herein refers to an enzyme catalyzing the reaction of Ribose-5-phosphate into phospho-alpha-D-ribosyl-1-pyrophosphate.

The term “anthranilate phosphoribosyl transferase” as used herein refers to an enzyme catalyzing the reaction of anthranilate and phospho-alpha-D-ribosyl-1-pyrophosphate into N-(5-phosphoribosyl)-anthranilate.

The term “N-(5′-phosphoribosyl)-anthranilate isomerase” as used herein refers to an enzyme catalyzing the reaction of N-(5-phosphoribosyl)-anthranilate into 1-(o-carboxyphenylamino)-1′-deoxyribulose 5′-phosphate.

The term “indole-3-glycerol phosphate synthase” as used herein refers to an enzyme catalyzing the reaction of converting 1-(O-carboxyphenylamino)-1′-deoxyribulose 5′-phosphate into (1S,2R)-1-C-(indol-3-yl)-glycerol 3-phosphate.

The term “tryptophan synthase” as used herein refers to an enzyme catalyzing the reaction of converting (1S,2R)-1-C-(indol-3-yl) glycerol 3-phosphate+Serine into L-Tryptophan.

The term “Tryptophan decarboxylase” as used herein refers to an enzyme catalyzing the reaction of L-Tryptophan into Tryptamine.

The term “chorismate mutase” as used herein refers to an enzyme catalyzing the reaction of chorismate to prephenate.

The term “prephenate dehydrogenase” as used herein refers to an enzyme catalyzing the reaction of prephenate to phenylpyruvate.

The term “aromatic aminotransferase” as used herein refers to an enzyme catalyzing the reaction of phenylpyruvate to phenylalanine.

The term “phenylalanine ammonium lyase” as used herein refers to an enzyme catalyzing the reaction of phenylalanine to cinnamate.

The term “Cinnamate 4-hydroxylase” as used herein refers to an enzyme catalyzing the reaction of cinnamate to coumarate.

The term “CPR” as used herein refers to a cytochrome P450 reductase catalyzing the electron transfer (from NADPH) to a cytochrome P450 enzyme, typically in the endoplasmic reticulum of a eukaryotic cell.

The term “Cytochrome P450 enzyme” or “P450 enzymes” or “P450” as used herein interchangeably refers to a family of enzymes containing heme as a cofactor. The P450's of the invention may catalyze oxidation, hydroxylation (hydroxylases) of substrates, and/or catalyze the addition of nitrogen dioxide (NO₂) to substrates.

The term “4-Coumoryl-CoA ligase” as used herein refers to an enzyme catalyzing the reaction of Coumarate to 4-coumoryl CoA.

The term “Tryptophanase” as used herein refers to an enzyme catalyzing the reaction of tryptophan or a derivative thereof into indole or a derivative thereof.

The term “Tryptophan synthase” as used herein refers to an enzyme catalyzing the reaction of indole or a derivative thereof and serine or a derivative thereof into Tryptophan or a derivative thereof.

The terms “Tryptophan decarboxylase” or “non-canonical aromatic amino acid decarboxylase” as used herein refers to an enzyme catalyzing the reaction of Tryptophan or a derivative thereof into Tryptamine or a derivative thereof.

The term “glycosyl transferase” or “GT” as used herein refers to enzymes (EC2.4) that catalyze formation of glycosides by transfer of a glycosyl group (sugar) from an activated glycosyl donor to a nucleophilic glycosyl acceptor molecule, the nucleophile of which can be oxygen-carbon-, nitrogen-, or sulfur-based. The product of glycosyl transfer may be an O-, N-, S-, or C-glycoside. In the context of the present invention the nucleophilic glycosyl acceptor is a tryptamine or tryptophan derivative or a glycosylated tryptamine or tryptophan derivative and the product of glycosyl transfer is an O- or C-glycoside.

Glycosyl transferases may further be divided into different GT families depending on the 3D structure and reaction mechanism. More specifically the GT1 superfamily refers to UDP glycosyl transferases (UGTs) containing the PSPG box binding UDP-sugars. UGT-superfamily members may further be divided into families and subfamilies as defined by the UGT Nomenclature Committee (Mackenzie et al., 1997) depending on the amino acid identity.

The terms “N-methyltransferase” and “O-methyltransferase” and “C-methyltransferase” as used herein refers to an enzyme catalyzing the reaction of methylation of an N, O or C moiety respectively.

The terms “strictosidine synthase” or “1-acetyl-β-carboline synthase” as used herein refers to an enzyme catalyzing the reaction of a tryptamine and an aldehyde into a β-carboline.

The term “N-acetyltransferase” as used herein refers to an enzyme catalyzing the reaction of a tryptamine or derivative thereof into N-acetyltryptamine or derivative thereof.

The term “hydroxytryptamine kinase” as used herein refers to an enzyme catalyzing the reaction of a hydroxytryptamine or derivative thereof into a phosphoryloxytryptamine or derivative thereof. The hydroxytryptamine may for example be a 4-hydroxytryptamine or a 7-hydroxytryptamine.

The term “N-hydroxycinnamoyltransferase” as used herein refers to an enzyme catalyzing the reaction of a tryptamine or derivative thereof and a cinnamoyl-CoA into a N-cinnamoyltryptamine or derivative thereof and CoA.

The term “psilocybin phosphatase” as used herein refers to an enzyme catalyzing the reaction of a R-phosphoryloxytryptamine or derivative thereof into a R-hydroxytryptamine or derivative thereof.

The term “psilocin laccase” as used herein refers to an enzyme catalyzing the reaction of a R-hydroxytryptamine or derivative thereof into a tryptamine quinoid or derivative thereof.

The term “Tryptophan halogenase” as used herein refers to an enzyme catalyzing the reaction of a tryptamine or derivative thereof into a halogenated tryptamine or derivative thereof.

The term “lyase” as used herein refers to an enzyme catalyzing the cleavage of bonds resulting in the splitting of molecules into separate components.

The term “flavin monooxygenase” as used herein refers to an enzyme catalyzing the oxidization of substrates using NADPH as a co-factor and flavin adenine dinucleotide (FAD) as a prothetic group.

The term “nucleotide glycoside” as used herein about glycosyl donors refers to compounds comprising a nucleotide moiety covalently linked to a glycosyl group, where the nucleotide comprises, a nucleoside covalently linked to one or more phosphate groups. Such compounds are also referred to as “activated glycosides” and where the glycosyl group is a sugar as “nucleotide sugars” or “activated sugars”.

The term “heterologous” or “recombinant” and its grammatical equivalents as used herein refers to entities “derived from a different species or cell”. For example, a heterologous or recombinant polynucleotide gene is a gene in a host cell not naturally containing that gene, i.e. the gene is from a different species or cell type than the host cell.

The term “genetically modified host cell” as used herein refers to host cell comprising and expressing heterologous or recombinant polynucleotide genes.

The term “pathway” or “metabolic pathway” or “biosynthetic metabolic pathway” or “operative biosynthetic metabolic pathway” as used herein interchangeably is intended to mean one or more enzymes acting in a live cell to convert a chemical substrate into a chemical product. A pathway may include one enzyme or multiple enzymes acting in sequence. A pathway including only one enzyme may also herein be referred to as “bioconversion”, which is particularly relevant where a cell is fed with a precursor or substrate to be converted by the enzyme into a desired product molecule. Enzymes are characterized by having catalytic activity, which can change the chemical structure of the substrate(s).

An enzyme may have more than one substrate and produce more than one product. The enzyme may also depend on cofactors, which can be inorganic chemical compounds or organic compounds (co-factor and/or co-enzymes). The NADPH-dependent cytochrome P450 reductase (CPR) is an electron donor to cytochromes P450 (CYPs). CPR shuttles electrons from NADPH through the Flavin Adenine Dinucleotide (FAD) and Flavin Mononucleotide (FMN) coenzymes into the iron of the prosthetic heme-group of the CYP.

The term “in vivo”, as used herein refers to within a living cell or organism, including, for example animal, a plant, or a microorganism.

The term “in vitro”, as used herein refers to outside a living cell or organism, including, without limitation, for example, in a microwell plate, a tube, a flask, a beaker, a tank, a reactor and the like.

The term “substrate” or “precursor”, as used herein refers to any compound that can be converted into a different compound. For example, thebaine can be a substrate for P450 and can be converted by demethuylation into Northebaine. For clarity, substrates and/or precursors include both compounds generated in situ by a enzymatic reaction in a cell or exogenously provided compounds, such as exogenously provided organic molecules which the host cell can metabolize into a desired compound.

Term “endogenous” or “native” as used herein refers to a gene or a polypepetide in a host cell which originates from the same host cell.

The term “deletion” as used herein refers to manipulation of a gene so that it is no longer expressed in a host cell.

The term “disruption” as used herein refers to manipulation of a gene or any of the machinery participating in the expression the gene, so that it is no longer expressed in a host cell.

The term “attenuation” as used herein refers to manipulation of a gene or any of the machinery participating in the expression the gene, so that it the expression of the gene is reduced as compared to expression without the manipulation.

The terms “substantially” or “approximately” or “about”, as used herein refers to a reasonable deviation around a value or parameter such that the value or parameter is not significantly changed. These terms of deviation from a value should be construed as including a deviation of the value where the deviation would not negate the meaning of the value deviated from. For example, in relation to a reference numerical value the terms of degree can include a range of values plus or minus 10% from that value. For example, using these deviating terms can also include a range deviation plus or minus such as plus or minus 9%, 8%, 7%, 6%, 5%, 4%, 3%, 2%, or 1% from a specified value.

The term “and/or” as used herein is intended to represent an inclusive “or”. The wording X and/or Y is meant to mean both X or Y and X and Y. Further the wording X, Y and/or Z is intended to mean X, Y and Z alone or any combination of X, Y, and Z.

The terms “isolated” or “purified” or “extracted” or “recovered” as used herein interchangably about a compound, refers to any compound, which by means of human intervention, has been put in a form or environment that differs from the form or environment in which it is found in nature. Isolated compounds include but is not limited to compounds of the invention for which the ratio of the compounds relative to other constituents with which they are associated in nature is increased or decreased. In an important embodiment the amount of compound is increased relative to other constituents with which the compound is associated in nature. In an embodiment the compound of the invention may be isolated into a pure or substantially pure form. In this context a substantially pure compound means that the compound is separated from other exogenous or unwanted material present from the onset of producing the compound or generated in the manufacturing process. Such a substantially pure compound preparation contains less than 10%, such as less than 8%, such as less than 6%, such as less than 5%, such as less than 4%, such as less than 3%, such as less than 2%, such as less than 1%, such as less than 0.5% by weight of other exogenous or unwanted material usually associated with the compound when expressed natively or recombinantly. In an embodiment the isolated compound is at least 90% pure, such as at least 91% pure, such as at least 92% pure, such as at least 93% pure, such as at least 94% pure, such as at least 95% pure, such as at least 96% pure, such as at least 97% pure, such as at least 98% pure, such as at least 99% pure, such as at least 99.5% pure, such as 100% pure by weight.

The term “% identity” is used herein about the relatedness between two amino acid sequences or between two nucleotide sequences. “% identity” as used herein about amino acid sequences refers to the degree of identity in percent between two amino acid sequences obtained when using the Needleman-Wunsch algorithm (Needleman and Wunsch, 1970, J. Mol. Biol. 48: 443-453) as implemented in the Needle program of the EMBOSS package (EMBOSS: The European Molecular Biology Open Software Suite, Rice et al., 2000, Trends Genet. 16: 276-277), preferably version 5.0.0 or later. The parameters used are gap open penalty of 10, gap extension penalty of 0.5, and the EBLOSUM62 (EMBOSS version of BLOSUM62) substitution matrix. The output of Needle labeled “longest identity” (obtained using the -nobrief option) is used as the percent identity and is calculated as follows:

$$\frac{identical amino acid residues}{Length of alignment - total number of gaps in alignment} \times 100$$

“% identity” as used herein about nucleotide sequences refers to the degree of identity in percent between two nucleotide sequences obtained when using the Needleman-Wunsch algorithm (Needleman and Wunsch, 1970, supra) as implemented in the Needle program of the EMBOSS package (EMBOSS: The European Molecular Biology Open Software Suite, Rice et al., 2000, supra), preferably version 5.0.0 or later. The parameters used are gap open penalty of 10, gap extension penalty of 0.5, and the EDNAFULL (EMBOSS version of NCBI NUC4.4) substitution matrix. The output of Needle labeled “longest identity” (obtained using the -nobrief option) is used as the percent identity and is calculated as follows:

$$\frac{identical deoxyribonucleotides}{Length of alignment - total number of gaps in alignment} \times 100$$

The protein sequences of the present invention can further be used as a “query sequence” to perform a search against sequence databases, for example to identify other family members or related sequences.

Such searches can be performed using the BLAST programs. Software for performing BLAST analyses is publicly available through the National Center for Biotechnology Information (http://www.ncbi.nlm.nih.gov). BLASTP is used for amino acid sequences and BLASTN for nucleotide sequences. The BLAST program uses as defaults:
Cost to open gap: default=5 for nucleotides/11 for proteins
Cost to extend gap: default=2 for nucleotides/1 for proteins
Penalty for nucleotide mismatch: default=−3
Reward for nucleotide match: default=1
Expect value: default=10
Word size: default=11 for nucleotides/28 for megablast/3 for proteins.

Furthermore, the degree of local identity between the amino acid sequence query or nucleic acid sequence query and the retrieved homologous sequences is determined by the BLAST program. However only those sequence segments are compared that give a match above a certain threshold. Accordingly, the program calculates the identity only for these matching segments. Therefore, the identity calculated in this way is referred to as local identity.

The term “cDNA” refers to a DNA molecule that can be prepared by reverse transcription from a mature, spliced, mRNA molecule obtained from a eukaryotic or prokaryotic cell. cDNA lacks intron sequences that may be present in the corresponding genomic DNA. The initial, primary RNA transcript is a precursor to mRNA that is processed through a series of steps, including splicing, before appearing as mature spliced mRNA.

The term “coding sequence” refers to a nucleotide sequence, which directly specifies the amino acid sequence of a polypeptide. The boundaries of the coding sequence are generally determined by an open reading frame, which begins with a start codon such as ATG, GTG, or TTG and ends with a stop codon such as TAA, TAG, or TGA. The coding sequence may be a genomic DNA, cDNA, synthetic DNA, or a combination thereof.

The term “control sequence” as used herein refers to a nucleotide sequence necessary for expression of a polynucleotide encoding a polypeptide. A control sequence may be native (i.e., from the same gene) or heterologous or foreign (i.e., from a different gene) to the polynucleotide encoding the polypeptide. Control sequences include, but are not limited to leader sequences, polyadenylation sequence, pro-peptide coding sequence, promoter sequences, signal peptide coding sequence, translation terminator (stop) sequences and transcription terminator (stop) sequences. To be operational control sequences usually must include promoter sequences, transcriptional and translational stop signals. Control sequences may be provided with linkers for the purpose of introducing specific restriction sites facilitating ligation of the control sequences with a coding region of a polynucleotide encoding a polypeptide.

The term “expression” includes any step involved in the production of a polypeptide including, but not limited to, transcription, post-transcriptional modification, translation, post-translational modification, and secretion.

The term “expression vector” refers to a linear or circular DNA molecule that comprises a polynucleotide encoding a polypeptide and is operably linked to control sequences that provide for its expression.

The term “host cell” refers to any cell type that is susceptible to transformation, transfection, transduction, or the like with a polynucleotide construct or expression vector comprising a polynucleotide of the present invention. The term “host cell” encompasses any progeny of a parent cell that is not identical to the parent cell due to mutations that occur during replication.

The term “polynucleotide construct” refers to a polynucleotide, either single- or double stranded, which is isolated from a naturally occurring gene or is modified to contain segments of nucleic acids in a manner that would not otherwise exist in nature, or which is synthetic, and which comprises one or more control sequences.

The term “operably linked” refers to a configuration in which a control sequence is placed at an appropriate position relative to the coding polynucleotide such that the control sequence directs expression of the coding polynucleotide.

The terms “nucleotide sequence” and “polynucleotide” are used herein interchangeably.

The term “comprise” and “include” as used throughout the specification and the accompanying claims as well as variations such as “comprises”, “comprising”, “includes” and “including” are to be interpreted inclusively. These words are intended to convey the possible inclusion of other elements or integers not specifically recited, where the context allows.

The articles “a” and “an” are used herein refers to one or to more than one (i.e. to one or at least one) of the grammatical object of the article. By way of example, “an element” may mean one element or more than one element.

Terms like “preferably”, “commonly”, “particularly”, and “typically” are not utilized herein to limit the scope of the claimed invention or to imply that certain features are critical, essential, or even important to the structure or function of the claimed invention. Rather, these terms are merely intended to highlight alternative or additional features that can or cannot be utilized in a particular embodiment of the present invention.

The term “cell culture” as used herein refers to a culture medium comprising a plurality of genetically modified host cells of the invention. A cell culture may comprise a single strain of genetically modified host cells or may comprise two or more distinct strains of genetically modified host cells. The culture medium may be any medium suitable for the genetically modified host cells, e.g., a liquid medium (i.e., a culture broth) or a semi-solid medium, and may comprise additional components, e.g., a carbon source such as dextrose, sucrose, glycerol, or acetate; a nitrogen source such as ammonium sulfate, urea, or amino acids; a phosphate source; vitamins; trace elements; salts; amino acids; nucleobases; yeast extract; aminoglycoside antibiotics such as G418 and hygromycin B.

### Method for Producing a Tryptamine Derivative

The invention provides a method for producing a tryptamine derivative of formula (I):

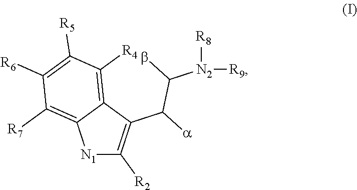

wherein the tryptamine derivative (I) is not tryptophan, 4-hydroxytryptamine, N-acetyl-4-hydroxytryptamine, norbaeocystin, baeocystin; psilocybin, psilocin, aeruginascin, halogenated tryptophan, halogenated tryptamine, halogenated N-methylated tryptamine, halogenated N,N-dimethyltryptamine or halogenated N,N,N-trimethyltryptamine; said method comprising providing an indole acceptor of the formula (II):

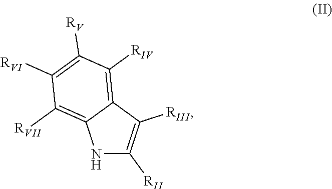

wherein one or more of RII to R_{VII} is not H and R_{III}, is H or CH₂CH₂NH₂ or CH₂CHCOOHNH₂; and contacting the indole acceptor with a substituent donor in the presence of one or more enzymes substituting one or more H, OH and/or COOH in the indole acceptor with one or more substituents of the substituent donor.

In some embodiments the one or more of positions R_{II}, R_{IV}, R_{V}, R_{VI} and/or R_{VII} of the indole acceptor (II) is OH, Cl, Br, F, I, CH₃, NO₂, PO₄, or CH₃—O. In particular tryptamine derivatives wherein R₄ and/or R₅ of Formula I is OH are useful. More specifically the the indole acceptor (II) is selected from 4-hydroxytryptamine, 5-hydroxytryptamine, Psilocybin, Psilocin, Norpsilocin, Baeocystin, Norbaeocystin, Aeruginascin, 4-hydroxy-N,N,N-trimethyltryptamine, Bufotenine, Norbufotenine, 5-hydroxy-N,N,N-trimethyltryptamine, 4-methoxytyptamine, 5-methoxytryptamine N-acetylserotonin, Ibogaine, Ibogamine, Noribogaine, Mitragynine, 7-OH-mitragynine, 4-HO-DET, 4-HO-DiPT, 4-HO-MET, 4-HO-MiPT, 4-HO-McPT, 4-HO-DPT, 4-HO-DSBT, and/or Harmalol, so that these indole acceptors is further derivatized with at least one further substitution generating enhance properties to the molecule.

The substituent transferred to the indole acceptor is in some embodiments an alkyl group, an acetyl group, a glycosyl group, a phosphate group, an oxygenyl group, a hydroxyl group or a halogenyl group.

Where the substituent is a glycosyl group the glycosyl moiety of the glycosyl group suitably comprises one or more of sugars selected from glucose, galactose, xylose, mannose, galactofuranose, arabinose, rhamnose, apiose, fucose, glucosamine, galactosamine, N-acetylglucosamine, N-acetylgalactosamine, xylosamine, mannosamine, arabinosamine, rhamnosamine, apiosamine, fucosamine, glucuronate, galacturonate, mannuronate, arabinate, apionate or a combination thereof. The substitution by a glycosyl group can suitably be an O-glycosylation, such as a β-O-glycosylation.

Where the substituent is an alkyl group, the alkyl group is suitably an ethyl or a methyl group. The alkylation reaction is suitably an O-alkylation, a N-alkylation, or a C-alkylation, optionally ethylation or methylation.

Where the substituent is an acetyl group, the acetylation reaction is suitably an N-acetylation. Examples of acetyl donors are acetyl-CoA.

The donor donating the substituent to the indole acceptor may be an aldehyde, a ketone, an ether and/or an amine.

Suitable aldehydes include acetaldehyde, oxaloacetaldehyde and/or secologanin, while suitable ketones include cinnamoyl-CoA or pyruvate.

The ether may be a glycoside, in particular a nucleotide glycoside, such as NTP-glycosides, NDP-glycosides or NMP-glycosides. In some embodiments the nucleoside of the nucleotide glycoside is selected from Uridine, Adenosin, Guanosin, Cytidin and/or deoxythymidine. In particular the nucleotide glycoside includes UDP-glycosides, ADP-glycosides, CDP-glycosides, CMP-glycosides, dTDP-glycosides and/or GDP-glycosides. Particularly useful nucleotide glycosides are UDP-D-glucose (UDP-Glc); UDP-galactose (UDP-Gal); UDP-D-xylose (UDP-Xyl); UDP-N-acetyl-D-glucosamine (UDP-GIcNAc); UDP-N-acetyl-D-galactosamine (UDP-GaINAc); UDP-D-glucuronic acid (UDP-GIcA); UDP-D-galactofuranose (UDP-Galf); UDP-arabinose; UDP-rhamnose, UDP-apiose; UDP-2-acetamido-2-deoxy-α-D-mannuronate; UDP-N-acetyl-D-galactosamine 4-sulfate; UDP-N-acetyl-D-mannosamine; UDP-2,3-bis(3-hydroxytetra-decanoyl)-glucosamine; UDP-4-deoxy-4-formamido-β-L-arabinopyranose; UDP-2,4-bis(acetamido)-2,4,6trideoxy-α-D-glucopyranose; UDP-galacturonate; UDP-3-amino-3-deoxy-α-D-glucose; guanosine diphospho-D-mannose (GDP-Man); guanosine diphospho-L-fucose (GDP-Fuc); guanosine diphospho-L-rhamnose (GDP-Rha); cytidine monophospho-N-acetylneuraminic acid (CMP-Neu5Ac); cytidine monophospho-2-keto-3-deoxy-D-mannooctanoic acid (CMP-Kdo); and ADP-glucose.

The amine may be S-Adenosyl methionine (SAM) or S-Adenosyl ethionine (SAE).

In the method of the invention the one or more enzymes is suitably includes glycosyltransferases, alkyltransferases, synthases, acetyltransferases, kinases, cinnamoyltransferases, phosphatases, laccases, halogenases, P450 enzymes, flavin monooxygenases, and/or lyases.

The glycosyltransferase of the invention may be derived from a plant, such as Oryza sativa, Crocus sativus, Nicotiana tabacum, Stevia rebaudiana, Nicotiana benthatamiana and/or Arabidopsis thaliana or from a fungus. The glycosyl transferase can be an O-glycoside transferase transferring a glycosyl group to an O of the indole acceptor and/or a C-glycoside transferase transferring a glycosyl group to a C of the indole acceptor. In some embodiments the glycosyl transferase can O-glycosylate an aglycone acceptor or a glycosylated acceptor or both. In either case the glycosyl group can be glucose, rhamnose, xylose, arabinose, N-acetylgalactosamin, or N-acetylglucosamin. In further embodiments the glycosyl transferase can transfer a monosaccharide, a disaccharide, a trisaccharide or a tetrasacharide to the indole acceptor such as an aglycone/glycoside mono-O-glycosyltransferase, di-O-glycosyltransferase, tri-O-glycosyltransferase, or tetra-O-glycosyltransferase, respectively. In a preferred embodiment the glycosyl transferase is a hydroxytryptamine glycosyltransferase. In alternative embodiments the glycosyl transferase of the invention may be selected from EC classes EC2.4.1.-, and/or EC2.4.2.-, such as EC2.4.1.17, EC2.4.1.35, EC2.4.1.159, EC2.4.1.203. EC2.4.1.234, EC2.4.1.236, EC2.4.1.294, and/or EC2.4.2.40. Examples of suitable glycosyl transferases are the glycosyl transferases comprised in SEQ ID NO: 80, 82, 84, or 86, 188, 190, 192, 194, 196, 198, 200, 202, 204, 206, 208, 210, 212, 214, 216, 218, 220, 222, 224, 226, 228, 230, 232, 234, 236, 238, 240, 242, 244, 246, 248, 250 and/or 252. The glycosyl transferase of the invention preferably has at least 70%, such at least 75%, such as at least 80%, such as at least 90%, such as at least 95%, such as at least 99%, such as 100% identity to the glycosyl transferase comprised in anyone of SEQ ID NO: 80, 82, 84, or 86, 188, 190, 192, 194, 196, 198, 200, 202, 204, 206, 208, 210, 212, 214, 216, 218, 220, 222, 224, 226, 228, 230, 232, 234, 236, 238, 240, 242, 244, 246, 248, 250 and/or 252.

In some embodiments the glycosyl transferase has at least 70%, such at least 75%, such as at least 80%, such as at least 90%, such as at least 95%, such as at least 99%, such as 100% identity to the glycosyl transferase comprised in anyone of SEQ ID NO: 194 and/or 204.

The alkyl transferase of the invention may be a methyltransferase, such as an O-methyltransferase, a N-methyltransferase or a C-methyltransferase. Suitable O-methyltransferases include those having at least 70%, such at least 75%, such as at least 80%, such as at least 90%, such as at least 95%, such as at least 99%, such as 100% identity to the O-methyltransferase comprised in anyone of SEQ ID NO: 114, 116, 118 and/or 120. Suitable N-methyltransferases include those having at least 70%, such at least 75%, such as at least 80%, such as at least 90%, such as at least 95%, such as at least 99%, such as 100% identity to the N-methyltransferase comprised in anyone of SEQ ID NO: 122, 124, 126, 128, 130, 132, 134 136 and/or 138. Suitable C-methyltransferases include those having at least 70%, such at least 75%, such as at least 80%, such as at least 90%, such as at least 95%, such as at least 99%, such as 100% identity to the C-methyltransferase comprised in SEQ ID NO: 140.

The synthase of the invention may be a Strictosidine synthase or a 1-acetyl-β-carboline synthase. Suitable Strictosidine synthases include those having at least 70%, such at least 75%, such as at least 80%, such as at least 90%, such as at least 95%, such as at least 99%, such as 100% identity to the Strictosidine synthase comprised in anyone of SEQ ID NO: 144 146, 148 and/or 150. Suitable 1-acetyl-β-carboline synthases include those having at least 70%, such at least 75%, such as at least 80%, such as at least 90%, such as at least 95%, such as at least 99%, such as 100% identity to the 1-acetyl-β-carboline synthase comprised in anyone of SEQ ID NO: 152 and/or 154.

The acetyltransferase may be an aralkylamine N-acetyltransferase including those having at least 70%, such at least 75%, such as at least 80%, such as at least 90%, such as at least 95%, such as at least 99%, such as 100% identity to the aralkylamine N-acetyltransferase comprised in SEQ ID NO: 142.

The kinase of the invention may be a 4-Hydroxytryptamine kinase and/or a 7-hydroxytryptamine kinase including those having at least 70%, such at least 75%, such as at least 80%, such as at least 90%, such as at least 95%, such as at least 99%, such as 100% identity to the 4-Hydroxytryptamine kinase and/or 7-hydroxytryptamine kinase comprised in anyone of SEQ ID NO: 156, 158, and/or 160.

The cinnamoyltransferase of the invention may be a N-hydroxycinnamoyltransferase including those having at least 70%, such at least 75%, such as at least 80%, such as at least 90%, such as at least 95%, such as at least 99%, such as 100% identity to the N-hydroxycinnamoyltransferase comprised in SEQ ID NO: 162.

The phosphatase of the invention may be a psilocybin phosphatase including those having at least 70%, such at least 75%, such as at least 80%, such as at least 90%, such as at least 95%, such as at least 99%, such as 100% identity to the psilocybin phosphatase comprised in SEQ ID NO: 164.

The laccase of the invention may be a psilocin laccase including those having at least 70%, such at least 75%, such as at least 80%, such as at least 90%, such as at least 95%, such as at least 99%, such as 100% identity to the psilocin laccase comprised in SEQ ID NO: 166.

The halogenase may be a tryptophan halogenase such as a Tryptophan 2-halogenase, a Tryptophan 5-halogenase, a Tryptophan 6-halogenase or a Tryptophan 7-halogenase. Suitable Tryptophan 2-halogenases inclide those having at least 70%, such at least 75%, such as at least 80%, such as at least 90%, such as at least 95%, such as at least 99%, such as 100% identity to the Tryptophan 2-halogenase comprised in SEQ ID NO: 168. Suitable Tryptophan 5-halogenases include those having at least 70%, such at least 75%, such as at least 80%, such as at least 90%, such as at least 95%, such as at least 99%, such as 100% identity to the Tryptophan 5-halogenase comprised in SEQ ID NO: 170. Suitable Tryptophan 6-halogenase include those having at least 70%, such at least 75%, such as at least 80%, such as at least 90%, such as at least 95%, such as at least 99%, such as 100% identity to the Tryptophan 6-halogenase comprised in SEQ ID NO: 172. Suitable Tryptophan 7-halogenase include those having at least 70%, such at least 75%, such as at least 80%, such as at least 90%, such as at least 95%, such as at least 99%, such as 100% identity to the Tryptophan 7-halogenase comprised in SEQ ID NO: 174.

The P450 enzyme of the invention include those having at least 70%, such at least 75%, such as at least 80%, such as at least 90%, such as at least 95%, such as at least 99%, such as 100% identity to the P450 enzymes comprised in anyone of SEQ ID NO: 88, 90, 92, 94, 96, 100 and/or 178 and the method can optionally further include contacting the P450 Enzymes with a P450 reductase (CPR). In particular embodiments the P450 enzyme has at least 70%, such at least 75%, such as at least 80%, such as at least 90%, such as at least 95%, such as at least 99%, such as 100% identity to the P450 enzyme comprised in SEQ ID NO:96 (OsT5H) and optionally the P450 reductase has at least 70%, such at least 75%, such as at least 80%, such as at least 90%, such as at least 95%, such as at least 99%, such as 100% identity to the P450 reductase comprised in SEQ ID NO: 112 (FoCPR).

The flavin monooxygenase of the invention include those having at least 70%, such at least 75%, such as at least 80%, such as at least 90%, such as at least 95%, such as at least 99%, such as 100% identity to the flavin monooxygenase comprised in SEQ ID NO: 98.

The lyase of the invention include those having at least 70%, such at least 75%, such as at least 80%, such as at least 90%, such as at least 95%, such as at least 99%, such as 100% identity to the lyase comprised in anyone of SEQ ID NO: 52 or 176.

The sequence identities for the above-mentioned substituting enzymes may even be at least 90%, such as at least 95%, such as at least 99%, such as 100%, such as specifically at least 99%, such as 100%.

The tryptamine derivative (I) of the invention may be a hydroxytryptamine β-O-glycoside, such as a hydroxytryptamine β-O-glucoside, including but not limited to 4-hydroxytryptamine-β-O-glycoside, Psilocin-β-O-glycoside, Norpsilocin-β-O-glycoside, 4-hydroxy-N,N,N-trimethyltryptamine-β-O-glycoside, Serotonin-β-O-glycoside, Bufotenine-β-O-glycoside, Norbufotenine-β-O-glycoside, 5-hydroxy-N,N,N-trimethyl-tryptamine-β-O-glycoside, N-acetylserotonin-β-O-glycoside, Noribogaine-β-O-glycoside, 7-OH-mitragynine-β-O-glycoside, 4-HO-DET-β-O-glycoside, 4-HO-DiPT-β-O-glycoside, 4-HO-MET-β-O-glycoside, 4-HO-MiPT-β-O-glycoside, 4-HO-McPT-β-O-glycoside, 4-HO-DPT-β-O-glycoside, 4-HO-DSBT-β-O-glycoside, and/or Harmalol-β-O-glycoside.

In further embodiments the method of the invention comprises one or more steps selected from:
a) converting an indole or indole derivative into tryptophan or a tryptophan derivative; and
b) converting tryptophan or tryptophan derivative into tryptamine or tryptamine derivative.

These steps a) and/or b) may be performed in vitro.

In particular the conversion of the indole or indole derivative into the tryptophan or tryptophan derivative may include contacting the indole or indole derivative with a tryptophan synthase enzyme, such as a tryptophan synthase which has at least 70%, such at least 75%, such as at least 80%, such as at least 90%, such as at least 95%, such as at least 99%, such as 100% identity to the tryptophan synthase comprised in SEQ ID NO: 60, 62, 64, 66, 68, 180, 182 and/or 256. In special embodiments the tryptophan synthase has at least 70%, such at least 75%, such as at least 80%, such as at least 90%, such as at least 95%, such as at least 99%, such as 100% identity to the tryptophan synthase comprised in SEQ ID NO: 256.

Further, the conversion of the tryptophan or tryptophan derivative into the tryptamine or tryptamine derivative may comprise contacting the tryptophan or tryptophan derivative with a tryptophan decarboxylase enzyme, such as a tryptophan decarboxylase which has at least 70%, such at least 75%, such as at least 80%, such as at least 90%, such as at least 95%, such as at least 99%, such as 100% identity to the tryptophan decarboxylase comprised in SEQ ID NO: 26, 70, 72, 74, 76 and/or 78. In particular embodiments the conversion of the indole or indole derivative into the tryptophan or tryptophan derivative comprises contacting the indole or indole derivative with a tryptophan synthase enzyme which has at least 70%, such at least 75%, such as at least 80%, such as at least 90%, such as at least 95%, such as at least 99%, such as 100% identity to the tryptophan synthase comprised in SEQ ID NO: 180 and/or 256 in the presence of a tryptophan decarboxylase which has at least 70%, such at least 75%, such as at least 80%, such as at least 90%, such as at least 95%, such as at least 99%, such as 100% identity to the tryptophan decarboxylase comprised in SEQ ID NO: 72 and/or 78.

In important embodiments the indole acceptor is serotonin and the tryptamine derivative (I) is a derivative of serotonin, optionally Melatonin, Normelatonin or hydroxycinnamoylserotonin such as 4-coumaroylserotonin and the one or more enzymes substituting one or more H, OH and/or COOH in the indole acceptor with one or more substituents of the substituent donor are selected from
a) an acetyl transferase which has at least 70% to the identity to the acetyl transferase comprised in SEQ ID NO: 142
b) an O-methyl transferase which has at least 70% to the identity to the O-methyl transferase comprised in SEQ ID NO: 118; and/or
c) a N-hydroxycinnamoyl transferase which has at least 70% to the identity to the Cin trans comprised in SEQ ID NO: 162.

The method of the invention can further comprise one or more additional steps selected from:
a) Glycosylation;
b) Methylation;
c) Hydroxylation;
d) Condensation;
e) Nitration;
f) Oxidation;
g) Lyase deamidation; or
h) Dephosphorylation

The hydroxylation step may comprise contacting the indole or indole derivative or tryptophan or tryptophan derivative with a hydroxylase including those having at least 70%, such at least 75%, such as at least 80%, such as at least 90%, such as at least 95%, such as at least 99%, such as 100% identity to the hydroxylase comprised in anyone of SEQ ID NO: 88, 90, 92, 94, 96, 98, and/or 100, optionally in the presence of a Cytochrome P450 reductase (CRP) which has at least 70%, such at least 75%, such as at least 80%, such as at least 90%, such as at least 95%, such as at least 99%, such as 100% identity to the CPR comprised in anyone of SEQ ID NO: 102, 104, 106, 108, 110 and/or 112.

The lyase deamidation step may comprise contacting the indole or indole derivative or tryptophan or tryptophan derivative with a lyase including those having at least 70%, such at least 75%, such as at least 80%, such as at least 90%, such as at least 95%, such as at least 99%, such as 100% identity to the lyase comprised in anyone of SEQ ID NO: 52 and/or 176.

In preferred embodiments the method comprises in vitro enzymatic substitution steps and/or optionally in vivo enzymatic substitution steps. In particular the method can comprise expressing a glycosyl transferase in E. coli and performing in vitro glycosylation of the indole acceptor.

### Tryptamine Derivatives

The invention also provides novel tryptamines derivatives resulting from performing the method of the invention. Such tryptamine derivatives include those of formula (I):

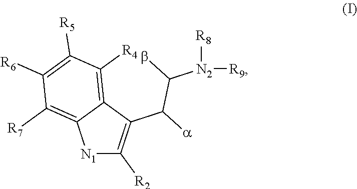

wherein at least one of R₂, R₄ to R₉, α and β is a glycosyl group, wherein particularly R₂, R4 to R₇, α and β may be an O-glycosyl group. Such tryptamine derivatives include but are not limited to 4-hydroxytryptamine-β-O-glycoside, Psilocin-β-O-glycoside, Norpsilocin-β-O-glycoside, 4-hydroxy-N,N,N-trimethyltryptamine-β-O-glycoside, Serotonin-β-O-glycoside, Bufotenine-β-O-glycoside, Norbufotenine-β-O-glycoside, 5-hydroxy-N,N,N-trimethyltryptamine-β-O-glycoside, N-acetylserotonin-β-O-glycoside, Noribogaine-β-O-glycoside, 7-OH-mitragynine-β-O-glycoside, 4-HO-DET-β-O-glycoside, 4-HO-DiPT-β-O-glycoside, 4-HO-MET-β-O-glycoside, 4-HO-MiPT-β-O-glycoside, 4-HO-McPT-β-O-glycoside, 4-HO-DPT-β-0-glycoside, 4-HO-DSBT-β-O-glycoside, and wherein the glycoside of Serotonin-β-O-glycoside, Bufotenine-β-O-glycoside, Norbufotenine-β-O-glycoside or 5-hydroxy-N,N,N-trimethyltryptamine-β-O-glycoside is not a glucoside.

### Genetically Modified Host Cells and Cell Cultures

In a further aspect of the invention the method as described, supra, is performed in a host cell genetically modified to produce tryptamine derivative of the invention. Accordingly, the present invention also provides host cells, which are genetically modified to produce the tryptamine derivative in the presence of the substitution group donor, wherein the host cell expresses one or more heterologous genes encoding the one or more substituting enzymes, which in the presence of the substitution group donor and the indole acceptor, transfers a substitution group from the donor to the acceptor and thereby produces the substituted tryptamine. Substituting enzymes and donors for performing the method in the genetically modified cell are suitably those described, supra, for the method.

The genetically modified microbial host cell suitably expresses one or more genes selected from:
a) genes encoding a UGT said genes which are at least 70%, such at least 75%, such as at least 80%, such as at least 90%, such as at least 95%, such as at least 99%, such as 100% identical to the UGT encoding polynucleotide comprised in anyone of SEQ ID NO: 79, 81, 83, 85, 187, 189, 191, 193, 195, 197, 199, 201, 203, 205, 207, 209, 211, 213, 215, 217, 219, 221, 223, 225, 227, 229, 231, 233, 235, 237, 239, 241, 243, 245, 247, 249, and/or 251 or genomic DNA thereof;
b) genes encoding an O-methyltransferase said genes which are at least 70%, such at least 75%, such as at least 80%, such as at least 90%, such as at least 95%, such as at least 99%, such as 100% identical to the O-methyltransferase encoding polynucleotide comprised in anyone of SEQ ID NO: 113, 115, 117, and/or 119 or genomic DNA thereof;
c) genes encoding an N-methyltransferase said genes which are at least 70%, such at least 75%, such as at least 80%, such as at least 90%, such as at least 95%, such as at least 99%, such as 100% identical to the N-methyltransferase encoding polynucleotide comprised in anyone of SEQ ID NO: 121, 123, 125, 127, 129, 131, 133, 135, and/or 137 or genomic DNA thereof;
d) genes encoding a C-methyltransferase said genes which are at least 70%, such at least 75%, such as at least 80%, such as at least 90%, such as at least 95%, such as at least 99%, such as 100% identical to the C-methyltransferase encoding polynucleotide comprised in SEQ ID NO: 139 or genomic DNA thereof;
e) genes encoding a strictosidine synthase said genes which are at least 70%, such at least 75%, such as at least 80%, such as at least 90%, such as at least 95%, such as at least 99%, such as 100% identical to the strictosidine synthase encoding polynucleotide comprised in anyone of SEQ ID NO: 143, 145, 147, and/or 149 or genomic DNA thereof;
f) genes encoding a 1-acetyl-β-carboline synthase said genes which are at least 70%, such at least 75%, such as at least 80%, such as at least 90%, such as at least 95%, such as at least 99%, such as 100% identical to the 1-acetyl-β-carboline synthase encoding polynucleotide comprised in anyone of SEQ ID NO: 151, and/or 153 or genomic DNA thereof;
g) genes encoding a 1-acetyl-β-carboline synthase said genes which are at least 70%, such at least 75%, such as at least 80%, such as at least 90%, such as at least 95%, such as at least 99%, such as 100% identical to the aralkylamine N-acetyltransferase encoding polynucleotide comprised in SEQ ID NO: 141 or genomic DNA thereof;
h) genes encoding a 4-Hydroxytryptamine kinase and/or a 7-hydroxytryptamine kinase said genes which are at least 70%, such at least 75%, such as at least 80%, such as at least 90%, such as at least 95%, such as at least 99%, such as 100% identical to the 4-Hydroxytryptamine kinase and/or a 7-hydroxytryptamine kinase encoding polynucleotide comprised in anyone of SEQ ID NO: 155, 157, and/or 159 or genomic DNA thereof;
i) genes encoding an N-hydroxycinnamoyltransferase said genes which are at least 70%, such at least 75%, such as at least 80%, such as at least 90%, such as at least 95%, such as at least 99%, such as 100% identical to the N-hydroxycinnamoyltransferase encoding polynucleotide comprised in SEQ ID NO: 161 or genomic DNA thereof;
j) genes encoding a psilocybin phosphatase said genes which are at least 70%, such at least 75%, such as at least 80%, such as at least 90%, such as at least 95%, such as at least 99%, such as 100% identical to the psilocybin phosphatase encoding polynucleotide comprised in SEQ ID NO: 163 or genomic DNA thereof;
k) genes encoding a psilocin laccase said genes which are at least 70%, such at least 75%, such as at least 80%, such as at least 90%, such as at least 95%, such as at least 99%, such as 100% identical to the psilocin laccase encoding polynucleotide comprised in SEQ ID NO: 165 or genomic DNA thereof;
I) genes encoding a Tryptophan 2-halogenase said genes which are at least 70%, such at least 75%, such as at least 80%, such as at least 90%, such as at least 95%, such as at least 99%, such as 100% identical to the Tryptophan 2-halogenase encoding polynucleotide comprised in SEQ ID NO: 167 or genomic DNA thereof;
m) genes encoding a Tryptophan 5-halogenase said genes which are at least 70%, such at least 75%, such as at least 80%, such as at least 90%, such as at least 95%, such as at least 99%, such as 100% identical to the Tryptophan 5-halogenase encoding polynucleotide comprised in SEQ ID NO: 169 or genomic DNA thereof;
n) genes encoding a Tryptophan 6-halogenase said genes which are at least 70%, such at least 75%, such as at least 80%, such as at least 90%, such as at least 95%, such as at least 99%, such as 100% identical to the Tryptophan 6-halogenase encoding polynucleotide comprised in SEQ ID NO: 171 or genomic DNA thereof;
o) genes encoding a Tryptophan 7-halogenase said genes which are at least 70%, such at least 75%, such as at least 80%, such as at least 90%, such as at least 95%, such as at least 99%, such as 100% identical to the Tryptophan 7-halogenase encoding polynucleotide comprised in SEQ ID NO: 173 or genomic DNA thereof;
p) genes encoding a P450 enzyme said genes which are at least 70%, such at least 75%, such as at least 80%, such as at least 90%, such as at least 95%, such as at least 99%, such as 100% identical to the P450 enzyme encoding polynucleotide comprised in anyone of SEQ ID NO: 87, 89, 91, 93, 95, 99, and/or 177 or genomic DNA thereof;
q) genes encoding a P450 reductase said genes which are at least 70%, such at least 75%, such as at least 80%, such as at least 90%, such as at least 95%, such as at least 99%, such as 100% identical to the P450 reductase (CPR) encoding polynucleotide comprised in anyone of SEQ ID NO: 101, 103, 105, 107, 109, and/or 111 or genomic DNA thereof;
r) genes encoding a flavin monooxygenase said genes which are at least 70%, such at least 75%, such as at least 80%, such as at least 90%, such as at least 95%, such as at least 99%, such as 100% identical to the flavin monooxygenase encoding polynucleotide comprised in SEQ ID NO: 97 or genomic DNA thereof; and/or
s) genes encoding a lyase said genes which are at least 70%, such at least 75%, such as at least 80%, such as at least 90%, such as at least 95%, such as at least 99%, such as 100% identical to the lyase encoding polynucleotide comprised in anyone of SEQ ID NO: 51 and/or 175 or genomic DNA thereof.

The host cell of the invention may further comprise an operative biosynthetic pathway producing the indole acceptor, wherein the host cell expresses one or more pathway genes encoding polypeptides selected from:
a) one or more enzymes converting glucose to fructose-6-phosphate;
b) a fructose-6-phosphate phosphoketolase converting fructose-6-phosphate to Erythrose-4-phosphate and acetyl phosphate;
c) a Phosphotransacetylase converting Acetyl phosphate to Acetyl-CoA;
d) one or more enzymes converting Fructose-6-phosphate to Phosphoenolpyruvate;
e) a 3-deoxy-D-arabino-heptulosonate 7-phosphate synthase (DAHP synthase) converting Phosphoenolpyruvate and Erythrose-4-phosphate to 3-deoxy-D-arabino-heptulosonate-7-phosphate (DAHP);
f) one or more enzymes converting 3-deoxy-D-arabino-heptulosonate-7-phosphate to 5-enolpyruvoyl-shikimate 3-phosphate;
g) a Shikimate kinase converting Shikimate to Shikimate-3-phosphate;
h) a Chorismate synthase converting 5-enolpyruvoyl-shikimate 3-phosphate to Chorismate;
i) a Anthranilate synthase converting Chorismate to Anthranilate;
j) a Ribose-phosphate pyrophosphokinase converting Ribose-5-phosphate to Phospho-alpha-D-ribosyl-1-pyrophosphate;
k) a Anthranilate phosphoribosyl transferase converting Anthranilate and Phospho-alpha-D-ribosyl-1-pyrophosphate to N-(5-phosphoribosyl)-anthranilate;
l) a N-(5′-phosphoribosyl)-anthranilate isomerase converting N-(5-phosphoribosyl)-anthranilate to 1-(o-carboxyphenylamino)-1′-deoxyribulose 5′-phosphate;
m) a Indole-3-glycerol phosphate synthase converting 1-(o-carboxyphenylamino)-1′-deoxyribulose 5′-phosphate to (1S,2R)-1-C-(indol-3-yl)-glycerol 3-phosphate;
n) a Tryptophan synthase converting (1S,2R)-1-C-(indol-3-yl) glycerol 3-phosphate and Serine to L-Tryptophan;
o) a Tryptophan decarboxylase converting L-Tryptophan to Tryptamine;
p) a Chorismate mutase converting Chorismate to Prephenate;
q) a Prephenate dehydrogenase converting Prephenate to Phenylpyruvate;
r) an Aromatic aminotransferase converting Phenylpyruvate to phenylalanine;
s) a Phenylalanine ammonium lyase converting Phenylalanine to cinnamate;
t) a Cinnamate 4-hydroxylase converting Cinnamate to coumarate;
u) a cytochrome b5 assisting Cytochrome P450 reductases reducing hydroxylase enzymes;
v) a Cytochrome P450 reductase reducing cytochrome P450 enzymes;
w) a 4-Coumoryl-CoA ligase converting Coumarate to 4-coumoryl CoA;
x) a Tryptophanase converting tryptophan or a derivative thereof into indole or a derivative thereof;
y) a Tryptophan synthase converting Indole or a derivative thereof and Serine or a derivative thereof into Tryptophan or a derivative thereof;
z) a Tryptophan decarboxylase or a non-canonical aromatic amino acid decarboxylase converting Tryptophan or a derivative thereof into Tryptamine or a derivative thereof;
aa) a Tryptamine 5-hydroxylase converting tryptamine to serotonin;
bb) a Tryptamine 4-hydroxylase converting tryptamine to 4-hydroxytryptamine.
cc) 4-hydroxytryptamine kinase converting 4-hydroxytryptamine to Norbaeocystin; and/or
dd) Psilocybin synthase converting Norbaeocystin to Psilocybin.

In some embodiments of the host cell the corresponding:
a) fructose-6-phosphate phosphoketolase has at least 70%, such at least 75%, such as at least 80%, such as at least 90%, such as at least 95%, such as at least 99%, such as 100% identity to the fructose-6-phosphate phosphoketolase comprised in SEQ ID NO: 2;
b) Phosphotransacetylase has at least 70%, such at least 75%, such as at least 80%, such as at least 90%, such as at least 95%, such as at least 99%, such as 100% identity to the Phosphotransacetylase comprised in SEQ ID NO: 4;
c) 3-deoxy-D-arabino-heptulosonate 7-phosphate synthase has at least 70%, such at least 75%, such as at least 80%, such as at least 90%, such as at least 95%, such as at least 99%, such as 100% identity to the 3-deoxy-D-arabino-heptulosonate 7-phosphate synthase comprised in SEQ ID NO: 6;
d) Enzyme converting 3-deoxy-D-arabino-heptulosonate-7-phosphate to 5-enolpyruvoyl-shikimate 3-phosphate has at least 70%, such at least 75%, such as at least 80%, such as at least 90%, such as at least 95%, such as at least 99%, such as 100% identity to the Enzyme comprised in SEQ ID NO: 8;
e) Shikimate kinase has at least 70%, such at least 75%, such as at least 80%, such as at least 90%, such as at least 95%, such as at least 99%, such as 100% identity to the Shikimate kinase comprised in SEQ ID NO: 10;
f) Chorismate synthase has at least 70%, such at least 75%, such as at least 80%, such as at least 90%, such as at least 95%, such as at least 99%, such as 100% identity to the Chorismate synthase comprised in SEQ ID NO: 12;
g) Anthranilate synthase has at least 70%, such at least 75%, such as at least 80%, such as at least 90%, such as at least 95%, such as at least 99%, such as 100% identity to the Anthranilate synthase comprised in SEQ ID NO: 14;
h) Ribose-phosphate pyrophosphokinase has at least 70%, such at least 75%, such as at least 80%, such as at least 90%, such as at least 95%, such as at least 99%, such as 100% identity to the Ribose-phosphate pyrophosphokinase comprised in SEQ ID NO: 16;
i) Anthranilate phosphoribosyl transferase has at least 70%, such at least 75%, such as at least 80%, such as at least 90%, such as at least 95%, such as at least 99%, such as 100% identity to the Anthranilate phosphoribosyl transferase comprised in SEQ ID NO: 18;
j) N-(5′-phosphoribosyl)-anthranilate isomerase has at least 70%, such at least 75%, such as at least 80%, such as at least 90%, such as at least 95%, such as at least 99%, such as 100% identity to the N-(5′-phosphoribosyl)-anthranilate isomerase comprised in SEQ ID NO: 20;
k) Indole-3-glycerol phosphate synthase has at least 70%, such at least 75%, such as at least 80%, such as at least 90%, such as at least 95%, such as at least 99%, such as 100% identity to the Indole-3-glycerol phosphate synthase comprised in SEQ ID NO: 22;
l) Tryptophan synthase has at least 70%, such at least 75%, such as at least 80%, such as at least 90%, such as at least 95%, such as at least 99%, such as 100% identity to the Tryptophan synthase comprised in anyone of SEQ ID NO: 24, 60, 62, 64, 66, 68, 180 and/or 182;
m) Tryptophan decarboxylase or a non-canonical aromatic amino acid decarboxylase has at least 70%, such at least 75%, such as at least 80%, such as at least 90%, such as at least 95%, such as at least 99%, such as 100% identity to the Tryptophan decarboxylase or a non-canonical aromatic amino acid decarboxylase comprised in anyone of SEQ ID NO: 26, 70, 72, 74, 76, and/or 78;
n) Chorismate mutase has at least 70%, such at least 75%, such as at least 80%, such as at least 90%, such as at least 95%, such as at least 99%, such as 100% identity to the Chorismate mutase comprised in SEQ ID NO: 46;
o) Prephenate dehydrogenase has at least 70%, such at least 75%, such as at least 80%, such as at least 90%, such as at least 95%, such as at least 99%, such as 100% identity to the Prephenate dehydrogenase comprised in SEQ ID NO: 48;
p) Aromatic aminotransferase has at least 70%, such at least 75%, such as at least 80%, such as at least 90%, such as at least 95%, such as at least 99%, such as 100% identity to the Aromatic aminotransferase comprised in SEQ ID NO: 50;
q) Phenylalanine ammonium lyase has at least 70%, such at least 75%, such as at least 80%, such as at least 90%, such as at least 95%, such as at least 99%, such as 100% identity to the Phenylalanine ammonium lyase comprised in SEQ ID NO: 52;
r) Cinnamate 4-hydroxylase has at least 70%, such at least 75%, such as at least 80%, such as at least 90%, such as at least 95%, such as at least 99%, such as 100% identity to the Cinnamate 4-hydroxylase comprised in SEQ ID NO: 54;
s) cytochrome b5 has at least 70%, such at least 75%, such as at least 80%, such as at least 90%, such as at least 95%, such as at least 99%, such as 100% identity to the cytochrome b5 comprised in SEQ ID NO: 254;
t) Cytochrome P450 reductase has at least 70%, such at least 75%, such as at least 80%, such as at least 90%, such as at least 95%, such as at least 99%, such as 100% identity to the Cytochrome P450 reductase comprised in SEQ ID NO: 56, 102, 104, 106, 108, 110 and/or 112;
u) 4-Coumoryl-CoA ligase has at least 70%, such at least 75%, such as at least 80%, such as at least 90%, such as at least 95%, such as at least 99%, such as 100% identity to the 4-Coumoryl-CoA ligase comprised in SEQ ID NO: 58;
v) Tryptamine 5-hydroxylase has at least 70%, such at least 75%, such as at least 80%, such as at least 90%, such as at least 95%, such as at least 99%, such as 100% identity to the 5-hydroxylase comprised in SEQ ID NO: 96;
w) Typtamine 4-hydroxylase has at least 70%, such at least 75%, such as at least 80%, such as at least 90%, such as at least 95%, such as at least 99%, such as 100% identity to the Tryptamine 4-hydroxylase comprised in SEQ ID NO 94:
x) 4-hydroxytryptamine kinase has at least 70%, such at least 75%, such as at least 80%, such as at least 90%, such as at least 95%, such as at least 99%, such as 100% identity to the 4-hydroxytryptamine kinase comprised in SEQ ID NO: 160; and/or
y) Psilocybin synthase has at least 70%, such at least 75%, such as at least 80%, such as at least 90%, such as at least 95%, such as at least 99%, such as 100% identity to the Psilocybin synthase comprised in SEQ ID NO: 128.

In other embodiments of the host cell the one or more expressed genes are selected from:
a) genes encoding a fructose-6-phosphate phosphoketolase said genes being at least 70%, such at least 75%, such as at least 80%, such as at least 90%, such as at least 95%, such as at least 99%, such as 100% identical to the fructose-6-phosphate phosphoketolase encoding polynucleotide comprised in SEQ ID NO: 1 or genomic DNA thereof;
b) genes encoding a Phosphotransacetylase said genes being at least 70%, such at least 75%, such as at least 80%, such as at least 90%, such as at least 95%, such as at least 99%, such as 100% identical to the Phosphotransacetylase encoding polynucleotide comprised in SEQ ID NO: 3 or genomic DNA thereof;
c) genes encoding a 3-deoxy-D-arabino-heptulosonate 7-phosphate synthase said genes being at least 70%, such at least 75%, such as at least 80%, such as at least 90%, such as at least 95%, such as at least 99%, such as 100% identical to the 3-deoxy-D-arabino-heptulosonate 7-phosphate synthase encoding polynucleotide comprised in SEQ ID NO: 5 or genomic DNA thereof;
d) genes encoding an enzyme converting 3-deoxy-D-arabino-heptulosonate-7-phosphate to 5-enolpyruvoyl-shikimate 3-phosphate said genes being at least 70%, such at least 75%, such as at least 80%, such as at least 90%, such as at least 95%, such as at least 99%, such as 100% identical to the polynucleotide encoding the enzyme converting 3-deoxy-D-arabino-heptulosonate-7-phosphate to 5-enolpyruvoyl-shikimate 3-phosphate comprised in SEQ ID NO: 7 or genomic DNA thereof;
e) genes encoding a Shikimate kinase said genes being at least 70%, such at least 75%, such as at least 80%, such as at least 90%, such as at least 95%, such as at least 99%, such as 100% identical to the Shikimate kinase encoding polynucleotide comprised in SEQ ID NO: 9 or genomic DNA thereof;
f) genes encoding a Shikimate kinase said genes being at least 70%, such at least 75%, such as at least 80%, such as at least 90%, such as at least 95%, such as at least 99%, such as 100% identical to the Chorismate synthase encoding polynucleotide comprised in SEQ ID NO: 11 or genomic DNA thereof;
g) genes encoding a Anthranilate synthase said genes being at least 70%, such at least 75%, such as at least 80%, such as at least 90%, such as at least 95%, such as at least 99%, such as 100% identical to the Anthranilate synthase encoding polynucleotide comprised in SEQ ID NO: 13 or genomic DNA thereof;
h) genes encoding a Ribose-phosphate pyrophosphokinase said genes being at least 70%, such at least 75%, such as at least 80%, such as at least 90%, such as at least 95%, such as at least 99%, such as 100% identical to the Ribose-phosphate pyrophosphokinase encoding polynucleotide comprised in SEQ ID NO: 15 or genomic DNA thereof;
i) genes encoding a anthranilate phosphoribosyl transferase said genes being at least 70%, such at least 75%, such as at least 80%, such as at least 90%, such as at least 95%, such as at least 99%, such as 100% identical to the Anthranilate phosphoribosyl transferase encoding polynucleotide comprised in SEQ ID NO: 17 or genomic DNA thereof;
j) genes encoding a N-(5′-phosphoribosyl)-anthranilate isomerase said genes being at least 70%, such at least 75%, such as at least 80%, such as at least 90%, such as at least 95%, such as at least 99%, such as 100% identical to the N-(5′-phosphoribosyl)-anthranilate isomerase encoding polynucleotide comprised in SEQ ID NO: 19 or genomic DNA thereof;
k) genes encoding a Indole-3-glycerol phosphate synthase said genes being at least 70%, such at least 75%, such as at least 80%, such as at least 90%, such as at least 95%, such as at least 99%, such as 100% identical to the Indole-3-glycerol phosphate synthase encoding polynucleotide comprised in SEQ ID NO: 21 or genomic DNA thereof;
l) genes encoding a Tryptophan synthase said genes being at least 70%, such at least 75%, such as at least 80%, such as at least 90%, such as at least 95%, such as at least 99%, such as 100% identical to the Tryptophan synthase encoding polynucleotide comprised in anyone of SEQ ID NO: 23, 59, 61, 63, 65, 67, 179, and/or 181 or genomic DNA thereof;
m) genes encoding a Tryptophan decarboxylase, or a non-canonical aromatic amino acid decarboxylase said genes being at least 70%, such at least 75%, such as at least 80%, such as at least 90%, such as at least 95%, such as at least 99%, such as 100% identical to the Tryptophan decarboxylase or a non-canonical aromatic amino acid decarboxylase encoding polynucleotide comprised in SEQ ID NO: 25, 69, 71, 73, 75, and/or 77 or genomic DNA thereof;
n) genes encoding a Chorismate mutase said genes being at least 70%, such at least 75%, such as at least 80%, such as at least 90%, such as at least 95%, such as at least 99%, such as 100% identical to the Chorismate mutase encoding polynucleotide comprised in SEQ ID NO: 45 or genomic DNA thereof;
o) genes encoding a Prephenate dehydrogenase said genes being at least 70%, such at least 75%, such as at least 80%, such as at least 90%, such as at least 95%, such as at least 99%, such as 100% identical to the Prephenate dehydrogenase encoding polynucleotide comprised in SEQ ID NO: 47 or genomic DNA thereof;
p) genes encoding an aromatic aminotransferase said genes being at least 70%, such at least 75%, such as at least 80%, such as at least 90%, such as at least 95%, such as at least 99%, such as 100% identical to the Aromatic aminotransferase encoding polynucleotide comprised in SEQ ID NO: 49 or genomic DNA thereof;
q) genes encoding a Phenylalanine ammonium lyase said genes being at least 70%, such at least 75%, such as at least 80%, such as at least 90%, such as at least 95%, such as at least 99%, such as 100% identical to the Phenylalanine ammonium lyase encoding polynucleotide comprised in SEQ ID NO: 51 or genomic DNA thereof;
r) genes encoding a cinnamate 4-hydroxylase said genes being at least 70%, such at least 75%, such as at least 80%, such as at least 90%, such as at least 95%, such as at least 99%, such as 100% identical to the Cinnamate 4-hydroxylase encoding polynucleotide comprised in SEQ ID NO: 53 or genomic DNA thereof;
s) genes encoding a cytochrome b5 said genes being at least 70%, such at least 75%, such as at least 80%, such as at least 90%, such as at least 95%, such as at least 99%, such as 100% identical to the C cytochrome b5 encoding polynucleotide comprised in SEQ ID NO: 253 or genomic DNA thereof;
t) genes encoding a Cytochrome P450 reductase said genes being at least 70%, such at least 75%, such as at least 80%, such as at least 90%, such as at least 95%, such as at least 99%, such as 100% identical to the Cytochrome P450 reductase encoding polynucleotide comprised in SEQ ID NO: 55, 101, 103, 105, 107, 109 and/or 111 or genomic DNA thereof;
u) genes encoding a 4-Coumoryl-CoA ligase said genes being at least 70%, such at least 75%, such as at least 80%, such as at least 90%, such as at least 95%, such as at least 99%, such as 100% identical to the 4-Coumoryl-CoA ligase encoding polynucleotide comprised in SEQ ID NO: 57 or genomic DNA thereof;
v) genes encoding a Tryptamine 5-hydroxylase said genes being at least 70% identical to the Tryptamine 5-hydroxylase encoding polynucleotide comprised in SEQ ID NO: 96 or genomic DNA thereof;
w) genes encoding a Cytochrome p450 reductase said genes being at least 70% identical to the Cytochrome p450 reductase encoding polynucleotide comprised in SEQ ID NO: 111 or genomic DNA thereof.
x) genes encoding a 4-hydroxytryptamine kinase said genes being at least 70% identitical to the 4-hydroxytryptamine kinase encoding polynucleotide comprised SEQ ID NO 159; and/or y) genes encoding a psilocybin synthase said genes being at least 70% identitical to the psilocybin synthase encoding polynucleotide comprised in SEQ ID NO 127.

In a preferred embodiment the host cell of the invention further expresses:
a) genes encoding a Psilocybin synthase said genes which are at least 70%, such at least 75%, such as at least 80%, such as at least 90%, such as at least 95%, such as at least 99%, such as 100% identical to the Psilocybin synthase encoding polynucleotide comprised in anyone of SEQ ID NO: 127 and/or 123 or genomic DNA thereof;
b) genes encoding a 4-Hydroxytryptamine kinase said genes which are at least 70%, such at least 75%, such as at least 80%, such as at least 90%, such as at least 95%, such as at least 99%, such as 100% identical to the 4-Hydroxytryptamine kinase encoding polynucleotide comprised in anyone of SEQ ID NO: 159 or genomic DNA thereof;
c) genes encoding a P450 reductase said genes which are at least 70%, such at least 75%, such as at least 80%, such as at least 90%, such as at least 95%, such as at least 99%, such as 100% identical to the P450 reductase (CPR) encoding polynucleotide comprised in SEQ ID NO: 105 and/or 101 or genomic DNA thereof;
d) genes encoding a P450 enzyme said genes which are at least 70%, such at least 75%, such as at least 80%, such as at least 90%, such as at least 95%, such as at least 99%, such as 100% identical to the P450 enzyme encoding polynucleotide comprised in SEQ ID NO: 93 and/or 87 or genomic DNA thereof; and
e) genes encoding a Tryptophan decarboxylase said genes being at least 70%, such at least 75%, such as at least 80%, such as at least 90%, such as at least 95%, such as at least 99%, such as 100% identical to the Tryptophan decarboxylase encoding polynucleotide comprised in SEQ ID NO: 77 and/or 71 or genomic DNA thereof.

The host cell may comprise at least two copies of the one or more of the heterologous genes encoding the one or more substituting enzymes or pathway enzymes and such genes may also be overexpressed. The host cell may also be modified to provide an increased amount of a substrate for at least one enzyme of the indole acceptor pathway. Still further the host cell may be further genetically modified to exhibit increased tolerance towards one or more substrates, intermediates, or product molecules from the indole acceptor pathway.

The host cell of the invention is suitably a eukaryotic, prokaryotic or archaic cell. The eukaryote cell is preferably mammalian, insect, plant, or fungal. Fungal host cells include those from phylas Ascomycota, Basidiomycota, Neocallimastigomycota, Glomeromycota, Blastocladiomycota, Chytridiomycota, Zygomycota, Oomycota and Microsporidia. The fungal host cell may be a yeast selected from ascosporogenous yeast (Endomycetales), basidiosporogenous yeast, and Fungi Imperfecti yeast (Blastomycetes). More specifically the yeast host cell is from the genera of Saccharomyces, Kluveromyces, Candida, Pichia, Debaromyces, Hansenula, Yarrowia, Zygosaccharomyces, or Schizosaccharomyces. Even more specifically the yeast host cell is from the species of Kluyveromyces lactis, Saccharomyces carlsbergensis, Saccharomyces cerevisiae, Saccharomyces diastaticus, Saccharomyces douglasii, Saccharomyces kluyveri, Saccharomyces norbensis, Saccharomyces oviformis, Saccharomyces boulardii or Yarrowia lipolytica. The fungal host cell amy also be a filamentous fungus, including those from the phylas consisting of Ascomycota, Eumycota and Oomycota. More specifically the filamentous fungal host cell can be from the genera consisting of Acremonium, Aspergillus, Aureobasidium, Bjerkandera, Ceriporiopsis, Chrysosporium, Coprinus, Corio/us, Cryptococcus, Filibasidium, Fusarium, Humicola, Magnaporthe, Mucor, Myceliophthora, Neocallimastix, Neurospora, Paecilomyces, Penicillium, Phanerochaete, Phlebia, Piromyces, Pleurotus, Schizophyllum, Talaromyces, Thermoascus, Thielavia, Tolypocladium, Trametes, and Trichoderma. Even more specifically the filamentous fungal host cell is from the species consisting of Aspergillus awamori, Aspergillus foetidus, Aspergillus fumigatus, Aspergillus japonicus, Aspergillus nidulans, Aspergillus niger, Aspergillus oryzae, Bjerkandera adusta, Ceriporiopsis aneirina, Ceriporiopsis caregiea, Ceriporiopsis gilvescens, Ceriporiopsis pannocinta, Ceriporiopsis rivulosa, Ceriporiopsis subrufa, Ceriporiopsis subvermispora, Chrysosporiuminops, Chrysosporiumkeratinophilum, Chrysosporium lucknowense, Chrysosporium merdarium, Chrysosporium pannicola, Chrysosporium queenslandicum, Chrysosporium tropicum, Chrysosporium zonatum, Coprinus cinereus, Coriolus hirsutus, Fusarium bactridioides, Fusarium cerealis, Fusarium crookwellense, Fusarium culmorum, Fusarium graminearum, Fusarium graminum, Fusarium heterosporum, Fusarium negundi, Fusarium oxysporum, Fusarium reticulatum, Fusarium roseum, Fusarium sambucinum, Fusarium sarcochroum, Fusarium sporotrichioides, Fusarium sulphureum, Fusarium torulosum, Fusarium trichothecioides, Fusarium venenatum, Humicola insolens, Humicola lanuginosa, Mucor miehei, Myceliophthora thermophila, Neurospora crassa, Penicillium purpurogenum, Phanerochaete chrysosporium, Phlebia radiata, Pleurotus eryngii, Thielavia terrestris, Trametes villosa, Trametes versicolor, Trichoderma harzianum, Trichoderma koningii, Trichoderma longibrachiatum, Trichoderma reesei, and Trichoderma viride. Alternatively, the host cell is a prokaryotic cell, such as E. coli or an archaic cell, such as an algae.

In important embodiments one or more native genes of the host may be attenuated, disrupted and/or deleted to optimize the production of the indole acceptor and/or the tryptamine derivative.

Particular target genes for being attenuated, disrupted and/or deleted are those encoding phosphatase shunting psilocybin to psilocin.

Where the host cell is a yeast strain it may suitably be modified by attenuating, disrupting and/or deleting one or more native genes selected from:
a) The pyruvate kinase gene comprised in anyone of SEQ ID NO: 27 or any of its paralogs or orthologs having at least 70%, such at least 75%, such as at least 80%, such as at least 90%, such as at least 95%, such as at least 99%, such as 100% identity to SEQ ID NO: 27;
b) The phosphofructokinase gene comprised in anyone of SEQ ID NO: 29 or 31 or any of its paralogs or orthologs having at least 70%, such at least 75%, such as at least 80%, such as at least 90%, such as at least 95%, such as at least 99%, such as 100% identity to anyone of SEQ ID NO: 29 or 31;
c) The transporters gene comprised in SEQ ID NO: 33 or any of its paralogs or orthologs having at least 70%, such at least 75%, such as at least 80%, such as at least 90%, such as at least 95%, such as at least 99%, such as 100% identity to SEQ ID NO: 33;
d) The DL-glycerol-3-phosphate phosphatase gene comprised in SEQ ID NO: 34 or any of its paralogs or orthologs having at least 70%, such at least 75%, such as at least 80%, such as at least 90%, such as at least 95%, such as at least 99%, such as 100% identity to SEQ ID NO: 34;
e) The tryptophan 2,3-dioxygenase gene comprised in SEQ ID NO: 35 or any of its paralogs or orthologs having at least 70%, such at least 75%, such as at least 80%, such as at least 90%, such as at least 95%, such as at least 99%, such as 100% identity to SEQ ID NO: 35;
f) The cystathionine beta-synthase gene comprised in SEQ ID NO: 36 or any of its paralogs or orthologs having at least 70%, such at least 75%, such as at least 80%, such as at least 90%, such as at least 95%, such as at least 99%, such as 100% identity to SEQ ID NO: 36;
g) The phenylpyruvate decarboxylase gene comprised in SEQ ID NO: 37 or any of its paralogs or orthologs having at least 70%, such at least 75%, such as at least 80%, such as at least 90%, such as at least 95%, such as at least 99%, such as 100% identity to SEQ ID NO: 37;
h) The pyruvate decarboxylase gene comprised in SEQ ID NO: 38 or any of its paralogs or orthologs having at least 70%, such at least 75%, such as at least 80%, such as at least 90%, such as at least 95%, such as at least 99%, such as 100% identity to SEQ ID NO: 38;
i) The histone variant H2AZ gene comprised in SEQ ID NO: 39 or any of its paralogs or orthologs having at least 70%, such at least 75%, such as at least 80%, such as at least 90%, such as at least 95%, such as at least 99%, such as 100% identity to SEQ ID NO: 39;
j) The phosphatase gene comprised in SEQ ID NO: 40 or any of its paralogs or orthologs having at least 70%, such at least 75%, such as at least 80%, such as at least 90%, such as at least 95%, such as at least 99%, such as 100% identity to SEQ ID NO: 40;
k) The repressible acid phosphatase gene comprised in anyone of SEQ ID NO: 41, 42, or 43 or any of its paralogs or orthologs having at least 70%, such at least 75%, such as at least 80%, such as at least 90%, such as at least 95%, such as at least 99%, such as 100% identity to anyone of SEQ ID NO: 41, 42, or 43;
l) The constitutively expressed acid phosphatase gene comprised in SEQ ID NO: 44 or any of its paralogs or orthologs having at least 70%, such at least 75%, such as at least 80%, such as at least 90%, such as at least 95%, such as at least 99%, such as 100% identity to SEQ ID NO: 44;
m) The sterol reductase gene comprised in SEQ ID NO: 183 or any of its paralogs or orthologs having at least 70%, such at least 75%, such as at least 80%, such as at least 90%, such as at least 95%, such as at least 99%, such as 100% identity to SEQ ID NO: 183; and/or
n) The S-adenosylmethionine decarboxylase gene comprised in SEQ ID NO: 184 or any of its paralogs or orthologs having at least 70%, such at least 75%, such as at least 80%, such as at least 90%, such as at least 95%, such as at least 99%, such as 100% identity to SEQ ID NO: 184.

Where the host cell is a yeast strain it may also suitably be modified to overexpress one or more native genes selected from the NADH kinase gene comprised in SEQ ID NO: 185 or any of its paralogs or orthologs having at least 70%, such at least 75%, such as at least 80%, such as at least 90%, such as at least 95%, such as at least 99%, such as 100% identity to SEQ ID NO: 185.

The invention also provides a cell culture, comprising host cell of the invention and a growth medium. Suitable growth mediums for host cells such as plant cell lines, filamentous fungi and/or yeast are known in the art.

### Nucleotide Constructs

In a further aspect the invention provides a polynucleotide construct comprising a polynucleotide sequence encoding the of the invention, operably linked to one or more control sequences heterologous to the substituting enzyme encoding polynucleotide.

Polynucleotides may be manipulated in a variety of ways to allow expression of a polypeptide. Manipulation of the polynucleotide prior to its insertion into an expression vector may be desirable or necessary depending on the expression vector. The techniques for modifying polynucleotides utilizing recombinant DNA methods are well known in the art.

The control sequence may be a promoter, which is a polynucleotide that is recognized by a host cell for expression of a polynucleotide. The promoter contains transcriptional control sequences that mediate the expression of the polypeptide. The promoter may be any polynucleotide that shows transcriptional activity in the host cell including mutant, truncated, and hybrid promoters, and may be obtained from genes encoding extracellular or intracellular polypeptides either homologous or heterologous to the host cell. The promoter may be an inducible promoter. Useful promotors for expression e.g. in fungi including yeast are known in the art.

The control sequence may also be a transcription terminator, which is recognized by a host cell to terminate transcription. The terminator is operably linked to the 3′-terminus of the polynucleotide encoding the polypeptide. Any terminator that is functional in the host cell may be used. Useful terminators for expression e.g. in fungi including yeast are known in the art.

The control sequence may also be an mRNA stabilizer region downstream of a promoter and upstream of the coding sequence of a gene which increases expression of the gene.

The control sequence may also be a leader, a non-translated region of an mRNA that is important for translation by the host cell. The leader is operably linked to the 5′-terminus of the polynucleotide encoding the polypeptide. Any leader that is functional in the host cell may be used. Useful leaders for expression e.g. in fungi including yeast are known in the art.

The control sequence may also be a polyadenylation sequence; a sequence operably linked to the 3′-terminus of the polynucleotide and, when transcribed, is recognized by the host cell as a signal to add polyadenosine residues to transcribed mRNA. Any polyadenylation sequence that is functional in the host cell may be used. Useful polyadenylation sequences for expression e.g. in fungi including yeast are known in the art.

It may also be desirable to add regulatory sequences that regulate expression of the polypeptide relative to the growth of the host cell. Examples of regulatory systems are those that cause expression of the gene to be turned on or off in response to a chemical or physical stimulus, including the presence of a regulatory compound.

In a further aspect the invention provides an expression vector comprising the polynucleotide construct of the invention. Various nucleotide sequences in addition to the polynucleotide construct of the invention may be joined together to produce a recombinant expression vector, which may include one or more convenient restriction sites to allow for insertion or substitution of the polynucleotide sequence encoding the relevant polypeptide at such sites. The recombinant expression vector may be any vector (e.g., a plasmid or virus or chromosome) that can be conveniently subjected to recombinant DNA procedures and can bring about expression of the relevant polypeptide encoding polynucleotide.

The choice of the vector will typically depend on the compatibility of the vector with the host cell into which the vector is to be introduced. The vector may be a linear or closed circular plasmid. The vector may be an autonomously replicating vector, i.e., a vector that exists as an extrachromosomal entity, the replication of which is independent of chromosomal replication, e.g., a plasmid, an extrachromosomal element, a mini-chromosome, or an artificial chromosome. The vector may contain any means for assuring self-replication. Alternatively, the nucleotide construct may, when introduced into the host cell, integrate into the genome, and replicate together with the chromosome(s) into which it has been integrated. Furthermore, a single vector or plasmid or two or more vectors or plasmids that together contain the total DNA to be introduced into the genome of the host cell, or a transposon, may be used. The vector may contain one or more selectable markers that permit easy selection of transformed, transfected, transduced, or the like cells. A selectable marker is a gene from which the product provides for biocide or viral resistance, resistance to heavy metals, prototrophy to auxotrophs, and the like.

The vector preferably contains element(s) that permits integration of the vector into the host cell's genome or permits autonomous replication of the vector in the cell independent of the genome. For integration into the host cell genome, the vector may rely on the polynucleotide encoding the polypeptide or any other element of the vector for integration into the genome by homologous or non-homologous recombination. Alternatively, the vector may contain additional polynucleotides for directing integration by homologous recombination into the genome of the host cell at precise location(s) in the chromosome(s). To increase the likelihood of integration at a precise location, the integrational elements should contain a sufficient number of nucleic acids, such as 35 to 10,000 base pairs, such as 100 to 10,000 base pairs, such as 400 to 10,000 base pairs, and such as 800 to 10,000 base pairs, which have a high degree of sequence identity to the corresponding target sequence to enhance the probability of homologous recombination. The integrational elements may be any sequence that is homologous with the target sequence in the genome of the host cell. Furthermore, the integrational elements may be non-encoding or encoding polynucleotides. On the other hand, the vector may be integrated into the genome of the host cell by non-homologous recombination.

The origin of replication may be any plasmid replicator mediating autonomous replication that functions in a cell. The term “origin of replication” or “plasmid replicator” refers to a polynucleotide that enables a plasmid or vector to replicate in vivo.

More than one copy of a polynucleotide encoding the substituting enzyme or other pathway polypeptides of the invention may be inserted into a host cell to increase production of a polypeptide. An increase in the copy number can be obtained by integrating one or more additional copies of the enzyme coding sequence into the host cell genome or by including an amplifiable selectable marker gene with the polynucleotide, so that cells containing amplified copies of the selectable marker gene—and thereby additional copies of the polynucleotide—can be selected by cultivating the cells in the presence of the appropriate selectable agent. The procedures used to ligate the elements described above to construct the recombinant expression vectors of the present invention are well known to one skilled in the art (see, e.g., Green & Sambrook, 2012, Molecular cloning: A laboratory Manual, Fourth Edition, Cold Spring Harbor Laboratory, New York, USA).

### Fermentation Methods for Producing Compounds of the Invention.

Where the method of the invention is wholly or partially performed by fermenting a cell culture of the the genetically modified host cell of the invention, the method claims suitably further comprise:
a) culturing the cell culture described herein at conditions allowing the host cell to produce the tryptamine derivative (I); and
b) optionally recovering and/or isolating the tryptamine derivative (I).

The cell culture can be cultivated in a nutrient medium and at conditions suitable for production of the tryptamine derivative of the invention and/or propagating cell count using methods known in the art. For example, the culture may be cultivated by shake flask cultivation, or small-scale or large-scale fermentation (including continuous, batch, fed-batch, feed and draw, or solid-state fermentations) in laboratory or industrial fermentors in a suitable medium and under conditions allowing the host cells to grow and/or propagate, optionally to be recovered and/or isolated.

The cultivation can take place in a suitable nutrient medium comprising carbon and nitrogen sources and inorganic salts, using procedures known in the art. Suitable media are available from commercial suppliers or may be prepared according to published recipes (e.g. from catalogues of the American Type Culture Collection). The selection of the appropriate medium may be based on the choice of host cell and/or based on the regulatory requirements for the host cell. Such media are available in the art. The medium may, if desired, contain additional components favoring the transformed expression hosts over other potentially contaminating microorganisms. Accordingly, in an embodiment a suitable nutrient medium comprises a carbon source (e.g. glucose, maltose, molasses, starch, cellulose, xylan, pectin, lignocellolytic biomass hydrolysate, etc.), a nitrogen source (e. g. ammonium sulphate, ammonium nitrate, ammonium chloride, etc.), an organic nitrogen source (e.g. yeast extract, malt extract, peptone, etc.) and inorganic nutrient sources (e.g. phosphate, magnesium, potassium, zinc, iron, etc.).

The cultivation of the host cell may be performed over a period of from about 0.5 to about 30 days. The cultivation process may be a batch process, continuous or fed-batch process, suitably performed at a temperature in the range of 0-100° C. or 0-80° C., for example, from about 0° C. to about 50° C. and/or at a pH, for example, from about 2 to about 10. Preferred fermentation conditions for yeast and filamentous fungi are a temperature in the range of from about 25° C. to about 55° C. and at a pH of from about 3 to about 9. The appropriate conditions are usually selected based on the choice of host cell. Accordingly, in an embodiment the method of the invention further comprises one or more elements selected from:
a) culturing the cell culture in a nutrient growth medium;
b) culturing the cell culture under aerobic or anaerobic conditions
c) culturing the cell culture under agitation;
d) culturing the cell culture at a temperature of between 25 to 50° C.;
e) culturing the cell culture at a pH of between 3-9;
f) culturing the cell culture for between 10 hours to 30 days; and
g) culturing the cell culture under fed-batch, repeated fed-batch, continuous, or semi-continuous conditions.

The fermentation method of the invention, further suitably comprise feeding one or more exogenous indole acceptor or precursors thereof and/or substituent donors to the cell culture.

The cell culture and/or the the metabolites comprised therein may be recovered and or isolated using methods known in the art. For example, the cells and or metabolites may be recovered from the nutrient medium by conventional procedures including, but not limited to, centrifugation, filtration, spray-drying, or lyophilization. In a particular embodiment the method includes a recovery and/or isolation step comprising separating a liquid phase of the cell or cell culture from a solid phase of the cell or cell culture to obtain a supernatant comprising the tryptamine derivative and subjecting the supernatant to one or more steps selected from:
a) disrupting the host cell to release intracellular tryptamine derivative into the supernatant;
b) separating the supernatant form the solid phase of the host cell, such as by filtration or gravity separation;
c) contacting the supernatant with one or more adsorbent resins in order to obtain at least a portion of the produced tryptamine derivative;
d) contacting the supernatant with one or more ion exchange or reversed-phase chromatography columns in order to obtain at least a portion of the tryptamine derivative;
e) extracting the tryptamine derivative; and
f) precipitating the tryptamine derivative by crystallization or evaporating the solvent of the liquid phase; and optionally isolating the tryptamine derivative by filtration or gravity separation; thereby recovering and/or isolating the tryptamine derivative.

### Fermentation Liquids/Compositions

The invention further provides a fermentation liquid/composition comprising the cell culture of the invention and the tryptamine derivative comprised therein.

In one embodiment at least 10%, 25%, 50%, such as at least 75%, such as at least 95%, such as at least 99% of the cells of the fermentation liquid/composition of the invention are lysed. Further in the fermentation liquid/composition of the invention at least 10%, 25%, 50%, such as at least 75%, such as at least 95%, such as at least 99% of solid cellular material may have been removed and separated from a liquid phase. Moreover, in addition to the tryptamine derivative the fermentation liquid/composition of the invention may also comprise precursors, products, metabolites of the indole acceptor pathway, in particular tryptophan and/or tryptamine or comprise one or more compounds selected from trace metals, vitamins, salts, yeast nitrogen base, carbon source, YNB, and/or amino acids of the fermentation. In particular the fermentation liquid/composition comprises a concentration of tryptamine derivative of at least 1 mg/kg or mg/L composition, such as at least 5 mg/kg or mg/L, such as at least 10 mg/kg or mg/L, such as at least 20 mg/kg or mg/L, such as at least 50 mg/kg or mg/L, such as at least 100 mg/kg or mg/L, such as at least 500 mg/kg or mg/L, such as at least 1000 mg/kg or mg/L, such as at least 5000 mg/kg or mg/L, such as at least 10000 mg/kg or mg/L, such as at least 50000 mg/kg or mg/L.

### Compositions

In a further aspect the invention further provides a composition comprising the fermentation liquid/composition of the invention and one or more carriers, agents, additives and/or excipients. Carriers, agents, additives and/or excipients includes formulation additives, stabilising agent, fillers, and the like. The composition may be formulated into a dry solid form, such as powders, tablets, capsules, hard chewables and or soft lozenges or a gums by using methods known in the art, such as spray drying, spray cooling, lyophilization, flash freezing, granulation, microgranulation, encapsulation or microencapsulation. The composition may also be formulated into liquid stabilized form using methods known in the art, such as formulation into a stabilized liquid comprising one or more stabilizers such as sugars and/or polyols (e.g. sugar alcohols) and/or organic acids (e.g. lactic acid).

### Further Aspects

In addition to the method and host cells described herein producing tryptamine derivatives of formula (I), a further aspect relates to methods and host cells for producing serotonin (an indole acceptor), which for example are useful in the methods described herein for making tryptamine derivatives of formula (I). The known route to serotonin is long and complicated and requires addition or engineering of an essential co-factor, the present inventor have now fould that serotonin can be much more efficiently produced in engineered host cells producing or being fed tryptamine and expressing both a tryptamine 5-hydroxylase converting tryptamine to serotonin; and a Cytochrome p450 reductase assisting the Tryptamine 5-hydroxylase conversion of tryptamine to serotonin.

Accordingly, provided for herein is genetically modified microbial host cell producing a serotonin indole acceptor expressing one or more genes encoding polypeptides selected from
a) one or more genes encoding polypeptides selected from
   i) one or more enzymes converting glucose to fructose-6-phosphate;
   ii) oa fructose-6-phosphate phosphoketolase converting fructose-6-phosphate to Erythrose-4-phosphate and acetyl phosphate;
   iii) a Phosphotransacetylase converting Acetyl phosphate to Acetyl-CoA;
   iv) one or more enzymes converting Fructose-6-phosphate to Phosphoenolpyruvate;
   v) a 3-deoxy-D-arabino-heptulosonate 7-phosphate synthase (DAHP synthase) converting Phosphoenolpyruvate and Erythrose-4-phosphate to 3-deoxy-D-arabino-heptulosonate-7-phosphate (DAHP);
   vi) one or more enzymes converting 3-deoxy-D-arabino-heptulosonate-7-phosphate to 5-enolpyruvoyl-shikimate 3-phosphate;
   vii) a Shikimate kinase converting Shikimate to Shikimate-3-phosphate;
   viii) a Chorismate synthase converting 5-enolpyruvoyl-shikimate 3-phosphate to Chorismate;
   ix) a Anthranilate synthase converting Chorismate to Anthranilate;
   x) a Ribose-phosphate pyrophosphokinase converting Ribose-5-phosphate to Phospho-alpha-D-ribosyl-1-pyrophosphate;
   xi) a Anthranilate phosphoribosyl transferase converting Anthranilate and Phospho-alpha-D-ribosyl-1-pyrophosphate to N-(5-phosphoribosyl)-anthranilate;
   xii) a N-(5′-phosphoribosyl)-anthranilate isomerase converting N-(5-phosphoribosyl)-anthranilate to 1-(o-carboxyphenylamino)-1′-deoxyribulose 5′-phosphate;
   xiii) an Indole-3-glycerol phosphate synthase converting 1-(o-carboxyphenylamino)-1′-deoxyribulose 5′-phosphate to (1S,2R)-1-C-(indol-3-yl)-glycerol 3-phosphate;
   xiv) a Tryptophan synthase converting (1S,2R)-1-C-(indol-3-yl) glycerol 3-phosphate and Serine to L-Tryptophan; and/or
   xv) a Tryptophan decarboxylase converting L-Tryptophan to Tryptamine; and
b) a heterologous Tryptamine 5-hydroxylase converting tryptamine to serotonin; and
c) a heterologous Cytochrome p450 reductase assisting the conversion of tryptamine to serotonin by a Tryptamine 5-hydroxylase.

Where the host cell is S. cerevisiae this species contains a native CPR enzyme (Ncp1), but it does not function with heterologous CYP enzymes such as tryptamine 5-hydroxylases.

Herein it has been shown that such engineered host cells can efficiently produce serotonin by an alternative biosynthetic pathway not previously described in the art. This production method offers significant improvement over the art by being more efficient but also alleviating the need to produce tetrahydrobiopterin or add it exogenously.

In important embodiments in the host cell producing serotonin the corresponding:
a) fructose-6-phosphate phosphoketolase has at least 70%, such at least 75%, such as at least 80%, such as at least 90%, such as at least 95%, such as at least 99%, such as 100% identity to the fructose-6-phosphate phosphoketolase comprised in SEQ ID NO: 2;
b) Phosphotransacetylase has at least 70%, such at least 75%, such as at least 80%, such as at least 90%, such as at least 95%, such as at least 99%, such as 100% identity to the Phosphotransacetylase comprised in SEQ ID NO: 4;
c) 3-deoxy-D-arabino-heptulosonate 7-phosphate synthase has at least 70%, such at least 75%, such as at least 80%, such as at least 90%, such as at least 95%, such as at least 99%, such as 100% identity to the 3-deoxy-D-arabino-heptulosonate 7-phosphate synthase comprised in SEQ ID NO: 6;
d) Enzyme converting 3-deoxy-D-arabino-heptulosonate-7-phosphate to 5-enolpyruvoyl-shikimate 3-phosphate has at least 70%, such at least 75%, such as at least 80%, such as at least 90%, such as at least 95%, such as at least 99%, such as 100% identity to the Enzyme comprised in SEQ ID NO: 8;
e) Shikimate kinase has at least 70%, such at least 75%, such as at least 80%, such as at least 90%, such as at least 95%, such as at least 99%, such as 100% identity to the Shikimate kinase comprised in SEQ ID NO: 10;
f) Chorismate synthase has at least 70%, such at least 75%, such as at least 80%, such as at least 90%, such as at least 95%, such as at least 99%, such as 100% identity to the Chorismate synthase comprised in SEQ ID NO: 12;
g) Anthranilate synthase has at least 70%, such at least 75%, such as at least 80%, such as at least 90%, such as at least 95%, such as at least 99%, such as 100% identity to the Anthranilate synthase comprised in SEQ ID NO: 14;
h) Ribose-phosphate pyrophosphokinase has at least 70%, such at least 75%, such as at least 80%, such as at least 90%, such as at least 95%, such as at least 99%, such as 100% identity to the Ribose-phosphate pyrophosphokinase comprised in SEQ ID NO: 16;
i) Anthranilate phosphoribosyl transferase has at least 70%, such at least 75%, such as at least 80%, such as at least 90%, such as at least 95%, such as at least 99%, such as 100% identity to the Anthranilate phosphoribosyl transferase comprised in SEQ ID NO: 18;
j) N-(5′-phosphoribosyl)-anthranilate isomerase has at least 70%, such at least 75%, such as at least 80%, such as at least 90%, such as at least 95%, such as at least 99%, such as 100% identity to the N-(5′-phosphoribosyl)-anthranilate isomerase comprised in SEQ ID NO: 20;
k) Indole-3-glycerol phosphate synthase has at least 70%, such at least 75%, such as at least 80%, such as at least 90%, such as at least 95%, such as at least 99%, such as 100% identity to the Indole-3-glycerol phosphate synthase comprised in SEQ ID NO: 22;
l) Tryptophan synthase has at least 70%, such at least 75%, such as at least 80%, such as at least 90%, such as at least 95%, such as at least 99%, such as 100% identity to the Tryptophan synthase comprised in anyone of SEQ ID NO: 24, 60, 62, 64, 66, 68, 180 and/or 182;
m) Tryptophan decarboxylase or a non-canonical aromatic amino acid decarboxylase has at least 70%, such at least 75%, such as at least 80%, such as at least 90%, such as at least 95%, such as at least 99%, such as 100% identity to the Tryptophan decarboxylase or a non-canonical aromatic amino acid decarboxylase comprised in anyone of SEQ ID NO: 26, 70, 72, 74, 76, and/or 78;
n) Tryptamine 5-hydroxylase has at least 70%, such at least 75%, such as at least 80%, such as at least 90%, such as at least 95%, such as at least 99%, such as 100% identity to the Tryptamine 5-hydroxylase comprised in SEQ ID NO: 96 (OsT5H); and
o) Cytochrome p450 reductase has at least 70%, such at least 75%, such as at least 80%, such as at least 90%, such as at least 95%, such as at least 99%, such as 100% identity to the Cytochrome p450 reductase comprised in SEQ ID NO: 112 (FoCPR).

In the host cell producing serotonin the one or more expressed genes are preferably selected from:
a) genes encoding a fructose-6-phosphate phosphoketolase said genes being at least 70%, such at least 75%, such as at least 80%, such as at least 90%, such as at least 95%, such as at least 99%, such as 100% identical to the fructose-6-phosphate phosphoketolase encoding polynucleotide comprised in SEQ ID NO: 1 or genomic DNA thereof;
b) genes encoding a Phosphotransacetylase said genes being at least 70%, such at least 75%, such as at least 80%, such as at least 90%, such as at least 95%, such as at least 99%, such as 100% identical to the Phosphotransacetylase encoding polynucleotide comprised in SEQ ID NO: 3 or genomic DNA thereof;
c) genes encoding a 3-deoxy-D-arabino-heptulosonate 7-phosphate synthase said genes being at least 70%, such at least 75%, such as at least 80%, such as at least 90%, such as at least 95%, such as at least 99%, such as 100% identical to the 3-deoxy-D-arabino-heptulosonate 7-phosphate synthase encoding polynucleotide comprised in SEQ ID NO: 5 or genomic DNA thereof;
d) genes encoding an enzyme converting 3-deoxy-D-arabino-heptulosonate-7-phosphate to 5-enolpyruvoyl-shikimate 3-phosphate said genes being at least 70%, such at least 75%, such as at least 80%, such as at least 90%, such as at least 95%, such as at least 99%, such as 100% identical to the polynucleotide encoding the enzyme converting 3-deoxy-D-arabino-heptulosonate-7-phosphate to 5-enolpyruvoyl-shikimate 3-phosphate comprised in SEQ ID NO: 7 or genomic DNA thereof;
e) genes encoding a Shikimate kinase said genes being at least 70%, such at least 75%, such as at least 80%, such as at least 90%, such as at least 95%, such as at least 99%, such as 100% identical to the Shikimate kinase encoding polynucleotide comprised in SEQ ID NO: 9 or genomic DNA thereof;
f) genes encoding a Shikimate kinase said genes being at least 70%, such at least 75%, such as at least 80%, such as at least 90%, such as at least 95%, such as at least 99%, such as 100% identical to the Chorismate synthase encoding polynucleotide comprised in SEQ ID NO: 11 or genomic DNA thereof;
g) genes encoding a Anthranilate synthase said genes being at least 70%, such at least 75%, such as at least 80%, such as at least 90%, such as at least 95%, such as at least 99%, such as 100% identical to the Anthranilate synthase encoding polynucleotide comprised in SEQ ID NO: 13 or genomic DNA thereof;
h) genes encoding a Ribose-phosphate pyrophosphokinase said genes being at least 70%, such at least 75%, such as at least 80%, such as at least 90%, such as at least 95%, such as at least 99%, such as 100% identical to the Ribose-phosphate pyrophosphokinase encoding polynucleotide comprised in SEQ ID NO: 15 or genomic DNA thereof;
i) genes encoding a nthranilate phosphoribosyl transferase said genes being at least 70%, such at least 75%, such as at least 80%, such as at least 90%, such as at least 95%, such as at least 99%, such as 100% identical to the Anthranilate phosphoribosyl transferase encoding polynucleotide comprised in SEQ ID NO: 17 or genomic DNA thereof;
j) genes encoding a N-(5′-phosphoribosyl)-anthranilate isomerase said genes being at least 70%, such at least 75%, such as at least 80%, such as at least 90%, such as at least 95%, such as at least 99%, such as 100% identical to the N-(5′-phosphoribosyl)-anthranilate isomerase encoding polynucleotide comprised in SEQ ID NO: 19 or genomic DNA thereof;
k) genes encoding a Indole-3-glycerol phosphate synthase said genes being at least 70%, such at least 75%, such as at least 80%, such as at least 90%, such as at least 95%, such as at least 99%, such as 100% identical to the Indole-3-glycerol phosphate synthase encoding polynucleotide comprised in SEQ ID NO: 21 or genomic DNA thereof;
l) genes encoding a Tryptophan synthase said genes being at least 70%, such at least 75%, such as at least 80%, such as at least 90%, such as at least 95%, such as at least 99%, such as 100% identical to the Tryptophan synthase encoding polynucleotide comprised in anyone of SEQ ID NO: 23, 59, 61, 63, 65, 67, 179, and/or 181 or genomic DNA thereof;
m) genes encoding a Tryptophan decarboxylase, or a non-canonical aromatic amino acid decarboxylase said genes being at least 70%, such at least 75%, such as at least 80%, such as at least 90%, such as at least 95%, such as at least 99%, such as 100% identical to the Tryptophan decarboxylase or a non-canonical aromatic amino acid decarboxylase encoding polynucleotide comprised in SEQ ID NO: 25, 69, 71, 73, 75, and/or 77 or genomic DNA thereof;
n) genes encoding a Tryptamine 5-hydroxylase said genes being at least 70%, such at least 75%, such as at least 80%, such as at least 90%, such as at least 95%, such as at least 99%, such as 100% identical to the Tryptamine 5-hydroxylase encoding polynucleotide comprised in SEQ ID NO: 96 or genomic DNA thereof;
o) genes encoding a Cytochrome p450 reductase said genes being at least 70%, such at least 75%, such as at least 80%, such as at least 90%, such as at least 95%, such as at least 99%, such as 100% identical to the Cytochrome p450 reductase encoding polynucleotide comprised in SEQ ID NO: 111 or genomic DNA thereof.

Where the host cell producing the serotonin a yeast strain, such as S. cerevisiae, the host cell is preferably further modified to attenuate, disrupt and/or delete one or more native genes selected from:
a) The pyruvate kinase gene comprised in anyone of SEQ ID NO: 27 or any of its paralogs or orthologs having at least 70%, such at least 75%, such as at least 80%, such as at least 90%, such as at least 95%, such as at least 99%, such as 100% identity to SEQ ID NO: 27;
b) The phosphofructokinase gene comprised in anyone of SEQ ID NO: 29 and/or 31 or any of its paralogs or orthologs having at least 70%, such at least 75%, such as at least 80%, such as at least 90%, such as at least 95%, such as at least 99%, such as 100% identity to anyone of SEQ ID NO: 29 and/or 31;
c) The transporters gene comprised in SEQ ID NO: 33 or any of its paralogs or orthologs having at least 70%, such at least 75%, such as at least 80%, such as at least 90%, such as at least 95%, such as at least 99%, such as 100% identity to SEQ ID NO: 33;
d) The DL-glycerol-3-phosphate phosphatase gene comprised in SEQ ID NO: 34 or any of its paralogs or orthologs having at least 70%, such at least 75%, such as at least 80%, such as at least 90%, such as at least 95%, such as at least 99%, such as 100% identity to SEQ ID NO: 34; and/or
e) The tryptophan 2,3-dioxygenase gene comprised in SEQ ID NO: 35 or any of its paralogs or orthologs having at least 70%, such at least 75%, such as at least 80%, such as at least 90%, such as at least 95%, such as at least 99%, such as 100% identity to SEQ ID NO: 35;
f) The cystathionine beta-synthase gene comprised in SEQ ID NO: 36 or any of its paralogs or orthologs having at least 70%, such at least 75%, such as at least 80%, such as at least 90%, such as at least 95%, such as at least 99%, such as 100% identity to SEQ ID NO: 36;
g) The phenylpyruvate decarboxylase gene comprised in SEQ ID NO: 37 or any of its paralogs or orthologs having at least 70%, such at least 75%, such as at least 80%, such as at least 90%, such as at least 95%, such as at least 99%, such as 100% identity to SEQ ID NO: 37;
h) The pyruvate decarboxylase gene comprised in SEQ ID NO: 38 or any of its paralogs or orthologs having at least 70%, such at least 75%, such as at least 80%, such as at least 90%, such as at least 95%, such as at least 99%, such as 100% identity to SEQ ID NO: 38; and/or
i) The histone variant H2AZ gene comprised in SEQ ID NO: 39 or any of its paralogs or orthologs having at least 70%, such at least 75%, such as at least 80%, such as at least 90%, such as at least 95%, such as at least 99%, such as 100% identity to SEQ ID NO: 39.

In such yeast host cells it is also preferred to further modify it to overexpress one or more native genes selected from the NADH kinase gene comprised in SEQ ID NO: 185 or any of its paralogs or orthologs having at least 70%, such at least 75%, such as at least 80%, such as at least 90%, such as at least 95%, such as at least 99%, such as 100% identity to SEQ ID NO: 185.

In a still further aspect provided herein is a cell culture, comprising host cell producing serotonin (an indole acceptor) and a growth medium.

In a still further aspect provided herein is a method of producing serotonin (an indole acceptor) comprising:
a) culturing the said cell culture at conditions allowing the host cell to produce the serotonin; and
b) optionally recovering and/or isolating the serotonin.

Such method can in further embodiments include one or more elements selected from:
a) culturing the cell culture in a nutrient growth medium;
b) culturing the cell culture under aerobic or anaerobic conditions
c) culturing the cell culture under agitation;
d) culturing the cell culture at a temperature of between 25 to 50° C.;
e) culturing the cell culture at a pH of between 3-9;
f) culturing the cell culture for between 10 hours to 30 days; and
g) culturing the cell culture under fed-batch, repeated fed-batch, continuous, or semi-continuous conditions.

The method can also comprise feeding one or more exogenous precursors of the serotonin pathway to the cell culture.

The recovering and/or isolation step can include separating a liquid phase of host cell or cell culture from a solid phase of host cell or cell culture to obtain a supernatant comprising the serotonin by one or more steps selected from:
a) disrupting the host cell to release intracellular serotonin indole acceptor into the supernatant;
b) separating the supernatant from the solid phase of the host cell, such as by filtration or gravity separation;
c) contacting the supernatant with one or more adsorbent resins in order to obtain at least a portion of the produced serotonin;
d) contacting the supernatant with one or more ion exchange or reversed-phase chromatography columns in order to obtain at least a portion of the serotonin;
e) extracting the serotonin; and
f) precipitating the serotonin indole acceptor by crystallization or evaporating the solvent of the liquid phase; and optionally isolating the serotonin by filtration or gravity separation; thereby recovering and/or isolating the serotonin.

In a still further aspect provided herein is a fermentation liquid/composition comprising the serotonin comprised in the cell culture or the growth medium. In such fermentation liquids at least 50%, such as at least 75%, such as at least 95%, such as at least 99% of the host cells may be disrupted and further at least 50%, such as at least 75%, such as at least 95%, such as at least 99% of solid cellular material has separated from the liquid. Moreover, in addition to the serotonin the fermentation liquid/composition of the invention may also comprise precursors, products, metabolites of the serotonin pathway, in particular tryptophan and/or tryptamine or comprise one or more compounds selected from trace metals, vitamins, salts, yeast nitrogen base, carbon source, YNB, and/or amino acids of the fermentation. In particular the fermentation liquid/composition comprises a concentration of serotonin of at least 1 mg/kg or mg/L composition, such as at least 5 mg/kg or mg/L, such as at least 10 mg/kg or mg/L, such as at least 20 mg/kg or mg/L, such as at least 50 mg/kg or mg/L, such as at least 100 mg/kg or mg/L, such as at least 500 mg/kg or mg/L, such as at least 1000 mg/kg or mg/L, such as at least 5000 mg/kg or mg/L, such as at least 10000 mg/kg or mg/L, such as at least 50000 mg/kg or mg/L.

In a further aspect provided herein is a composition comprising the fermentation liquid/composition of the invention and one or more carriers, agents, additives and/or excipients. Carriers, agents, additives and/or excipients includes formulation additives, stabilising agent, fillers, and the like. The composition may be formulated into a dry solid form, such as powders, tablets, capsules, hard chewables and or soft lozenges or a gums by using methods known in the art, such as spray drying, spray cooling, lyophilization, flash freezing, granulation, microgranulation, encapsulation or microencapsulation. The composition may also be formulated into liquid stabilized form using methods known in the art, such as formulation into a stabilized liquid comprising one or more stabilizers such as sugars and/or polyols (e.g. sugar alcohols) and/or organic acids (e.g. lactic acid).

In addition to the method and host cells described herein producing tryptamine derivatives of formula (I), a further aspect relates to methods and host cells for producing psilocybin (an indole acceptor), which for example are useful in the methods described herein for making tryptamine derivatives of formula (I).

Accordingly, provided for herein is genetically modified microbial host cell producing a psilocybin expressing one or more genes encoding polypeptides selected from
a) one or more genes encoding polypeptides selected from
   i) one or more enzymes converting glucose to fructose-6-phosphate;
   ii) oa fructose-6-phosphate phosphoketolase converting fructose-6-phosphate to Erythrose-4-phosphate and acetyl phosphate;
   iii) a Phosphotransacetylase converting Acetyl phosphate to Acetyl-CoA;
   iv) one or more enzymes converting Fructose-6-phosphate to Phosphoenolpyruvate;
   v) a 3-deoxy-D-arabino-heptulosonate 7-phosphate synthase (DAHP synthase) converting Phosphoenolpyruvate and Erythrose-4-phosphate to 3-deoxy-D-arabino-heptulosonate-7-phosphate (DAHP);
   vi) one or more enzymes converting 3-deoxy-D-arabino-heptulosonate-7-phosphate to 5-enolpyruvoyl-shikimate 3-phosphate;
   vii) a Shikimate kinase converting Shikimate to Shikimate-3-phosphate;
   viii) a Chorismate synthase converting 5-enolpyruvoyl-shikimate 3-phosphate to Chorismate;
   ix) a Anthranilate synthase converting Chorismate to Anthranilate;
   x) a Ribose-phosphate pyrophosphokinase converting Ribose-5-phosphate to Phospho-alpha-D-ribosyl-1-pyrophosphate;
   xi) a Anthranilate phosphoribosyl transferase converting Anthranilate and Phospho-alpha-D-ribosyl-1-pyrophosphate to N-(5-phosphoribosyl)-anthranilate;
   xii) a N-(5′-phosphoribosyl)-anthranilate isomerase converting N-(5-phosphoribosyl)-anthranilate to 1-(o-carboxyphenylamino)-1′-deoxyribulose 5′-phosphate;
   xiii) an Indole-3-glycerol phosphate synthase converting 1-(o-carboxyphenylamino)-1′-deoxyribulose 5′-phosphate to (1S,2R)-1-C-(indol-3-yl)-glycerol 3-phosphate;
   xiv) a Tryptophan synthase converting (1S,2R)-1-C-(indol-3-yl) glycerol 3-phosphate and Serine to L-Tryptophan; and/or
   xv) a Tryptophan decarboxylase converting L-Tryptophan to Tryptamine; and
b) a Tryptamine 4-hydroxylase converting tryptamine to 4-hydroxytryptamine;
c) a Cytochrome p450 reductase assisting tryptamine 4-hydroxylase in converting tryptamine to 4-hydroxytryptamine;
d) a Cytochrome b5 assisting tryptamine 4-hydroxylase in converting tryptamine to 4-hydroxytryptamine;
e) 4-hydroxytryptamine kinase converting 4-hydroxytryptamine to Norbaeocystin; and
f) Psilocybin synthase converting Norbaeocystin to Psilocybin.

Herein it has been shown that such engineered host cells can efficiently produce psilocybin by an alternative biosynthetic pathway not previously described in the art. This production method offers significant improvement over the art by being more efficient.

In important embodiments in the host cell producing psilocybin the corresponding:
a) fructose-6-phosphate phosphoketolase has at least 70%, such at least 75%, such as at least 80%, such as at least 90%, such as at least 95%, such as at least 99%, such as 100% identity to the fructose-6-phosphate phosphoketolase comprised in SEQ ID NO: 2;
b) Phosphotransacetylase has at least 70%, such at least 75%, such as at least 80%, such as at least 90%, such as at least 95%, such as at least 99%, such as 100% identity to the Phosphotransacetylase comprised in SEQ ID NO: 4;
c) 3-deoxy-D-arabino-heptulosonate 7-phosphate synthase has at least 70%, such at least 75%, such as at least 80%, such as at least 90%, such as at least 95%, such as at least 99%, such as 100% identity to the 3-deoxy-D-arabino-heptulosonate 7-phosphate synthase comprised in SEQ ID NO: 6;
d) Enzyme converting 3-deoxy-D-arabino-heptulosonate-7-phosphate to 5-enolpyruvoyl-shikimate 3-phosphate has at least 70%, such at least 75%, such as at least 80%, such as at least 90%, such as at least 95%, such as at least 99%, such as 100% identity to the Enzyme comprised in SEQ ID NO: 8;
e) Shikimate kinase has at least 70%, such at least 75%, such as at least 80%, such as at least 90%, such as at least 95%, such as at least 99%, such as 100% identity to the Shikimate kinase comprised in SEQ ID NO: 10;
f) Chorismate synthase has at least 70%, such at least 75%, such as at least 80%, such as at least 90%, such as at least 95%, such as at least 99%, such as 100% identity to the Chorismate synthase comprised in SEQ ID NO: 12;
g) Anthranilate synthase has at least 70%, such at least 75%, such as at least 80%, such as at least 90%, such as at least 95%, such as at least 99%, such as 100% identity to the Anthranilate synthase comprised in SEQ ID NO: 14;
h) Ribose-phosphate pyrophosphokinase has at least 70%, such at least 75%, such as at least 80%, such as at least 90%, such as at least 95%, such as at least 99%, such as 100% identity to the Ribose-phosphate pyrophosphokinase comprised in SEQ ID NO: 16;
i) Anthranilate phosphoribosyl transferase has at least 70%, such at least 75%, such as at least 80%, such as at least 90%, such as at least 95%, such as at least 99%, such as 100% identity to the Anthranilate phosphoribosyl transferase comprised in SEQ ID NO: 18;
j) N-(5′-phosphoribosyl)-anthranilate isomerase has at least 70%, such at least 75%, such as at least 80%, such as at least 90%, such as at least 95%, such as at least 99%, such as 100% identity to the N-(5′-phosphoribosyl)-anthranilate isomerase comprised in SEQ ID NO: 20;
k) Indole-3-glycerol phosphate synthase has at least 70%, such at least 75%, such as at least 80%, such as at least 90%, such as at least 95%, such as at least 99%, such as 100% identity to the Indole-3-glycerol phosphate synthase comprised in SEQ ID NO: 22;
l) Tryptophan synthase has at least 70%, such at least 75%, such as at least 80%, such as at least 90%, such as at least 95%, such as at least 99%, such as 100% identity to the Tryptophan synthase comprised in anyone of SEQ ID NO: 24, 60, 62, 64, 66, 68, 180 and/or 182;
m) Tryptophan decarboxylase or a non-canonical aromatic amino acid decarboxylase has at least 70%, such at least 75%, such as at least 80%, such as at least 90%, such as at least 95%, such as at least 99%, such as 100% identity to the Tryptophan decarboxylase or a non-canonical aromatic amino acid decarboxylase comprised in anyone of SEQ ID NO: 26, 70, 72, 74, 76, and/or 78;
n) tryptamine 4-hydroxylase has at least 70%, such at least 75%, such as at least 80%, such as at least 90%, such as at least 95%, such as at least 99%, such as 100% identity to the Tryptamine 4-hydroxylase comprised in anyone of SEQ ID NO 94 and/or 88;
o) cytochrome p450 reductase has at least 70%, such at least 75%, such as at least 80%, such as at least 90%, such as at least 95%, such as at least 99%, such as 100% identity to the Cytochrome p450 reductase comprised in anyone of SEQ ID NO 106 and/or 102;
p) cytochrome b5 has at least 70%, such at least 75%, such as at least 80%, such as at least 90%, such as at least 95%, such as at least 99%, such as 100% identity to the Cytochrome b5 comprised in anyone of SEQ ID NO 254;
q) 4-hydroxytryptamine kinase has at least 70%, such at least 75%, such as at least 80%, such as at least 90%, such as at least 95%, such as at least 99%, such as 100% identity to the 4-hydroxytryptamine kinase comprised in anyone of SEQ ID NO 160 or 156; and
r) psilocybin synthase has at least 70%, such at least 75%, such as at least 80%, such as at least 90%, such as at least 95%, such as at least 99%, such as 100% identity to the psilocybin synthase comprised in anyone of SEQ ID NO 128 or 124.

In the host cell producing psilocybin the one or more expressed genes are preferably selected from:
a) genes encoding a fructose-6-phosphate phosphoketolase said genes being at least 70%, such at least 75%, such as at least 80%, such as at least 90%, such as at least 95%, such as at least 99%, such as 100% identical to the fructose-6-phosphate phosphoketolase encoding polynucleotide comprised in SEQ ID NO: 1 or genomic DNA thereof;
b) genes encoding a Phosphotransacetylase said genes being at least 70%, such at least 75%, such as at least 80%, such as at least 90%, such as at least 95%, such as at least 99%, such as 100% identical to the Phosphotransacetylase encoding polynucleotide comprised in SEQ ID NO: 3 or genomic DNA thereof;
c) genes encoding a 3-deoxy-D-arabino-heptulosonate 7-phosphate synthase said genes being at least 70%, such at least 75%, such as at least 80%, such as at least 90%, such as at least 95%, such as at least 99%, such as 100% identical to the 3-deoxy-D-arabino-heptulosonate 7-phosphate synthase encoding polynucleotide comprised in SEQ ID NO: 5 or genomic DNA thereof;
d) genes encoding an enzyme converting 3-deoxy-D-arabino-heptulosonate-7-phosphate to 5-enolpyruvoyl-shikimate 3-phosphate said genes being at least 70%, such at least 75%, such as at least 80%, such as at least 90%, such as at least 95%, such as at least 99%, such as 100% identical to the polynucleotide encoding the enzyme converting 3-deoxy-D-arabino-heptulosonate-7-phosphate to 5-enolpyruvoyl-shikimate 3-phosphate comprised in SEQ ID NO: 7 or genomic DNA thereof;
e) genes encoding a Shikimate kinase said genes being at least 70%, such at least 75%, such as at least 80%, such as at least 90%, such as at least 95%, such as at least 99%, such as 100% identical to the Shikimate kinase encoding polynucleotide comprised in SEQ ID NO: 9 or genomic DNA thereof;
f) genes encoding a Shikimate kinase said genes being at least 70%, such at least 75%, such as at least 80%, such as at least 90%, such as at least 95%, such as at least 99%, such as 100% identical to the Chorismate synthase encoding polynucleotide comprised in SEQ ID NO: 11 or genomic DNA thereof;
g) genes encoding a Anthranilate synthase said genes being at least 70%, such at least 75%, such as at least 80%, such as at least 90%, such as at least 95%, such as at least 99%, such as 100% identical to the Anthranilate synthase encoding polynucleotide comprised in SEQ ID NO: 13 or genomic DNA thereof;
h) genes encoding a Ribose-phosphate pyrophosphokinase said genes being at least 70%, such at least 75%, such as at least 80%, such as at least 90%, such as at least 95%, such as at least 99%, such as 100% identical to the Ribose-phosphate pyrophosphokinase encoding polynucleotide comprised in SEQ ID NO: 15 or genomic DNA thereof;
i) genes encoding a nthranilate phosphoribosyl transferase said genes being at least 70%, such at least 75%, such as at least 80%, such as at least 90%, such as at least 95%, such as at least 99%, such as 100% identical to the Anthranilate phosphoribosyl transferase encoding polynucleotide comprised in SEQ ID NO: 17 or genomic DNA thereof;
j) genes encoding a N-(5′-phosphoribosyl)-anthranilate isomerase said genes being at least 70%, such at least 75%, such as at least 80%, such as at least 90%, such as at least 95%, such as at least 99%, such as 100% identical to the N-(5′-phosphoribosyl)-anthranilate isomerase encoding polynucleotide comprised in SEQ ID NO: 19 or genomic DNA thereof;
k) genes encoding a Indole-3-glycerol phosphate synthase said genes being at least 70%, such at least 75%, such as at least 80%, such as at least 90%, such as at least 95%, such as at least 99%, such as 100% identical to the Indole-3-glycerol phosphate synthase encoding polynucleotide comprised in SEQ ID NO: 21 or genomic DNA thereof;
l) genes encoding a Tryptophan synthase said genes being at least 70%, such at least 75%, such as at least 80%, such as at least 90%, such as at least 95%, such as at least 99%, such as 100% identical to the Tryptophan synthase encoding polynucleotide comprised in anyone of SEQ ID NO: 23, 59, 61, 63, 65, 67, 179, and/or 181 or genomic DNA thereof;
m) genes encoding a Tryptophan decarboxylase or a non-canonical aromatic amino acid decarboxylase said genes being at least 70%, such at least 75%, such as at least 80%, such as at least 90%, such as at least 95%, such as at least 99%, such as 100% identical to the Tryptophan decarboxylase or a non-canonical aromatic amino acid decarboxylase encoding polynucleotide comprised in SEQ ID NO: 25, 69, 71, 73, 75, and/or 77 or genomic DNA thereof;
n) genes encoding a tryptamine 4-hydroxylase said genes at least 70%, such at least 75%, such as at least 80%, such as at least 90%, such as at least 95%, such as at least 99%, such as 100% identical to the tryptamine 4-hydroxylase encoding polynucleotide comprised in SEQ ID NO 93 and/or 87;
o) genes encoding a cytochrome p450 reductase said genes being at least 70%, such at least 75%, such as at least 80%, such as at least 90%, such as at least 95%, such as at least 99%, such as 100% identical to the cytochrome p450 reductase comprised in anyone of SEQ ID NO 105 and/or 101;
p) genes encoding a cytochrome b5 said genes being at least 70%, such at least 75%, such as at least 80%, such as at least 90%, such as at least 95%, such as at least 99%, such as 100% identical to the Cytochrome b5 encoding polynucleotide comprised in SEQ ID NO 253;
q) genes encoding a 4-hydroxytryptamine kinase said genes being at least 70%, such at least 75%, such as at least 80%, such as at least 90%, such as at least 95%, such as at least 99%, such as 100% identical to the 4-hydroxytryptamine kinase encoding polynucleotide comprised SEQ ID NO 159 and/or 155; and/or
r) genes encoding a psilocybin synthase said genes being at least 70%, such at least 75%, such as at least 80%, such as at least 90%, such as at least 95%, such as at least 99%, such as 100% identical to the psilocybin synthase encoding polynucleotide comprised in SEQ ID NO 127 and/or 123.

Where the host cell producing the psilocybin a yeast strain, such as S. cerevisiae, the host cell is preferably further modified to attenuate, disrupt and/or delete one or more native genes selected from:
a) The pyruvate kinase gene comprised in anyone of SEQ ID NO: 27 or any of its paralogs or orthologs having at least 70%, such at least 75%, such as at least 80%, such as at least 90%, such as at least 95%, such as at least 99%, such as 100% identity to SEQ ID NO: 27;
b) The phosphofructokinase gene comprised in anyone of SEQ ID NO: 29 and/or 31 or any of its paralogs or orthologs having at least 70%, such at least 75%, such as at least 80%, such as at least 90%, such as at least 95%, such as at least 99%, such as 100% identity to anyone of SEQ ID NO: 29 and/or 31;
c) The transporters gene comprised in SEQ ID NO: 33 or any of its paralogs or orthologs having at least 70%, such at least 75%, such as at least 80%, such as at least 90%, such as at least 95%, such as at least 99%, such as 100% identity to SEQ ID NO: 33;
d) The DL-glycerol-3-phosphate phosphatase gene comprised in SEQ ID NO: 34 or any of its paralogs or orthologs having at least 70%, such at least 75%, such as at least 80%, such as at least 90%, such as at least 95%, such as at least 99%, such as 100% identity to SEQ ID NO: 34; and/or
e) The tryptophan 2,3-dioxygenase gene comprised in SEQ ID NO: 35 or any of its paralogs or orthologs having at least 70%, such at least 75%, such as at least 80%, such as at least 90%, such as at least 95%, such as at least 99%, such as 100% identity to SEQ ID NO: 35;
f) The cystathionine beta-synthase gene comprised in SEQ ID NO: 36 or any of its paralogs or orthologs having at least 70%, such at least 75%, such as at least 80%, such as at least 90%, such as at least 95%, such as at least 99%, such as 100% identity to SEQ ID NO: 36;
g) The phenylpyruvate decarboxylase gene comprised in SEQ ID NO: 37 or any of its paralogs or orthologs having at least 70%, such at least 75%, such as at least 80%, such as at least 90%, such as at least 95%, such as at least 99%, such as 100% identity to SEQ ID NO: 37;
h) The pyruvate decarboxylase gene comprised in SEQ ID NO: 38 or any of its paralogs or orthologs having at least 70%, such at least 75%, such as at least 80%, such as at least 90%, such as at least 95%, such as at least 99%, such as 100% identity to SEQ ID NO: 38;
i) The histone variant H2AZ gene comprised in SEQ ID NO: 39 or any of its paralogs or orthologs having at least 70%, such at least 75%, such as at least 80%, such as at least 90%, such as at least 95%, such as at least 99%, such as 100% identity to SEQ ID NO: 39;
j) the phosphatase gene comprised in SEQ ID NO: 40 or any of its paralogs or orthologs having at least 70%, such at least 75%, such as at least 80%, such as at least 90%, such as at least 95%, such as at least 99%, such as 100% identity to SEQ ID NO: 40;
k) the repressible acid phosphatase gene comprised in anyone of SEQ ID NO: 41, 42, or 43 and/or any of its paralogs or orthologs having at least 70%, such at least 75%, such as at least 80%, such as at least 90%, such as at least 95%, such as at least 99%, such as 100% identity to anyone of SEQ ID NO: 41, 42, and/or 43; and/or
l) the constitutively expressed acid phosphatase gene comprised in SEQ ID NO: 44 and/or any of its paralogs or orthologs having at least 70%, such at least 75%, such as at least 80%, such as at least 90%, such as at least 95%, such as at least 99%, such as 100% identity to SEQ ID NO: 44.

Attenuating, disrupting and/or deleting one or more of the native phosphatase, repressible acid phosphatase and/or constitutively expressed acid phosphatase genes is particularly useful, because it has bee found that expression of these phosphatases shunts tryptamine away from psilocybin to psilocin.

In such yeast host cells it is also preferred to further modify it to overexpress one or more native genes selected from the NADH kinase gene comprised in SEQ ID NO: 185 or any of its paralogs or orthologs having at least 70%, such at least 75%, such as at least 80%, such as at least 90%, such as at least 95%, such as at least 99%, such as 100% identity to SEQ ID NO: 185.

In a still further aspect provided herein is a cell culture, comprising host cell producing psilocybin (an indole acceptor) and a growth medium.

In a still further aspect provided herein is a method of producing psilocybin (an indole acceptor) comprising:
c) culturing the said cell culture at conditions allowing the host cell to produce the psilocybin; and
d) optionally recovering and/or isolating the psilocybin.

Such method can in further embodiments include one or more elements selected from:
h) culturing the cell culture in a nutrient growth medium;
i) culturing the cell culture under aerobic or anaerobic conditions
j) culturing the cell culture under agitation;
k) culturing the cell culture at a temperature of between 25 to 50° C.;
I) culturing the cell culture at a pH of between 3-9;
m) culturing the cell culture for between 10 hours to 30 days; and
n) culturing the cell culture under fed-batch, repeated fed-batch, continuous, or semi-continuous conditions.

The method can also comprise feeding one or more exogenous precursors of the psilocybin pathway to the cell culture.

The recovering and/or isolation step can include separating a liquid phase of host cell or cell culture from a solid phase of host cell or cell culture to obtain a supernatant comprising the psilocybin by one or more steps selected from:
g) disrupting the host cell to release intracellular psilocybin indole acceptor into the supernatant;
h) separating the supernatant from the solid phase of the host cell, such as by filtration or gravity separation;
i) contacting the supernatant with one or more adsorbent resins in order to obtain at least a portion of the produced psilocybin;
j) contacting the supernatant with one or more ion exchange or reversed-phase chromatography columns in order to obtain at least a portion of the psilocybin;
k) extracting the psilocybin; and
l) precipitating the psilocybin indole acceptor by crystallization or evaporating the solvent of the liquid phase; and optionally isolating the psilocybin by filtration or gravity separation; thereby recovering and/or isolating the psilocybin.

In a still further aspect provided herein is a fermentation liquid/composition comprising the psilocybin comprised in the cell culture or the growth medium. In such fermentation liquids at least 50%, such as at least 75%, such as at least 95%, such as at least 99% of the host cells may be disrupted and further at least 50%, such as at least 75%, such as at least 95%, such as at least 99% of solid cellular material has separated from the liquid. Moreover, in addition to the psilocybin the fermentation liquid/composition of the invention may also comprise precursors, products, metabolites of the psilocybin pathway, in particular tryptophan and/or tryptamine or comprise one or more compounds selected from trace metals, vitamins, salts, yeast nitrogen base, carbon source, YNB, and/or amino acids of the fermentation. In particular the fermentation liquid/composition comprises a concentration of psilocybin of at least 1 mg/kg or mg/L composition, such as at least 5 mg/kg or mg/L, such as at least 10 mg/kg or mg/L, such as at least 20 mg/kg or mg/L, such as at least 50 mg/kg or mg/L, such as at least 100 mg/kg or mg/L, such as at least 500 mg/kg or mg/L, such as at least 1000 mg/kg or mg/L, such as at least 5000 mg/kg or mg/L, such as at least 10000 mg/kg or mg/L, such as at least 50000 mg/kg or mg/L.

In a further aspect provided herein is a composition comprising the fermentation liquid/composition of the invention and one or more carriers, agents, additives and/or excipients. Carriers, agents, additives and/or excipients includes formulation additives, stabilising agent, fillers, and the like. The composition may be formulated into a dry solid form, such as powders, tablets, capsules, hard chewables and or soft lozenges or a gums by using methods known in the art, such as spray drying, spray cooling, lyophilization, flash freezing, granulation, microgranulation, encapsulation or microencapsulation. The composition may also be formulated into liquid stabilized form using methods known in the art, such as formulation into a stabilized liquid comprising one or more stabilizers such as sugars and/or polyols (e.g. sugar alcohols) and/or organic acids (e.g. lactic acid).

### Sequences

The present application contains a Sequence Listing prepared in PatentIn version 3.5.1, which is also submitted electronically in ST25 format which is hereby incorporated by reference in its entirety. For further reference, the following sequences are included with this application:

| | | | | | | |
|---|---|---|---|---|---|---|
| SEQ ID | DNA | sequence | Fructose-6-phosphate | BfXfpk | From | Bifidobacterium |
| NO: 1 | | of | Phosphoketolase | | | breve |
| SEQ ID | Protein | sequence | Fructose-6-phosphate | BfXfpk | From | Bifidobacterium |
| NO: 2 | | of | Phosphoketolase | | | breve |
| SEQ ID | DNA | sequence | Phosphotransacetylase | CkPta | From | Clostridium kluyveri |
| NO: 3 | | of | | | | |
| SEQ ID | Protein | sequence | Phosphotransacetylase | CkPta | From | Clostridium kluyveri |
| NO: 4 | | of | | | | |
| SEQ ID | DNA | sequence | DAHP synthase | ARO4(K229L) | From | Saccharomyces |
| NO: 5 | | of | | | | cerevisiae |
| SEQ ID | Protein | sequence | DAHP synthase | ARO4(K229L) | From | Saccharomyces |
| NO: 6 | | of | | | | cerevisiae |
| SEQ ID | DNA | sequence | Multiple native enzymatic | ARO1 | From | Saccharomyces |
| NO: 7 | | of | steps | | | cerevisiae |
| SEQ ID | Protein | sequence | Multiple native enzymatic | ARO1 | From | Saccharomyces |
| NO: 8 | | of | steps | | | cerevisiae |
| SEQ ID | DNA | sequence | Shikimate kinase | EcAroL | From | Escherichia coli |
| NO: 9 | | of | | | | |
| SEQ ID | Protein | sequence | Shikimate kinase | EcAroL | From | Escherichia coli |
| NO: 10 | | of | | | | |
| SEQ ID | DNA | sequence | Chorismate synthase | ARO2 | From | Saccharomyces |
| NO: 11 | | of | | | | cerevisiae |
| SEQ ID | Protein | sequence | Chorismate synthase | ARO2 | From | Saccharomyces |
| NO: 12 | | of | | | | cerevisiae |
| SEQ ID | DNA | sequence | Anthranilate synthase | TRP2(S65R, S76L) | From | Saccharomyces |
| NO: 13 | | of | | | | cerevisiae |
| SEQ ID | Protein | sequence | Anthranilate synthase | TRP2(S65R, S76L) | From | Saccharomyces |
| NO: 14 | | of | | | | cerevisiae |
| SEQ ID | DNA | sequence | Ribose-phosphate | BsPrs | From | Bacillus subtilis |
| NO: 15 | | of | pyrophosphokinase | | | |
| SEQ ID | Protein | sequence | Ribose-phosphate | BsPrs | From | Bacillus subtilis |
| NO: 16 | | of | pyrophosphokinase | | | |
| SEQ ID | DNA | sequence | Anthranilate | TRP4 | From | Saccharomyces |
| NO: 17 | | of | phosphoribosyl | | | cerevisiae |
| | | | transferase | | | |
| SEQ ID | Protein | sequence | Anthranilate | TRP4 | From | Saccharomyces |
| NO: 18 | | of | phosphoribosyl | | | cerevisiae |
| | | | transferase | | | |
| SEQ ID | DNA | sequence | N-(5′-phosphoribosyl)- | TRP1 | From | Saccharomyces |
| NO: 19 | | of | anthranilate isomerase | | | cerevisiae |
| SEQ ID | Protein | sequence | N-(5′-phosphoribosyl)- | TRP1 | From | Saccharomyces |
| NO: 20 | | of | anthranilate isomerase | | | cerevisiae |
| SEQ ID | DNA | sequence | Indole-3-glycerol | TRP3 | From | Saccharomyces |
| NO: 21 | | of | phosphate synthase | | | cerevisiae |
| SEQ ID | Proteir | sequence | Indole-3-glycerol | TRP3 | From | Saccharomyces |
| NO: 22 | | of | phosphate synthase | | | cerevisiae |
| SEQ ID | DNA | sequence | Tryptophan synthase | TRP5 | From | Saccharomyces |
| NO: 23 | | of | | | | cerevisiae |
| SEQ ID | Protein | sequence | Tryptophan synthase | TRP5 | From | Saccharomyces |
| NO: 24 | | of | | | | cerevisiae |
| SEQ ID | DNA | sequence | Tryptophan | CrTdc | From | Catharanthus roseus |
| NO: 25 | | of | decarboxylase | | | |
| SEQ ID | Protein | sequence | Tryptophan | CrTdc | From | Catharanthus roseus |
| NO: 26 | | of | decarboxylase | | | |
| SEQ ID | DNA | sequence | Pyruvate kinase | CDC19 | From | Saccharomyces |
| NO: 27 | | of | | | | cerevisiae |
| SEQ ID | Protein | sequence | Pyruvate kinase | CDC19 | From | Saccharomyces |
| NO: 28 | | of | | | | cerevisiae |
| SEQ ID | DNA | sequence | Phosphofructokinase | PFK1 | From | Saccharomyces |
| NO: 29 | | of | | | | cerevisiae |
| SEQ ID | Protein | sequence | Phosphofructokinase | PFK1 | From | Saccharomyces |
| NO: 30 | | of | | | | cerevisiae |
| SEQ ID | DNA | sequence | Phosphofructokinase | PFK2 | From | Saccharomyces |
| NO: 31 | | of | | | | cerevisiae |
| SEQ ID | Protein | sequence | Phosphofructokinase | PFK2 | From | Saccharomyces |
| NO: 32 | | of | | | | cerevisiae |
| SEQ ID | DNA | sequence | cis-Golgi network | RIC1 | From | Saccharomyces |
| NO: 33 | | of | transporter protein | | | cerevisiae |
| SEQ ID | DNA | sequence | DL-glycerol-3-phosphate | GPP1 | From | Saccharomyces |
| NO: 34 | | of | phosphatase | | | cerevisiae |
| SEQ ID | DNA | sequence | Tryptophan 2,3- | BNA2 | From | Saccharomyces |
| NO: 35 | | of | dioxygenase | | | cerevisiae |
| SEQ ID | DNA | sequence | Cystathionine beta- | CYS4 | From | Saccharomyces |
| NO: 36 | | of | synthase | | | cerevisiae |
| SEQ ID | DNA | sequence | Phenylpyruvate | ARO10 | From | Saccharomyces |
| NO: 37 | | of | decarboxylase | | | cerevisiae |
| SEQ ID | DNA | sequence | Pyruvate decarboxylase | PDC5 | From | Saccharomyces |
| NO: 38 | | of | | | | cerevisiae |
| SEQ ID | DNA | sequence | Histone variant H2AZ | HTZ1 | From | Saccharomyces |
| NO: 39 | | of | | | | cerevisiae |
| SEQ ID | DNA | sequence | Unknown phosphatase | DIA3 | From | Saccharomyces |
| NO: 40 | | of | | | | cerevisiae |
| SEQ ID | DNA | sequence | Repressible acid | PHO5 | From | Saccharomyces |
| NO: 41 | | of | phosphatase | | | cerevisiae |
| SEQ ID | DNA | sequence | Repressible acid | PHO12 | From | Saccharomyces |
| NO: 42 | | of | phosphatase | | | cerevisiae |
| SEQ ID | DNA | sequence | Repressible acid | PHO11 | From | Saccharomyces |
| NO: 43 | | of | phosphatase | | | cerevisiae |
| SEQ ID | DNA | sequence | Constitutive acid | PHO3 | From | Saccharomyces |
| NO: 44 | | of | phosphatase | | | cerevisiae |
| SEQ ID | DNA | sequence | Chorismate mutase | ARO7(G141S) | From | Saccharomyces |
| NO: 45 | | of | | | | cerevisiae |
| SEQ ID | Protein | sequence | Chorismate mutase | ARO7(G141S) | From | Saccharomyces |
| NO: 46 | | of | | | | cerevisiae |
| SEQ ID | DNA | sequence | Prephenate | PHA2 | From | Saccharomyces |
| NO: 47 | | of | dehydrogenase | | | cerevisiae |
| SEQ ID | Protein | sequence | Prephenate | PHA2 | From | Saccharomyces |
| NO: 48 | | of | dehydrogenase | | | cerevisiae |
| SEQ ID | DNA | sequence | Aromatic | ARO8 | From | Saccharomyces |
| NO: 49 | | of | aminotransferase | | | cerevisiae |
| SEQ ID | Protein | sequence | Aromatic | ARO8 | From | Saccharomyces |
| NO: 50 | | of | aminotransferase | | | cerevisiae |
| SEQ ID | DNA | sequence | Phenylalanine | AtPAL2 | From | Arabidopsis thaliana |
| NO: 51 | | of | ammonium lyase | | | |
| SEQ ID | Protein | sequence | Phenylalanine | AtPAL2 | From | Arabidopsis thaliana |
| NO: 52 | | of | ammonium lyase | | | |
| SEQ ID | DNA | sequence | Cinnimate 4-hydroxylase | AtC4H | From | Arabidopsis thaliana |
| NO: 53 | | of | | | | |
| SEQ ID | Protein | sequence | Cinnimate 4-hydroxylase | AtC4H | From | Arabidopsis thaliana |
| NO: 54 | | of | | | | |
| SEQ ID | DNA | sequence | Cytochrome P450 | AtATR2 | From | Arabidopsis thaliana |
| NO: 55 | | of | reductase | | | |
| SEQ ID | Protein | sequence | Cytochrome P450 | AtATR2 | From | Arabidopsis thaliana |
| NO: 56 | | of | reductase | | | |
| SEQ ID | DNA | sequence | 4-Coumoryl-CoA ligase | At4CL2 | From | Arabidopsis thaliana |
| NO: 57 | | of | | | | |
| SEQ ID | Protein | sequence | 4-Coumoryl-CoA ligase | At4CL2 | From | Arabidopsis thaliana |
| NO: 58 | | of | | | | |
| SEQ ID | DNA | sequence | Tryptophan synthase | PfTrpB(2B9) | From | Pyrococcus furiosus |
| NO: 59 | | of | beta subunit | | | |
| SEQ ID | Protein | sequence | Tryptophan synthase | PfTrpB(2B9) | From | Pyrococcus furiosus |
| NO: 60 | | of | beta subunit | | | |
| SEQ ID | DNA | sequence | Tryptophan synthase | SeTrpB | From | Salmonella enterica |
| NO: 61 | | of | beta subunit | | | |
| SEQ ID | Protein | sequence | Tryptophan synthase | SeTrpB | From | Salmonella enterica |
| NO: 62 | | of | beta subunit | | | |
| SEQ ID | DNA | sequence | Tryptophan synthase | PfTrpB | From | Pyrococcus furiosus |
| NO: 63 | | of | beta subunit | | | |
| SEQ ID | Protein | sequence | Tryptophan synthase | PfTrpB | From | Pyrococcus furiosus |
| NO: 64 | | of | beta subunit | | | |
| SEQ ID | DNA | sequence | Tryptophan synthase | TRP5 | From | Saccharomyces |
| NO: 65 | | of | | | | cerevisiae |
| SEQ ID | Protein | sequence | Tryptophan synthase | TRP5 | From | Saccharomyces |
| NO: 66 | | of | | | | cerevisiae |
| SEQ ID | DNA | sequence | Tryptophan synthase | TmTrpB (M145T, | From | Thermotoga maritima |
| NO: 67 | | of | beta subunit | N167D) | | |
| SEQ ID | Protein | sequence | Tryptophan synthase | TmTrpB (M145T, | From | Thermotoga maritima |
| NO: 68 | | of | beta subunit | N167D) | | |
| SEQ ID | DNA | sequence | Tryptophan | RgTdc | From | Ruminococcus gnavus |
| NO: 69 | | of | decarboxylase | | | |
| SEQ ID | Protein | sequence | Tryptophan | RgTdc | From | Ruminococcus gnavus |
| NO: 70 | | of | decarboxylase | | | |
| SEQ ID | DNA | sequence | Tryptophan | PcPsiD | From | Psilocybe cubensis |
| NO: 71 | | of | decarboxylase | | | |
| SEQ ID | Protein | sequence | Tryptophan | PcPsiD | From | Psilocybe cubensis |
| NO: 72 | | of | decarboxylase | | | |
| SEQ ID | DNA | sequence | aromatic amino acid | PcncAAAD | From | Psilocybe cubensis |
| NO: 73 | | of | decarboxylase | | | |
| SEQ ID | Protein | sequence | aromatic amino acid | PcncAAAD | From | Psilocybe cubensis |
| NO: 74 | | of | decarboxylase | | | |
| SEQ ID | DNA | sequence | Tryptophan | PsPsiD | From | Psilocybe serbica |
| NO: 75 | | of | decarboxylase | | | |
| SEQ ID | Protein | sequence | Tryptophan | PsPsiD | From | Psilocybe serbica |
| NO: 76 | | of | decarboxylase | | | |
| SEQ ID | DNA | sequence | Tryptophan | PanCyPsiD | From | Panaeolus |
| NO: 77 | | of | decarboxylase | | | cyanescens |
| SEQ ID | Protein | sequence | Tryptophan | PanCyPsiD | From | Panaeolus |
| NO: 78 | | of | decarboxylase | | | cyanescens |
| SEQ ID | DNA | sequence | UGT | Cs73Y | From | Crocus sativus |
| NO: 79 | | of | | | | |
| SEQ ID | Protein | sequence | UGT | Cs73Y | From | Crocus sativus |
| NO: 80 | | of | | | | |
| SEQ ID | DNA | sequence | UGT | Pt73Y | From | Populus trichocarpa |
| NO: 81 | | of | | | | |
| SEQ ID | Protein | sequence | UGT | Pt73Y | From | Populus trichocarpa |
| NO: 82 | | of | | | | |
| SEQ ID | DNA | sequence | UGT | Ha88B_2 | From | Helianthus annuus |
| NO: 83 | | of | | | | |
| SEQ ID | Protein | sequence | UGT | Ha88B_2 | From | Helianthus annuus |
| NO: 84 | | of | | | | |
| SEQ ID | DNA | sequence | UGT | PtIGS | From | Persicaria tinctoria |
| NO: 85 | | of | | | | |
| SEQ ID | Protein | sequence | UGT | PtIGS | From | Persicaria tinctoria |
| NO: 86 | | of | | | | |
| SEQ ID | DNA | sequence | Hydroxylase | PcPsiH | From | Psilocybe cubensis |
| NO: 87 | | of | | | | |
| SEQ ID | Protein | sequence | Hydroxylase | PcPsiH | From | Psilocybe cubensis |
| NO: 88 | | of | | | | |
| SEQ ID | DNA | sequence | Hydroxylase | PsPsiH1 | From | Psilocybe serbica |
| NO: 89 | | of | | | | |
| SEQ ID | Protein | sequence | Hydroxylase | PsPsiH1 | From | Psilocybe serbica |
| NO: 90 | | of | | | | |
| SEQ ID | DNA | sequence | Hydroxylase | PsPsiH2 | From | Psilocybe serbica |
| NO: 91 | | of | | | | |
| SEQ ID | Protein | sequence | Hydroxylase | PsPsiH2 | From | Psilocybe serbica |
| NO: 92 | | of | | | | |
| SEQ ID | DNA | sequence | Hydroxylase | PanCyPsiH | From | Panaeolus |
| NO: 93 | | of | | | | cyanescens |
| SEQ ID | Protein | sequence | Hydroxylase | PanCyPsiH | From | Panaeolus |
| NO: 94 | | of | | | | cyanescens |
| SEQ ID | DNA | sequence | Hydroxylase | OsT5H | From | Orzya Sativa |
| NO: 95 | | of | | | | |
| SEQ ID | Protein | sequence | Hydroxylase | OsT5H | From | Orzya Sativa |
| NO: 96 | | of | | | | |
| SEQ ID | DNA | sequence | Hydroxylase | PtFMO | From | Persicaria tinctoria |
| NO: 97 | | of | | | | |
| SEQ ID | Protein | sequence | Hydroxylase | PtFMO | From | Persicaria tinctoria |
| NO: 98 | | of | | | | |
| SEQ ID | DNA | sequence | Hydroxylase | Til10H | From | Tabernanthe iboga |
| NO: 99 | | of | | | | |
| SEQ ID | Protein | sequence | Hydroxylase | Til10H | From | Tabernanthe iboga |
| NO: 100 | | of | | | | |
| SEQ ID | DNA | sequence | CPR | PcCPR | From | Psilocybe cubensis |
| NO: 101 | | of | | | | |
| SEQ ID | Protein | sequence | CPR | PcCPR | From | Psilocybe cubensis |
| NO: 102 | | of | | | | |
| SEQ ID | DNA | sequence | CPR | PsCPR | From | Psilocybe serbica |
| NO: 103 | | of | | | | |
| SEQ ID | Protein | sequence | CPR | PsCPR | From | Psilocybe serbica |
| NO: 104 | | of | | | | |
| SEQ ID | DNA | sequence | CPR | PanCyCPR | From | Panaeolus |
| NO: 105 | | of | | | | cyanescens |
| SEQ ID | Protein | sequence | CPR | PanCyCPR | From | Panaeolus |
| NO: 106 | | of | | | | cyanescens |
| SEQ ID | DNA | sequence | CPR | CrCPR1 | From | Catharanthus roseus |
| NO: 107 | | of | | | | |
| SEQ ID | Protein | sequence | CPR | CrCPR1 | From | Catharanthus roseus |
| NO: 108 | | of | | | | |
| SEQ ID | DNA | sequence | CPR | CrCPR2 | From | Catharanthus roseus |
| NO: 109 | | of | | | | |
| SEQ ID | Protein | sequence | CPR | CrCPR2 | From | Catharanthus roseus |
| NO: 110 | | of | | | | |
| SEQ ID | DNA | sequence | CPR | FoCPR | From | Fusarium oxysporum |
| NO: 111 | | of | | | | |
| SEQ ID | Protein | sequence | CPR | FoCPR | From | Fusarium oxysporum |
| NO: 112 | | of | | | | |
| SEQ ID | DNA | sequence | O-methyl transferase | AtCOMT1 | From | Arabidopsis thaliana |
| NO: 113 | | of | | | | |
| SEQ ID | Protein | sequence | O-methyl transferase | AtCOMT1 | From | Arabidopsis thaliana |
| NO: 114 | | of | | | | |
| SEQ ID | DNA | sequence | O-methyl transferase | AtIGMT1 | From | Arabidopsis thaliana |
| NO: 115 | | of | | | | |
| SEQ ID | Protein | sequence | O-methyl transferase | AtIGMT1 | From | Arabidopsis thaliana |
| NO: 116 | | of | | | | |
| SEQ ID | DNA | sequence | O-methyl transferase | HsASMT | From | Homo sapiens |
| NO: 117 | | of | | | | |
| SEQ ID | Protein | sequence | O-methyl transferase | HsASMT | From | Homo sapiens |
| NO: 118 | | of | | | | |
| SEQ ID | DNA | sequence | O-methyl transferase | TiN10OMT | From | Tabernanthe iboga |
| NO: 119 | | of | | | | |
| SEQ ID | Protein | sequence | O-methyl transferase | TiN10OMT | From | Tabernanthe iboga |
| NO: 120 | | of | | | | |
| SEQ ID | DNA | sequence | N-methyl transferase | OcINMT | From | Oryctolagus cuniculus |
| NO: 121 | | of | | | | |
| SEQ ID | Protein | sequence | N-methyl transferase | OcINMT | From | Oryctolagus cuniculus |
| NO: 122 | | of | | | | |
| SEQ ID | DNA | sequence | N-methyl transferase | PcPsiM | From | Psilocybe cubensis |
| NO: 123 | | of | | | | |
| SEQ ID | Protein | sequence | N-methyl transferase | PcPsiM | From | Psilocybe cubensis |
| NO: 124 | | of | | | | |
| SEQ ID | DNA | sequence | N-methyl transferase | PsPsiM | From | Psilocybe serbica |
| NO: 125 | | of | | | | |
| SEQ ID | Protein | sequence | N-methyl transferase | PsPsiM | From | Psilocybe serbica |
| NO: 126 | | of | | | | |
| SEQ ID | DNA | sequence | N-methyl transferase | PanCyPsiM | From | Panaeolus |
| NO: 127 | | of | | | | cyanescens |
| SEQ ID | Protein | sequence | N-methyl transferase | PanCyPsiM | From | Panaeolus |
| NO: 128 | | of | | | | cyanescens |
| SEQ ID | DNA | sequence | N-methyl transferase | PcPsiM(H210A) | From | Psilocybe cubensis |
| NO: 129 | | of | | | | |
| SEQ ID | Protein | sequence | N-methyl transferase | PcPsiM(H210A) | From | Psilocybe cubensis |
| NO: 130 | | of | | | | |
| SEQ ID | DNA | sequence | N-methyl transferase | PsTrpM | From | Psilocybe serbica |
| NO: 131 | | of | | | | |
| SEQ ID | Protein | sequence | N-methyl transferase | PsTrpM | From | Psilocybe serbica |
| NO: 132 | | of | | | | |
| SEQ ID | DNA | sequence | N-methyl transferase | MsEgtD (M252A, | From | Mycobacterium |
| NO: 133 | | of | | E282A) | | smegmatis |
| SEQ ID | Protein | sequence | N-methyl transferase | MsEgtD (M252A, | From | Mycobacterium |
| NO: 134 | | of | | E282A) | | smegmatis |
| SEQ ID | DNA | sequence | N-methyl transferase | CsSNMT | From | Citrus sinensis |
| NO: 135 | | of | | | | |
| SEQ ID | Protein | sequence | N-methyl transferase | CsSNMT | From | Citrus sinensis |
| NO: 136 | | of | | | | |
| SEQ ID | DNA | sequence | N-methyl transferase | CsSNMT1(ΔN-term) | From | Citrus sinensis |
| NO: 137 | | of | | | | |
| SEQ ID | Protein | sequence | N-methyl transferase | CsSNMT1(ΔN-term) | From | Citrus sinensis |
| NO: 138 | | of | | | | |
| SEQ ID | DNA | sequence | C-methyl transferase | SITsrM | From | Streptomyces |
| NO: 139 | | of | | | | laurentii |
| SEQ ID | Protein | sequence | C-methyl transferase | SITsrM | From | Streptomyces |
| NO: 140 | | of | | | | laurentii |
| SEQ ID | DNA | sequence | Acetyl transferase | BtAANAT | From | Bos Taurus |
| NO: 141 | | of | | | | |
| SEQ ID | Protein | sequence | Acetyl transferase | BtAANAT | From | Bos Taurus |
| NO: 142 | | of | | | | |
| SEQ ID | DNA | sequence | Syntase | OpSTR | From | Ophiorrhiza pumila |
| NO: 143 | | of | | | | |
| SEQ ID | Protein | sequence | Syntase | OpSTR | From | Ophiorrhiza pumila |
| NO: 144 | | of | | | | |
| SEQ ID | DNA | sequence | Syntase | RsSTR1 (V208A) | From | Rauvolfia serpentina |
| NO: 145 | | of | | | | |
| SEQ ID | Protein | sequence | Syntase | RsSTR1 (V208A) | From | Rauvolfia serpentina |
| NO: 146 | | of | | | | |
| SEQ ID | DNA | sequence | Syntase | CrSTR1 | From | Catharanthus roseus |
| NO: 147 | | of | | | | |
| SEQ ID | Protein | sequence | Syntase | CrSTR1 | From | Catharanthus roseus |
| NO: 148 | | of | | | | |
| SEQ ID | DNA | sequence | Syntase | CrSTR1 (D177A) | From | Catharanthus roseus |
| NO: 149 | | of | | | | |
| SEQ ID | Protein | sequence | Syntase | CrSTR1 (D177A) | From | Catharanthus roseus |
| NO: 150 | | of | | | | |
| SEQ ID | DNA | sequence | Syntase | MtMcbB | From | Marinactinospora |
| NO: 151 | | of | | | | thermotolerans |
| SEQ ID | Protein | sequence | Syntase | MtMcbB | From | Marinactinospora |
| NO: 152 | | of | | | | thermotolerans |
| SEQ ID | DNA | sequence | Syntase | MtMcbB (R72A, | From | Marinactinospora |
| NO: 153 | | of | | H87A) | | thermotolerans |
| SEQ ID | Protein | sequence | Syntase | MtMcbB (R72A, | From | Marinactinospora |
| NO: 154 | | of | | H87A) | | thermotolerans |
| SEQ ID | DNA | sequence | Kinase | PcPsiK | From | Psilocybe cubensis |
| NO: 155 | | of | | | | |
| SEQ ID | Protein | sequence | Kinase | PcPsiK | From | Psilocybe cubensis |
| NO: 156 | | of | | | | |
| SEQ ID | DNA | sequence | Kinase | PsPsiK | From | Psilocybe serbica |
| NO: 157 | | of | | | | |
| SEQ ID | Protein | sequence | Kinase | PsPsiK | From | Psilocybe serbica |
| NO: 158 | | of | | | | |
| SEQ ID | DNA | sequence | Kinase | PanCyPsiK | From | Panaeolus |
| NO: 159 | | of | | | | cyanescens |
| SEQ ID | Protein | sequence | Kinase | PanCyPsiK | From | Panaeolus |
| NO: 160 | | of | | | | cyanescens |
| SEQ ID | DNA | sequence | Cin trans (N-hydroxy- | CaSHT | From | Capsicum annuum |
| NO: 161 | | of | cinnamoyl transferase) | | | |
| SEQ ID | Protein | sequence | Cin trans (N-hydroxy- | CaSHT | From | Capsicum annuum |
| NO: 162 | | of | cinnamoyl transferase) | | | |
| SEQ ID | DNA | sequence | Phosphatase | PcPsiL | From | Psilocybe cubensis |
| NO: 163 | | of | | | | |
| SEQ ID | Protein | sequence | Phosphatase | PcPsiL | From | Psilocybe cubensis |
| NO: 164 | | of | | | | |
| SEQ ID | DNA | sequence | Lacccase | PcPsiP | From | Psilocybe cubensis |
| NO: 165 | | of | | | | |
| SEQ ID | Protein | sequence | Lacccase | PcPsiP | From | Psilocybe cubensis |
| NO: 166 | | of | | | | |
| SEQ ID | DNA | sequence | Halogenase | CcCmdE | From | Chondromyces |
| NO: 167 | | of | | | | crocatus |
| SEQ ID | Protein | sequence | Halogenase | CcCmdE | From | Chondromyces |
| NO: 168 | | of | | | | crocatus |
| SEQ ID | DNA | sequence | Halogenase | SrPyrH | From | Streptomyces |
| NO: 169 | | of | | | | rugosporus |
| SEQ ID | Protein | sequence | Halogenase | SrPyrH | From | Streptomyces |
| NO: 170 | | of | | | | rugosporus |
| SEQ ID | DNA | sequence | Halogenase | Stth | From | Streptomyces |
| NO: 171 | | of | | | | toxytricini |
| SEQ ID | Protein | sequence | Halogenase | Stth | From | Streptomyces |
| NO: 172 | | of | | | | toxytricini |
| SEQ ID | DNA | sequence | Halogenase | LaRebH | From | Lechevalieria |
| NO: 173 | | of | | | | aerocolonigenes |
| SEQ ID | Protein | sequence | Halogenase | LaRebH | From | Lechevalieria |
| NO: 174 | | of | | | | aerocolonigenes |
| SEQ ID | DNA | sequence | Lyase | EcTnaA | From | Escherichia coli |
| NO: 175 | | of | | | | |
| SEQ ID | Protein | sequence | Lyase | EcTnaA | From | Escherichia coli |
| NO: 176 | | of | | | | |
| SEQ ID | DNA | sequence | P450 monooxygesase | SsTxtE | From | Streptomyces scabies |
| NO: 177 | | of | | | | |
| SEQ ID | Protein | sequence | P450 monooxygesase | SsTxtE | From | Streptomyces scabies |
| NO: 178 | | of | | | | |
| SEQ ID | DNA | sequence | Tryptophan synthase | PcTrpB | From | Psilocybe cubensis |
| NO: 179 | | of | | | | |
| SEQ ID | Protein | sequence | Tryptophan synthase | PcTrpB | From | Psilocybe cubensis |
| NO: 180 | | of | | | | |
| SEQ ID | DNA | sequence | Tryptophan synthase | PcTrpB (M439T, | From | Psilocybe cubensis |
| NO: 181 | | of | | N459D) | | |
| SEQ ID | Protein | sequence | Tryptophan synthase | PcTrpB (M439T, | From | Psilocybe cubensis |
| NO: 182 | | of | | N459D) | | |
| SEQ ID | DNA | sequence | C-24(28) sterol reductase | ERG4 | From | Saccharomyces |
| NO: 183 | | of | | | | cerevisiae |
| SEQ ID | DNA | sequence | S-adenosylmethionine | SPE2 | From | Saccharomyces |
| NO: 184 | | of | decarboxylase | | | cerevisiae |
| SEQ ID | DNA | sequence | NADH kinase | POS5 | From | Saccharomyces |
| NO: 185 | | of | | | | cerevisiae |
| SEQ ID | Protein | sequence | NADH kinase | POS5 | From | Saccharomyces |
| NO: 186 | | of | | | | cerevisiae |
| SEQ ID | DNA | sequence | UGT | Ha72B | From | H. annuus |
| NO: 187 | | of | | | | |
| SEQ ID | Protein | sequence | UGT | Ha72B | From | H. annuus |
| NO: 188 | | of | | | | |
| SEQ ID | DNA | sequence | UGT | Ha72T | From | H. annuus |
| NO: 189 | | of | | | | |
| SEQ ID | Protein | sequence | UGT | Ha72T | From | H. annuus |
| NO: 190 | | of | | | | |
| SEQ ID | DNA | sequence | UGT | Sp72T | From | S. pennellii |
| NO: 191 | | of | | | | |
| SEQ ID | Protein | sequence | UGT | Sp72T | From | S. pennellii |
| NO: 192 | | of | | | | |
| SEQ ID | DNA | sequence | UGT | At71C2 | From | A. thaliana |
| NO: 193 | | of | | | | |
| SEQ ID | Protein | sequence | UGT | At71C2 | From | A. thaliana |
| NO: 194 | | of | | | | |
| SEQ ID | DNA | sequence | UGT | RsAS | From | R. serpentina |
| NO: 195 | | of | | | | |
| SEQ ID | Protein | sequence | UGT | RsAS | From | R. serpentina |
| NO: 196 | | of | | | | |
| SEQ ID | DNA | sequence | UGT | At72B1 | From | A. thaliana |
| NO: 197 | | of | | | | |
| SEQ ID | Protein | sequence | UGT | At72B1 | From | A. thaliana |
| NO: 198 | | of | | | | |
| SEQ ID | DNA | sequence | UGT | At71C1-Sr71E1_354 | From | A. thaliana-S. |
| NO: 199 | | of | | | | rebaudiana hybrid |
| SEQ ID | Protein | sequence | UGT | At71C1-Sr71E1_354 | From | A. thaliana-S. |
| NO: 200 | | of | | | | rebaudiana hybrid |
| SEQ ID | DNA | sequence | UGT | At73B5 | From | A. thaliana |
| NO: 201 | | of | | | | |
| SEQ ID | Protein | sequence | UGT | At73B5 | From | A. thaliana |
| NO: 202 | | of | | | | |
| SEQ ID | DNA | sequence | UGT | Pt73Y | From | P. trichocarpa |
| NO: 203 | | of | | | | |
| SEQ ID | Protein | sequence | UGT | Pt73Y | From | P. trichocarpa |
| NO: 204 | | of | | | | |
| SEQ ID | DNA | sequence | UGT | Sp72Q | From | S. pennellii |
| NO: 205 | | of | | | | |
| SEQ ID | Protein | sequence | UGT | Sp72Q | From | S. pennellii |
| NO: 206 | | of | | | | |
| SEQ ID | DNA | sequence | UGT | Si72V | From | S. indicum |
| NO: 207 | | of | | | | |
| SEQ ID | Protein | sequence | UGT | Si72V | From | S. indicum |
| NO: 208 | | of | | | | |
| SEQ ID | DNA | sequence | UGT | Ad72X | From | A. duranensis |
| NO: 209 | | of | | | | |
| SEQ ID | Protein | sequence | UGT | Ad72X | From | A. duranensis |
| NO: 210 | | of | | | | |
| SEQ ID | DNA | sequence | UGT | Zj71A | From | Z. jujube |
| NO: 211 | | of | | | | |
| SEQ ID | Protein | sequence | UGT | Zj71A | From | Z. jujube |
| NO: 212 | | of | | | | |
| SEQ ID | DNA | sequence | UGT | OsEUGT11 | From | O. sativa |
| NO: 213 | | of | | | | |
| SEQ ID | Protein | sequence | UGT | OsEUGT11 | From | O. sativa |
| NO: 214 | | of | | | | |
| SEQ ID | DNA | sequence | UGT | Tc74Z | From | T. cacao |
| NO: 215 | | of | | | | |
| SEQ ID | Protein | sequence | UGT | Tc74Z | From | T. cacao |
| NO: 216 | | of | | | | |
| SEQ ID | DNA | sequence | UGT | At73C5 | From | A. thaliana |
| NO: 217 | | of | | | | |
| SEQ ID | Protein | sequence | UGT | At73C5 | From | A. thaliana |
| NO: 218 | | of | | | | |
| SEQ ID | DNA | sequence | UGT | At76E11 | From | A. thaliana |
| NO: 219 | | of | | | | |
| SEQ ID | Protein | sequence | UGT | At76E11 | From | A. thaliana |
| NO: 220 | | of | | | | |
| SEQ ID | DNA | sequence | UGT | CrUGT-2 | From | C. roseus |
| NO: 221 | | of | | | | |
| SEQ ID | Protein | sequence | UGT | CrUGT-2 | From | C. roseus |
| NO: 222 | | of | | | | |
| SEQ ID | DNA | sequence | UGT | Eg85S | From | E. grandis |
| NO: 223 | | of | | | | |
| SEQ ID | Protein | sequence | UGT | Eg85S | From | E. grandis |
| NO: 224 | | of | | | | |
| SEQ ID | DNA | sequence | UGT | Si85B | From | S. indicum |
| NO: 225 | | of | | | | |
| SEQ ID | Protein | sequence | UGT | Si85B | From | S. indicum |
| NO: 226 | | of | | | | |
| SEQ ID | DNA | sequence | UGT | Cs73Y | From | C. sativus |
| NO: 227 | | of | | | | |
| SEQ ID | Protein | sequence | UGT | Cs73Y | From | C. sativus |
| NO: 228 | | of | | | | |
| SEQ ID | DNA | sequence | UGT | Ha74B | From | H. annuus |
| NO: 229 | | of | | | | |
| SEQ ID | Protein | sequence | UGT | Ha74B | From | H. annuus |
| NO: 230 | | of | | | | |
| SEQ ID | DNA | sequence | UGT | Ha76H_2 | From | H. annuus |
| NO: 231 | | of | | | | |
| SEQ ID | Protein | sequence | UGT | Ha76H_2 | From | H. annuus |
| NO: 232 | | of | | | | |
| SEQ ID | DNA | sequence | UGT | Bv73P | From | B. vulgaris |
| NO: 233 | | of | | | | |
| SEQ ID | Protein | sequence | UGT | Bv73P | From | B. vulgaris |
| NO: 234 | | of | | | | |
| SEQ ID | DNA | sequence | UGT | Cp73B | From | C. papaya |
| NO: 235 | | of | | | | |
| SEQ ID | Protein | sequence | UGT | Cp73B | From | C. papaya |
| NO: 236 | | of | | | | |
| SEQ ID | DNA | sequence | UGT | Ac73H | From | A. commosus |
| NO: 237 | | of | | | | |
| SEQ ID | Protein | sequence | UGT | Ac73H | From | A. commosus |
| NO: 238 | | of | | | | |
| SEQ ID | DNA | sequence | UGT | Qs85H | From | Q. suber |
| NO: 239 | | of | | | | |
| SEQ ID | Protein | sequence | UGT | Qs85H | From | Q. suber |
| NO: 240 | | of | | | | |
| SEQ ID | DNA | sequence | UGT | Vv83A | From | V. vinifera |
| NO: 241 | | of | | | | |
| SEQ ID | Protein | sequence | UGT | Vv83A | From | V. vinifera |
| NO: 242 | | of | | | | |
| SEQ ID | DNA | sequence | UGT | At71C1_At71C2_353 | From | A. thaliana-S. |
| NO: 243 | | of | | | | rebaudiana hybrid |
| SEQ ID | Protein | sequence | UGT | At71C1_At71C2_353 | From | A. thaliana-S. |
| NO: 244 | | of | | | | rebaudiana hybrid |
| SEQ ID | DNA | sequence | UGT | Pt72B | From | P. trichocarpa |
| NO: 245 | | of | | | | |
| SEQ ID | Protein | sequence | UGT | Pt72B | From | P. trichocarpa |
| NO: 246 | | of | | | | |
| SEQ ID | DNA | sequence | UGT | Cc91D1 | From | C. cardunculus |
| NO: 247 | | of | | | | |
| SEQ ID | Protein | sequence | UGT | Cc91D1 | From | C. cardunculus |
| NO: 248 | | of | | | | |
| SEQ ID | DNA | sequence | UGT | Pg91B1 | From | P. ginseng |
| NO: 249 | | of | | | | |
| SEQ ID | Protein | sequence | UGT | Pg91B1 | From | P. ginseng |
| NO: 250 | | of | | | | |
| SEQ ID | DNA | sequence | UGT | Ha91 | From | H. annuus |
| NO: 251 | | of | | | | |
| SEQ ID | Protein | sequence | UGT | Ha91 | From | H. annuus |
| NO: 252 | | of | | | | |
| SEQ ID | DNA | sequence | Cytochrome b5 | PanCyCYB5 | From | Panaeolus |
| NO: 253 | | of | | | | cyanescens |
| SEQ ID | Protein | sequence | Cytochrome b5 | PanCyCYB5 | From | Panaeolus |
| NO: 254 | | of | | | | cyanescens |
| SEQ ID | DNA | sequence | Tryptophan synthase | PanCyTrpB | From | Panaeolus |
| NO: 255 | | of | | | | cyanescens |
| SEQ ID | Protein | sequence | Tryptophan synthase | PanCyTrpB | From | Panaeolus |
| NO: 256 | | of | | | | cyanescens |

### REFERENCES

Gietz, R. D., & Woods, R. A. (2002). Transformation of yeast by lithium acetate/single-stranded carrier DNA/polyethylene glycol method. Methods in Enzymology, 350(2001), 87-96. https://doi.org/10.1016/SO076-6879(02)50957-5.
Krengel, F., Mijangos, M. V, Reyes-lezama, M., & Reyes-chilpa, R. (2019). Extraction and Conversion Studies of the Antiaddictive Alkaloids Coronaridine, Ibogamine, Voacangine, and Ibogaine from Two Mexican Tabernaemontana Species (Apocynaceae). https://doi.org/10.1002/cbdv.201900175.
Maury, J., Germann, S. M., Baallal Jacobsen, S. A., Jensen, N. B., Kildegaard, K. R., Herrgàrd, M. J., Schneider, K., Koza, A., Forster, J., Nielsen, J., & Borodina, I. (2016). EasyCloneMulti: A set of vectors for simultaneous and multiple genomic integrations in Saccharomyces cerevisiae. PLoS ONE, 11(3), 1-22. https:H/doi.org/10.1371/J*ourna1.pone.0150394.
Mikkelsen, M. D., Buron, L. D., Salomonsen, B., Olsen, C. E., Hansen, B. G., Mortensen, U. H., & Halkier, B. A. (2012). Microbial production of indolylglucosinolate through engineering of a multi-gene pathway in a versatile yeast expression platform. Metabolic Engineering, 14(2), 104-111. https://doi.org/10.1016/j.ymben.2012.01.006.
Janis Fricke, D. A. (2019). Enzymatic Route toward 6-Methylated Baeocystin and Psilocybin. ChemBioChem, 2824-2829.
Michael E. Lee, W. C. (2015). A Highly Characterized Yeast Toolkit for Modular, Multipart Assembly. ACS Synthetic Biology, 975-986.

### Examples

### Materials and Methods

### Materials

Chemicals used in the examples herein e.g. for buffers and substrates are commercial products of at least reagent grade.

### Background Strains

BY4741 is a common strain of S. cerevisiae derived from S288C and available e.g. from American Type Culture Collection (ATCC #200885). DH5a and XJb (DE3) are common strains of E. coli available from E.g. Zymo Research.

### Example 1—Construction of Genetically Modified S. cerevisiae Strains for De Novo Production of Tryptamine and Substituted Tryptamine Derivatives

### Part 1

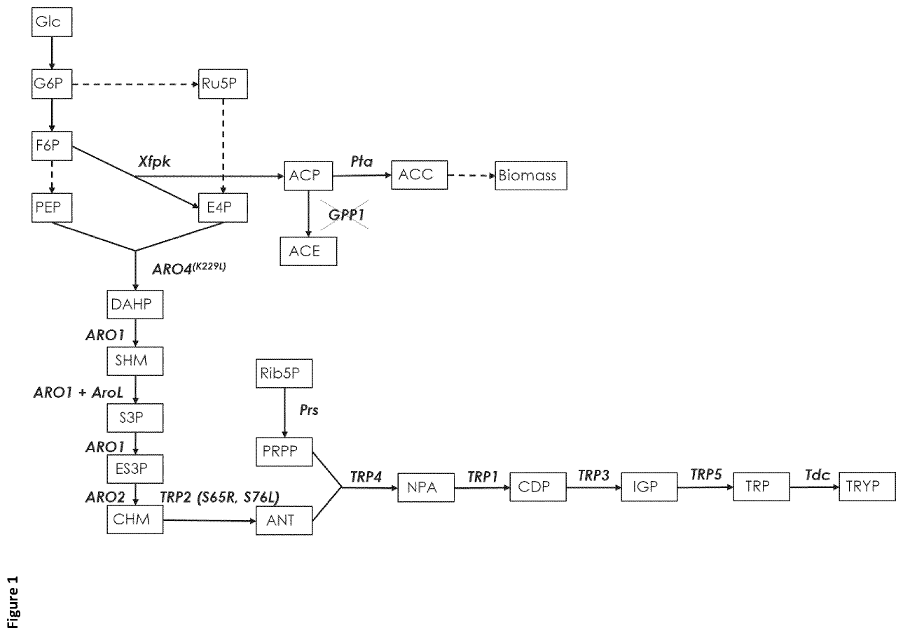

High-level de novo production of tryptamine and substituted tryptamine derivatives in S. cerevisiae was achieved by introducing a heterologous tryptophan decarboxylase (CrTdc) allowing conversion of the native S. cerevisiae metabolite tryptophan into tryptamine. To further increase the flux towards tryptamine a series of native genes were deleted to reduce diversion of flux away from the target pathway, as well as to remove regulation of the tryptophan biosynthetic pathway. Native S. cerevisiae genes involved in the biosynthetic pathway were overexpressed as well as feedback insensitive mutants. Additionally, a heterologous fructose-6-phosphate “bypass” was introduced to divert flux away from central glycolysis and towards Erythrose 4-phosphate. Finally, to divert carbon flux away from central glycolysis and into the shikimate pathway, down regulation of CDCl9, PFK1 and PFK2 was performed by promoter replacement of the native promoters with truncated versions of the URA3 promoter. Truncation of the URA3 promoter resulted in a decrease in promoter activity thereby achieving a reduction in enzyme activity of the target genes. A general scheme of the tryptamine biosynthetic pathway is shown in FIG. 1.

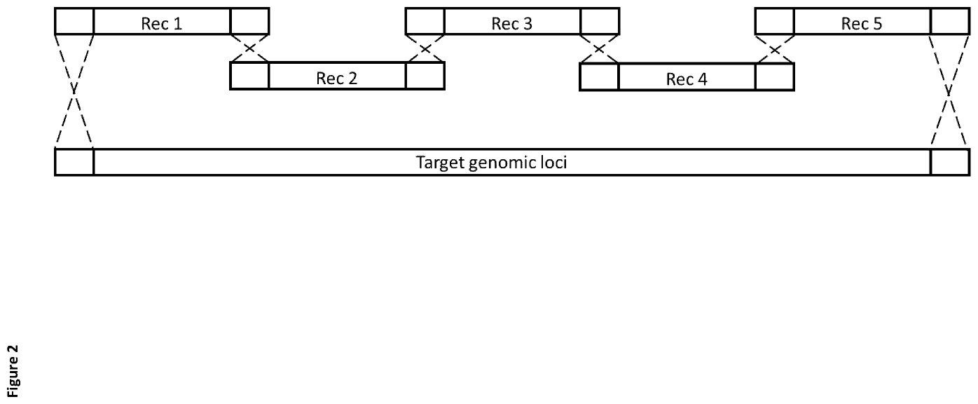

Genes for the tryptamine biosynthetic pathway were integrated into pre-defined genomic “landing pads” using custom-made overexpression plasmids similar to the system described by (Mikkelsen et al., 2012). Linear integration fragments are produced by NotI digestion of custom designed plasmids containing strong constitutive S. cerevisiae promoters and terminators and are flanked by upstream and downstream homology regions to facilitate assembly by homologous recombination. To facilitate assembly of multiple integration plasmids at a single genomic locus, upstream and downstream homology arms are designed so that after NotI digestion (New England Bio Labs Inc.), linear integration fragments can recombine into a single linear integration fragment and integrate in the target genomic loci. To select for transformants that have successfully integrated the fragments of interest, an endonuclease such as MAD7 can be used as described above or alternatively a selection marker such as LEU2 can be incorporated into the linear integration fragments and transformed into S. cerevisiae strains that are auxotrophic for Leucine as is known in the art. To reduce the occurrence of false positives the selection marker can be split across 2 linear integration fragments such as Rec 1 and Rec 2 such that a functional LEU2 selection marker can only be generated upon successful homologous recombination of the Rec 1 and Rec 2 integration fragments as shown in FIG. 2.

Genes were codon-optimized for expression in yeast and synthesized and cloned into custom integration plasmids by Twist Biosciences (Table 1). After linearization by restriction digestion with NotI (New England Bio Labs Inc.) plasmids are transformed into S. cerevisiae according to (Gietz & Woods, 2002). Transformants are plated on selective media.

TABLE 1
Integration plasmids used to construct tryptamine and tryptamine
derivative producing S. cerevisiae strains.

| Plasmid name | SEQ ID NO |
|---|---|
| PL-444(Rec1-XI-5-LEU: OsT5H) | SEQ ID NO: 95 |
| PL-445(Rec2-LEU: FoCPR) | SEQ ID NO: 111 |
| PL-447(Rec5-XI-5: RgTdc) | SEQ ID NO: 69 |
| PL-448(Rec4: HsASMT) | SEQ ID NO: 117 |
| PL-462(Rec4: CsSNMT1) | SEQ ID NO: 135 |
| PL-478(Rec2-URA: BbXfpk) | SEQ ID NO: 1 |
| PL-479(Rec1-XII-1-URA: CkPta) | SEQ ID NO: 3 |
| PL-480(Rec5-XII-1: BsPrs) | SEQ ID NO: 15 |
| PL-481(Rec4: ARO1) | SEQ ID NO: 7 |
| PL-482(Rec5-XI-5: ARO2) | SEQ ID NO: 11 |
| PL-483(Rec1-XI-5-LEU2: CrTdc) | SEQ ID NO: 25 |
| PL-484(Rec3: EcAroL) | SEQ ID NO: 9 |
| PL-485(Rec4: POS5) | SEQ ID NO: 185 |
| PL-486(Rec2-LEU: ARO4 (K229L)) | SEQ ID NO: 5 |
| PL-487(Rec3: TRP2 (S65R, S76L)) | SEQ ID NO: 13 |
| PL-533(Rec 2-LEU: Til10H) | SEQ ID NO: 99 |
| PL-534(Rec 1-XI-5-LEU: TIN10OMT) | SEQ ID NO: 119 |
| PL-535(Rec 3x-XI-5: CrCPR1) | SEQ ID NO: 107 |
| PL-536(Rec 3x-XI-5: CrCPR2) | SEQ ID NO: 109 |
| PL-556(Rec1-XVI-21-URA: PcPsiD) | SEQ ID NO: 71 |
| PL-557(Rec1-XVI-21-URA: PsPsiD) | SEQ ID NO: 75 |
| PL-558(Rec1-XVI-21-URA: PanCyPsiD) | SEQ ID NO: 77 |
| PL-569(Rec 5-XVI-21: PcPsiH) | SEQ ID NO: 87 |
| PL-570(Rec 5-XVI-21: PsPsiH1) | SEQ ID NO: 89 |
| PL-571(Rec 5-XVI-21: PsPsiH2) | SEQ ID NO: 91 |
| PL-572(Rec 5-XVI-21: PanCyPsiH) | SEQ ID NO: 93 |
| PL-(X-4 MF: CrTdc) | SEQ ID NO: 25 |
| PL-(XII-5 MF: OsT5H-FoCpr) | SEQ ID NO: 95, 111 |
| PL-(X-2 Ass1 MF: CaSHT) | SEQ ID NO: 161 |
| PL-(Ass 2: AtPAL2-At4CH-AtATR2) | SEQ ID NO: 51, 53, 55 |
| PL-(X-2 Ass3: At4CL2 | SEQ ID NO: 57 |
| PL-(Ty1c-tURA3: CaSHT) | SEQ ID NO: 161 |
| PL-494(XV1-21 Int TRP4 Split KlUra + DR L) | SEQ ID NO: 17 |
| PL-495(XVI-21 Int Split KlUra + DR R) | NA |
| PL-500(Rec 2-URA: PcPsiK) | SEQ ID NO: 155 |
| PL-501(Rec 2-URA: PsPsiK) | SEQ ID NO: 157 |
| PL-502(Rec 2-URA: PanCyPsiK) | SEQ ID NO: 159 |
| PL-507(Rec 4: PcCPR) | SEQ ID NO: 101 |
| PL-508(Rec 4: PsCPR) | SEQ ID NO: 103 |
| PL-509(Rec 4: PanCyCPR) | SEQ ID NO: 105 |
| PL-510(Rec 3: PcPsiM) | SEQ ID NO: 123 |
| PL-511(Rec 3: PsPsiM) | SEQ ID NO: 125 |
| PL-512(Rec 3: PanCyPsiM) | SEQ ID NO: 127 |
| PL-513(Rec 3: PcPsiM (H210A)) | SEQ ID NO: 129 |
| PL-530(Rec 3: PcPsiL) | SEQ ID NO: 163 |
| PL-531(Rec 1-XI-5-LEU: EcTnaA) | SEQ ID NO: 175 |
| PL-581(Ass1-XII-2-SpHIS5: TRP4-TRP5) | SEQ ID NO: 17, 23 |
| PL-582(Single Int-XII-2-SpHIS5: TRP4-TRP5) | SEQ ID NO: 17, 23 |
| PL-583(Ass2: TRP1-TRP3) | SEQ ID NO: 19, 21 |
| PL-584(Ass3-XII-2: TRP2 (S65R, S76L)- ARO4 (K229L)) | SEQ ID NO: 13, 5 |
| PL-446(p413TEF: PsTrpM) | SEQ ID NO: 131 |
| PL-529(PcPsiP) | SEQ ID NO: 165 |

### Part 2

Alternatively, integration plasmids we're constructed based on the MoClo system similar to (Michael E. Lee, 2015). Here, DNA part libraries containing promoters, terminators, genes of interest, and overlapping homology arms (for genomic integration and homologous recombination with adjacent integration cassettes) were cloned into entry vectors using standard Type IIs BsmBI restriction cloning reactions. To construct full integration expression cassettes overexpressing a gene of interest with strong constitutive promoter and terminator and overlapping integration homology arms, entry part plasmids were combined in a one-pot Type IIs BsaI restriction cloning reaction to generate the final integration plasmid. Pre-defined genomic “landing pads” were used as integration sites as described above. Counter-selectable markers (e.g. URA3 and AmdS) were integrated along with expression cassettes of interest in order to select for transformants with correctly integrated cassettes. Marker cassettes were split into two halves and encoded on overlapping homology arms between integration cassettes so that correct integration of more than one integration set would be required for functional expression of the marker. Marker cassettes were flanked by 500 bp homologous sequences to facilitate counter selection of the marker (by plating on 5-FOA or fluoroacetamide). A list of MoClo based yeast integration plasmids used to overproduce tryptamine and other substituted tryptamine derivatives is given in Table 2.

TABLE 2
Additional MoClo based integration plasmids used to construct tryptamine
and tryptamine derivative producing S. cerevisiae strains.

| Plasmid name | SEQ ID NO |
|---|---|
| PL-1583 (MoClo: X-2 int At4CL2) | SEQ ID NO: 57 |
| PL-606(Single Int-XII-2-SpHIS5: TRP4) | SEQ ID NO: 17 |
| PL-646(MoClo: RIC1 Up-TRP2 (S65R, S76L)-Split KlUra3 + DR L) | SEQ ID NO: 13 |
| PL-647(MoClo: Split KlUra3 + DR R-ARO4 (K229L)-RIC1 Down) | SEQ ID NO: 5 |
| PL-660(MoClo: X-3-TRP1-Split KlUra + DR L) | SEQ ID NO: 19 |
| PL-661(MoClo: Split KlUra3 + DR R-TRP3-X-3) | SEQ ID NO: 21 |
| PL-688(MoClo: XVI-21 int-OsT5H-Split KlUra3 + DR L) | SEQ ID NO: 95 |
| PL-690(MoClo: Split KlUra3 + DR R-FoCPR-XVI-21 int) | SEQ ID NO: 111 |
| PL-862(MoClo: X-4 int-BtAANAT-Split AmdS + DR L) | SEQ ID NO: 141 |
| PL-866(MoClo: Split AmdS + DR R-HsASMT-X-4 int) | SEQ ID NO: 117 |
| PL-1864(MoClo: X-4 int-PcPsiK-split KlUra3 + DR L) | SEQ ID NO: 155 |
| PL-1865(MoClo: X-4 int-PanCyPsiK-split KlUra3 + DR L) | SEQ ID NO: 159 |
| PL-1874(MoClo: split KlUra3 + DR R hom-PcPsiM-X-4 int) | SEQ ID NO: 123 |
| PL-1876(MoClo: split KlUra3 + DR R-PanCyPsiM-X-4-int) | SEQ ID NO: 127 |
| PL-1782(XI-1 KlUra3: PcCPR-PcPsiH) | SEQ ID NO: 101, 87 |
| PL-1896(XI-1 KlUra3: PanCyCPR-PanCyPsiH) | SEQ ID NO: 105, 93 |
| PL-1893(MoClo: X-2 int-At71C2-AmdS-X-2 int) | SEQ ID NO: 193 |
| PL-1897(MoClo: X-2 int-PanCyCyB5-KlUra3-X-2 int) | SEQ ID NO: 253 |
| PL-747(MoClo: XVI-21 int-PcPsiK- KlUra3 + DR L) | SEQ ID NO: 155 |
| PL-749(MoClo: KlUra3 + DR R-PcPsiM-XVI-21 int) | SEQ ID NO: 123 |
| PL-1720(MoClo: XVI-21 int-PanCyPsiK- KlUra3 + DR L) | SEQ ID NO: 159 |
| PL-1721(MoClo: KlUra3 + DR R-PanCyPsiM-XVI-21 int) | SEQ ID NO: 123 |
| PL-1722(MoClo: split KlUra3 + DR R hom-PcPsiM(H210A)-X-4 int) | SEQ ID NO: 129 |

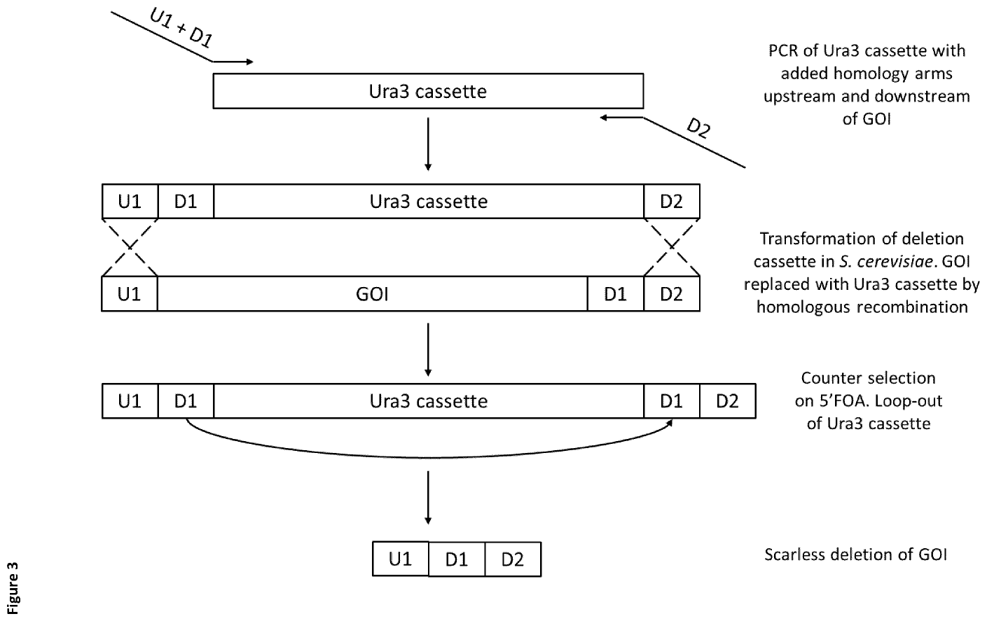

Native S. cerevisiae genes were deleted by marker replacement in S. cerevisiae strains which were auxotrophic for essential amino acids e.g., BY4741. Deletion cassettes were prepared by PCR amplification of auxotrophic marker cassettes with primers which included upstream and downstream homology to the coding region of the gene of interest to be deleted. Subsequent transformation of the integration cassette into S. cerevisiae strains and selection on media lacking the required amino acid resulted in integration of the marker cassette at the gene of interest. In some cases, additional direct repeats were introduced flanking the marker cassette to subsequently counter select the marker for repeated use. E.g. the Ura3 cassette was flanked with direct repeats so that the marker could be looped out upon incubation of the S. cerevisiae strain on media containing 5-Fluoroorotic acid (5-FOA). A diagram demonstrating PCR amplification of knockout cassettes is shown in FIG. 3. Finally, in some cases an endonuclease such as MAD7 was used to induce a DNA break at a gene of interest and the gene removed by replacement with a DNA cassette consisting of DNA flanking the gene of interest. A list of gene deletion cassettes used in later examples is shown in Table 3.

TABLE 3
Gene deletion cassettes generated by PCR amplification of
auxotrophic markers from prototrophic S. cerevisiae.

| Knockout cassette | Target gene KO | SEQ ID NO |
|---|---|---|
| BB-500(HIS3 RIC1 hom) | RIC1 | SEQ ID NO: 33 |
| BB-501(MET17 GPP1 hom) | GPP1 | SEQ ID NO: 34 |
| BB-508(URA3 BNA2 hom) | BNA2 | SEQ ID NO: 35 |
| BB-509(URA3 ARO10 hom) | ARO10 | SEQ ID NO: 37 |
| BB-510(URA3 PDC5 hom) | PDC5 | SEQ ID NO: 38 |
| BB-511(URA3 HTZ1 hom) | HTZ1 | SEQ ID NO: 39 |
| BB-512(URA3 DIA3 hom) | DIA3 | SEQ ID NO: 40 |
| BB-513(URA3 PHO5 hom) | PHO5 | SEQ ID NO: 41 |
| BB-514(URA3 PHO12 hom) | PHO12 | SEQ ID NO: 42 |
| BB-515(URA3 PHO11 hom) | PHO11 | SEQ ID NO: 43 |
| BB-516(URA3 PHO3 hom) | PHO3 | SEQ ID NO: 44 |
| BB-517(URA3 CYS4 hom) | CYS4 | SEQ ID NO: 36 |
| BB-591(URA3 ERG4 hom) | ERG4 | SEQ ID NO: 183 |
| BB-592(URA3 SPE2 hom) | SPE2 | SEQ ID NO: 184 |

To produce substituted tryptamine derivatives de novo in S. cerevisiae, derivatizing enzymes were introduced into strains producing indole acceptors suchas tryptamine or other substituted tryptamines. To achieve this, genes encoding derivatizing enzymes were codon optimized, synthesized and cloned by Twist into centromeric plasmids derived from p415TEF containing a LEU2 auxotrophic marker. Expression plasmids were subsequently transformed into S. cerevisiae strains auxotrophic for leucine. Alternatively, genes were integrated into pre-defined genomic landing pads using integration cassettes as described above. Plasmids expressing derivatizing enzymes are listed in Table 1, Table 2 and Table 4. Plasmid DNA or DNA for integration into the genome was introduced into S. cerevisiae using the LiAc method known in the art.

### Example 2—Construction of Genetically Modified S. cerevisiae Strains for Production of Substituted Tryptamine Derivatives by Feeding Substituted Indole Acceptors

### Part 1

S. cerevisiae strains producing substituted tryptamine derivatives from substituted indole acceptors were constructed in three steps. In step one, genes encoding enzymes catalysing the conversion of (substituted) indole to (substituted) tryptophan (Tryptophan synthase and Tryptophan synthase beta sub-unit) were codon optimized, synthesized and cloned by Twist into centromeric plasmids derived from p413TEF containing a HIS3 auxotrophic marker. Expression plasmids were subsequently transformed into S. cerevisiae strains auxotrophic for histidine. In step two, genes encoding enzymes catalysing the conversion of (substituted) tryptophan to (substituted) tryptamine (Tryptophan decarboxylase) were codon optimized and synthesized by Twist then cloned into Ty integration vectors with truncated Ura3 markers as described by (Maury et al., 2016) To drive expression of these tryptophan decarboxylase genes the galactose inducible GAL10 promoter was used so that the enzymes would only be active in media containing galactose. Integration plasmids were subsequently transformed into S. cerevisiae strains auxotrophic for uracil. In step three, genes encoding enzymes catalysing the conversion of (substituted) tryptamine to (substituted) tryptamine derivatives (derivatising enzymes) were codon optimized, synthesized and cloned by Twist into centromeric plasmids derived from p415TEF containing a LEU2 auxotrophic marker. Expression plasmids were subsequently transformed into S. cerevisiae strains auxotrophic for leucine. Different combinations of genes from each of the three steps can be used to produce a range of substituted tryptamine derivatives.

TABLE 4
Expression plasmids used to construct
substituted indole conversion strains.

| | |
|---|---|
| Plasmid name | SEQ ID NO |

(Substituted) indole to (substituted) tryptophan

| | |
|---|---|
| PL-449(p413TEF: PfTrpB(2B9)) | SEQ ID NO: 59 |
| p413TEF (SeTrpB) | SEQ ID NO: 61 |
| p413TEF (PfTrpB) | SEQ ID NO: 63 |
| p413TEF (TRP5) | SEQ ID NO: 65 |
| p413TEF (TmTrpB (M145T, N167D)) | SEQ ID NO: 67 |
| p413TEF (PcTrpB (M439T, N459D) | SEQ ID NO: 181 |

(Substituted) tryptophan to (substituted) tryptamine

| | |
|---|---|
| pTy-tUra3 (CrTdc) | SEQ ID NO: 25 |
| pTy-tUra3 (RgTdc) | SEQ ID NO: 69 |
| pTy-tUra3 (PcPsiD) | SEQ ID NO: 71 |
| pTy-tUra3 (PcncAAAD) | SEQ ID NO: 73 |
| pTy-tUra3 (PanCyPsiD) | SEQ ID NO: 77 |

(Substituted) tryptamine to (substituted) tryptamine derivative

| | |
|---|---|
| PL-537(p416TEF: PtIGS) | SEQ ID NO: 85 |
| p415TEF (CsSNMT1) | SEQ ID NO: 135 |
| p415TEF (HsASMT) | SEQ ID NO: 117 |

A list of all S. cerevisiae strains constructed by Example 1 and 2 is shown in Table 5 and Table 6 along with the corresponding genotypes. For simplicity the constitutive terminators used to overexpress each gene is omitted but consisted of any one of the following strong terminators, tADH1, tCYC1, tSSA1, tENO2, tTDH1, tPGK1, tTDH2, tPG11.

TABLE 5
S. cerevisiae strains constructed in Example 1 and 2.

| Strain | |
|---|---|
| name | Genotype |
| BY4741 | MATa his3Δ1 leu2Δ0 met15Δ0 ura3Δ0 |
| SC-17 | MATa his3Δ1 leu2Δ0 met15Δ0 ura3Δ0 XI-5::(pTDH3 −> CrTdc, KlLeu2 <− pKlLeu2, pTEF1 −> ARO4(K229L), TRP2 (S65R, |
| | S76L) <− pTEF2, pFBA1 −> ARO1, ARO2 <− pPGK1) |
| SC-18 | MATa his3Δ1 leu2Δ0 met15Δ0 ura3Δ0 XI-5::(pTDH3 −> CrTdc, KlLeu2 <− pKlLeu2, pTEF1 −> ARO4(K229L), TRP2 (S65R, |
| | S76L) <− pTEF2, pFBA1 −> ARO1, ARO2 <− pPGK1), XII-1::(pTDH3 −> CkPta, KlUra3 <− pKlUra3, pTEF1 −> BbXfpk, EcAroL <− |
| | pTEF2, BsPrs <− pPGK1) |
| SC-25 | MATa his3Δ1 leu2Δ0 met15Δ0 ura3Δ0 XI-5::(pTDH3 −> CrTdc, KlLeu2 <− pKlLeu2, pTEF1 −> ARO4(K229L), TRP2 (S65R, |
| | S76L) <− pTEF2, pFBA1 −> ARO1, ARO2 <− pPGK1), XII-1::(pTDH3 −> CkPta, KlUra3 <− pKlUra3, pTEF1 −> BbXfpk, EcAroL <− |
| | pTEF2, BsPrs <− pPGK1), gpp1Δ::HIS3 |
| SC-27 | MATa his3Δ1 leu2Δ0 met15Δ0 ura3Δ0 XI-5::(pTDH3 −> CrTdc, KlLeu2 <− pKlLeu2, pTEF1 −> ARO4(K229L), TRP2 (S65R, |
| | S76L) <− pTEF2, pFBA1 −> ARO1, ARO2 <− pPGK1), XII-1::(pTDH3 −> CkPta, KlUra3 <− pKlUra3, pTEF1 −> BbXfpk, EcAroL <− |
| | pTEF2, BsPrs <− pPGK1), gpp1Δ::HIS3, ric1Δ::MET17 |
| SC-43 | MATa his3Δ1 leu2Δ0 met15Δ0 ura3Δ0 XI-5::(pTDH3 −> CrTdc, KlLeu2 <− pKlLeu2, pTEF1 −> ARO4(K229L), TRP2 (S65R, |
| | S76L) <− pTEF2, pFBA1 −> ARO1, ARO2 <− pPGK1), XII-1::(pTDH3 −> CkPta, pTEF1 −> BbXfpk, EcAroL <− pTEF2, BsPrs <− |
| | pPGK1), gpp1Δ::HIS3 |
| SC-44 | MATa his3Δ1 leu2Δ0 met15Δ0 ura3Δ0 XI-5::(pTDH3 −> CrTdc, KlLeu2 <− pKlLeu2, pTEF1 −> ARO4(K229L), TRP2 (S65R, |
| | S76L) <− pTEF2, pFBA1 −> ARO1, ARO2 <− pPGK1), XII-1::(pTDH3 −> CkPta, pTEF1 −> BbXfpk, EcAroL <− pTEF2, BsPrs <− |
| | pPGK1), gpp1Δ::HIS3, XV1-21::(pTDH3 −> TRP4, pKlUra3 −> KlUra3) |
| SC-21 | MATa his3Δ1 leu2Δ0 met15Δ0 ura3Δ0 XI-5::(pTDH3 −> OsT5H, KlLeu2 <− pKlLeu2, pTEF1 −> FoCPR, RgTdc <− pPGK1) |
| SC-41 | MATa his3Δ1 leu2Δ0 met15Δ0 ura3Δ0 P413TEF::(PfTrpB(2B9)), Ty1C::(pGAL10 −> RgTdc, pURA3d −> URA3) |
| SC-34 | MATa his3Δ1 leu2Δ0 met15Δ0 ura3Δ0 XI-5::(pTDH3 −> OsT5H, KlLeu2 <− pKlLeu2, pTEF1 −> FoCPR, pFBA1 −> HsASMT, |
| | RgTdc <− pPGK1) |
| SC-36 | MATa his3Δ1 leu2Δ0 met15Δ0 ura3Δ2 XI-5::(pTDH3 −> OsT5H, KlLeu2 <− pKlLeu2, pTEF1 −> FoCPR, pFBA1 −> CsSNMT1, |
| | RgTdc <− pPGK1) |
| ST-4CS1 | Mat alpha, ura3Delta::X-4::(pTEF −> CrTdc), XII-5::(OsT5H <− pPGK1-pTEF −> FoCpr), X-2::(CaSHT <− pPGK1, AtPAL2 <− |
| | pTPl1-pPDC1 −> AtC4H-AtATR2, pPDC1 −> At4CL2), |
| ST-4CS2 | Mat alpha, ura3Delta::X-4::(pTEF −> CrTdc), XII-5::(OsT5H <− pPGK1-pTEF −> FoCpr), X-2::(CaSHT <− pPGK1, AtPAL2 <− |
| | pTPl1-pPDC1 −> AtC4H-AtATR2, pPDC1 −> At4CL2), Ty1C::(URA3d-CaSHT <− pPGK1) |

### Part 2

An additional list of S. cerevisiae strains constructed in Example 1 and 2 is given in Table 6.

TABLE 6
Additional S. cerevisiae strains constructed in Example 1 and 2.

| Strain name | Genotype |
|---|---|
| SC-NFC | Mat alpha, ura3Delta:: X-4::(pTEF−>CrTdc), XII-5::(OsT5H<−pPGK1 − pTEF−>FoCpr), X- |
| | 2::(Nt4CL2<−pTEF1), Ty1C::(URA3d-CaSHT<−pPGK1) |
| SC-49 | MATa his3Δ1 leu2Δ0 met15Δ0 ura3Δ0 XI-5::(pTDH3−>CrTdc, KiLeu2<−pKiLeu2, |
| | pTEF1−>ARO4(K229L), TRP2 (S65R, S76L)<−pTEF2, pFBA1−>ARO1, ARO2<−pPGK1), |
| | XII-1::(pTDH3−>CkPta, pTEF1−>BbXfpk, EcAroL<−pTEF2, BsPrs<−pPGK1), gpp1Δ::MET17, |
| | XII-2::(pTDH3−>TRP4, SpHis5) |
| SC-56 | MATa his3Δ1 leu2Δ0 met15Δ0 ura3Δ0 XI-5::(pTDH3−>CrTdc, KiLeu2<−pKiLeu2, |
| | pTEF1−>ARO4(K229L), TRP2 (S65R, S76L)<−pTEF2, pFBA1−>ARO1, ARO2<−pPGK1), |
| | XII-1::(pTDH3−>CkPta, pTEF1−>BbXfpk, EcAroL<−pTEF2, BsPrs<−pPGK1), gpp1Δ::MET17, |
| | XII-2::(pTDH3−>TRP4, SpHis5), aro10Δ::KIUra3 |
| SC-58 | MATa his3Δ1 leu2Δ0 met15Δ0 ura3Δ0 XI-5::(pTDH3−>CrTdc, KiLeu2<−piLeu2, |
| | pTEF1−>ARO4(K229L), TRP2 (S65R, S76L)<−pTEF2, pFBA1−>ARO1, ARO2<−pPGK1), |
| | XII-1::(pTDH3−>CkPta, pTEF1−>BbXfpk, EcAroL<−pTEF2, BsPrs<−pPGK1), gpp1Δ::MET17, |
| | XII-2::(pTDH3−>TRP4, SpHis5), aro10Δ |
| SC-59 | MATa his3Δ1 leu2Δ0 met15Δ0 ura3Δ0 XI-5: (pTDH3−>CrTdc, KiLeu2<−pKiLeu2, |
| | pTEF1−>ARO4(K229L), TRP2 (S65R, S76L)<−pTEF2, pFBA1−>ARO1, ARO2<−pPGK1), |
| | XII-1::(pTDH3−>CkPta, pTEF1−>BbXfpk, EcAroL<−pTEF2, BsPrs<−pPGK1), gpp1Δ::MET17, |
| | XII-2::(pTDH3−>TRP4, SpHis5), aro10Δ, ric1Δ::(pTDH3−>TRP2(S65R, S76L), pPGK1−>ARO4 |
| | (K229L), KIUra3) |
| SC-60 | MATa his3Δ1 leu2Δ0 met15Δ0 ura3Δ0 XI-5::(pTDH3−>CrTdc, KiLeu2<−pKiLeu2, |
| | pTEF1−>ARO4(K229L), TRP2 (S65R, S76L)<−pTEF2, pFBA1−>ARO1, ARO2<−pPGK1), |
| | XII-1::(pTDH3−>CkPta, pTEF1−>BbXfpk, EcAroL<−pTEF2, BsPrs<−pPGK1), gpp1Δ::MET17, |
| | XII-2::(pTDH3−>TRP4, SpHis5), aro10Δ, ric1Δ::(pTDH3−>TRP2(S65R, S76L), pPGK1−>ARO4 (K229L)) |
| SC-62 | MATa his3Δ1 leu2Δ0 met15Δ0 ura3Δ0 XI-5::(pTDH3−>CrTdc, KiLeu2<−pKiLeu2, |
| | pTEF1−>ARO4(K229L), TRP2 (S65R, S76L)<−pTEF2, pFBA1−>ARO1, ARO2<−pPGK1), |
| | XII-1::(pTDH3−>CkPta, pTEF1−>BbXfpk, EcAroL<−pTEF2, BsPrs<−pPGK1), gpp1Δ::MET17, |
| | XII-2::(pTDH3−>TRP4, SpHis5), aro10Δ, ric1Δ::(pTDH3−>TRP2(S65R, S76L), pPGK1−>ARO4 (K229L)), |
| | X-3::(pTEF2−>TRP1, pCCW12−>TRP3, KIUra3) |
| SC-106 | MATa his3Δ1 leu2Δ0 met15Δ0 ura3Δ0 XI-5::(pTDH3−>CrTdc, KiLeu2<−pKiLeu2, |
| | pTEF1−>ARO4(K229L), TRP2 (S65R, S76L)<−pTEF2, pFBA1−>ARO1, ARO2<−pPGK1), |
| | XII-1::(pTDH3−>CkPta, pTEF1−>BbXfpk, EcAroL<−pTEF2, BsPrs<−pPGK1), gpp1Δ::MET17, |
| | XII-2::(pTDH3−>TRP4, SpHis5), aro10Δ, ric1Δ::(pTDH3−>TRP2(S65R, S76L), pPGK1−>ARO4 (K229L)), |
| | X-3::(pTEF2−>TRP1, pCCW12−>TRP3), |
| SC-75 | MATa his3Δ1 leu2Δ0 met15Δ0 ura3Δ0 XI-5::(pTDH3−>CrTdc, KiLeu2<−pKiLeu2, |
| | pTEF1−>ARO4(K229L), TRP2 (S65R, S76L)<−pTEF2, pFBA1−>ARO1, ARO2<−pPGK1), |
| | XII-1::(pTDH3−>CkPta, pTEF1−>BbXfpk, EcAroL<−pTEF2, BsPrs<−pPGK1), gpp1Δ::MET17, |
| | XII-2::(pTDH3−>TRP4, SpHis5), aro10Δ, ric1Δ::(pTDH3−>TRP2(S65R, S76L), pPGK1−>ARO4 (K229L)), |
| | X-3::(pTEF2−>TRP1, pCCW12−>TRP3), XVI-21::(pPGK1−>OsT5H, pTEF1−>FoCPR) |
| SC-259 | MATa his3Δ1 leu2Δ0 met15Δ0 ura3Δ0 XI-5::(pTDH3−>CrTdc, KiLeu2<−pKiLeu2, |
| | pTEF1−>ARO4(K229L), TRP2 (S65R, S76L)<−pTEF2, pFBA1−>ARO1, ARO2<−pPGK1), |
| | XII-1::(pTDH3−>CkPta, pTEF1−>BbXfpk, EcAroL<−pTEF2, BsPrs<−pPGK1), gpp1Δ::MET17, |
| | XII-2::(pTDH3−>TRP4, SpHis5), aro10Δ, ric1Δ::(pTDH3−>TRP2(S65R, S76L), pPGK1−>ARO4 (K229L)), |
| | X-3::(pTEF2−>TRP1, pCCW12−>TRP3), XVI-21::(pPGK1−>OsT5H) |
| SC-124 | MATa his3Δ1 leu2Δ0 met15Δ0 ura3Δ0 XI-5::(pTDH3−>CrTdc, KiLeu2<−pKiLeu2, |
| | pTEF1−>ARO4(K229L), TRP2 (S65R, S76L)<−pTEF2, pFBA1−>ARO1, ARO2<−pPGK1), |
| | XII-1::(pTDH3−>CkPta, pTEF1−>BbXfpk, EcAroL<−pTEF2, BsPrs<−pPGK1), gpp1Δ::MET17, |
| | XII-2::(pTDH3−>TRP4, SpHis5), aro10Δ, ric1Δ::(pTDH3−>TRP2(S65R, S76L), pPGK1−>ARO4 (K229L)), |
| | X-3::(pTEF2−>TRP1, pCCW12−>TRP3), XVI-21::(pPGK1−>OST5H, pTEF1−>FoCPR), X-4::(pTDH3−>BtAANT, |
| | pENO2−>HsASMT) |
| SC-268 | MATa his3Δ1 leu2Δ0 met15Δ0 ura3Δ0 XI-5::(pTDH3−>CrTdc, KiLeu2<−pKiLeu2, |
| | pTEF1−>ARO4(K229L), TRP2 (S65R, S76L)<−pTEF2, pFBA1−>ARO1, ARO2<−pPGK1), |
| | XII-1::(pTDH3−>CkPta, pTEF1−>BbXfpk, EcAroL<−pTEF2, BsPrs<−pPGK1), gpp1Δ::MET17, |
| | XII-2::(pTDH3−>TRP4, SpHis5), aro10, ric1Δ::(pTDH3−>TRP2(S65R, S76L), pPGK1−>ARO4 (K229L)), |
| | X-3::(pTEF2−>TRP1, pCCW12−>TRP3), X-4::(pTDH3−>PanCyPsik, pCCW12−>PanCyPsiM), |
| | XI-1::(pTEF1−>PanCyCPR, PanCyPsiH<−pPGK1, KIUra3) |
| SC-269 | MATa his3Δ1 leu2Δ0 met15Δ0 ura3Δ0 XI-5::(pTDH3−>CrTdc, KiLeu2<−pKiLeu2, |
| | pTEF1−>ARO4(K229L), TRP2 (S65R, S76L)<−pTEF2, pFBA1−>ARO1, ARO2<−pPGK1), |
| | XII-1::(pTDH3−>CkPta, pTEF1−>BbXfpk, EcAroL<−pTEF2, BsPrs<−pPGK1), gpp1Δ::MET17, |
| | XII-2::(pTDH3−>TRP4, SpHis5), aro10Δ, ric1Δ:(pTDH3−>TRP2(S65R, S76L), pPGK1−>ARO4 (K229L)), |
| | X-3::(pTEF2−>TRP1, pCCW12−>TRP3), X-4::(pTDH3−>PanCyPsiK, pCCW12−>PanCyPsiM), |
| | XI-1::(pTEF1−>PanCyCPR, PanCyPsiH<−pPGK1) |
| SC-275 | MATa his3Δ1 leu2Δ0 met15Δ0 ura3Δ0 XI-5::(pTDH3−>CrTdc, KiLeu2<−pKiLeu2, |
| | pTEF1−>ARO4(K229L), TRP2 (S65R, S76L)<−pTEF2, pFBA1−>ARO1, ARO2<−pPGK1), |
| | XII-1::(pTDH3−>CkPta, pTEF1−>BbXfpk, EcAroL<−pTEF2, BsPrs<−pPGK1), gpp1Δ::MET17, |
| | XII-2::(pTDH3−>TRP4, SpHis5), aro10Δ, ric1Δ::(pTDH3−>TRP2(S65R, S76L), pPGK1−>ARO4 (K229L)), |
| | X-3::(pTEF2−>TRP1, pCCW12−>TRP3), X-4::(pTDH3−>PcPsik, pCCW12−>PcPsiM), XI-1::(pTEF1−>PcCPR, |
| | PcPsiH<−pPGK1, KIUra3) |
| SC-276 | MATa his3Δ1 leu2Δ0 met15Δ0 ura3Δ0 XI-5::(pTDH3−>CrTdc, KiLeu2<−pKiLeu2, |
| | pTEF1−>ARO4(K229L), TRP2 (S65R, S76L)<−pTEF2, pFBA1−>ARO1, ARO2<−pPGK1), |
| | XII-1::(pTDH3−>CkPta, pTEF1−>BbXfpk, EcAroL<−pTEF2, BsPrs<−pPGK1), gpp1Δ::MET17, |
| | XII-2::(pTDH3−>TRP4, SpHis5), aro10Δ, ric1Δ::(pTDH3−>TRP2(S65R, S76L), pPGK1−>ARO4 (K229L)), |
| | X-3::(pTEF2−>TRP1, pCCW12−>TRP3), X-4::(pTDH3−>PcPsik, pCCW12−>PcPsiM), XI-1::(pTEF1−>PcCPR, |
| | PcPsiH<−pPGK1) |
| SC-301 | MATa his3Δ1 leu2Δ0 met15Δ0 ura3Δ0 XI-5::(pTDH3−>CrTdc, KiLeu2<−pKiLeu2, |
| | pTEF1−>ARO4(K229L), TRP2 (S65R, S76L)<−pTEF2, pFBA1−>ARO1, ARO2<−pPGK1), |
| | XII-1::(pTDH3−>CkPta, pTEF1−>BbXfpk, EcAroL<−pTEF2, BsPrs<−pPGK1), gpp1Δ::MET17, |
| | XII-2::(pTDH3−>TRP4, SpHis5), aro10Δ, ric1Δ::(pTDH3−>TRP2(S65R, S76L), pPGK1−>ARO4 (K229L)), |
| | X-3::(pTEF2−>TRP1, pCCW12−>TRP3), X-4::(pTDH3−>PcPsik, pCCW12−>PcPsiM), XI-1::(pTEF1−>PcCPR, |
| | PcPsiH<−pPGK1), pho3Δ::KIUra3 |
| SC-302 | MATa his3Δ1 leu2Δ0 met15Δ0 ura3Δ0 XI-5::(pTDH3−>CrTdc, KiLeu2<−pKiLeu2, |
| | pTEF1−>ARO4(K229L), TRP2 (S65R, S76L)<−pTEF2, pFBA1−>ARO1, ARO2<−pPGK1), |
| | XII-1::(pTDH3−>CkPta, pTEF1−>BbXfpk, EcAroL<−pTEF2, BsPrs<−pPGK1), gpp1Δ::MET17, |
| | XII-2::(pTDH3−>TRP4, SpHis5), aro10, ric1Δ::(pTDH3−>TRP2(S65R, S76L), pPGK1−>ARO4 (K229L)), |
| | X-3::(pTEF2−>TRP1, pCCW12−>TRP3), X-4::(pTDH3−>PcPsik, pCCW12−>PcPsiM), XI-1::(pTEF1−>PcCPR, |
| | PcPsiH<−pPGK1), dia3Δ::KIUra3 |
| SC-303 | MATa his3Δ1 leu2Δ0 met15Δ0 ura3Δ0 XI-5::(pTDH3−>CrTdc, KiLeu2<−pKiLeu2, |
| | pTEF1−>ARO4(K229L), TRP2 (S65R, S76L)<−pTEF2, pFBA1−>ARO1, ARO2<−pPGK1), |
| | XII-1::(pTDH3−>CkPta, pTEF1−>BbXfpk, EcAroL<−pTEF2, BsPrs<−pPGK1), gpp1Δ::MET17, |
| | XII-2::(pTDH3−>TRP4, SpHis5), aro10Δ, ric1Δ:(pTDH3−>TRP2(S65R, S76L), pPGK1−>ARO4 (K229L)), |
| | X-3::(pTEF2−>TRP1, pCCW12−>TRP3), X-4:(pTDH3−>PcPsik, pCCW12−>PcPsiM), XI-1::(pTEF1−>PcCPR, |
| | PcPsiH<−pPGK1), pho5Δ::KIUra3 |
| SC-382 | MATa his3Δ1 leu2Δ0 met15Δ0 ura3Δ0 XI-5:(pTDH3−>CrTdc, KiLeu2<−pKiLeu2, |
| | pTEF1−>ARO4(K229L), TRP2 (S65R, S76L)<−pTEF2, pFBA1−>ARO1, ARO2<−pPGK1), |
| | XII-1::(pTDH3−>CkPta, pTEF1−>BbXfpk, EcAroL<−pTEF2, BsPrs<−pPGK1), gpp1Δ::MET17, |
| | XII-2::(pTDH3−>TRP4, SpHis5), aro10Δ, ric1Δ::(pTDH3−>TRP2(S65R, S76L), pPGK1−>ARO4 (K229L)), |
| | X-3:(pTEF2−>TRP1, pCCW12−>TRP3), erg4Δ, |
| SC-402 | MATa his3Δ1 leu2Δ0 met15Δ0 ura3Δ0 XI-5::(pTDH3−>CrTdc, KiLeu2<−pKiLeu2, |
| | pTEF1−>ARO4(K229L), TRP2 (S65R, S76L)<−pTEF2, pFBA1−>ARO1, ARO2<−pPGK1), |
| | XII-1::(pTDH3−>CkPta, pTEF1−>BbXfpk, EcAroL<−pTEF2, BsPrs<−pPGK1), gpp1Δ::MET17, |
| | XII-2::(pTDH3−>TRP4, SpHis5), aro10Δ, ric1Δ::(pTDH3−>TRP2(S65R, S76L), pPGK1−>ARO4 (K229L)), |
| | X-3::(pTEF2−>TRP1, pCCW12−>TRP3), X-4::(pTDH3−>PcPsik, pCCW12−>PcPsiM), XI-1::(pTEF1−>PcCPR, |
| | PcPsiH<−pPGK1), X-2::(pTDH3−>At71C2, AmdS) |
| SC-412 | MATa his3Δ1 leu2Δ0 met15Δ0 ura3Δ0 XI-5::(pTDH3−>CrTdc, KiLeu2<−pKiLeu2, |
| | pTEF1−>ARO4(K229L), TRP2 (S65R, S76L)<−pTEF2, pFBA1−>ARO1, ARO2<−pPGK1), |
| | XII-1::(pTDH3−>CkPta, pTEF1−>BbXfpk, EcAroL<−pTEF2, BsPrs<−pPGK1), gpp1Δ::MET17, |
| | XII-2::(pTDH3−>TRP4, SpHis5), aro10Δ, ric1Δ::(pTDH3−>TRP2(S65R, S76L), pPGK1−>ARO4 (K229L)), |
| | X-3::(pTEF2−>TRP1, pCCW12−>TRP3), X-4::(pTDH3−>PcPsik, pCCW12−>PcPsiM), XI-1::(pTEF1−>PcCPR, |
| | PcPsiH<−pPGK1), pho12Δ::KIUra3 |
| SC-413 | MATa his3Δ1 leu2Δ0 met15Δ0 ura3Δ0 XI-5::(pTDH3−>CrTdc, KiLeu2<−pKiLeu2, |
| | pTEF1−>ARO4(K229L), TRP2 (S65R, S76L)<−pTEF2, pFBA1−>ARO1, ARO2<−pPGK1), |
| | XII-1::(pTDH3−>CkPta, pTEF1−>BbXfpk, EcAroL<−pTEF2, BsPrs<−pPGK1), gpp1Δ::MET17, |
| | XII-2::(pTDH3−>TRP4, SpHis5), aro10Δ, ric1Δ::(pTDH3−>TRP2(S65R, S76L), pPGK1−>ARO4 (K229L)), |
| | X-3::(pTEF2−>TRP1, pCCW12−>TRP3), X-4::(pTDH3−>PcPsik, pCCW12−>PcPsiM), XI-1::(pTEF1−>PcCPR, |
| | PcPsiH<−pPGK1), pho11Δ::KIUra3 |
| SC-394 | MATa his3Δ1 leu2Δ0 met15Δ0 ura3Δ0 X-2::(pTDH3−>At71C2, AmdS) |
| SC-403 | MATa his3Δ1 leu2Δ0 met15Δ0 ura3Δ0 XI-5::(pTDH3−>CrTdc, KiLeu2<−pKiLeu2, |
| | pTEF1−>ARO4(K229L), TRP2 (S65R, S76L)<−pTEF2, pFBA1−>ARO1, ARO2<−pPGK1), |
| | XII-1::(pTDH3−>CkPta, pTEF1−>BbXfpk, EcAroL<−pTEF2, BsPrs<−pPGK1), gpp1Δ::MET17, |
| | XII-2::(pTDH3−>TRP4, SpHis5), aro10Δ, ric1Δ:(pTDH3−>TRP2(S65R, S76L), pPGK1−>ARO4 (K229L)), |
| | X-3::(pTEF2−>TRP1, pCCW12−>TRP3), X-4::(pTDH3−>PanCyPsiK, pCCW12−>PanCyPsiM), |
| | XI-1::(pTEF1−>PanCyCPR, PanCyPsiH<−pPGK1), X-2::(pTDH3−>PanCyCYB5, KIUra3) |
| SC-270 | MATa his3Δ1 leu2Δ0 met15Δ0 ura3Δ0 XI-5::(pTDH3−>CrTdc, KiLeu2<−pKiLeu2, |
| | pTEF1−>ARO4(K229L), TRP2 (S65R, S76L)<−pTEF2, pFBA1−>ARO1, ARO2<−pPGK1), |
| | XII-1::(pTDH3−>CkPta, pTEF1−>BbXfpk, EcAroL<−pTEF2, BsPrs<−pPGK1), gpp1Δ::MET17, |
| | XII-2::(pTDH3−>TRP4, SpHis5), aro10, ric1Δ::(pTDH3−>TRP2(S65R, S76L), pPGK1−>ARO4 (K229L)), |
| | X-3::(pTEF2−>TRP1, pCCW12−>TRP3), X-4::(pTDH3−>PanCyPsik, pCCW12−>PanCyPsiM), |
| | XI-1::(pTEF1−>PanCyCPR, PanCyPsiH<−pPGK1), X-2::(pPGK1−>PanCyCYB5, KIUra3) |
| SC-415 | MATa his3Δ1 leu2Δ0 met15Δ0 ura3Δ0 XI-5::(pTDH3−>CrTdc, KiLeu2<−pKiLeu2, |
| | pTEF1−>ARO4(K229L), TRP2 (S65R, S76L)<−pTEF2, pFBA1−>ARO1, ARO2<−pPGK1), |
| | XII-1::(pTDH3−>CkPta, pTEF1−>BbXfpk, EcAroL<−pTEF2, BsPrs<−pPGK1), gpp1Δ::MET17, |
| | XII-2::(pTDH3−>TRP4, SpHis5), aro10Δ, ric1Δ::(pTDH3−>TRP2(S65R, S76L), pPGK1−>ARO4 (K229L)), |
| | X-3::(pTEF2−>TRP1, pCCW12−>TRP3), X-4::(pTDH3−>PcPsik, pCCW12−>PcPsiM), XI-1::(pTEF1−>PcCPR, |
| | PcPsiH<−pPGK1, KIUra3), X-2::(pPGK1−>PcCYB5), XVI-21::(pTDH3−>PcPsik, pCCW12−>PcPsiM), |
| | pdc5Δ, erg4Δ, dia3Δ, pho5Δ, pho3Δ |
| SC-416 | MATa his3Δ1 leu2Δ0 met15Δ0 ura3Δ0 XI-5::(pTDH3−>CrTdc, KiLeu2<−pKiLeu2, |
| | pTEF1−>ARO4(K229L), TRP2 (S65R, S76L)<−pTEF2, pFBA1−>ARO1, ARO2<−pPGK1), |
| | XII-1::(pTDH3−>CkPta, pTEF1−>BbXfpk, EcAroL<−pTEF2, BsPrs<−pPGK1), gpp1Δ::MET17, |
| | XII-2::(pTDH3−>TRP4, SpHis5), aro10Δ, ric1Δ::(pTDH3−>TRP2(S65R, S76L), pPGK1−>ARO4 (K229L)), |
| | X-3::(pTEF2−>TRP1, pCCW12−>TRP3), X-4::(pTDH3−>PanCyPsiK, pCCW12−>PanCyPsiM), |
| | XI-1::(pTEF1−>PanCyCPR, PanCyPsiH<−pPGK1), X-2::(pPGK1−>PanCyCYB5, KIUra3), XVI-21::(pTDH3−>PcPsik, |
| | pCCW12−>PcPsiM), pdc5Δ, erg4Δ, dia3Δ, pho5Δ, pho3Δ |
| SC-417 | MATa his3Δ1 leu2Δ0 met15Δ0 ura3Δ0 XI-5::(pTDH3−>CrTdc, KiLeu2<−pKiLeu2, |
| | pTEF1−>ARO4(K229L), TRP2 (S65R, S76L)<−pTEF2, pFBA1−>ARO1, ARO2<−pPGK1), |
| | XII-1::(pTDH3−>CkPta, pTEF1−>BbXfpk, EcAroL<−pTEF2, BsPrs<−pPGK1), gpp1Δ::MET17, |
| | XII-2::(pTDH3−>TRP4, SpHis5), aro10Δ, ric1Δ::(pTDH3−>TRP2(S65R, S76L), pPGK1−>ARO4 (K229L)), |
| | X-3::(pTEF2−>TRP1, pCCW12−>TRP3), X-4::(pTDH3−>PcPsik, pCCW12−>PcPsiM(H210A)), |
| | XI-1::(pTEF1−>PcCPR, PcPsiH<−pPGK1, KIUra3) |

### Example 3—Construction of E. coli Strains for Production of Substituted Tryptamine Derivatives by Feeding Substituted Indole Acceptors

### Part 1

E. coli strains expressing genes to convert exogenously fed substituted indole acceptors into substituted tryptamine derivatives were constructed as follows. Genes to convert the substituted indole to the substituted tryptophan, and the substituted tryptophan to the substituted tryptamine were synthesized by Twist and cloned into the pRSFDuet-1 expression plasmid. Transformants were selected by plating on media containing kanamycin. Genes to convert the substituted tryptamine further into substituted tryptamine derivatives were synthesized by Twist and cloned into a custom-made plasmid vector (pRSGLY, synthesized by GeneArt) using standard restriction ligation using SpeI/XhoI restriction sites. This custom-made vector contained a LacI operon, AmpR cassette, replication origin and a multiple cloning site flanked by the T7 promoter and terminator. Additionally, the 5′ end also contained a ribozyme binding site (RBS) and a 6×His tag for subsequent protein purification. Fully assembled plasmids were transformed into E. coli DH5a strains or E. coli XJb (DE3) autolysis strains (Zymo Research). Plasmids to convert substituted indoles into substituted tryptamines, and plasmids to convert substituted tryptamines into substituted tryptamine derivatives are shown in Table 7 and Table 8. In some cases, genes to convert substituted tryptamines into substituted tryptamine derivatives were expressed on their own to facilitate substituted tryptamine feeding experiments as well as to facilitate in vitro biocatalytic experiments where the E. coli strains were used to produce and purify enzymes for in vitro reactions.

TABLE 7
Expression plasmids to construct substituted tryptamine
derivative production strains in E. coli

| | |
|---|---|
| Plasmid name | SEQ ID NO |

(Substituted) indole to (substituted) tryptophan

| | |
|---|---|
| PL-541(TmTrpB (M145T, N167D)) | SEQ ID NO: 67 |
| PL-542(PfTrpB(2B9)) | SEQ ID NO: 59 |
| PL-543(PcTrpB) | SEQ ID NO: 179 |
| PL-544(PcTrpB (M439T, N459D) | SEQ ID NO: 181 |

(Substituted) tryptophan to (substituted) tryptamine

| | |
|---|---|
| PL-545(PcncAAAD) | SEQ ID NO: 73 |
| PL-546(RgTdc) | SEQ ID NO: 69 |
| PL-547(PcPsiD) | SEQ ID NO: 71 |
| PL-548(PanCysPsiD) | SEQ ID NO: 77 |

(Substituted) indole to (substituted) tryptamine

| | |
|---|---|
| pRSFDuet-1 (PfTrpB(2B9)-CrTdc) | SEQ ID NO: 59, 25 |
| pRSFDuet-1 (PfTrpB(2B9)-RgTdc) | SEQ ID NO: 59, 69 |
| pRSFDuet-1 (PfTrpB(2B9)-PcPsiD) | SEQ ID NO: 59, 71 |
| pRSFDuet-1 (PfTrpB(2B9)-PcncAAAD) | SEQ ID NO: 59, 73 |
| pRSFDuet-1 (PfTrpB-CrTdc) | SEQ ID NO: 63, 25 |
| pRSFDuet-1 (PfTrpB-RgTdc) | SEQ ID NO: 63, 69 |
| pRSFDuet-1 (PfTrpB-PcPsiD) | SEQ ID NO: 63, 71 |
| pRSFDuet-1 (PfTrpB-PcncAAAD) | SEQ ID NO: 63, 73 |
| pRSFDuet-1 (SeTrpB-CrTdc) | SEQ ID NO: 61, 25 |
| pRSFDuet-1 (SeTrpB-RgTdc) | SEQ ID NO: 61, 69 |
| pRSFDuet-1 (SeTrpB-PcPsiD) | SEQ ID NO: 61, 71 |
| pRSFDuet-1 (SeTrpB-PcncAAAD) | SEQ ID NO: 61, 73 |
| pRSFDuet-1 (TRP5-CrTdc) | SEQ ID NO: 65, 25 |
| pRSFDuet-1 (TRP5-RgTdc) | SEQ ID NO: 65, 69 |
| pRSFDuet-1 (TRP5-PcPsiD) | SEQ ID NO: 65, 71 |
| pRSFDuet-1 (TRP5-PcncAAAD) | SEQ ID NO: 65, 73 |
| pRSFDuet-1 (TmTrpB (M145T, N167D)-CrTdc) | SEQ ID NO: 67, 25 |
| pRSFDuet-1 (TmTrpB (M145T, N167D)-RgTdc) | SEQ ID NO: 67, 69 |
| pRSFDuet-1 (TmTrpB (M145T, N167D)-PcPsiD) | SEQ ID NO: 67, 71 |
| pRSFDuet-1 (TmTrpB (M145T, N167D)-PcncAAAD) | SEQ ID NO: 67, 73 |
| pRSFDuet-1 (PcTrpB (M439T, N459D)-CrTdc) | SEQ ID NO: 181, 25 |
| pRSFDuet-1 (PcTrpB (M439T, N459D)-RgTdc) | SEQ ID NO: 181, 69 |
| pRSFDuet-1 (PcTrpB (M439T, N459D)-PcPsiD) | SEQ ID NO: 181, 71 |
| pRSFDuet-1 (PcTrpB (M439T, N459D)-PcncAAAD) | SEQ ID NO: 181, 73 |

(Substituted) tryptamine to (substituted) tryptamine derivative

| | |
|---|---|
| PL-214(Cs73Y_GA) | SEQ ID NO: 79 |
| PL-226(Pt73Y_GA) | SEQ ID NO: 81 |
| PL-182(Ha88B_2_GA) | SEQ ID NO: 83 |
| PL-520(AtCOMT1) | SEQ ID NO: 113 |
| PL-521(AtIGMT1) | SEQ ID NO: 115 |
| PL-522(HsASMT) | SEQ ID NO: 117 |
| PL-523(OpSTR) | SEQ ID NO: 143 |
| PL-524(RsSTR1 (V208A)) | SEQ ID NO: 145 |
| PL-525(CrSTR1) | SEQ ID NO: 147 |
| PL-526(CrSTR1 (D177A)) | SEQ ID NO: 149 |
| PL-527(MtMcbB) | SEQ ID NO: 151 |
| PL-528(MtMcbB (R72A, H87A)) | SEQ ID NO: 153 |
| PL-532(SsTxtE) | SEQ ID NO: 177 |

### Part 2

Alternatively, genes were synthesized by Twist and cloned as N-terminal HIS-tagged genes into the pET-28a(+) expression vector. Plasmids were cloned as described above. An additional list of plasmids constructed in Example 3 is given in Table 8.

TABLE 8
Additional list of expression plasmids to construct substituted
tryptamine derivative production strains in E. coli

| Plasmid name | SEQ ID NO |
|---|---|
| PL-946(PanCyTrpB) | SEQ ID NO: 255 |
| PL-947(PanCyPsiK) | SEQ ID NO: 159 |
| PL-948(PanCyPsiM) | SEQ ID NO: 127 |

### Example 4—Cultivation of Genetically Modified S. cerevisiae Strains for the De Novo Production of Tryptamine and Substituted Tryptamine Derivatives

### Part 1

Genetically engineered yeast strains were pre-cultured in 500 μL of liquid Delft minimal media with 20 g/L glucose and relevant amino acid supplements for 48 h at 30° C. and 280 rpm in 2 mL microtiter plates with air-permeable sealing. Subsequently, 10 μL of yeast preculture was transferred to 490 μL Delft minimal media with 20 g/L glucose and relevant amino acid supplements and cultivated for 72 h at 30° C. and 280 rpm. After cultivation, extracellular metabolites were extracted by mixing whole cell broth 1:1 with 100% methanol, vortexing thoroughly and centrifuging at 4000×g for 5 min. The supernatant was subsequently diluted in MiliQ water to obtain a final methanol concentration of 12.5% in the samples, which were then analyzed using UHPLC or LC-MS/MS as described in Example 8. Where possible, authentic analytical standards were used for quantification of tryptamine and tryptamine derivatives.

### Part 2

Alternatively, genetically engineered yeast strains were cultivated as described above but extracellular metabolites were extracted as follows: Samples were centrifugated at 4000×g for 5 min, then supernatant was mixed 1:1 with acetonitrile 100%. The extracted samples were diluted as needed so the extracted metabolite was within the calibration range of the analytical method.

### Example 5—Cultivation of Genetically Modified S. cerevisiae Strains for the Production of Substituted Tryptamine Derivatives by Feeding Substituted Indoles

Genetically engineered yeast strains were pre-cultured in 500 μL liquid Synthetic Complete media with relevant drop-out combinations containing 20 g/L glucose for 48 h at 30° C. and 280 rpm in 2 mL microtiter plates with air-permeable sealing. Subsequently, 10 μL of yeast pre-culture was transferred to 490 μL Synthetic Defined (SD) media with 20 g/L glucose and supplemented with relevant amino acids. In some cases cultivation and pre-cultivation was carried out as described in Example 4. Ethanol solutions of substituted indoles were added to the cultivation media yielding a final concentration of 1 mM substituted indole and 2% ethanol, and the strains were cultivated for 72 h at 30° C. and 280 rpm. During this initial cultivation engineered strains converted biosynthetically produced serine and endogenously supplied substituted indoles to substituted tryptophan derivatives which remain inside the engineered cells. Afterwards, the cultivation broths were centrifuged (3000×g 5 min) and the supernatants were discarded. The cell pellets were resuspended in 500 μL SD or Delft media supplemented with relevant amino acids and 20 g/L galactose to induce expression from GAL-promoters and the strains were cultivated for additionally 72 h at 30° C. and 280 rpm. During this secondary cultivation galactose induced expression of promiscuous tryptophan decarboxylases converted the substituted tryptophan into its corresponding substituted tryptamine which could then be freely exported from the cell. In some cultivations, S. cerevisiae strains additionally contained derivatizing enzymes which could further convert the produced substituted tryptamine into a substituted tryptamine derivative. After cultivation, whole cell broths were transferred to 2 mL screw cap tubes containing glass beads (Ø1 mm) and bead-bashed on a FastPrep® FP120 Cell Disrupter (Thermo Savant) at 6.5 m/s for 45 sec. The lysed yeast cells were spun down by centrifugation (4000×g 5 min) and supernatants were analyzed by UHPLC or LC-MS/MS as described in Example 8. In some cases, whole cell broth was centrifuged at 4000×g for 5 min and the supernatant was extracted and analyzed by UHPLC or LC-MS/MS. Where possible authentic analytical standards were used to confirm the identity of the produced molecules.

### Example 6—Cultivation of Genetically Modified E. coli Strains for the Production of Substituted Tryptamine Derivatives by Feeding Substituted Indole Acceptors

E. coli strains were pre-cultured in 500 μL of liquid LB media supplemented with kanamycin and/or ampicillin as required for 24 h at 37° C., 300 rpm in 2 mL microtiter plates with air-permeable sealing. Subsequently 50 μL of pre-culture was transferred to 450 μl of LB media with 20 g/L glucose, polypeptide expression inducer (3 mM arabinose+0.1 mM IPTG) and ethanol solutions of various substituted indole molecules (1 mM indole 2% ethanol) and cultured for 24 h at 37° C., 300 rpm. After cultivation, whole cell broths were transferred to 2 mL screw cap tubes containing glass beads (Ø1 mm) and bead-bashed on a FastPrep® FP120 Cell Disrupter (Thermo Savant) at 6.5 m/s for 45 sec. The lysed E. coli cells were spun down by centrifugation (4000×g 5 min) and supernatants were analyzed by UHPLC or LC-MS/MS as described in Example 8. In some cases, whole cell broth was centrifuged at 4000×g for 5 min and the supernatant was extracted and analyzed by UHPLC or LC-MS/MS. Where possible authentic analytical standards were used to confirm the identity of the produced molecules.

### Example 7—Assay Conditions for the Biocatalytic Production of Substituted Tryptamine Derivatives by Feeding Indole or Tryptamine Substrates in In Vitro Enzyme Assays

### Part 1

In some instances, production of substituted tryptamine derivatives can be carried in vitro using purified enzymes with addition of required co-factors and substrates. Preparation and running of the biocatalytic reactions were performed as follows:

5 mL of 2× concentrated LB medium+Ampicillin (50 μg/mL) was inoculated with E. coli XJb (DE3) strains expressing a gene of interest and incubated overnight at 30° C. with shaking. The following day, cell cultures were transferred into 500 mL of 2× concentrated LB medium+Ampicillin (50 μg/mL) and incubated overnight at 30° C. with shaking. The following day, the cell cultures were transferred to 1 L of 2× concentrated LB medium+Ampicillin (50 μg/mL)+3 mM arabinose+0.1 mM IPTG. Cells were incubated for 24 h at 20° C. with shaking. The following day, the cells were collected by centrifugation at 6500×g for 10 mins at 4° C. Cells were resuspended in 20 mL ice-cold GT buffer (50 mM Tris-HCl pH7.4+1 mM phenylmethanesulfonyl fluoride+1 cOmplete™, mini, EDTA-free Protease Inhibitor Cocktail tablet (Roche)). The resuspended material was transferred to a 50 mL falcon tube and kept at −80° C. for at least 15 mins. Falcon tubes were then thawed at room temperature, as the tubes were thawing the following reagents were added; 2.6 mM MgCl₂, 1 mM CaCl₂, 250 μL of a 1.4 mg/ml DNase solution (Sigma) dissolved in MilliQ water. Tubes were gently inverted to mix then were incubated for 5 mins at 37° C. 4× Binding buffer (10 mL) was then added to the tubes (to final conc of 50 mM Tris-HCl pH7.4, 10 mM imidazole, 500 mM NaCl and the pH adjusted to 7.4 with HCl). The mix was centrifuged at 10000×g for 30 mins at 4° C., the supernatant transferred to a fresh 50 mL falcon tubes and centrifuged again to remove any remaining cellular debris at 10000×g for 30 minutes at 4° C. While the enzyme prep was centrifuging, 3 mL of HIS-Select (available from Sigma P6611) column material was added to a fresh 50 mL tube and washed by adding MilliQ water up to 50 mL, centrifuging at 2000×g for 2 mins and discarding the supernatant. This washing step was repeated. Finally, MilliQ water was added to the HIS-Select material to an approximate 50% volume. Collected supernatant from the centrifuged enzyme preparation was transferred to the tube containing the HIS-Select material through a Miracloth (available from Merck Millipore), and then incubated at 4° C. with gently shaking by inversion for 2 h. After 2 h the mix was centrifuged at 2000×g for 4 minutes at 4° C. and the supernatant discarded. The remaining HIS-Select material was washed twice with 1× binding buffer (50 mM Tris-HCl, 0.5M NaCl, 10 mM Imidazole, pH 7.4) with centrifugation at 2000×g for 4 minutes at 4° C. The HIS-Select material was resuspended in 5 mL 1× binding buffer and transferred to a Poly-Prep® Chromatography Column (available from BioRad, 7311550). The HIS-Select material was kept at 4° C. and washed twice with 1× binding buffer by filling up the column and allowing it to drip through. Finally, purified enzymes were eluted from the HIS-Select material by adding 7.5 mL of elution buffer (50 mM Tris-HCl, 500 mM Imidazole, pH7.4) and collecting the flow through. Enzymes were used immediately in in vitro enzyme assays or stored at −20° C. in 50% glycerol until needed.

In vitro conversion of various (substituted) indoles to (substituted) tryptophans and/or (substituted) tryptophans to (substituted) tryptamines and/or (substituted) tryptamines to (substituted) tryptamine derivatives was carried out according to Table 9. Substrates were dissolved in either water, methanol or DMSO depending on the substrate. Required co-factors were provided by a commercial supplier (e.g. Sigma).

TABLE 9
Assay set up for conversion of substituted indoles
to substituted tryptamine derivatives in vitro.

| Reagents | Volume (μL) |
|---|---|
| Purified enzymes | 5 |
| 25 mM substrate | 0.4 |
| 25 mM Co-factor/co-substrate | 0.4 |
| 1M Tris-HCl pH 7.8 | 2 |
| Milli-Q water | 12.2 |
| TOTAL | 20 |

The reaction mixture was scaled up or down as required. The reaction mixture was incubated without shaking at 30° C. for 24 hours. Samples were extracted by the addition of ice-cold 100% MeOH to a final concentration of 75% followed by centrifugation at 4000 rpm for 10 min. The supernatant was diluted to 12.5% with water prior to analysis as described in example 8. To confirm the identity of the produced substituted indoles LC-MS/QTOF was used as described in Example 8 to confirm the expected mass and fragmentation pattern of each detected molecule. Quantification of substituted indole production was done by comparing the peak area of the indole substrate and the substituted indole with authentic analytical standards (where available), where a substrate was unavailable, quantification was achieved by comparing with an authentic analytical standard of the indole substrate. % Conversion of substrates to substituted indoles by enzymatic biocatalysis was calculated by measuring the decrease in substrate and increase in product after 24 h incubation.

### Part 2

Alternatively, 5 mL of 2× concentrated LB medium+appropriate antibiotic (50 μg/mL) was inoculated with E. coli XJb (DE3) strains expressing a plasmid of interest and incubated overnight at 30° C. with shaking. The following day, 50 ml TB medium+antibiotic in 250 ml baffled flasks was inoculated with 0.5 mL overnight cell culture and incubated for 3.5 hrs at 30° C. The cell cultures were then induced with 3 mM arabinose, 0.1 mM IPTG and Incubated for 24 h at 25° C., with shaking. The following day, the cells were collected by centrifugation at 6500×g for 10 mins at 4° C. Cells were resuspended in 5 mL ice-cold protein extraction buffer (50 mM Tris-HCl pH7.4+1 mM phenylmethanesulfonyl fluoride+1 cOmplete™, mini, EDTA-free Protease Inhibitor Cocktail tablet (Roche). The resuspended material was transferred to five 1.5 mL 80ppendorf tubes and kept at −80° C. for at least 15 mins. The tubes were then thawed at room temperature, as the tubes were thawing the following reagents were added; 2.6 mM MgCl₂, 1 mM CaCl₂, 300 U/mL Dnase solution (DENARASE®, C-lecta) and 0.2 mg/mL lysozyme (Sigma) dissolved in MilliQ water. Tubes were gently inverted to mix then were incubated for 10-15 mins at 37° C. 3 volumes of 4× Binding buffer were then added to the tubes (to final concentration of 50 mM Tris-HCl pH7.4, 10 mM imidazole, 500 mM NaCl and the pH adjusted to 7.4 with HCl). The mix was centrifuged at 10000×g for 30 mins at 4° C., the supernatant transferred to a fresh 80ppendorf tubes and centrifuged again to remove any remaining cellular debris at 10000×g for 30 minutes at 4° C. While the enzyme prep was centrifuging, HisPur 0.2 ml spin column (Thermo Scientific) are prepared as instructed by manufacturer. HisPur column was washed with MilliQ water and equilibrated with two resin-bed volumes of 1×his-binding buffer two times. HisPur column was centrifuged at 700×g for 2 minutes to remove buffer. 600 μL of collected supernatant from the centrifuged enzyme preparation was transferred to the HisPur column and then incubated at 4° C. with gentle shaking by inversion for 30 minutes. After 30 minutes, the unbound protein was removed by centrifugation 700×g for 2 min. The remaining 600 μL of collected supernatant from the centrifuged enzyme preparation was loaded to the HisPur column and incubated again at 4° C. with shaking. Unspecific binding of other proteins was removed by washing twice with 1× binding buffer (50 mM Tris-HCl, 0.5M NaCl, 10 mM Imidazole, pH 7.4). Enzyme was resuspended in 200 μl elution buffer for 2 minutes before elution by centrifugation (700×g for 2 minutes at 4° C.). Enzymes were used immediately in in vitro enzyme assays or stored at −20° C. in 50% glycerol until needed.

TABLE 10
Assay set up for conversion of substituted indoles
to substituted tryptamine derivatives in vitro.

| Reagents | Volume (μL) |
|---|---|
| Purified enzymes | 5 |
| 50 mM substrate | 3 |
| 50 mM Co-factor/co-substrate | 3 |
| 1 mM Pyridoxal phosphate (PLP) | 0.05 |
| 1M Tris-HCl pH 8.0 | 2.5 |
| Milli-Q water | 36.45 |
| TOTAL | 50 |

### Example 8—Detection and Quantification Methods for Substituted Tryptamine Derivatives

### Part 1

LC-MS/QTOF was performed on a Dionex UltiMate 3000 Quaternary Rapid Separation UHPLC⁺ focused system (Thermo Fisher Scientific, Germering, Germany) coupled to a Compact micrOTOF-Q mass spectrometer (Bruker, Bremen, Germany) equipped with an electrospray ion source (ESI) operated in positive ion mode. Separation was achieved on a Kinetex XB-C18 column (150×2.1 mm, 1.7 μm, 100 Å, Phenomenex). For eluting 0.05% (v/v) formic acid in H₂O and 0.05% (v/v) formic acid in acetonitrile were employed as mobile phases A and B, respectively. Gradient conditions were as follows: 0.0-1.0 min 2% B; 1.0-24.0 min 2-75% B, 24.0-25.0 min 75-100% B, 25.0-27.5 min 100% B, 27.5-28.0 min 100-2% B, and 28.0-30.0 min 2% B. The flow rate of the mobile phase was 300 μL/min and the injection volume was 10 μL. The column oven temperature was maintained at 30° C. UV spectra for each sample were acquired at 220, 230, 240, and 280 nm. The ion spray voltage was maintained at +4500 V. Dry temperature was set to 250° C., and the dry gas flow was set to 8 L/min. Nitrogen was used as the dry gas, nebulizing gas, and collision gas. The nebulizing gas was set to 2.5 bar and collision energy to 10 eV. MS spectra were acquired in an m/z range from 50 to 1000 amu and MS/MS spectra in a range from 100-800 amu. Sampling rate was 2 Hz. Sodium formate clusters were used for mass calibration. All files were automatically calibrated by postprocessing. The data was processed using Bruker Compass DataAnalysis 4.3.

UHPLC analysis of tryptamine, serotonin, and other substituted tryptamine derivatives was performed on an Agilent 1290 Infinity II LC system (Agilent Technologies, Böblingen, Germany). Separation was achieved on a Kinetex XB-C18 column (100×2.1 mm, 1.7 μm, 100 Å, Phenomenex). Isocratic elution using 0.05% (v/v) formic acid in H₂O was employed using a flowrate of 0.60 ml/min and a run-time of 5 min. The column oven temperature was maintained at 35° C. and the injection volume was 5 μL. UV spectra for each sample were acquired at 220 nm. The data was processed using Agilent Openlab CDS Chemstation Rev. C.01.10.

### Part 2

Alternatively, UHPLC analysis of tryptamine, serotonin, and other substituted tryptamine derivatives was performed on an Agilent 1290 Infinity II LC system (Agilent Technologies, Böblingen, Germany). Separation was achieved on a Waters ACQUITY UPLC CSH Fluoro-Phenyl Column (100×2.1 mm, 1.7 μm, 130 Å) using gradient elution. For elution 10 mM ammonium acetate in H₂O at pH=7 and 10% water in 90% (v/v) methanol with 10 mM ammonium acetate were employed as mobile phases A and B, respectively. Gradient conditions were as follows: 0.0-5.0 min 5-95% B; 5.0-7.0 min 95% B, 7.0-7.1 min 95-5% B. The flow rate of the mobile phase was 0.400 ml/min and the injection volume was 2 μL. The column oven temperature was maintained at 35° C. UV spectra for each of the samples were acquired at 220 and 280 nm. The data was processed using Agilent Openlab CDS Chemstation Rev. C.01.10.

UHPLC analysis of baeocystin, norbaeocystin, psilocybin and other substituted tryptamine derivatives was performed on an Agilent 1290 Infinity II LC system (Agilent Technologies, Böblingen, Germany). Separation was achieved on a Waters ZORBAX RRHD HILIC Plus column (100×2.1 mm, 1.8 μm, 95 Å) using gradient elution. Mobile phase A consisted of 5% H₂O in 95% (v/v) acetonitrile with 10 mM Amonmium Acetate and 10 mM Formic acid, while mobile phase B consisted of 50% H₂O in 50% (v/v) acetonitrile with 10 mM Amonmium Acetate and 10 mM Formic acid. Gradient conditions were as follows: 0.0-6.0 min 5% B; 6.0-11.5 min 5-95% B, 11.5-12.0 min 95% B, 12.0-12.1 min 95-5% B, 12.1-12.2 min 5% B. The flow rate of the mobile phase was 0.400 ml/min and the injection volume was 1 μL. The column oven temperature was maintained at 40° C. UV spectra for each of the samples were acquired at 210 and 280 nm. The data was processed using Agilent Openlab CDS Chemstation Rev. C.01.10.

LC-MS analysis of psilocybin and related derivatives and metabolites was performed as follows. High resolution LC-MS measurements were carried out on a Dionex UltiMate 3000 UHPLC (Thermo Fisher Scientific, US), connected to an Orbitrap Fusion Mass Spectrometer (Thermo Fisher Scientific, US). The UHPLC was equipped with a SeQuant zic-Hilic column (Merck KgaA), 15 cm×2.1 mm, 3 μm. The temperature was 35° C. and the flow rate 0.5 mL/min. The system was running an isocratic gradient with a mobile phase consisting of 20% 10 mM ammonium formate (pH 3) and 80% acetonitrile, with 0.1% formic acid. The samples were passed on to the MS equipped with a heated electrospray ionization source (HESI) in positive-ion mode with sheath gas set to 50 (a.u.), aux gas to 10 (a.u.) and sweep gas to 1 (a.u.). The cone and probe temperature were 325° C. and 350° C., respectively, and spray voltage was 3500 V. Scan range was 100-800 Da and time between scans was 50 ms. In all cases, authentic analytical standards were used to the produced metabolites.

### Example 9—Test of Chemical Stability of Substituted Tryptamine Derivatives

Chemical stability of substituted tryptamine derivatives was determined under alkaline, acidic, oxidative and heat stress as follows. 25 mM stock solutions of the target tryptamine molecules were prepared in 100% methanol. 15 μL is mixed with 5 μL of 400 mM HCl solution (final pH=1.1), 400 mM NaOH solution (Final pH=12.5), 12% H₂O₂ solution (final concentration 3%), or H₂O pH 7.0. Acidic, alkaline and oxidative samples were incubated at 30° C. for 24 h while samples in water were incubated at 80° C. for 24 h. A control under ambient conditions was also prepared where 15 μL of the molecule was added to 5 μL H₂O pH 7.0 and incubated at 30° C. After 24 h samples were placed on ice and 60 μL of ice-cold 100% methanol is added to each sample. Samples were centrifuged and transferred to HPLC vials for analysis. The remaining concentration was quantified by comparing to authentic analytical standards. Determining the presence of degradation products were determined by comparing with authentic analytical standards.

### Example 10—In Vitro Production of Substituted Tryptamine Glycosides

For in vitro studies of glycosyl transferase performance in glycosylating substituted tryptamines, purified glycosyl transferases were prepared as described in Example 7 and in vitro enzyme assays run as described below in Table 11.

TABLE 11
Assay set up for producing substituted
tryptamine glycosides in vitro

| Reagent | Volume (μL) |
|---|---|
| Purified glycosyl transferase enzyme | 5 |
| 25 mM substituted tryptamine substrate | 0.4 |
| 1M Tris-HCl pH 7.4 | 2 |
| Milli-Q water | 11.9 |
| Alkaline phosphatase (1 U/μL) | 0.2 |
| 50 mM UDP-sugar | 0.5 |
| TOTAL | 20 |

The reaction mixture was scaled up or down as required. The reaction mixture was incubated without shaking at 30° C. for 24 hours. Extraction and analysis were performed as described (Examples 7 and 8) above for this example. To confirm the identity of the produced substituted tryptamine glycosides LC-MS/QTOF was used as described above (example 8) to confirm the expected mass and fragmentation pattern of each detected molecule. Quantification of substituted tryptamine glycoside production was done by comparing the peak area of the substituted tryptamine substrate and the substituted tryptamine glycoside with authentic analytical standards (where available), where a standard was unavailable, quantification was achieved by comparing with an authentic analytical standard of the substituted tryptamine aglycone. % Conversion of substituted tryptamine substrates to substituted tryptamine glycosides by specific Glycosyl transferases was calculated by measuring the decrease in substrate and increase in product after 24 h incubation. Quantification was also achieved using the UHPLC analytical methods described in Example 8.

### Example 11—High-Level De Novo Production of Tryptamine by Engineered S. cerevisiae Strains

### Part 1

S. cerevisiae strains producing high amounts of tryptamine were constructed as described above. Tryptamine producing strains are ideal “mother strains” for the introduction of derivatizing enzymes to produce a range of substituted tryptamine derivatives de novo. Cultivation, extraction, and analysis was carried out as described in Example 4 and 8 to quantify the amount of tryptamine produced by various engineered S. cerevisiae strains as shown in Table 12. It was found that overexpression of various heterologous and native S. cerevisiae genes resulted in high levels of tryptamine production.

TABLE 12
De novo tryptamine production titers by
engineered S. cerevisiae strains

| | Average tryptamine |
|---|---|
| Strain | titer (mg/L |
| BY4741 | ND |
| SC-17 | 85.2 |
| SC-18 | 31.5 |
| SC-25 | 149.3 |
| SC-43 | 123.7 |
| SC-44 | 202.7 |

### Part 2

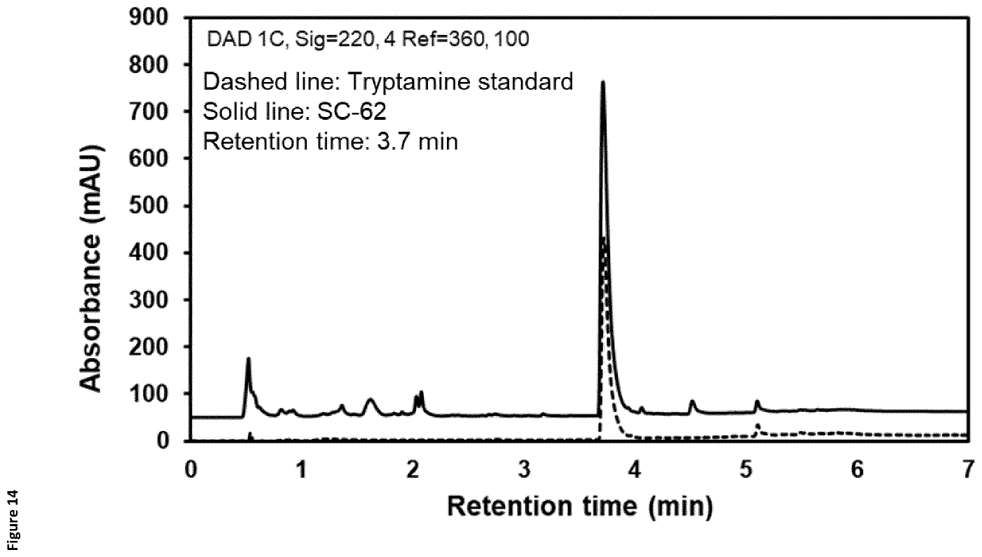

In a follow up experiment, tryptamine production strains were further engineered to increase titer. Strains were constructed according to Example 1 and a list of strains constructed is shown in Table 5 and Table 6. Strains were cultivated according to Example 4 and quantified according to Example 8. The results, shown in Table 13 and FIG. 14 demonstrate further genetic modifications significantly increase tryptamine titer. The resulting highest production strains serves as an ideal starting point to further engineer S. cerevisiae to produce substituted tryptamine derivatives.

TABLE 13
Further de novo tryptamine production by engineered
S. cerevisiae strains in mg/L.

| | Average |
|---|---|
| Strain | titer (mg/L |
| BY4741 | ND |
| SC-49 | 252.96 |
| SC-56 | 332.45 |
| SC-58 | 247.59 |
| SC-59 | 362.97 |
| SC-60 | 204.64 |
| SC-62 | 511.01 |
| SC-106 | 448.62 |

ND: Not detected

### Conclusion

Through the genetic modifications including native gene overexpressions, gene deletions, and heterologous gene expressions in S. cerevisiae, production of the non-native metabolite tryptamine could be produced in significant amounts far beyond anything reported in the art.

### Example 12—De Novo Production of Substituted Tryptamine Derivative Serotonin by Engineered S. cerevisiae Strains

### Part 1

De novo production of serotonin in yeast was achieved by combining the gene encoding RgTdc to convert L-tryptophan to tryptamine with genes encoding enzymes (OsT5H and FoCPR) to convert tryptamine to serotonin. Integrative plasmids harboring these genes were introduced into S. cerevisiae strain BY4741 as described previously. The genetically engineered yeast strains were cultivated, and extracellular metabolites extracted as described in Example 4 and 8. Production of serotonin was quantified by comparison to authentic analytical serotonin standards by UHPLC analysis. Cultivation results are shown in Table 14. Overexpression of these genes successfully resulted in the production of serotonin.

TABLE 14
Production of serotonin by genetically
engineered S. cerevisiae strain.

| | Average |
|---|---|
| Strain | serotonin titer (mg/L |
| BY4741 | ND |
| SC-21 | 25.8 |

It was found that yeast could be engineered to produce serotonin by conversion of tryptophan to tryptamine (by a tryptophan decarboxylase) then direct hydroxylation at the 5-position on the indole ring by a tryptamine hydroxylase and a cytochrome P450 reductase. This is a significant advancement on methods known in the art (e.g. WO2013127915) which utilizes the tetrahydrobiopterin dependent tryptophan hydroxylase enzyme to convert tryptophan to 5-hydroxytryptophan, then decarboxylation to serotonin by 5-hydroxytryptophan decarboxylase. Tetrahydrobiopterin is not natively produced by most microorganisms including yeast requiring either supplementation with this expensive co-factor, or extensive engineering efforts to engineer the microorganism to produce the cofactor de novo. Direct conversion from tryptamine as demonstrated here is not only more efficient but alleviates the need to consider this additional cofactor requirement. Also surprising is the observation that a CYP/CPR pair from different organisms works efficiently in yeast. Typically, cytochrome P450's (CYP's) requires the action of a specific cytochrome P450 reductase (CPR) to function. Many plant species for example have multiple CPR enzymes which function only with specific CYP's from that species. Interestingly a CYP from Orzya Sativa (OsT5H) is functional with a CPR from Fusarium oxysporum (FoCPR).

### Part 2

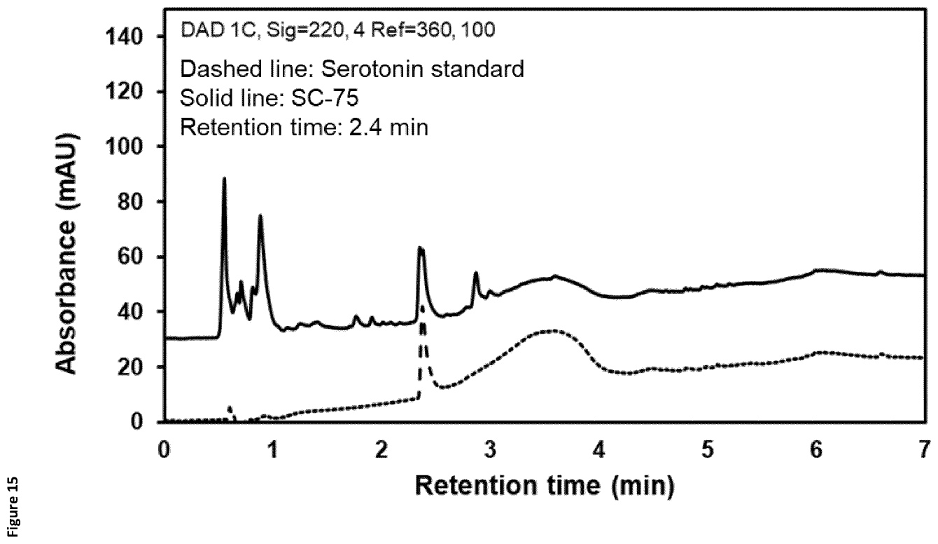

In a follow up experiment, serotonin production was further improved by introducing OsT5H (SEQ ID NO: 95) and FoCPR (SEQ ID NO: 111) into a higher tryptamine producing strain (SC-106). Strains were constructed according to Example 1, cultivated according to Example 4 and quantified according to Example 8. The results, shown in Table 15 and FIG. 15 demonstrate further improvement in serotonin titer with SC-75 producing significantly higher amounts in serotonin, indicating that the OsT5H/FoCPR enzyme pair has significant biocatalytic capacity to convert tryptamine into serotonin. The results also demonstrate that expression of tryptamine 5-hydroxylase (OsT5H) alone is insufficient to enable efficient serotonin production, and that the concerted action of a CYP/CPR pair is needed for full catalytic activity. In a strain expressing OsT5H without FoCPR (SC259), only trace amounts of serotonin are detected demonstrating the importance of co-expression of an efficient CPR. While S. cerevisiae does contain a native CPR enzyme (Ncp1), it typically does not function with heterologous CYP enzymes such as tryptamine 5-hydroxylases. Indeed, the results shown in Table 15 show that in SC-259, a strain expressing OsT5H but no heterologous CPR (relying on the native yeast Ncp1) minimal production of serotonin is observed.

TABLE 15
Further production of serotonin by engineered S. cerevisiae strains
in mg/L, data presented as the average of triplicate experiments.

| Strain | Genotype description | Tryptamine | Serotonin |
|---|---|---|---|
| BY4741 | Wild-type | ND | ND |
| SC-106 | Improved tryptamine | 410.29 | ND |
| | producer | | |
| SC-259 | Serotonin producer | 296.98 | 10 |
| | without FoCPR | | |
| SC-75 | Serotonin producer | 7.88 | 641.29 |
| | with FoCPR | | |

ND: Not detected

### Conclusion

This experiment demonstrates that S. cerevisiae can be engineered to efficiently produce serotonin by an alternative biosynthetic pathway not known in the art. This production method offers significant improvement over prior art by being more efficient but also alleviating the need to produce tetrahydrobiopterin or add it exogenously. This experiment also demonstrates that functional expression a tryptamine 5-hydroxylase requires co-expression of a cytochrome p450 reductase such as FoCPR for full catalytic activity. Finally, this experiment demonstrates the surprising observation that FoCPR significantly enhances the activity of OsT5H even though its from a completely different organism.

### Example 13—De Novo Production of Substituted Tryptamine Derivative 4-Coumaroyl Serotonin by Engineered S. cerevisiae Strains

De novo production of the high-value molecule 4-coumaroyl serotonin, derived from Safflower was achieved by combining enzymes for the de novo production of tryptamine (RgTdc), enzymes to derivatize tryptamine to serotonin (OsT5H, FoCPR), enzymes for the de novo production of 4-Coumoryl CoA (ARO7(G141S), ARO8, Pal2, C4 h, Atr2, 4CI), and finally an enzyme to derivatize serotonin with 4-Coumoryl CoA to produce 4-Coumoryl serotonin (CaSHT). Integration plasmids encoding these genes were introduced into S. cerevisiae as described above.

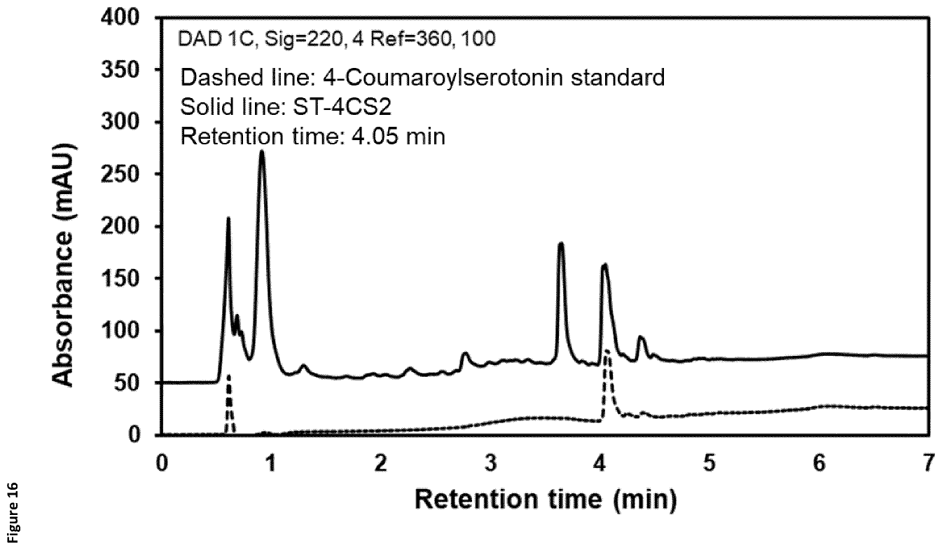

Genetically engineered strains were cultivated as described above and production of 4-Coumoryl serotonin as well as other pathway intermediates were quantified by comparison to authentic analytical standards by HPLC analysis. Genotypes for each strain tested is shown in Table 5, while results from the cultivation experiment shown in Table 16 and FIG. 16. Overexpression of these genes successfully resulted in the production of 4-coumorylserotonin.

TABLE 16
Production of 4-Coumorylserotonin by genetically engineered
S. cerevisiae strains. Shown is average titer
from triplicate cultivations of each strain.

Average titer (mg/L

| | | Phloretic | Coumaric | |
|---|---|---|---|---|
| Strain | Serotonin | acid | acid | 4CS |
| ST-4CS1 | 168 | 412 | 26 | 88 |
| ST-4CS2 | 0 | 215 | 31 | 410 |

4CS: 4-Coumorylserotonin.

### Conclusion

The results of this cultivation experiment show that yeast can be engineered to efficiently produce complex serotonin derivatives like 4-coumaroylserotonin (4CS), a molecule which has application as a potent anti-oxidant, tyrosinase inhibitor, and anti-hyperpigmentation agent.

### Example 14—De Novo Production of Other Substituted Tryptamine Derivatives by Engineered S. cerevisiae Strains

By combining one or several derivatizing enzymes with strains which produce tryptamine, a range of substituted tryptamine derivatives can be produced. This was demonstrated by constructing strains which produced tryptamine (expressing RgTdc), then converting tryptamine to serotonin (by expressing OsT5H and FoCPR), then converting serotonin into more complex derivatives such as O-methyl serotonin by expressing HsASMT and N-methyl serotonin by expressing CsSNMT1. Strains were cultivated, extracted and analyzed by LC-MS/QTOF as described previously to confirm the production of the substituted tryptamine derivative by the engineered strains as shown in Table 17.

TABLE 17
S. cerevisiae strains, retention time, calculated theoretical
m/z, experimentally observed m/z and fragmentation pattern
of O-methyl serotonin and N-methylserotonin produced de novo
by engineered S. cerevisiae strains

| | | Reten- | Calcu- | | |
|---|---|---|---|---|---|
| | | tion | lated | Observed | Fragmen- |
| | | time, | m/z | m/z | tation |
| Strain | Product | min. | [M + H]⁺ | [M + H]+ | pattern |
| SC-34 | O-methyl | 1.8 | 191.1179 | 191.1173 | MS2(191.1173): |
| | serotonin | | | | m/z 174.0906 |
| | | | | | corresponding |
| | | | | | to loss of the |
| | | | | | amino-group |
| SC-36 | N-methyl | 1.8 | 191.1179 | 191.1167 | MS2(191.1167): |
| | serotonin | | | | m/z 160.0749 |
| | | | | | corresponding |
| | | | | | to loss of the |
| | | | | | methylamine- |
| | | | | | group |

### Conclusion

The results of this experiment show that yeast strains engineered to production serotonin can be further engineered to produce more complex serotonin derivatives. The results also surprisingly demonstrate that HsASMT, and enzyme described as converting normelatonin into melatonin, also has catalytic activity for serotonin and can convert it into O-methylserotonin. Finally, these results demonstrate that a putative methyltransferase from Citrus sinensis (CsSNMT1) is also active on serotonin.

### Example 15—Production of Substituted Tryptamine Derivatives by Feeding Substituted Indoles to Engineered S. cerevisiae Strains

Production of 5-flouro-tryptamine and 5-methoxy-tryptophan was achieved by feeding substituted indoles to genetically engineered S. cerevisiae strains constructed as described in Example 2. The strains harboured centromeric plasmids containing the genes encoding PfTrpB(2B9) for conversion of substituted indoles to substituted tryptophan and RgTdc for conversion of substituted tryptophan to the corresponding substituted tryptamine. RgTdc was placed under the control of a GAL10-promoter and expression was induced with galactose after an initial cultivation with glucose. The yeast strains were cultivated as described in Example 5 with supplementation of either 1 mM 5-fluoro-indole or 1 mM 5-methoxy-indole resulting in the production of 5-fluoro-tryptamine and 5-methoxy-tryptophan, respectively. Metabolites were extracted and analyzed by LC-MS/MS as previously described. Results of the LC-MS/MS analysis are provided in Table 18. While it was found that strains expressing a heterologous tryptophan synthase could successfully convert substituted indoles to substituted tryptophans, surprisingly, it was found that a wild-type S. cerevisiae strain (BY4741) could also catalyze this reaction successfully converting 5-methoxy-indole into 5-methoxy-tryptophan. This indicates that native S. cerevisiae enzyme Trp5 also has capacity to convert substituted indoles into their corresponding substituted tryptophans. It was also found that tryptophan decarboxylases could convert substituted tryptophans into their corresponding substituted tryptamines as shown by the conversion of 5-fluoro-indole to 5-fluoro-tryptamine by combining a heterologous tryptophan synthase with a tryptophan decarboxylase.

TABLE 18
S. cerevisiae strains, retention time, calculated theoretical m/z, experimentally
observed m/z and fragmentation pattern of 5-fluorotryptamine and 5-methoxytryptophan
produced by the in vivo conversion of 5-fluoroindole and 5-methoxyindole, respectively
by engineered S. cerevisiae strains.

| | | | Retention | Calculated | Observed | |
|---|---|---|---|---|---|---|
| | | | time, | m/z | m/z | Fragmentation |
| Strain | Substrate | Product | min. | [M + H]⁺ | [M + H]⁺ | pattern |
| SC-41 | 5-fluoro- | 5-fluoro- | 5.5 | 179.0979 | 179.0983 | MS2(179.0983): m/z |
| | indole | tryptamine | | | | 162.0697 |
| | | | | | | corresponding to |
| | | | | | | loss of the amino- |
| | | | | | | group |
| SC-41 | 5-methoxy- | 5-methoxy- | 5.7 | 235.1077 | 235.1081 | MS2(235.1081): m/z |
| | indole | tryptophan | | | | 218.0807 |
| | | | | | | corresponding to |
| | | | | | | loss of the amino- |
| | | | | | | group, m/z |
| | | | | | | 174.0911, |
| | | | | | | corresponding to the |
| | | | | | | loss of the amino- |
| | | | | | | group and |
| | | | | | | decarboxylation. |
| BY4741 | 5-methoxy- | 5-methoxy- | 5.7 | 235.1077 | 235.1074 | MS2(235.1074): m/z |
| | indole | tryptophan | | | | 218.0807 |
| | | | | | | corresponding to |
| | | | | | | loss of the amino- |
| | | | | | | group, m/z |
| | | | | | | 174.0911, |
| | | | | | | corresponding to the |
| | | | | | | loss of the amino- |
| | | | | | | group and |
| | | | | | | decarboxylation. |

### Conclusion

This experiment demonstrated that in the absence of de novo production of a substituted tryptamine derivative of interest, yeast can also produce these compounds by importing substituted indole precursors supplied in the cultivation media and using the broad substrate scope of heterologous tryptophan synthase and tryptophan decarboxylase, produce and export the corresponding substituted tryptophan and/or tryptamine derivative.

### Example 16—Production of Iboga Alkaloids by Bioconversion Using Genetically Engineered S. cerevisiae

Iboga alkaloids including ibogaine and noribogaine are a therapeutically relevant class of molecules currently being investigated as promising treatments for addiction. Sourcing these molecules is currently difficult due to the scarcity of the native species which produces these molecules, Tabernanthe iboga, as well as the relatively low abundance of the molecules in these hosts. As an alternative, genetically engineered yeast can be used to convert precursor molecules from more abundant sources to the final iboga alkaloid(s) of interest.

As an example, production of ibogaine and noribogaine was achieved by bioconversion of alkaloids found in the root bark of abundant Tabernaemontana 901ba. Extraction and decarboxylation was carried out according to (Krengel et al., 2019) to obtain a methanolic extract of alkaloids lacking the methoxy moiety.

50 ml Falcon tubes containing 3 g of dried and powdered root bark from each species were added to 40 ml methanol and either macerated or sonicated for 60 min. During this period, the tubes were vortexed four times and finally centrifuged at 2400 rpm for 5 min. The supernatant was recovered by pipetting through degreased cotton and evaporated to dryness.

450 mg of each extract were dissolved separately in 30 ml of methanol and potassium hydroxide mixture (MeOH/KOH mix was made by dissolving 9 g of KOH in 2 ml of water and diluted with methanol to a final volume of 60 ml) in 50 ml Pyrex tubes which were then submerged in a hot water bath and maintained at an internal temperature of 72-75° C. for 2 h. The saponified methanolic extracts were evaporated to dryness. The latter were suspended separately in 25 ml of 2 M aqueous hydrochloric acid and gravity filtered through Whatman grade 2 filter papers. The filtrates were heated to 95° C. in 50 ml Pyrex tubes submerged in a hot water bath. After 15 min, the solutions could cool at room temperature before raising the pH to 10 with ammonium hydroxide and gravity filtering. The resulting filtrates and dried precipitated material were extracted five times with dichloromethane, respectively, which after evaporation yielded the saponified and decarboxylated alkaloid extracts.

The demethoxycarbonylated alkaloid extract was dissolved in ethanol and fed to engineered S. cerevisiae strains containing enzymes from the ibogaine biosynthetic pathway (2% final ethanol concentration). Cells expressed either the Tabernanthe iboga Ibogamine-10-hydroxylase (PL-533(Rec 2-LEU: Til10H), SEQ ID NO. 99) and Cytochrome P450 reductase (PL-535(Rec 3x-XI-5: CrCPR1) or PL-536(Rec 3x-XI-5: CrCPR2), SEQ ID NO's. 107 or 109) alone or in combination with the Tabernanthe iboga Noribogaine 10-O-methyltransferase (PL-534(Rec 1-XI-5-LEU: TiN100MT), SEQ ID NO. 119). Cultures were incubated in 500 μL of Delft media with appropriate amino acids added and incubated at 30° c. for 72 h. Whole cell broth was centrifuged at 4000×g for 5 min and the supernatant was extracted and analyzed by UHPLC or LC-MS/MS. Where possible authentic analytical standards were used to confirm the identity of the produced molecules.

### Example 17—Construction of E. coli Strains for Production of Substituted Tryptamine Glycosides from Substituted Tryptamines

E. coli strains expressing genes to convert substituted tryptamines into substituted tryptamine glycosides were constructed as follows. Glycosyltransferase genes of were codon-optimized for E. coli, synthesized by Twist and cloned into a custom-made plasmid vector (pRSGLY, synthesized by GeneArt) using standard restriction ligation using SpeI/XhoI restriction sites. This custom-made vector contained a LacI operon, AmpR cassette, replication origin and a multiple cloning site flanked by the T7 promoter and terminator. Additionally, the 5′ end also contained a ribozyme binding site (RBS) and a 6×His tag for subsequent protein purification. Fully assembled plasmids were transformed into E. coli DH5a strains or E. coli XJb (DE3) autolysis strains (Zymo Research). Plasmids encoding substituted tryptamine glycosyltransferases are shown in Table 19. In some cases, glycosyltransferases were expressed on their own to facilitate substituted tryptamine feeding experiments as well as to facilitate in vitro biocatalytic experiments where the E. coli strains were used to produce and purify enzymes for in vitro reactions.

TABLE 19
Expression plasmids to construct substituted tryptamine
glycoside production strains in E. coli

| | Gene | SEQ |
|---|---|---|
| Plasmid name | expressed | ID NO |
| PL-226(Pt73Y_GA) | Pt73Y | 203 |
| PL-85(At73B5_GA) | At73B5 | 201 |
| PL-28(At72B1_GA) | At72B1 | 197 |
| PL-364(Ha72T_GA) | Ha72T | 189 |
| PL-342(Cp73B_GA) | Cp73B | 235 |
| PL-326(Ha72B_GA) | Ha72B | 187 |
| PL-214(Cs73Y_GA) | Cs73Y | 227 |
| PL-19(RsAS_GA) | RsAS | 195 |
| PL-5(At73C5_GA) | At73C5 | 217 |
| PL-69(CrUGT-2_GA) | CrUGT-2 | 221 |
| PL-376(Sp72T_GA) | Sp72T | 191 |
| PL-32(OsEUGT11_GA) | OsEUGT11 | 213 |
| PL-341(Ad72X_GA) | Ad72X | 209 |
| PL-332(Bv73P_GA) | Bv73P | 233 |
| PL-206(Tc74Z_GA) | Tc74Z | 215 |
| PL-347(Zj71A_GA) | Zj71A | 211 |
| PL-96(Eg85S_GA) | Eg85S | 223 |
| PL-280(Ha76H_2_GA) | Ha76H_2 | 231 |
| PL-106(Si85B_GA) | Si85B | 225 |
| PL-355(Ac73H_GA) | Ac73H | 237 |
| PL-283(Vv83A_GA) | Vv83A | 241 |
| PL-274(Ha74B_GA) | Ha74B | 229 |
| PL-41(At76E11_GA) | At76E11 | 219 |
| PL-154(Qs85H_GA) | Qs85H | 239 |
| PL-78(At71C1-Sr71E1_354_GA) | At71C1-Sr71E1_354 | 199 |
| PL-338(Pt72B_GA) | Pt72B | 245 |
| PL-89(At71C1_At71C2_353_GA) | At71C1_At71C2_353 | 243 |
| PL-4(At71C2_GA) | At71C2 | 193 |
| PL-304(Sp72Q_GA) | Sp72Q | 205 |
| PL-305(Si72V_GA) | Si72V | 207 |
| PL-811(Cc91D1_GA) | Cc91D1 | 247 |
| PL-812(Pg91B1_GA) | Pg91B1 | 249 |
| PL-813(Ha91_GA) | Ha91 | 251 |

### Example 18—In Vitro Testing of Glycosyltransferase Performance in Glucosylating Substituted Tryptamines

For in vitro studies of glycosyltransferase performance in attaching glucose moieties onto free hydroxy groups of various substituted tryptamines, purified glycosyltransferases were prepared as described in Example 7 with enzyme assays carried out as described in Example 10, quantification of glucoside production was carried out as described in Example 8. Alternatively, where quantification by analysing peak areas by HPLC was not feasible due to insufficient separation of the substituted tryptamine and its corresponding glucoside, identification was carried out by LC-MS/QTOF from which the relative peak areas of both glucoside and substrate were used for calculating the % conversion of substituted tryptamine into its corresponding glucoside.

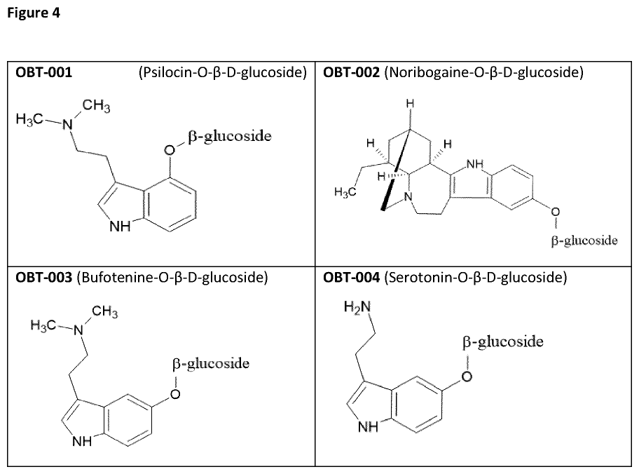

In an initial screen, glucosylation was tested with the substituted tryptamines Serotonin, Psilocin, Bufotenine and Noribogaine using UDP-glucose as the sugar donor. Corresponding structure ID's (OBT-001 to OBT-004) was given for each substituted tryptamine glucoside produced in this screen, structures of each molecule and common names are shown in FIG. 4. FIG. 4. Structures of substituted tryptamine glucosides validated by LC-MS/QTOF

### Substituted Tryptamine Glucosides Produced Using Psilocin as an Acceptor.

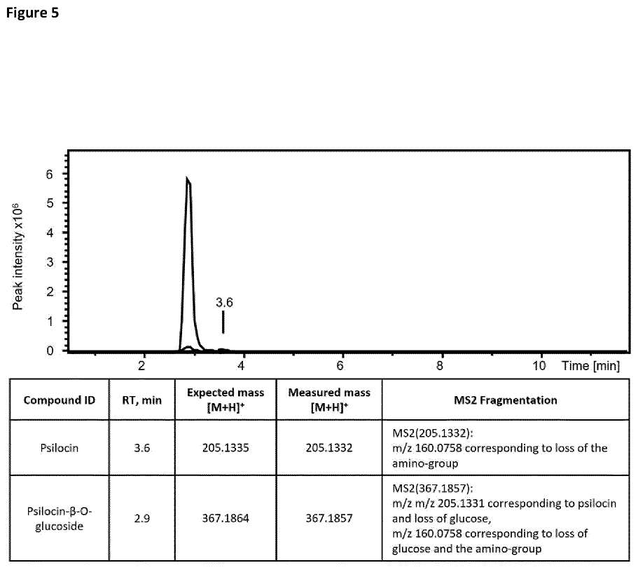

Numerous glycosyltransferases were found to catalyze the conversion of psilocin to OBT-001 (psilocin-O-β-D-glucoside) (FIG. 4). FIG. 5 shows a representative chromatogram produced by LC-MS/QTOF analysis of a reaction mixture containing the substituted tryptamine and an exemplary glycosyltransferase At71C2 (SEQ ID NO's. 193, 194). The figure further shows the retention time (RT), expected and measured mass of each compound and fragmentation pattern as determined by LC-MS/QTOF analysis thereby confirming the structure of the produced glucoside.

It was further found that multiple glycosyltransferases could catalyze the conversion of psilocin to OBT-001 (psilocin-O-β-D-glucoside) with varying conversion efficiencies. Table 20 shows glycosyltransferases which produced psilocin-O-β-D-glucoside along with the % conversion efficiency.

TABLE 20
Glycosyltransferases catalyzing the conversion
of psilocin to OBT-001 (psilocin-O-β-D-glucoside)
with calculated conversion efficiency.

| Enzyme name | % Conversion |
|---|---|
| At71C2 | 32.82 |
| At71C1-Sr71E1_354 | 28.05 |
| Sp72T | 22.57 |
| Pt73Y | 21.40 |
| At72B1 | 18.67 |
| RsAS | 17.27 |
| Ha72B | 16.21 |
| Ha72T | 16.18 |
| At73B5 | 1.00 |
| Sp72Q | 1.00 |
| Si72V | 1.00 |
| Ad72X | 1.00 |
| Zj71A | 1.00 |

### Substituted Tryptamine Glucosides Produced Using Noribogaine as an Acceptor.

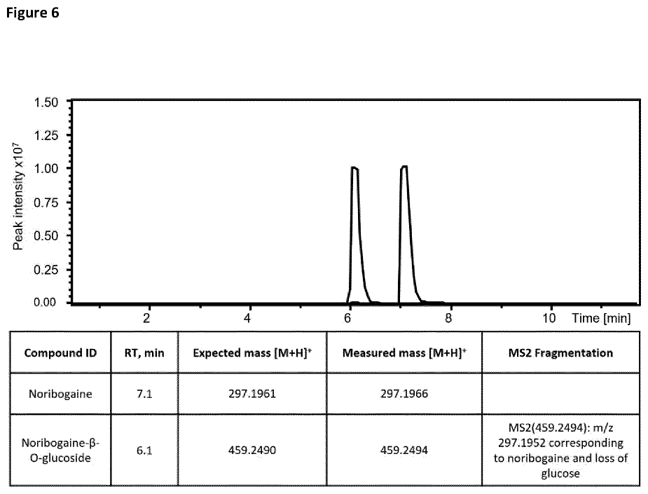

Numerous glycosyltransferases were found to catalyze the conversion of noribogaine to OBT-002 (noribogaine-O-β-D-glucoside) (FIG. 4). FIG. 6 shows a representative chromatogram produced by LC-MS/QTOF analysis of a reaction mixture containing the substituted tryptamine and an exemplary glycosyltransferase. The figure further shows the retention time (RT), expected and measured mass of each compound and fragmentation pattern as determined by LC-MS/QTOF analysis thereby confirming the structure of the produced glucoside.

It was further found that multiple glycosyltransferases could catalyze the conversion of noribogaine to OBT-002 (noribogaine-O-β-D-glucoside) with varying conversion efficiencies. Table 21 shows glycosyltransferases which produced noribogaine-O-β-D-glucoside along with the % conversion efficiency.

TABLE 21
Glycosyltransferases catalyzing the conversion
of noribogaine to OBT-002 (noribogaine-O-β-
D-glucoside) with calculated conversion efficiency.

| Enzyme name | % Conversion |
|---|---|
| Pt73Y | 108.53 |
| At73B5 | 64.28 |
| At72B1 | 62.58 |
| Ha72T | 59.37 |
| Cp73B | 31.45 |
| Ha72B | 27.61 |
| Cs73Y | 26.54 |
| RsAS | 8.97 |
| At73C5 | 7.42 |
| CrUGT-2 | 7.41 |
| Sp72T | 4.08 |
| OsEUGT11 | 2.64 |
| Ad72X | 2.07 |
| Bv73P | 2.00 |
| Tc74Z | 1.63 |
| Zj71A | 1.53 |
| Eg85S | 1.50 |
| Ha76H_2 | 1.35 |
| Si85B | 1.32 |
| Ac73H | 1.29 |
| Vv83A | 1.24 |
| Ha74B | 1.03 |
| At76E11 | 0.98 |
| Qs85H | 0.83 |

### Substituted Tryptamine Glucosides Produced Using Bufotenine as an Acceptor.

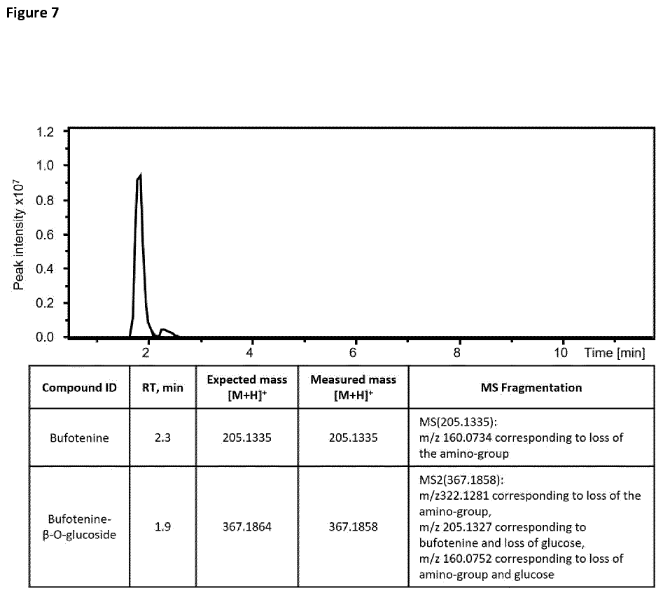

Numerous glycosyltransferases were found to catalyze the conversion of bufotenine to OBT-003 (bufotenine-O-β-D-glucoside) (FIG. 4). FIG. 7 shows a representative chromatogram produced by LC-MS/QTOF analysis of a reaction mixture containing the substituted tryptamine and an exemplary glycosyltransferase. The figure further shows the retention time (RT), expected and measured mass of each compound and fragmentation pattern as determined by LC-MS/QTOF analysis thereby confirming the structure of the produced glucoside.

It was further found that multiple glycosyltransferases could catalyze the conversion of bufotenine to OBT-003 (bufotenine-O-β-D-glucoside) with varying conversion efficiencies. Table 22 shows glycosyltransferases which produced bufotenine-O-β-D-glucoside along with the % conversion efficiency.

TABLE 22
Glycosyltransferases catalyzing the conversion of
bufotenine to OBT-003 (bufotenine-O-β-D-glucoside)
with calculated conversion efficiency.

| Enzyme name | % Conversion |
|---|---|
| At71C1_At71C2_353 | 94.18 |
| At73C5 | 5.68 |
| Tc74Z | 0.62 |
| At71C1-Sr71E1_354 | 18.44 |
| Pt73Y | 17.87 |
| At73B5 | 49.06 |

### Substituted Tryptamine Glucosides Produced Using Serotonin as an Acceptor.

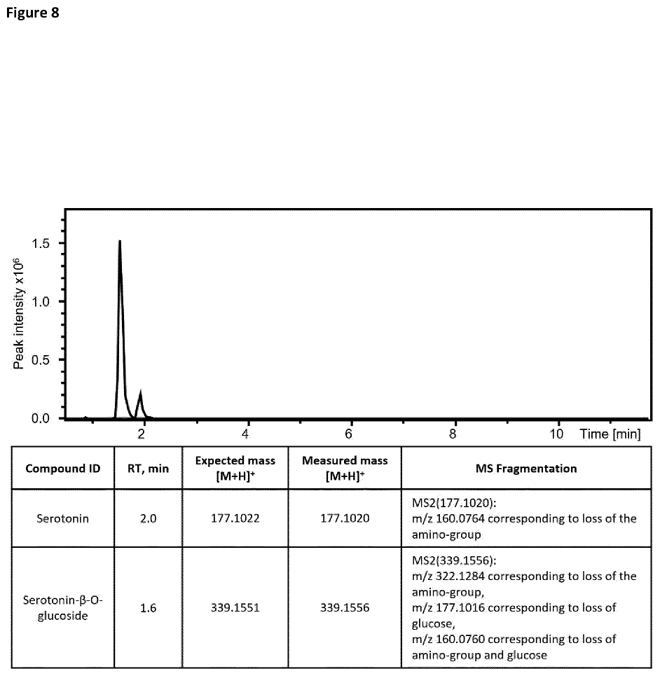

Numerous glycosyltransferases were found to catalyze the conversion of serotonin to OBT-004 (serotonin-O-β-D-glucoside) (FIG. 4). FIG. 8 shows a representative chromatogram produced by LC-MS/QTOF analysis of a reaction mixture containing the substituted tryptamine and an exemplary glycosyltransferase. The figure further shows the retention time (RT), expected and measured mass of each compound and fragmentation pattern as determined by LC-MS/QTOF analysis thereby confirming the structure of the produced glucoside.

It was further found that multiple glycosyltransferases could catalyze the conversion of serotonin to OBT-004 (serotonin-O-β-D-glucoside) with varying conversion efficiencies. Table 23 shows glycosyltransferases which produced serotonin-O-β-D-glucoside along with the % conversion efficiency.

TABLE 23
Glycosyltransferases catalyzing the conversion of
serotonin to OBT-004 (serotonin →
serotonin-O-β-D-glucoside) with calculated
conversion efficiency.

| Enzyme name | % Conversion |
|---|---|
| At71C1-Sr71E1_354 | 24.91 |
| Pt72B | 11.65 |
| At71C1_At71C2_353 | 11.63 |
| RsAS | 11.31 |

Overall, it was discovered that a range of glycosyltransferases could use various substituted tryptamines as sugar acceptors resulting in the production of a range of new substituted tryptamine glycosides. In the screen, enzymes were found which could catalyze a wide variety of different and highly specific reactions. Glycosyltransferases were found that could selectively attach a single glucose group onto the substituted tryptamine molecule producing a corresponding mono-glycosides, e.g. psilocin-O-β-D-glucoside (OBT-001) produced by Sp72T (SEQ ID NO's. 191, 192). Based on the calculated conversion % it was found many glycosyltransferases were highly active and could utilize UDP-glucose and efficiently glucosylate substituted tryptamines with high conversion efficiency. At71C1-Sr71E1_354 (SEQ ID NO's. 199, 200) for example was found to efficiently produce glucosides of psilocin, bufotenine, and serotonin, while the Pt73Y (SEQ ID NO's. 203, 204) was found to efficiently produce noribogaine glucoside. It was found that a large number of enzymes were found to catalyze glucosylation reactions on substituted tryptamines, in total this in vitro screen identified 30 glycosyltransferases active on substituted tryptamine molecules.

### Conclusion

The results of this experiment show that UGT enzymes from a diverse range of plant species surprisingly catalyze the glycosylation of substrates not encountered in their host species native environment, leading to the production of novel tryptamine glycosides. The experiment also revealed the surprisingly high catalytic activity of some of the tested UGT's with enzymes like At71C2, At71C1_At71C2_353, and Pt73Y converting a significant amount of available substrate into its corresponding glycoside

### Example 19—In Vitro Testing of Glycosyltransferase Performance in Glycosylating Substituted Tryptamines with Alternative UDP-Sugars

In Example 18, a range of glycosyltransferases were found which could accept UDP-glucose and catalyse production of a range of substituted tryptamine-glucosides. Top performing enzymes from this screen were further tested to determine whether they could accept alternative UDP-sugars, catalysing the production of substituted tryptamine-glycosides with different sugar groups attached. For in vitro studies of glycosyltransferase performance in glycosylating substituted tryptamines, purified glycosyltransferases were prepared as described in Example 7 with enzyme assays carried out as described in Example 10, quantification of glycoside production was carried out as described in Example 8. Alternatively, where quantification by analysing peak areas by HPLC was not feasible due to insufficient separation of the substituted tryptamine and its corresponding glycoside, quantification was carried out using LC-MS/QTOF data as described in example 18.

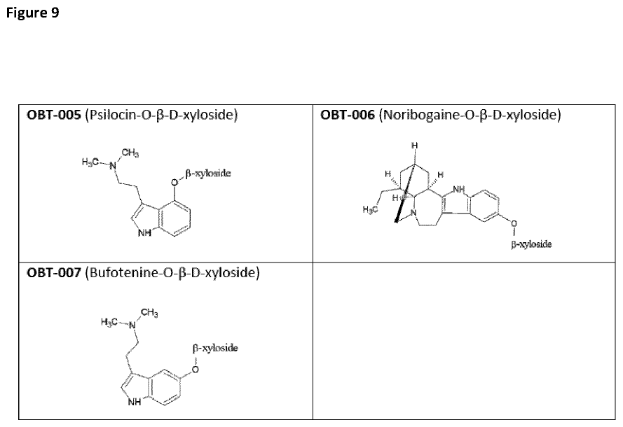

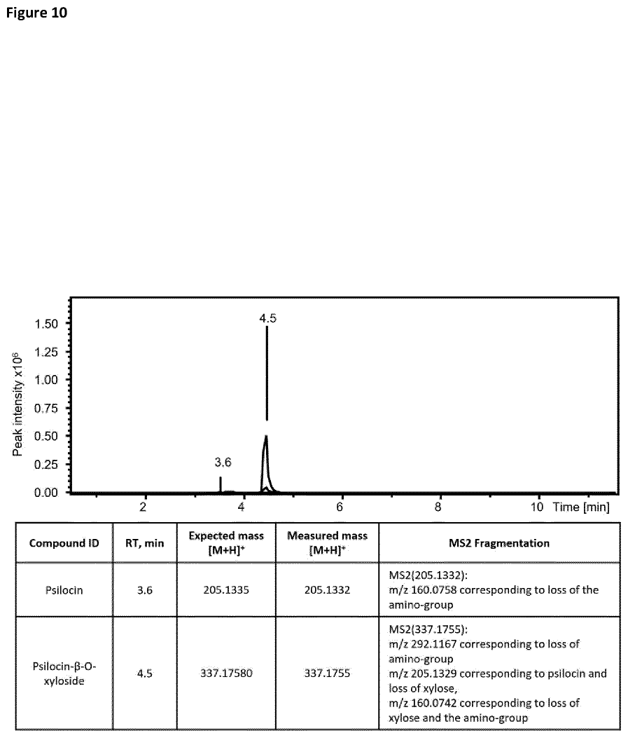

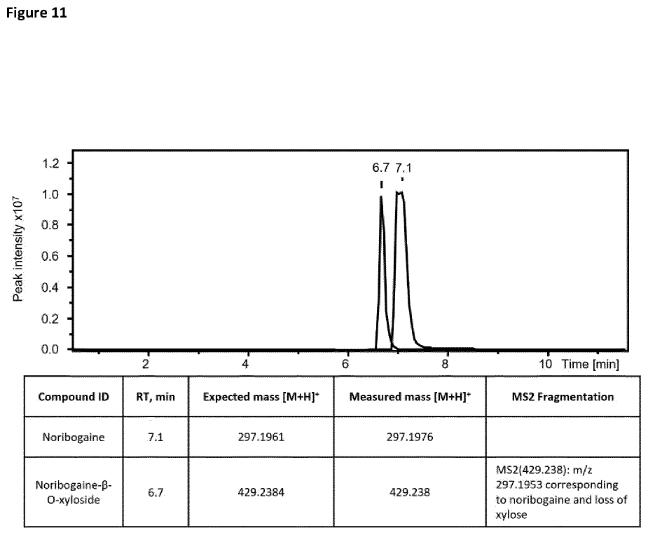

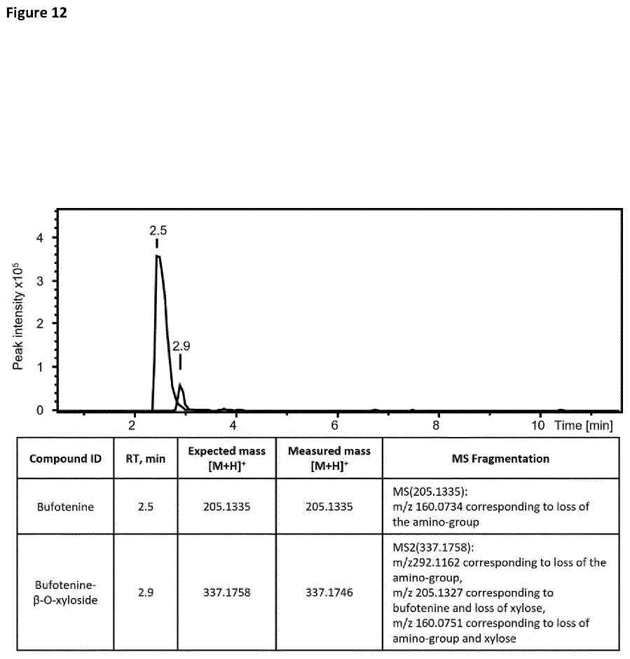

In an initial screen, glycosylation was tested using UDP-xylose as a sugar donor. Xylosylation of noribogaine and bufotenine was tested with Pt73Y (SEQ ID NO's. 203, 204), and xylosylation of psilocin was tested with At71C2 (SEQ ID NO's. 193, 194). A corresponding structure ID was given for each substituted tryptamine glycoside produced in this screen, structures of each molecule is shown in FIG. 9. The resulting LC-MS/QTOF chromatograms produced from each reaction are given in FIG. 10 for OBT-005, FIG. 11 for OBT-006 and FIG. 12 for OBT-007.

It was found that each tested enzyme could efficiently xylosylate their respective substrate producing the resulting xyloside. A calculation of the conversion % revealed that Pt73Y (SEQ ID NO's. 203, 204) converted 15% of available noribogaine and 7.9% of available bufotenine to their respective xylosides, while At71C2 (SEQ ID NO's. 193, 194) converted 8.2% of available psilocin to its respective xyloside. These results show that the discovered glycosyltransferase enzymes are capable of attaching not only glucose but other sugars as well, leading to a more diverse range of substituted tryptamine glycosides.

### Conclusion

The results of this experiment show that not only do these UGT's have wide substrate scope in terms of the aglycone substrate, but they also have a wide substrate scope for the UDP-sugar used. Not only could these UGT's accept UDP-glucose as a substrate but they can also accept other sugars like UDP-xylose as well.

### Example 21—Improved Psilocybin Production in Engineered S. cerevisiae Strains by Deletion of Native Phosphatase Genes

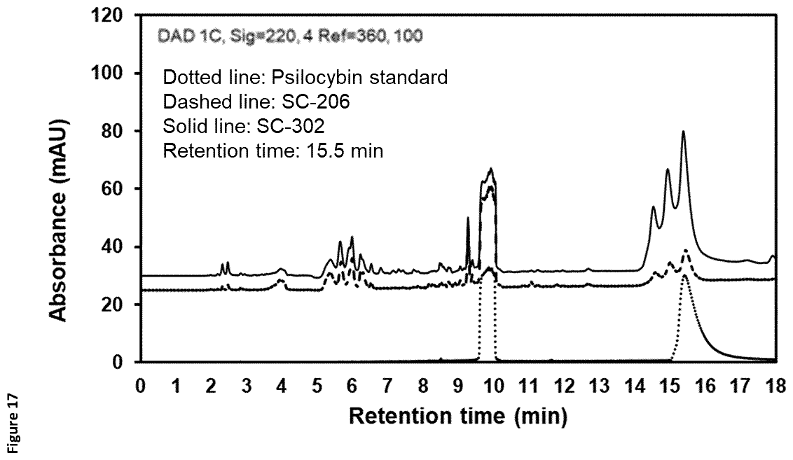

S. cerevisiae strains engineered to produce psilocybin by integration of heterologous psilocybin biosynthetic pathway genes is was observed that significant concentrations of psilocin was accumulated (e.g. Table 34). It was hypothesized that this accumulation was caused by cleavage of the phosphate group. It was further decided to test if the degradation of psilocybin to psilocin was caused by enzymatic hydrolysis by endogenous S. cerevisiae phosphatase enzymes. Psilocybin phosphatase from Psilocybe cubensis (PcPsiP) was found to share significant sequence homology with several native S. cerevisiae phosphatases. In P. cubensis, PcPsiP's native function was found to convert psilocybin to psilocin so it was further found that that the native S. cerevisiae enzymes shared significant sequence homology and that they could also have promiscuous activity towards psilocybin, similarly converting it to psilocin. To test this hypothesis several S. cerevisiae phosphatase genes were knocked out in psilocybin production strain SC-276 (according to Example 1), and the effect on the amount of psilocybin and psilocin produced measured from a standard cultivation (according to Example 4) and quantified using a standard HPLC analytical method with comparison to authentic analytical standards (according to Example 8). The results shown in Table 24 and FIG. 17 show significant improvements in psilocybin titer in several knockout strains. In general, knockout of these phosphatase genes results in significantly higher psilocybin production. In particular, DIA3, PHO5 and PHO3 knockout resulted in the biggest increase in psilocybin.

TABLE 24
Production of psilocybin and psilocin in engineered S. cerevisiae strains
with native phosphatase knockouts. Data shown in mg/L is
from triplicate experiments. ND: Not detected.

| Strain | Genotype description | SEQ ID NO: | Psilocybin | Psilocin |
|---|---|---|---|---|
| BY4741 | Wild-type | NA | ND | ND |
| SC-276 | Parental strain | NA | 177 | 72 |
| SC-302 | DIA3 | SEQ ID NO: 40 | 338 | ND |
| SC-303 | PHO5 | SEQ ID NO: 41 | 329 | ND |
| SC-412 | PHO12 | SEQ ID NO: 42 | 203 | 45 |
| SC-413 | PHO11 | SEQ ID NO: 43 | 213 | 32 |
| SC-301 | PHO3 | SEQ ID NO: 44 | 305 | ND |

### Conclusion

The results of this experiment demonstrated that the high psilocybin degradation observed in engineered yeast strains was caused by dephosphorylation by native S. cerevisiae phosphatase enzymes with homology to PsiP from P. cubensis. Deletion of these phosphatase genes resulted in significant improvement in psilocybin production through a significant reduction in psilocybin degradation to psilocin. This further demonstrated that deletion of one or more of these genes was a successful strategy to improve production.

### Example 22—In Vitro Production of Substituted Tryptamine Derivatives by One-Pot Biocatalytic Enzyme Cascade Using Serine and Substituted Indole Derivatives

### Part 1

In Example 15 production of substituted tryptamine derivatives 5-fluorotryptamine and 5-methoxytryptophan was achieved by S. cerevisiae strains overexpressing the engineered Tryptophan synthase PfTrpB(2B9) (and tryptophan decarboxylase RgTdc in the case of 5-fluorotryptamine) and feeding the respective substituted indoles (5-fluoroindole, 5-methoxyindole) in a cultivation experiment. In this experiment, a wild-type, non-engineered tryptophan synthase enzyme from Psilocybe cubensis (PcTrpB, SEQ ID NO: 179) was compared with engineered tryptophan synthases known in the art (PfTrpB(2B9), SEQ ID NO: 59 and TmTrpB(M145T,N167D), SEQ ID NO: 67). A PcTrpB enzyme containing comparable mutations to TmTrpB(M145T,N167D), was also generated to use as a comparison (PcTrpB(M439T,N459D), SEQ ID NO: 181). Genes were cloned into pET28a(+) expression plasmids and cloned into E. coli according to Example 3. Purified enzyme was prepared according to Example 7. In its native reaction, Tryptophan synthase converts indole and serine into tryptophan, in this assay we investigated whether different Tryptophan synthases could accept different substituted indoles and serine as substrate to produce the corresponding substituted tryptophan. In vitro enzyme reactions were set up according to Table 25, and incubated for X16 h at X30° c.

TABLE 25
Reaction components to test Tryptophan synthase
ability to accept substituted indole derivatives.

| Reagents | Volume (μL) |
|---|---|
| Purified enzyme | 5 |
| 50 mM substrate | 3 |
| 50 mM L-serine | 3 |
| 1 mM PLP | 0.05 |
| 1M Tris-HCl pH 8.0 | 2.5 |
| Milli-Q water | 36.45 |
| TOTAL | 50 |

After 16 h, the reaction was terminated by freezing. Samples were analyzed by HPLC according to Example 8. The amount of substituted indole converted to its corresponding substituted tryptophan (in percentage) was calculated by peak area, the results of the assay are presented in Table 26.

TABLE 26
% conversion of substituted indole and serine to its corresponding substituted tryptophan
derivative by engineered and wild-type tryptophan synthase enzymes. ND: Not detected.

| | Enzyme added | | | |
|---|---|---|---|---|
| | TmTrpB | | | PcTrpB |
| Substrate | (M145T, N167D) | PfTrpB (2B9) | PcTrpB | (M439T, N459D) |
| Indole | 30% | 20% | 100% | 100% |
| 4-hydroxyindole | ND | ND | 10% | 1% |
| 6-Hydroxyindole | 1% | 1% | 1% | 1% |
| 5-Methoxyindole | 5% | 1% | 80% | 60% |
| 4-Bromoindole | 2% | 1% | 1% | 1% |
| 5-Bromoindole | 5% | 5% | 80% | 80% |
| 6-Bromoindole | 2% | 2% | 25% | 40% |
| 7-bromoindole | 90% | 80% | 100% | 100% |
| 4-Chloroindole | 2% | 1% | 1% | 5% |
| 5-Chloroindole | 60% | 30% | 100% | 100% |
| 6-Chloroindole | 30% | 30% | 50% | 50% |
| 5,6-Dichloroindole | 1% | 1% | 2% | 2% |
| 4-Fluoroindole | 60% | 50% | 100% | 100% |
| 5-Fluoroindole | 60% | 10% | 100% | 100% |
| 6-Fluoroindole | 70% | 10% | 100% | 100% |
| 4-Nitroindole | ND | ND | ND | ND |
| 5-Nitroindole | ND | ND | 20% | 5% |
| 6-Nitroindole | ND | ND | ND | ND |
| 7-Nitroindole | ND | ND | 2% | ND |
| Azaindole | ND | ND | ND | ND |

The results showed that the tryptophan synthase enzymes tested could accept a wide range of substituted indole derivatives to produce the corresponding substituted tryptophan derivative. While as expected, the engineered tryptophan synthase enzymes, with amino acid modifications known in the art to substantially increase the substrate scope of these enzymes had significant activity with a broad panel of substituted indole derivatives, surprisingly, the wild-type tryptophan synthase from Psilocybe cubensis (PcTrpB), had comparable and in many cases superior activity than its engineered counterparts. PcTrpB and the engineered version PcTrpB (M439T, N459D) outperformed both TmTrpB (M145T, N167D) and PfTrpB (2B9), well known in the art to possess superior catalytic activity to wild-type tryptophan synthases with practically every substituted indole tested, while the wild-type PcTrpB outperformed even its engineered counterpart PcTrpB (M439T, N459D) with many substrates including 4-hydroxyindole, 5-methoxyindole, 5-nitroindole and 7-nitroindole. While no activity was detected using Azaindole as a substrate, this was likely due to the low solubility of the substrate. The results clearly show the surprisingly wide substrate scope of the wild-type tryptophan synthase (PcTrpB). While prior art describes extensive engineering and enzyme evolution efforts to increase the substrate scope and catalytic activity of tryptophan synthase enzymes, this study demonstrates that the non-engineered enzyme from Psilocybe cubensis significantly outperforms these engineered enzymes, opening the door for its use as a superior enzyme catalyst for the production of a diverse range of substituted tryptophan derivatives.

### Part 2

To further demonstrate the utility of fungal enzymes, a coupled reaction was set up using the wild-type Tryptophan synthase (PcTrpB), and a panel of Tryptophan decarboxylases. While tryptophan is the native substrate of tryptophan decarboxylases, which catalyse formation of the corresponding amine (tryptamine), prior art describes enzymes displaying a broader substrate scope and the ability to produce a range of substituted tryptamines from the corresponding substituted tryptophan derivative. RgTdc (SEQ ID NO: 69) in particular is known in the art to be particularly effective when combined with engineered Tryptophan synthases (TmTrpB (M145T, N167D) and PfTrpB (2B9)) in a one-pot reaction producing a substituted tryptamine derivative from the corresponding substituted indole derivative and serine. RgTdc was used as a benchmark to compare the activity and substrate scope of 2 fungal tryptophan decarboxylases from Psilocybe cubensis (PcPsiD, SEQ ID NO: 71) and related psilocybin producing fungus Panaeolus cyanescens (PanCyPsiD, SEQ ID NO: 77), as well as a non-canonical amino acid decarboxylase from P. cubensis (PcncAAD, SEQ ID NO: 73). In this assay, the ability of different tryptophan decarboxylases to convert a substituted tryptophan derivative, produced by conversion of a substituted indole and serine by PcTrpB was investigated. Purified enzymes were prepared according to Example 7, enzyme assays were set up according to Table 27 below and incubated at 30° cX for X16 h.

TABLE 27
Reaction components to test ability of tryptophan
decarboxylases to convert substituted tryptophan derivatives
produced by PcTrpB into substituted tryptamine derivatives

| Reagents | Volume (μL) |
|---|---|
| Purified enzymes | 10 |
| 50 mM substrate | 3 |
| 50 mM L-serine | 3 |
| 1 mM PLP | 0.05 |
| 1M Tris-HCl pH 8.0 | 2.5 |
| Milli-Q water | 31.45 |
| TOTAL | 50 |

After X16 h, the reaction was terminated by freezing. Samples were analyzed by HPLC according to Example 8. The amount of substituted indole converted to its corresponding substituted tryptamine (in percentage) was calculated by peak area, the results of the assay are presented in Table 28

TABLE 28
% conversion of substituted indole and serine to its corresponding substituted tryptamine
derivative by PcTrpB and different tryptophan decarboxylases. ND: Not detected.

| | Enzymes added: PcTrpB+ | | | |
|---|---|---|---|---|
| Substrate | PcncAAD | RgTdc | PcPsiD | PanCyPsiD |
| Indole | 1% | 100% | 100% | 75% |
| 4-Hydroxyindole | ND | 80% | 95% | 100% |
| 5-Methoxyindole | ND | 30% | 60% | 40% |
| 5-Bromoindole | ND | 100% | 100% | 20% |
| 6-Bromoindole | ND | 70% | 2% | 10% |
| 7-Bromoindole | 1% | ND | 75% | 75% |
| 5-Chloroindole | ND | 100% | 100% | 90% |
| 6-Chloroindole | ND | 20% | 25% | 40% |
| 4-Fluoroindole | 1% | 100% | 100% | 90% |
| 5-Fluoroindole | 1% | 100% | 100% | 100% |
| 6-Fluoroindole | 1% | 100% | 100% | 100% |

The results showed that while PcncAAD had negligible activity towards the substituted tryptophan derivatives produced by PcTrpB (presumably because indole molecules in general are probably outside of its substrate scope), the other 3 tryptophan decarboxylases had considerable activity. While unsurprisingly RgTdc, known in the art to possess significant substrate scope towards a range of substituted tryptophan derivatives, had good activity with most substrates tested, the 2 fungal tryptophan decarboxylases outperformed or equalled RgTdc with almost every substrate tested, with only 6-Bromoindole having higher activity with RgTdc. Interestingly, while RgTdc had no detectible activity towards 7-Bromoindole, both fungal enzymes were able to efficiently convert it to 7-Bromotryptamine with 75% conversion. Again, the results clearly show the wide substrate scope of wild-type enzymes from Psilocybe cubensis and Panaeolus cyanescens and their potential to efficiently produce a wide range of substituted tryptamine derivatives.

Combining these fungal tryptophan synthases and tryptophan decarboxylases enables the efficient one-pot production of a diverse range of substituted tryptamines from their corresponding substituted indoles and serine. From an industrial context this is important since substituted tryptamines are typically difficult to produce and often very expensive, in contrast substituted indoles are relatively easy to produce and often highly inexpensive.

### Conclusion

The results of this experiment showed that the wild-type tryptophan synthase from P. cubensis had a significantly broad substrate scope and could accept a diverse range of substituted indoles to produce the corresponding substituted tryptophan. This surprising observation overcomes prejudices in the art (e.g. US20160298152A1) claiming that native tryptophan synthase enzymes do not have wide substrate scope unless engineered with “activating mutations” corresponding to residue 144 and 166, or residue 292. These results showed that while indeed some tryptophan synthase enzymes such as TmTrpB and PfTrpB required these activation mutations to broaden their substrate scope, not all tryptophan synthases require them, and in fact the wild-type tryptophan synthase described herein from P. cubensis was more efficient and has broader substrate scope than the engineered enzymes described in the art. Furthermore, these results also showed that introducing the corresponding activating mutations into the wild-type Tryptophan synthase PcTrpB did not improve catalytic activity and substrate scope and in fact in some cases resulted in lower activity. Finally, these results showed that tryptophan decarboxylases from P. cubensis and P. cyanescens (PcPsiD and PanCyPsiD) also had surprisingly broad substrate scope and could at least match or in some cases outperform promiscuous tryptophan decarboxyles known in the art (RgTdc). Overall this indicates that psilocybin producing mushroom species are an underutilized source of valuable enzymes with exciting new bioindustrial applications offering advantages over methods known in the art.

### Example 23—Combining Additional Derivatizing Enzymes to a Tryptophan Synthase/Tryptophan Decarboxylase One-Pot Biocatalytic Enzyme Cascade Using Serine and Substituted Indole Derivatives Leads to More Complex Substituted Tryptamine Derivatives

In example 22 a biocatalytic cascade to produce diverse substituted tryptamine derivatives from serine and substituted indole derivatives was demonstrated using a range of tryptophan synthase and tryptophan decarboxylase enzymes. To further demonstrate the utility of this one-pot cascade, a reaction was set up containing wild-type tryptophan synthase and tryptophan decarboxylase from Panaeolus cyanescens (PanCyTrpB, SEQ ID NO: 255 and PanCyPsiD, SEQ ID NO: 77), coupled with additional enzymes which could further convert the substituted tryptamine derivative produced by PanCyTrpB and PanCyPsiD into more complex derivatives when fed with the corresponding substituted indole derivative and serine. In this example, PanCyPsiK (SEQ ID NO: 159) and PanCyPsiM (SEQ ID NO: 127) were added as additional derivatizing enzymes and serine and 4-hydroxyindole used as the fed substrates. The promiscuous PanCyTrpB and PanCyPsiD converts these substrates into 4-hydroxytryptamine which PanCyPsiK further derivatizes to norbaeocystin followed by PanCyPsiM mediated derivatization to baeocystin then psilocybin. The assay was set up according to Example 22 with X6 mM serine and X3 mM 4-hydroxyindole added, Table 29 shows the results from the experiment. The results showed that a one-pot biocatalytic cascade could be used to efficiently produce psilocybin from serine and 4-hydroxyindole. Due to the instability of the phosphate group of psilocybin, degradation to psilocin was also observed.

TABLE 29
Production of complex substituted tryptamine derivatives by one-pot in
vitro biocatalytic cascade from serine and 4-hydroxyindole (in mM).

| | | Products | | | | | |
|---|---|---|---|---|---|---|---|
| | Enzymes | 4- | 4- | | | | |
| Substrate | added | hydroxytryptophan | hydroxytryptamine | Norbaeocystin | Baeocystin | Psilocybin | Psilocin |
| 4- | PanCyTrpB | 0.66 | 0.13 | 0.06 | 0.29 | 0.69 | 0.37 |
| Hydroxyindole | PanCyPsiD | | | | | | |
| | PanCyPsiK | | | | | | |
| | PanCyPsiM | | | | | | |

### Conclusion

These results showed that the superior catalytic activity of PanCyTrpB and PanCyPsiD can be further utilized as a starting point to produce more complex substituted tryptamine derivatives.

### Example 24—Production of N-Feruloyl Serotonin by Engineered S. cerevisiae

In example 13, yeast was engineered to produce the potent antioxidant and anti-hyperpigmentation compound 4-coumaroyl serotonin by introducing genes for the production of serotonin from tryptophan (RgTdc, OsT5H, FoCPR), genes for the production of 4-coumaroyl-CoA from phenylalanine (ARO7(G141S), ARO8, Pal2, C4 h, Atr2, 4CI), and finally an enzyme to derivatize serotonin with 4-Coumoryl CoA to produce 4-Coumoryl serotonin (CaSHT). Functional expression of an N-(hydroxycinnamoyl) transferase (CaSHT), opened the possibility of producing other serotonin derivatives as this enzyme is known to functionalize serotonin was a range of hydroxycinnamoyl derivatives including coumaroyl-CoA, caffeoyl-CoA, cinnamoyl-CoA and feruloyl-CoA leading to the production of N-coumaroylserotonin (demonstrated above), N-caffeoylserotonin, N-cinnamoylserotonin and N-feruloylserotonin. To further demonstrate the utility of this enzyme, yeast was engineered to produce N-feruloylserotonin from exogenously fed ferulic acid by introduction of genes to produce serotonin from tryptophan (CrTdc, SEQ ID NO: 25, OsT5H, SEQ ID NO: 95, FoCPR, SEQ ID NO: 111), a gene to convert ferulic acid to feruloyl-CoA (4CL2, SEQ ID NO: 57), and the gene to conjugate feruloyl-CoA and serotonin to N-feruloylserotonin (CaSHT, SEQ ID NO: 161). The strain (SC-NFS) was constructed according to Example 1, cultivated according to Example 4 with the addition of 1 mM ferulic acid to the media, and the concentration of N-feruloylserotonin was quantified according to Example 8. As shown in Table 30, SC-NFS was able to convert a substantial amount of exogenously added ferulic acid into N-feruloylserotonin, thereby demonstrating the utility of the CaSHT enzyme and the use of yeast to produce complex serotonin derivatives.

TABLE 30
Production of N-feruloylserotonin (in mM) by engineered
yeast fed 1 mM ferulic acid. ND: Not detected.

| Strain | Ferulic acid | N-feruloylserotonin |
|---|---|---|
| BY4741 | 0.97 | ND |
| SC-NFS | 0.34 | 0.45 |

### Conclusion

These results show that instead of producing the corresponding cinnamic acid de novo (as demonstrated in the de novo production of 4-coumaroylserotonin), hydroxycinnamoyl derivatives such as N-feruloylserotonin can also be produced simply by feeding the inexpensive hydroxycinnamic acid (ferulic acid in this case).

### Example 25—Production of Melatonin by Engineered Yeast Strains

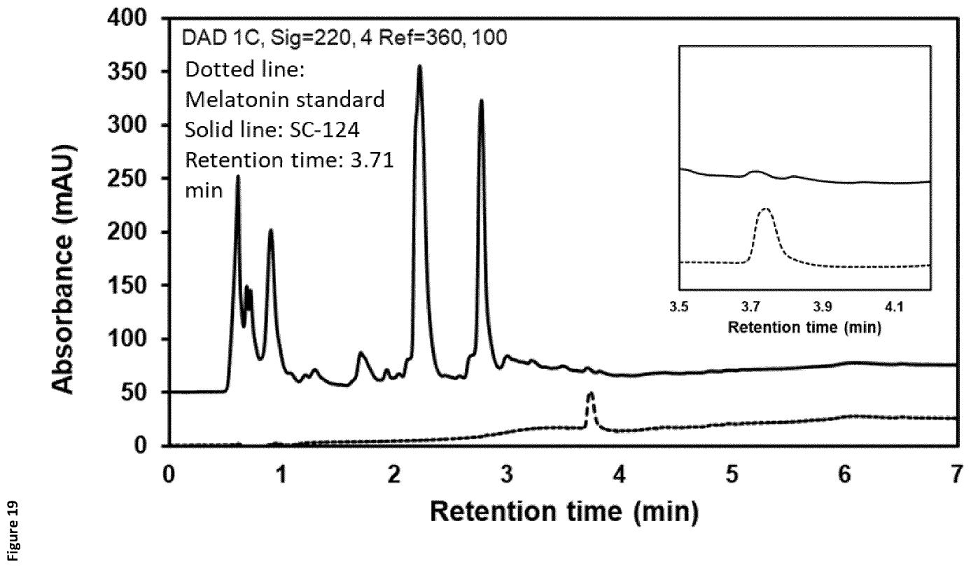

In example 12 it was shown that serotonin could be produced in yeast by integration and expression of a tryptophan decarboxylase (e.g. CrTdc) and a CYP/CPR pair, tryptamine 5-hydroxylase and cytochrome P450 reductase (OsT5H, FoCPR). Examples 13 and 24 further showed how this serotonin producing strain could be further engineered to produce more complex derivatives (4-coumaroylserotonin, N-feruloylserotonin). Here, the serotonin producing strain (SC-75) was further engineered to produce human hormone and nutraceutical melatonin by further integration and expression of an acetyl-serotonin methyltransferase (HsASMT, SEQ ID NO: 117) and a serotonin N-acetyltransferase (BtAANAT, SEQ ID NO: 141). Strains were constructed according to Example 1, cultivated according to Example 4 and quantified according to Example 8. As shown in Table 31 and FIG. 19, integration of HsASMT and BtAANAT into the serotonin producing strain resulted in the production of melatonin.

TABLE 31
Melatonin production by engineered S. cerevisiae strains in mg/L,
data presented as averages of triplicate experiments. ND: Not detected

| | Genotype | | | |
|---|---|---|---|---|
| Strain | description | Tryptamine | Serotonin | Melatonin |
| BY4741 | Wild-type | ND | ND | ND |
| SC-75 | Serotonin parental | 10.45 | 630.56 | ND |
| | strain | | | |
| SC-124 | Melatonin | ND | 422 | 26 |
| | producer | | | |

### Conclusion

The results of this experiment showed that efficient serotonin production by OsT5H and FoCPR can be further utilized to produce more complex serotonin derivatives such as melatonin. This offers significant improvements over microbial methods to produce melatonin known in the art (e.g. WO2013127915) which rely on the inefficient serotonin production pathway via 5-hydroxytryptophan requiring a tetrahydrobiopterin co-factor.

### Example 26—Production and Purification of Psilocin-O-β-Glucoside by One-Pot In Vitro Biocatalytic Cascade Using 4-Hydroxyindole, Serine and UDP-Glucose

To produce larger amounts of psilocin glucoside for further testing, a one-pot in vitro biocatalytic cascade was set up to produce psilocin glucoside directly from 4-hydroxyindole, serine and UDP-glucose. The reaction was set up in a similar fashion to Example 23 with PanCyTrpB, PanCyPsiD, PanCyPsiK and PanCyPsiM added to convert 4-hydroxyindole and serine to psilocybin. In Example 23, degradation of psilocybin to psilocin was observed which we utilized here to provide a substrate to the UGT enzyme At71C2 (SEQ ID NO's. 193, 194) also added in the one-pot reaction along with UDP-glucose to convert available psilocin into psilocin-O-β-glucoside as it degraded from psilocybin. The reaction was scaled-up to 20 mL with approximately 15 mg of each enzyme added to the reaction with 1 mM of 4-hydroxyindole, 1 mM of serine, and 3 mM of UDP-glucose, and alkaline phosphatase (as in Example 10). The reaction was incubated for 24 h at 30° C., then terminated by heating at 80° C. to degrade the proteins as well as any unreacted psilocybin and psilocin and then filtered through a 0.2 μm polyvinylidene fluoride (PVDF) filter prior to purification on Preparative HPLC. This yielded a final psilocin-O-β glucoside concentration of 0.3 mg/mL, indicating a yield from 4-hydroxyindole and serine of approximately 75%. Psilocin-O-β-glucoside was purified by preparative HPLC from the reaction mix as follows: The filtered assay mix was loaded on a Phenomenex Kinetex F5 column (250×21.2 mm, 5 um, 100 Å) on an Agilent 1290 preparative HPLC system equipped with a fraction collector and UV-detector. Gradient elution at a flow rate of 15 ml/min was employed using water with 0.01% trifluoroacetic acid (TFA) and methanol with 0.01% TFA as mobile phases A and B, respectively. The gradient was: 0-1 min: 2% B, 1-30 min: 2-98% B, 30-35 min: 98% B, 30-37 min: 98-2% B. Peaks with an area above 15 units and a up slope above 0.60 Units/s were collected using automatic peak detection at 230 nm. The fraction containing psilocin glucoside was dried in vacuo to yield a white powder (final. Yield: 7.9 mg). The identity of psilocin-O-β-glucoside was confirmed by LC-MS/QTOF: Calculated [M+H]+: m/z 367.1864. Observed [M+H]+: m/z 367.1865. MS2(367.1865): m/z 205.1339 corresponding to loss of the glucoside-moiety.

### Example 27—Testing the Chemical Stability of Psilocin Glucoside

Prior art demonstrates that psychedelic compound psilocin is highly unstable, with rapid degradation even under ambient conditions. While the added phosphate group of psilocybin helps protect the molecule from degradation, psilocybin is also relatively unstable, prone to dephosphorylation to psilocin and subsequent degradation. The instability of these molecules hinders formulation and delivery of the compound for therapeutic use. To improve the chemical stability of the active compound psilocin, glycosylation can be used as an effective strategy. To demonstrate the effectiveness of psilocin glycosylation, purified psilocin glucoside, along with psilocin and psilocybin was subjected to a range of harsh environmental conditions according to Example 9. Degradation of each compound was assessed by measuring the amount of compound remaining after 24 h exposure to each condition. As shown in Table 32 psilocin-O-β-glucoside shows remarkable stability in all conditions tested showing practically no signs of degradation even under particularly harsh conditions. In stark contrast, psilocin showed complete degradation under all conditions tested with zero psilocin detected after 24 h exposure, thereby demonstrating the high instability of the compound. While psilocybin showed much less degradation compared to psilocin, demonstrating that the added phosphate group does protect the molecule from degradation, it still showed signs of degradation and significantly underperformed psilocin glucoside in this stability study. These results show how glycosylation is a much better strategy for protecting psilocin from degradation than phosphorylation.

TABLE 32
Chemical stability of psilocybin, psilocin and psilocin
glucoside incubated under a range of conditions. Shown
is the % of compound remaining after incubation for 24 h.

| | | | Psilocin |
|---|---|---|---|
| | Psilocin | Psilocybin | glucoside |
| Condition | % left after 24 h | | |
| pH = 1.1 | 0.0 | 94.0 | 100.0 |
| pH = 12.5 | 0.0 | 93.0 | 100.0 |
| 3% H2O2 | 0.0 | 87.0 | 100.0 |
| 80° C. | 0.0 | 84.6 | 100.0 |
| 30° C. | 0.0 | 97.5 | 100.0 |
| 4° C. | 0.0 | 97.5 | 100.0 |
| Control | 100.0 | 100.0 | 100.0 |

### Conclusion

Psilocybin is a prodrug of psilocin, whereby the added phosphate group acts to stabilize the molecule and prevent degradation. This experiment shows that glycosylation of psilocin is a better strategy to stabilize the molecule than phosphorylation (psilocybin), thereby providing opportunity to develop new prodrugs of this active molecule.

### Example 28—Production of Psilocin Glucoside by Engineered Yeast

In previous examples, psilocin-O-β-glucoside was produced in vitro using the highly active UGT At71C2 (SEQ ID NO: 193) which converted psilocin to psilocin glucoside when exogenous UDP-glucose was added. Psilocin glucoside was produced either by feeding psilocin directly to the UGT reaction, or by producing it in a one-pot biocatalytic cascade from 4-hydroxyindole and serine, and relying on the inherent instability of psilocybin to degrade to psilocin to provide the psilocin substrate for the glycosylation reaction. In this example, psilocin glucoside was produced in yeast by two different production methods; Bioconversion, whereby At71C2 was introduced into a wild-type yeast strain (BY4741) and psilocin glucoside produced by feeding psilocin to the strain during cultivation, and de novo production, whereby At71C2 was introduced into a yeast strain engineered to efficiently produce psilocybin (SC-276). Strains were constructed according to Example 1, cultivated according to Example 4 and quantified according to Example 8. For the bioconversion cultivation, strains were fed with 100 mg/L psilocin. As shown in Table 33, expression of At71C2 resulted in production of psilocin glucoside by both production methods. While SC-276 produces psilocybin with psilocin produced as a byproduct, when At71C2 is introduced (SC-402) psilocin is further converted into psilocin glucoside. For the bioconversion strain (SC-394) only psilocin glucoside is detected (15.59 mg/L, even though 100 mg/L of psilocin was fed during the cultivation. The absence of further psilocin is likely due to the high instability of the molecule.

TABLE 33
Production of psilocin glucoside in yeast strains expressing UGT At71C2 in either a wild-type
background (Bioconversion strain) or in a psilocybin producing background (De novo strain).
Data presented in mg/L and as the average of triplicate experiments. ND: Not detected.

| | | | | Psilocin |
|---|---|---|---|---|
| Strain | Genotype description | Psilocybin | Psilocin | glucoside |
| BY4741 | Wild-type | ND | ND | ND |
| SC-276 | Psilocybin parental strain | 131.2 | 56.4 | ND |
| SC-394 | Bioconversion strain | ND | ND | 15.59 |
| SC-402 | De novo strain | 98.4 | ND | 10.2 |

### Conclusion

This experiment demonstrated another method for producing tryptamine glycosides. While previous examples demonstrated tryptamine glycoside production using an in vitro biocatalytic approach, this experiment showed that in vivo bioconversion and de novo production in yeast is also feasible. This opens multiple avenues for industrially producing such new molecules.

### Example 29—Production of Psilocybin by Engineered Yeast Expression Genes from Panaeolus Cyanescens

### Part 1

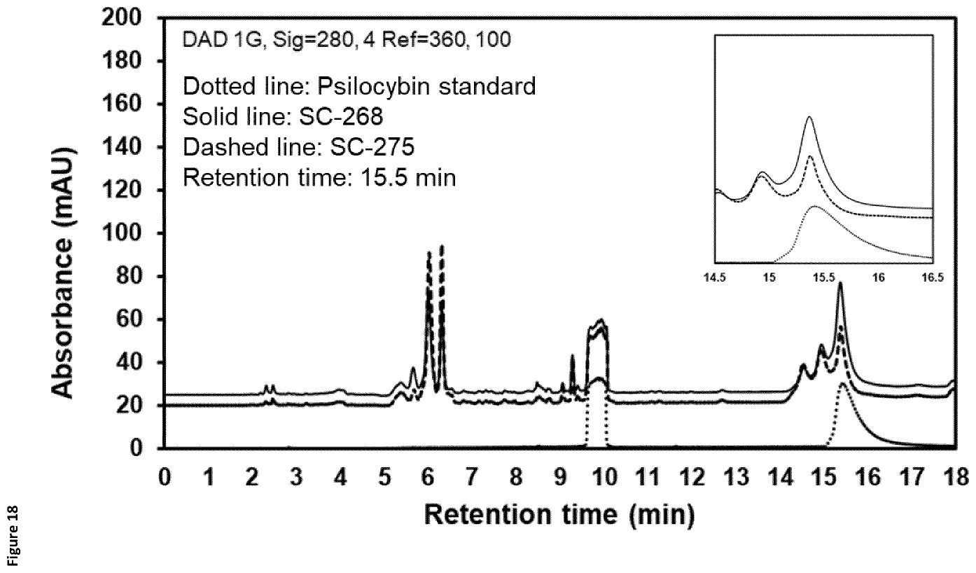

While Psilocybe cubensis is perhaps the most well-known species to produce psilocybin, a diverse number of species are also known to produce psilocybin. However, to date, only the psilocybin biosynthetic pathway from P. cubensis has been elucidated, with genes discovered to convert tryptophan to psilocybin in 5 enzymatic steps. Mining the genomes of other psilocybin producing species can uncover new gene homologues which could have improved activity compared to the well-known gene candidates from P. cubensis. Through genome mining, a putative set of psilocybin biosynthetic genes were identified in the fungal species Panaeolus cyanescens, including a putative tryptophan decarboxylase (PanCyPsiD, SEQ ID NO: 77), tryptamine 4-hydroxylase (PanCyPsiH, SEQ ID NO: 87), cytochrome P450 reductase (PanCyCPR, SEQ ID NO: 105), 4-hydroxytryptamine kinase (PanCyPsiK, SEQ ID NO: 159), and psilocybin synthase (PanCyPsiM, SEQ ID NO: 127). Interestingly, these putative genes had only approximately 80% amino acid sequence homology to their homologues from P. cubensis, potentially indicating differential activity. To test the functionality of these enzymes and to compare their activity against homolog enzymes from P. cubensis, each gene set was introduced into a high producing tryptamine background (SC-106) according to Example 1, cultivated according to Example 4, and the concentration of psilocybin and other byproducts and intermediates quantified according to Example 8 to directly compare the biocatalytic capacity of each set of enzymes. The results shown in Table 34 and FIG. 18 show that introducing genes from both species results in the production of psilocybin, however introducing genes from P. cyanescens results in higher production than genes from P. cubensis. Overall demonstrating the superior catalytic activity of enzymes sourced from P. cyanescens. A lower accumulation of tryptamine was also observed in SC-268 compared to SC-275, again supporting the improved activity of the P. cyanescens biosynthetic pathway.

TABLE 34
Production of psilocybin in S. cerevisiae strains containing the
psilocybin biosynthetic pathway from P. cubensis and P. cyanscens.
Data is presented in mg/L and is the average of
triplicate expriments. ND: Not detected.

| | Genotype | | | |
|---|---|---|---|---|
| Strain | description | Tryptamine | Psilocybin | Psilocin |
| BY4741 | Wild-type | ND | ND | ND |
| SC-106 | Tryptamine | 448.62 | ND | ND |
| | producer parental | | | |
| | strain | | | |
| SC-275 | Psilocybin pathway | 13.56 | 345.14 | 50.12 |
| | from Psilocybe | | | |
| | cubensis | | | |
| SC-268 | Psilocybin pathway | 9.03 | 431.23 | 78.67 |
| | from Panaeolus | | | |
| | cyanescens | | | |

### Part 2

Further genome mining identified a gene encoding a putative cytochrome b5 (PanCyCYB5, SEQ ID NO: 253). Heterologous expression of cytochrome b5 proteins has previously been shown to improve CYP/CPR reactions (WO/2021/052989) but is usually specific for expression of cytochrome b5 from the same species as the CYP/CPR pair, thereby necessitating the expression of the Panaeolus cyanescens cytochrome b5 in order to improve the activity of the PanCyPsiH/PanCyCPR CYP/CPR pair. The putative cytochrome b5 (PanCyCYB5) was integrated and expressed in a strain expressing the psilocybin biosynthetic pathway from Panaeolus cyanescens according to Example 1, cultivated according to Example 4, and the concentration of psilocybin and other byproducts and intermediates quantified according to Example 8. The results shown in Table 35 show that additional expression of PanCyCYB5 improves psilocybin production by improving the conversion of tryptamine to 4-hydroxytryptamine. While some tryptamine accumulation was observed in SC-269, tryptamine was not detected in SC-270 indicating improved hydroxylation activity and full conversion of available tryptamine to 4-hydroxytryptamine.

TABLE 35
Increased production of psilocybin in stains expressing the psilocybin biosynthetic pathway
from P. cyanescens with additional expression of PanCyCYB5. Data is presented
in mg/L and is the average of triplicate experiments. ND: Not detected.

| Strain | Genotype description | Tryptamine | Psilocybin | Psilocin |
|---|---|---|---|---|
| BY4741 | Wild-type | ND | ND | ND |
| SC-269 | Psilocybin pathway | 18.45 | 424.43 | 68.12 |
| | from Panaeolus | | | |
| | cyanescens | | | |
| SC-270 | Psilocybin pathway | ND | 498.34 | 79.43 |
| | from Panaeolus | | | |
| | cyanescens with CYB5 | | | |

### Conclusion

This experiment shows that enzymes from other psilocybin species can be introduced into yeast to enable the heterologous production of psilocybin, even enzymes with relatively low sequence homology to genes known in the art. The results also demonstrate that introducing genes from Panaeolus cyanescens into yeast to produce psilocybin is a better strategy than introducing genes from Psilocybe cubensis with higher production observed. These results also demonstrate that cytochrome b5 expression can work in conjunction with the cytochrome p450 reductase to further enhance the catalytic activity of tryptamine 4-hydroxylase.

### Example 30—High Level Production of Psilocybin by Engineered S. cerevisiae

In previous examples a range of substituted tryptamine derivatives were produced by engineered S. cerevisiae strains. This was achieved by integrating derivatizing enzymes into yeast strains engineered to efficiently produce high amounts of the precursor tryptamine. For example, integration of OsT5H and FoCPR into high tryptamine producer SC-106 resulted in high level production of serotonin. In this example high producing psilocybin strains using genes from P. cubensis and P. cyanscens were constructed by combining beneficial genetic modifications from previous examples. These strains combined various optimized pathways leading to psilocybin and included the introduction of a phosphoketolase bypass (CkPta, BbXfpk, gpp1Δ) to increase Erythrose 4-phosphate, an optimized Chorismate pathway (ARO4(K229 L), ARO1, ARO2, RIC1Δ) to improve converse of Erythrose 4-phosphate and Phosphoenolpyruvate to Chorismate, and optimized tryptamine pathway (TRP2 (S65R, S76 L), BsPrs, TRP4, TRP1, TRP3, CrTdc) to convert Chorismate to Tryptophan then decarboxylation to Tryptamine, with deletion of ARO10 and PDC5 to eliminate production of Tryptophol from Tryptophan, an optimized psilocybin pathway (PsiH, CPR, PsiK, PsiM, CYB5) to convert Tryptamine to Psilocybin with deletion of ERG4 to increase S-adenosylmethionine availability (a substrate for methylation by PsiM), and finally deletion of native phosphatase genes (DIA3Δ, PHO5Δ, PHO3Δ) to prevent degradation of psilocybin to psilocin. During the course of the strain construction process it was found that some biosynthetic pathway steps were rate-limiting, in these cases it was found that overexpression of a 2^{nd} copy of the corresponding gene significantly improved production (ARO4(K229 L), TRP2 (S65R, S76 L), PsiK, PsiM). A summary of all the genetic modifications made leading to optimized psilocybin production strains is shown in Table 36.

TABLE 36
Summary of genetic modifications for the optimized production
of psilocybin in S. cerevisiae. Δ denotes the gene was
knocked-out, ×2 denotes the gene was overexpressed two times.

| | Genes | |
|---|---|---|
| Pathway | overexpressed/ | |
| optimized | KO’d | SEQ ID NO’s |
| Phosphoketolase | CkPta | SEQ ID NO: 3 |
| bypass | BbXfpk | SEQ ID NO: 1 |
| | GPP1Δ | SEQ ID NO: 34 |
| Chorismate | ARO4 | SEQ ID NO: 5 |
| pathway | (K229L) × 2 | |
| | ARO1 | SEQ ID NO: 7 |
| | ARO2 | SEQ ID NO: 11 |
| | RIC1Δ | SEQ ID NO: 33 |
| Tryptamine | TRP2 | SEQ ID NO: 13 |
| pathway | (S65R, S76L) × 2 | |
| | BsPrs | SEQ ID NO: 15 |
| | TRP4 | SEQ ID NO: 17 |
| | TRP1 | SEQ ID NO: 19 |
| | TRP3 | SEQ ID NO: 21 |
| | CrTdc | SEQ ID NO: 25 |
| | PDC5Δ | SEQ ID NO: 38 |
| | ARO10Δ | SEQ ID NO: 37 |
| Psilocybin | PsiH | SEQ ID NO: 87, 93 |
| pathway | CPR | SEQ ID NO: 101, 105 |
| | PsiK × 2 | SEQ ID NO: 155, 159 |
| | PsiM × 2 | SEQ ID NO: 123, 127 |
| | CYB5 | SEQ ID NO: 253 |
| | ERG4Δ | SEQ ID NO: 183 |
| Psilocybin | DIA3Δ | SEQ ID NO: 40 |
| degradation | PHO5Δ | SEQ ID NO: 41 |
| | PHO3Δ | SEQ ID NO: 44 |

Strains were constructed according to Example 1, cultivated according to Example 4 and metabolites quantified according to Example 8. As shown in Table 37 combining beneficial genetic modifications led to significant improvement in psilocybin titer with a more than 2-fold improvement in titer and almost no psilocin accumulation.

TABLE 37
High level production of psilocybin in optimized production
strains expression the psilocybin biosynthetic pathway from
P. cubensis and P. cyanascens. The date is presented in mg/L
and is the average of triplicate experiments. ND: Not detected.

| Strain | Genotype description | Tryptamine | Psilocybin | Psilocin |
|---|---|---|---|---|
| BY4741 | Wild-type | ND | ND | ND |
| SC-106 | Tryptamine producer | 546.6 | ND | ND |
| | parental strain | | | |
| SC-275 | Psilocybin pathway | 9.67 | 313.34 | 47.56 |
| | from Psilocybe | | | |
| | cubensis | | | |
| SC-268 | Psilocybin pathway | 13.57 | 410.23 | 61.48 |
| | from Panaeolus | | | |
| | cyanescens | | | |
| SC-415 | Improved psilocybin | ND | 869.96 | 9.45 |
| | pathway from | | | |
| | Psilocybe cubensis | | | |
| SC-416 | Improved psilocybin | ND | 986.56 | 4.65 |
| | pathway from | | | |
| | Panaeolus cyanescens | | | |

### Conclusion

The results of this experiment showed that combining beneficial genetic modifications together resulted in significant improvement in psilocybin production. These results surprisingly showed that different highly engineered metabolic pathways could act synergistically to improve production of the overall final product. Combining strategies to improve flux through the main metabolic pathway (tryptophan pathway), decreased flux through competing pathways (pentose phosphate pathway, phenylalanine/tyrosine pathway, ergosterol pathway), improved co-substrate availability (SAM), and removed degradation pathways (tryptophol, psilocin) and led to significant improvements in psilocybin production that could only be achieved through this combined engineering effort.

### Example 31—Production of baeocystin by engineered S. cerevisiae

Wild-type psilocybin synthase (PcPsiM, SEQ ID NO: 123) efficiently catalyzes the iterative methylation of norbaeocystin to baeocystin then to psilocybin. However, a variant of this enzymes is known in the art which only catalyzes the first methylation of norbaeocystin to baeocystin (PcPsiM(H210A), SEQ ID NO: 129) (Janis Fricke, 2019). When integrated into S. cerevisiae, this variant could be used to produce baeocystin instead of psilocybin. To demonstrate the applicability of this gene variant the full psilocybin biosynthetic pathway from P. cubensis was integrated into high producing tryptamine strain SC-106 but instead of wild-type PcPsiM, the variant PcPsiM(H210A) was instead integrated. Strains were constructed according to Example 1, cultivated according to Example 4 and the resulting metabolites quantified according to Example 8. The results shown in Table 38 demonstrate how integration of PcPsiM(H210) results in only the production of baeocystin with no psilocybin detected. The dephosphorylated degradation production of baeocystin, norpsilocin was also detected which is to be expected given the high concentration of baeocystin produced and the presumably low stability of the phosphorylated molecule, as has been observed for psilocybin.

TABLE 38
Production of baeocystin by engineered S. cerevisiae strains
in mg/L. Data presented as averages of triplicate experiments.

| | Genotype | | | | | |
|---|---|---|---|---|---|---|
| Strain | description | Tryptamine | Psilocybin | Psilocin | Baeocystin | Norpsilocin |
| BY4741 | Wild-type | ND | ND | ND | ND | ND |
| SC-106 | Tryptamine | 548.45 | ND | ND | ND | ND |
| | producer parental | | | | | |
| | strain | | | | | |
| SC-275 | Psilocybin | 10.34 | 312.23 | 39.43 | 2.45 | 0.12 |
| | pathway from | | | | | |
| | Psilocybe cubensis | | | | | |
| SC-417 | Baeocystin | 8.34 | ND | ND | 218.34 | 23.5 |
| | pathway from | | | | | |
| | Psilocybe cubensis | | | | | |

ND: Not detected.

### Conclusion

These results showed that yeast could also serve as a production host to produce this mono-methylated intermediate, alleviating the need to add expensive substrates and co-factors as described for in vitro methods known in the art.

1. A method for producing a tryptamine derivative of formula (I): 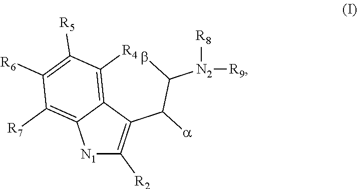
wherein the tryptamine derivative (I) is not tryptophane, 4-hydroxytryptamine, N-acetyl-4-hydroxytryptamine, norbaeocystin, baeocystin; psilocybin, psilocin, aeruginascin, halogenated tryptophan, halogenated tryptamine, halogenated N-methylated tryptamine, halogenated N,N-dimethyltryptamine or halogenated N,N,N-trimethyltryptamine; said method comprising providing an indole acceptor of the formula (II): 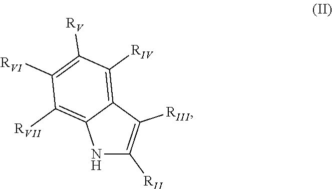
wherein one or more of R_{II}, R_{IV}, R_{V}, R_{VI} or R_{VII} is not H and R_{III} is H or CH₂CH₂NH₂ or CH₂CHCOOHNH₂; and contacting the indole acceptor with a substituent donor in the presence of one or more enzymes substituting one or more H, OH and/or COOH in the indole acceptor with one or more substituents of the substituent donor.

2. The method of claim 1, wherein one or more of RII, RIV, RV, RVI and/or RVII of the indole acceptor (II) is OH, Cl, Br, F, I, CH3, NO2, or CH3-O.

3. The method of claim 1, wherein R4 and/or R5 is OH.

4. The method of claim 1 or 2, wherein the indole acceptor (II) is selected from 4-hydroxytryptamine, 5-hydroxytryptamine, Psilocybin, Psilocin, Norpsilocin, Baeocystin, Norbaeocystin, Aeruginascin, 4-hydroxy-N,N,N-trimethyltryptamine, Bufotenine, Norbufotenine, 5-hydroxy-N,N,N-trimethyltryptamine, 4-methoxytyptamine, 5-methoxytryptamine N-acetylserotonin, Ibogaine, Ibogamine, Noribogaine, Mitragynine, 7-OH-mitragynine, 4-HO-DET, 4-HO-DiPT, 4-HO-MET, 4-HO-MiPT, 4-HO-McPT, 4-HO-DPT, and 4-HO-DSBT, Harmalol.

5. The method of any preceding claim, wherein the substituent is an alkyl group, an acetyl group, a glycosyl group, a phosphate group, an oxygenyl group, a hydroxyl group or a halogenyl group.

6. The method of any preceding claim, wherein the alkyl group is an ethyl or a methyl group.

7. The method of claim 5, wherein the glycosyl group moiety of the glycosyl group comprises one or more of glucose, galactose, xylose, mannose, galactofuranose, arabinose, rhamnose, apiose, fucose, glucosamine, galactosamine, N-acetylglucosamine, N-acetylgalactosamine, xylosamine, mannosamine, arabinosamine, rhamnosamine, apiosamine, fucosamine, glucuronate, galacturonate, mannuronate, arabinate, apionate or a combination thereof.

8. The method of claim 5, wherein the substitution is an O-alkylation, a N-alkylation, or a C-alkylation.

9. The method of claim 8, wherein the alkylation is an ethylation or a methylation.

10. The method of claim 5, wherein the substitution is an N-acetylation

11. The method of claim 5, wherein the substitution is an O-glycosylation, optionally a β-O-glycosylation.

12. The method of any preceding claim, wherein the substituent donor is an aldehyde, a ketone, an ether and/or an amine.

13. The method of claim 12, wherein the aldehyde is acetaldehyde, oxaloacetaldehyde or secologanin.

14. The method of claim 12, wherein the ketone is cinnamoyl-CoA or pyruvate

15. The method of claim 12, wherein the ether is a glycoside.

16. The method of claim 15, wherein glycoside is a nucleotide glycoside.

17. The method of claim 16, wherein the nucleotide glycoside is NTP-glycoside, NDP-glycoside or NMP-glycoside.

18. The method of claim 17, wherein the nucleoside of the nucleotide glycoside is selected from Uridine, Adenosin, Guanosin, Cytidin and deoxythymidine.

19. The method of claim 18, wherein the nucleotide glycoside is selected from UDP-glycosides, ADP-glycosides, CDP-glycosides, CMP-glycosides, dTDP-glycosides and GDP-glycosides.

20. The method of claim 19, wherein the nucleotide glycoside is selected from UDP-D-glucose (UDP-Glc); UDP-galactose (UDP-Gal); UDP-D-xylose (UDP-Xyl); UDP-N-acetyl-D-glucosamine (UDP-GIcNAc); UDP-N-acetyl-D-galactosamine (UDP-GaINAc); UDP-D-glucuronic acid (UDP-GIcA); UDP-D-galactofuranose (UDP-Galf); UDP-arabinose; UDP-rhamnose, UDP-apiose; UDP-2-acetamido-2-deoxy-α-D-mannuronate; UDP-N-acetyl-D-galactosamine 4-sulfate; UDP-N-acetyl-D-mannosamine; UDP-2,3-bis(3-hydroxytetradecanoyl)-glucosamine; UDP-4-deoxy-4-formamido-β-L-arabinopyranose; UDP-2,4-bis(acetamido)-2,4,6-trideoxy-α-D-glucopyranose; UDP-galacturonate; UDP-3-amino-3-deoxy-α-D-glucose; guanosine diphospho-D-mannose (GDP-Man); guanosine diphospho-L-fucose (GDP-Fuc); guanosine diphospho-L-rhamnose (GDP-Rha); cytidine monophospho-N-acetylneuraminic acid (CMP-Neu5Ac); cytidine monophospho-2-keto-3-deoxy-D-mannooctanoic acid (CMP-Kdo); and ADP-glucose.

21. The method of claim 12, wherein the amine is S-Adenosyl methionine (SAM) or S-Adenosyl ethionine (SAE).

22. The method of any preceding claim, wherein the one or more enzymes is selected from glycosyltransferases, alkyltransferases, synthases, acetyltransferases, kinases, cinnamoyltransferases, phosphatases, laccases, halogenases, P450 enzymes, flavin monooxygenases, and/or lyases.

23. The method of claim 22, wherein the glycosyltransferase is derived from a plant or a fungus.

24. The method of claim 23, wherein the plant is selected from Oryza sativa, Crocus sativus, Nicotiana tabacum, Stevia rebaudiana, Nicotiana benthatamiana and Arabidopsis thaliana.

25. The method of claim 22 to 24, wherein the glycosyl transferase is an O-glycoside transferase and/or a C-glycoside transferase.

26. The method of claim 25, wherein the glycosyl transferase is an aglycone O-glycosyltransferase.

27. The method of claim 25, wherein the glycosyl transferase is a glycoside O-glycosyltransferase.

28. The method of claim 25, wherein the glycosyl transferase is an aglycone O-glucosyltransferase.

29. The method of claim 25, wherein the glycosyl transferase is an aglycone O-rhamnosyltransferase.

30. The method of claim 25, wherein the glycosyl transferase is an aglycone O-xylosyltransferase.

31. The method of claim 25, wherein the glycosyl transferase is an aglycone O-arabinosyltransferase.

32. The method of claim 25, wherein the glycosyl transferase is an aglycone O-N-acetylgalactosaminyltransferase.

33. The method of claim 25, wherein the glycosyl transferase is an aglycone O-N-acetylglucosaminyltransferase.

34. The method of claim 25, wherein the glycosyl transferase is an aglycone/glycoside mono-O-glycosyltransferase.

35. The method of claim 25, wherein the glycosyl transferase is an aglycone/glycoside di-O-glycosyltransferase.

36. The method of claim 25, wherein the the glycosyl transferase is an aglycone/glycoside tri-O-glycosyltransferase.

37. The method of claim 25, wherein the glycosyl transferase is an aglycone/glycoside tetra-O-glycosyltransferase.

38. The method of claim 25, wherein the glycosyl transferase is a hydroxytryptamine glycosyltransferase.

39. The method of claim 25, wherein the glycosyl transferase is selected from EC2.4.1.-, and EC2.4.2.

40. The method of claim 39, wherein the glycosyl transferase is selected from EC2.4.1.17, EC2.4.1.35, EC2.4.1.159, EC2.4.1.203, EC2.4.1.234, EC2.4.1.236 and EC2.4.1.294.

41. The method of claim 39, wherein the glycosyl transferase is selected from EC2.4.2.40.

42. The method of claim 25, wherein the glycosyl transferase has at least 70%, such at least 75%, such as at least 80%, such as at least 90%, such as at least 95%, such as at least 99%, such as 100% identity to the glycosyl transferase comprised in anyone of SEQ ID NO: 80, 82, 84, 86, 188, 190, 192, 194, 196, 198, 200, 202, 204, 206, 208, 210, 212, 214, 216, 218, 220, 222, 224, 226, 228, 230, 232, 234, 236, 238, 240, 242, 244, 246, 248, 250 and/or 252.

43. The method of claim 42, wherein the glycosyl transferase has at least 70%, such at least 75%, such as at least 80%, such as at least 90%, such as at least 95%, such as at least 99%, such as 100% identity to the glycosyl transferase comprised in anyone of SEQ ID NO: 194 and/or 204.

44. The method of claim 22, wherein the alkyl transferase is a methyltransferase.

45. The method of claim 44, wherein the methyltransferase is an O-methyltransferase, a N-methyltransferase or a C-methyltransferase.

46. The method of claim 44, wherein the O-methyltransferase has at least 70%, such at least 75%, such as at least 80%, such as at least 90%, such as at least 95%, such as at least 99%, such as 100% identity to the O-methyltransferase comprised in anyone of SEQ ID NO: 114, 116, 118 and/or 120.

47. The method of claim 44, wherein the N-methyltransferase has at least 70%, such at least 75%, such as at least 80%, such as at least 90%, such as at least 95%, such as at least 99%, such as 100% identity to the N-methyltransferase comprised in anyone of SEQ ID NO: 122, 124, 126, 128, 130, 132, 134 136 and/or 138.

48. The method of claim 44, wherein the C-methyltransferase has at least 70%, such at least 75%, such as at least 80%, such as at least 90%, such as at least 95%, such as at least 99%, such as 100% identity to the C-methyltransferase comprised in SEQ ID NO: 140.

49. The method of claim 22, wherein the synthase is a Strictosidine synthase or a 1-acetyl-β-carboline synthase.

50. The method of claim 49, wherein the Strictosidine synthase has at least 70%, such at least 75%, such as at least 80%, such as at least 90%, such as at least 95%, such as at least 99%, such as 100% identity to the Strictosidine synthase comprised in anyone of SEQ ID NO: 144 146, 148 and/or 150.

51. The method of claim 49, wherein the 1-acetyl-β-carboline synthase has at least 70%, such at least 75%, such as at least 80%, such as at least 90%, such as at least 95%, such as at least 99%, such as 100% identity to the 1-acetyl-β-carboline synthase comprised in anyone of SEQ ID NO: 152 and/or 154.

52. The method of claim 22, wherein the acetyltransferase is an aralkylamine N-acetyltransferase, optionally an aralkylamine N-acetyltransferase which has at least 70%, such at least 75%, such as at least 80%, such as at least 90%, such as at least 95%, such as at least 99%, such as 100% identity to the aralkylamine N-acetyltransferase comprised in SEQ ID NO: 142.

53. The method of claim 22, wherein the kinase is a 4-Hydroxytryptamine kinase and/or a 7-hydroxytryptamine kinase optionally a 4-Hydroxytryptamine kinase and/or a 7-hydroxytryptamine kinase which has at least 70%, such at least 75%, such as at least 80%, such as at least 90%, such as at least 95%, such as at least 99%, such as 100% identity to the 4-Hydroxytryptamine kinase and/or 7-hydroxytryptamine kinase comprised in anyone of SEQ ID NO: 156, 158, and/160.

54. The method of claim 22, wherein the cinnamoyltransferase is a N-hydroxycinnamoyltransferase, optionally a N-hydroxycinnamoyltransferase which has at least 70%, such at least 75%, such as at least 80%, such as at least 90%, such as at least 95%, such as at least 99%, such as 100% identity to the N-hydroxycinnamoyl transferase comprised in SEQ ID NO: 162.

55. The method of claim 22, wherein the phosphatase is a psilocybin phosphatase, optionally a psilocybin phosphatase which has at least 70%, such at least 75%, such as at least 80%, such as at least 90%, such as at least 95%, such as at least 99%, such as 100% identity to the psilocybin phosphatase comprised in SEQ ID NO: 164.

56. The method of claim 22, wherein the laccase is a psilocin laccase, optionally a psilocybin laccase which has at least 70%, such at least 75%, such as at least 80%, such as at least 90%, such as at least 95%, such as at least 99%, such as 100% identity to the psilocin laccase comprised in SEQ ID NO: 166.

57. The method of claim 22, wherein the halogenase is a Tryptophan 2-halogenase, a Tryptophan 5-halogenase, a Tryptophan 6-halogenase or a Tryptophan 7-halogenase.

58. The method of claim 57, wherein the Tryptophan 2-halogenase has at least 70%, such at least 75%, such as at least 80%, such as at least 90%, such as at least 95%, such as at least 99%, such as 100% identity to the Tryptophan 2-halogenase comprised in SEQ ID NO: 168.

59. The method of claim 57, wherein the Tryptophan 5-halogenase has at least 70%, such at least 75%, such as at least 80%, such as at least 90%, such as at least 95%, such as at least 99%, such as 100% identity to the Tryptophan 5-halogenase comprised in SEQ ID NO: 170.

60. The method of claim 57, wherein the Tryptophan 6-halogenase has at least 70%, such at least 75%, such as at least 80%, such as at least 90%, such as at least 95%, such as at least 99%, such as 100% identity to the Tryptophan 6-halogenase comprised in SEQ ID NO: 172.

61. The method of claim 57, wherein the Tryptophan 7-halogenase has at least 70%, such at least 75%, such as at least 80%, such as at least 90%, such as at least 95%, such as at least 99%, such as 100% identity to the Tryptophan 7-halogenase comprised in SEQ ID NO: 174.

62. The method of claim 22, wherein the P450 enzyme has at least 70%, such at least 75%, such as at least 80%, such as at least 90%, such as at least 95%, such as at least 99%, such as 100% identity to the P450 enzymes comprised in anyone of SEQ ID NO: 88, 90, 92, 94, 96, 100 and/or 178 and optionally further comprises contacting the P450 Enzymes with a P450 reductase (CPR).

63. The method of claim 62, wherein the P450 enzyme has at least 70%, such at least 75%, such as at least 80%, such as at least 90%, such as at least 95%, such as at least 99%, such as 100% identity to the P450 enzyme comprised in SEQ ID NO:96 (OsT5H) and optionally the P450 reductase has at least 70%, such at least 75%, such as at least 80%, such as at least 90%, such as at least 95%, such as at least 99%, such as 100% identity to the P450 reductase comprised in SEQ ID NO: 112 (FoCPR).

64. The method of claim 22, wherein the flavin monooxygenase has at least 70%, such at least 75%, such as at least 80%, such as at least 90%, such as at least 95%, such as at least 99%, such as 100% identity to the flavin monooxygenase comprised in SEQ ID NO: 98

65. The method of claim 22, wherein the lyase has at least 70%, such at least 75%, such as at least 80%, such as at least 90%, such as at least 95%, such as at least 99%, such as 100% identity to the lyase comprised in anyone of SEQ ID NO: 52 or 176

66. The method of claims 42 to 61, wherein the sequence identity is at least 90%, such as at least 95%, such as at least 99%, such as 100%.

67. The method of claim 66, wherein the sequence identity is at least 99%, such as 100%.

68. The method of claim 22, wherein the tryptamine derivative (I) is a hydroxytryptamine β-O-glycoside, such as a hydroxytryptamine β-O-glucoside.

69. The method of claim 68, wherein the hydroxytryptamine β-O-glycoside is selected from 4-hydroxytryptamine-β-O-glycoside, Psilocin-β-O-glycoside, Norpsilocin-β-O-glycoside, 4-hydroxy-N,N,N-trimethyltryptamine-β-O-glycoside, Serotonin-β-O-glycoside, Bufotenine-β-O-glycoside, Norbufotenine-β-O-glycoside, 5-hydroxy-N,N,N-trimethyltryptamine-β-O-glycoside, N-acetylserotonin-β-O-glycoside, Noribogaine-β-O-glycoside, 7-OH-mitragynine-β-O-glycoside, 4-HO-DET-β-O-glycoside, 4-HO-DiPT-β-O-glycoside, 4-HO-MET-β-O-glycoside, 4-HO-MiPT-β-O-glycoside, 4-HO-McPT-β-O-glycoside, 4-HO-DPT-β-O-glycoside, 4-HO-DSBT-β-O-glycoside, Harmalol-β-O-glycoside.

70. The method of any preceding claim, further comprising one or more steps selected from:
a) converting an indole or indole derivative into tryptophan or a tryptophan derivative; and
b) converting tryptophan or tryptophan derivative into tryptamine or tryptamine derivative.

71. The method of claim 70 wherein the steps are performed in vitro.

72. The method of claim 70, wherein the conversion of the indole or indole derivative into the tryptophan or tryptophan derivative comprises contacting the indole or indole derivative with a tryptophan synthase enzyme, optionally a tryptophan synthase which has at least 70%, such at least 75%, such as at least 80%, such as at least 90%, such as at least 95%, such as at least 99%, such as 100% identity to the tryptophan synthase comprised in SEQ ID NO: 60, 62, 64, 66, 68, 180, 182 and/or 256.

73. The method of claim 72, wherein the tryptophan synthase has at least 70%, such at least 75%, such as at least 80%, such as at least 90%, such as at least 95%, such as at least 99%, such as 100% identity to the tryptophan synthase comprised in SEQ ID NO: 256.

74. The method of claim 70 or 72, wherein the conversion of the tryptophan or tryptophan derivative into the tryptamine or tryptamine derivative comprises contacting the tryptophan or tryptophan derivative with a tryptophan decarboxylase enzyme, optionally a tryptophan decarboxylase which has at least 70%, such at least 75%, such as at least 80%, such as at least 90%, such as at least 95%, such as at least 99%, such as 100% identity to the tryptophan decarboxylase comprised in SEQ ID NO: 26, 70, 72, 74,76 and/or 78.

75. The method of claim 70 to 74, wherein the conversion of the indole or indole derivative into the tryptophan or tryptophan derivative comprises contacting the indole or indole derivative with a tryptophan synthase enzyme which has at least 70%, such at least 75%, such as at least 80%, such as at least 90%, such as at least 95%, such as at least 99%, such as 100% identity to the tryptophan synthase comprised in SEQ ID NO: 180 and/or 256 in the presence of a tryptophan decarboxylase which has at least 70%, such at least 75%, such as at least 80%, such as at least 90%, such as at least 95%, such as at least 99%, such as 100% identity to the tryptophan decarboxylase comprised in SEQ ID NO: 72 and/or 78.

76. The method of claim 69 to 75, wherein the indole acceptor is serotonin and the tryptamine derivative (I) is a derivative of serotonin, optionally Melatonin, Normelatonin or hydroxycinnamoylserotonin derivatives such as 4-coumaroylserotonin, and the one or more enzymes substituting one or more H, OH and/or COOH in the indole acceptor with one or more substituents of the substituent donor are selected from
a) an acetyl transferase which has at least 70% to the identity to the acetyl transferase comprised in SEQ ID NO: 142
b) an O-methyl transferase which has at least 70% to the identity to the O-methyl transferase comprised in SEQ ID NO: 118; and/or
c) a N-hydroxycinnamoyl transferase which has at least 70% to the identity to the N-hydroxycinnamoyl transferase comprised in SEQ ID NO: 162.

77. The method of claim 70, further comprising one or more further steps of
a) Glycosylation;
b) Methylation;
c) Hydroxylation;
d) Condensation;
e) Nitration;
f) Oxidation;
g) Lyase deamidation; or
h) Dephosphorylation

78. The method of claim 77, wherein the hydroxylation step comprises contacting the indole or indole derivative or tryptophan or tryptophan derivative with a hydroxylase, optionally a hydroxylase which has at least 70%, such at least 75%, such as at least 80%, such as at least 90%, such as at least 95%, such as at least 99%, such as 100% identity to the hydroxylase comprised in anyone of SEQ ID NO: 88, 90, 92, 94, 96, 98, and/or 100, optionally in the presence of a Cytochrome P450 reductase (CRP) which has at least 70%, such at least 75%, such as at least 80%, such as at least 90%, such as at least 95%, such as at least 99%, such as 100% identity to the CPR comprised in anyone of SEQ ID NO: 102, 104, 106, 108, 110 and/or 112.

79. The method of claim 77, wherein the lyase deamidation step comprises contacting the indole or indole derivative or tryptophan or tryptophan derivative with a lyase, optionally a lyase, which has at least 70%, such at least 75%, such as at least 80%, such as at least 90%, such as at least 95%, such as at least 99%, such as 100% identity to the lyase comprised in anyone SEQ ID NO: 52 and/or 176.

80. The method of any preceding claim comprising in vitro enzymatic substitution steps and/or optionally in vivo enzymatic substitution steps.

81. The method of claim 80 comprising expressing a glycosyl transferase in E. coli and performing in vitro glycosylation of the indole acceptor.

82. A tryptamine derivative of formula (I): 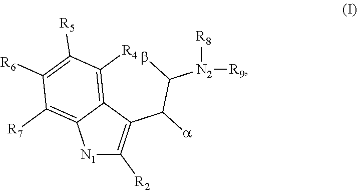
wherein at least one of R2, R4 to R9, α and β is a glycosyl group.

83. The tryptamine derivative of claim 80, selected from the group of 4-hydroxytryptamine-β-O-glycoside, Psilocin-β-O-glycoside, Norpsilocin-β-O-glycoside, 4-hydroxy-N,N,N-trimethyltryptamine-β-O-glycoside, Serotonin-β-O-glycoside, Bufotenine-β-O-glycoside, Norbufotenine-β-O-glycoside, 5-hydroxy-N,N,N-trimethyltryptamine-β-O-glycoside, N-acetylserotonin-β-O-glycoside, Noribogaine-β-O-glycoside, 7-OH-mitragynine-β-O-glycoside, 4-HO-DET-β-O-glycoside, 4-HO-DiPT-β-O-glycoside, 4-HO-MET-β-O-glycoside, 4-HO-MiPT-β-O-glycoside, 4-HO-McPT-β-O-glycoside, 4-HO-DPT-β-O-glycoside, 4-HO-DSBT-β-0-glycoside, and wherein the glycoside of Serotonin-β-O-glycoside, Bufotenine-β-O-glycoside, Norbufotenine-β-O-glycoside or 5-hydroxy-N,N,N-trimethyltryptamine-β-O-glycoside is not a glucoside.

84. A microbial host cell genetically modified to perform the method of claims 1 to 79 and produce the tryptamine derivative, wherein the host cell expresses one or more heterologous genes encoding the one or more enzymes, which in the presence of the indole acceptor and one or more substituent donors, transfers one or more substituents to the one or more H, OH and/or COOH of the indole acceptor.

85. The host cell of claim 82, expressing one or more genes selected from:
a) genes encoding a UGT said genes which are at least 70% identical to the UGT encoding polynucleotide comprised in anyone of SEQ ID NO: 79, 81, 83, 85, 187, 189, 191, 193, 195, 197, 199, 201, 203, 205, 207, 209, 211, 213, 215, 217, 219, 221, 223, 225, 227, 229, 231, 233, 235, 237, 239, 241, 243, 245, 247, 249, and/or 251 or genomic DNA thereof;
b) genes encoding an O-methyltransferase said genes which are at least 70% identical to the O-methyltransferase encoding polynucleotide comprised in anyone of SEQ ID NO: 113, 115, 117, and/or 119 or genomic DNA thereof;
c) genes encoding an N-methyltransferase said genes which are at least 70% identical to the N-methyltransferase encoding polynucleotide comprised in anyone of SEQ ID NO: 121, 123, 125, 127, 129, 131, 133, 135, and/or 137 or genomic DNA thereof;
d) genes encoding a C-methyltransferase said genes which are at least 70% identical to the C-methyltransferase encoding polynucleotide comprised in SEQ ID NO: 139 or genomic DNA thereof;
e) genes encoding a strictosidine synthase said genes which are at least 70% identical to the strictosidine synthase encoding polynucleotide comprised in anyone of SEQ ID NO: 143, 145, 147, and/or 149 or genomic DNA thereof;
f) genes encoding a 1-acetyl-β-carboline synthase said genes which are at least 70% identical to the 1-acetyl-β-carboline synthase encoding polynucleotide comprised in anyone of SEQ ID NO: 151, and/or 153 or genomic DNA thereof;
g) genes encoding a 1-acetyl-β-carboline synthase said genes which are at least 70% identical to the aralkylamine N-acetyltransferase encoding polynucleotide comprised in SEQ ID NO: 141 or genomic DNA thereof;
h) genes encoding a 4-Hydroxytryptamine kinase and/or a 7-hydroxytryptamine kinase said genes which are at least 70% identical to the 4-Hydroxytryptamine kinase and/or a 7-hydroxytryptamine kinase encoding polynucleotide comprised in anyone of SEQ ID NO: 155, 157, and/or 159 or genomic DNA thereof;
i) genes encoding an N-hydroxycinnamoyltransferase said genes which are at least 70% identical to the N-hydroxycinnamoyltransferase encoding polynucleotide comprised in SEQ ID NO: 161 or genomic DNA thereof;
j) genes encoding a psilocybin phosphatase said genes which are at least 70% identical to the psilocybin phosphatase encoding polynucleotide comprised in SEQ ID NO: 163 or genomic DNA thereof;
k) genes encoding a psilocin laccase said genes which are at least 70% identical to the psilocin laccase encoding polynucleotide comprised in SEQ ID NO: 165 or genomic DNA thereof;
l) genes encoding a Tryptophan 2-halogenase said genes which are at least 70% identical to the Tryptophan 2-halogenase encoding polynucleotide comprised in SEQ ID NO: 167 or genomic DNA thereof;
m) genes encoding a Tryptophan 5-halogenase said genes which are at least 70% identical to the Tryptophan 5-halogenase encoding polynucleotide comprised in SEQ ID NO: 169 or genomic DNA thereof;
n) genes encoding a Tryptophan 6-halogenase said genes which are at least 70% identical to the Tryptophan 6-halogenase encoding polynucleotide comprised in SEQ ID NO: 171 or genomic DNA thereof;
o) genes encoding a Tryptophan 7-halogenase said genes which are at least 70% identical to the Tryptophan 7-halogenase encoding polynucleotide comprised in SEQ ID NO: 173 or genomic DNA thereof;
p) genes encoding a P450 enzyme said genes which are at least 70% identical to the P450 enzyme encoding polynucleotide comprised in anyone of SEQ ID NO: 87, 89, 91, 93, 95, 99, and/or 177 or genomic DNA thereof;
q) genes encoding a P450 reductase said genes which are at least 70% identical to the P450 reductase (CPR) encoding polynucleotide comprised in anyone of SEQ ID NO: 101, 103, 105, 107, 109, and/or 111 or genomic DNA thereof;
r) genes encoding a flavin monooxygenase said genes which are at least 70% identical to the flavin monooxygenase encoding polynucleotide comprised in SEQ ID NO: 97 or genomic DNA thereof; and/or
s) genes encoding a lyase said genes which are at least 70% identical to the lyase encoding polynucleotide comprised in anyone of SEQ ID NO: 51 and/or 175 or genomic DNA thereof.

86. The host cell of claim 82 to 83, further comprising an operative biosynthetic pathway producing the indole acceptor, wherein the host cell expresses one or more pathway genes encoding polypeptides selected from:
a) one or more enzymes converting glucose to fructose-6-phosphate;
b) a fructose-6-phosphate phosphoketolase converting fructose-6-phosphate to Erythrose-4-phosphate and acetyl phosphate;
c) a Phosphotransacetylase converting Acetyl phosphate to Acetyl-CoA;
d) one or more enzymes converting Fructose-6-phosphate to Phosphoenolpyruvate;
e) a 3-deoxy-D-arabino-heptulosonate 7-phosphate synthase (DAHP synthase) converting Phosphoenolpyruvate and Erythrose-4-phosphate to 3-deoxy-D-arabino-heptulosonate-7-phosphate (DAHP);
f) one or more enzymes converting 3-deoxy-D-arabino-heptulosonate-7-phosphate to 5-enolpyruvoyl-shikimate 3-phosphate;
g) a Shikimate kinase converting Shikimate to Shikimate-3-phosphate;
h) a Chorismate synthase converting 5-enolpyruvoyl-shikimate 3-phosphate to Chorismate;
i) a Anthranilate synthase converting Chorismate to Anthranilate;
j) a Ribose-phosphate pyrophosphokinase converting Ribose-5-phosphate to Phospho-alpha-D-ribosyl-1-pyrophosphate;
k) a Anthranilate phosphoribosyl transferase converting Anthranilate and Phospho-alpha-D-ribosyl-1-pyrophosphate to N-(5-phosphoribosyl)-anthranilate;
l) a N-(5′-phosphoribosyl)-anthranilate isomerase converting N-(5-phosphoribosyl)-anthranilate to 1-(o-carboxyphenylamino)-1′-deoxyribulose 5′-phosphate;
m) a Indole-3-glycerol phosphate synthase converting 1-(o-carboxyphenylamino)-1′-deoxyribulose 5′-phosphate to (1S,2R)-1-C-(indol-3-yl)-glycerol 3-phosphate;
n) a Tryptophan synthase converting (1S,2R)-1-C-(indol-3-yl) glycerol 3-phosphate and Serine to L-Tryptophan;
o) a Tryptophan decarboxylase converting L-Tryptophan to Tryptamine;
p) a Chorismate mutase converting Chorismate to Prephenate;
q) a Prephenate dehydrogenase converting Prephenate to Phenylpyruvate;
r) an Aromatic aminotransferase converting Phenylpyruvate to phenylalanine;
s) a Phenylalanine ammonium lyase converting Phenylalanine to cinnamate;
t) a Cinnamate 4-hydroxylase converting Cinnamate to coumarate;
u) a cytochrome b5 assisting Cytochrome P450 reductases reducing hydroxylase enzymes;
v) a Cytochrome P450 reductase reducing cytochrome P450 enzymes;
w) a 4-Coumoryl-CoA ligase converting Coumarate to 4-coumoryl CoA;
x) a Tryptophanase converting tryptophan or a derivative thereof into indole or a derivative thereof;
y) a Tryptophan synthase converting Indole or a derivative thereof and Serine or a derivative thereof into Tryptophan or a derivative thereof;
z) a Tryptophan decarboxylase or a non-canonical aromatic amino acid decarboxylase converting Tryptophan or a derivative thereof into Tryptamine or a derivative thereof;
aa) a Tryptamine 5-hydroxylase converting tryptamine to serotonin;
bb) a Tryptamine 4-hydroxylase converting tryptamine to 4-hydroxytryptamine.
cc) 4-hydroxytryptamine kinase converting 4-hydroxytryptamine to Norbaeocystin; and/or
dd) Psilocybin synthase converting Norbaeocystin to Psilocybin.

87. The host cell of claim 84, wherein the corresponding:
a) fructose-6-phosphate phosphoketolase has at least 70% identity to the fructose-6-phosphate phosphoketolase comprised in SEQ ID NO: 2;
b) Phosphotransacetylase has at least 70% identity to the Phosphotransacetylase comprised in SEQ ID NO: 4;
c) 3-deoxy-D-arabino-heptulosonate 7-phosphate synthase has at least 70% identity to the 3-deoxy-D-arabino-heptulosonate 7-phosphate synthase comprised in SEQ ID NO: 6;
d) Enzyme converting 3-deoxy-D-arabino-heptulosonate-7-phosphate to 5-enolpyruvoyl-shikimate 3-phosphate has at least 70% identity to the Enzyme comprised in SEQ ID NO: 8;
e) Shikimate kinase has at least 70% identity to the Shikimate kinase comprised in SEQ ID NO: 10;
f) Chorismate synthase has at least 70% identity to the Chorismate synthase comprised in SEQ ID NO: 12;
g) Anthranilate synthase has at least 70% identity to the Anthranilate synthase comprised in SEQ ID NO: 14;
h) Ribose-phosphate pyrophosphokinase has at least 70% identity to the Ribose-phosphate pyrophosphokinase comprised in SEQ ID NO: 16;
i) Anthranilate phosphoribosyl transferase has at least 70% identity to the Anthranilate phosphoribosyl transferase comprised in SEQ ID NO: 18;
j) N-(5′-phosphoribosyl)-anthranilate isomerase has at least 70% identity to the N-(5′-phosphoribosyl)-anthranilate isomerase comprised in SEQ ID NO: 20;
k) Indole-3-glycerol phosphate synthase has at least 70% identity to the Indole-3-glycerol phosphate synthase comprised in SEQ ID NO: 22;
l) Tryptophan synthase has at least 70% identity to the Tryptophan synthase comprised in anyone of SEQ ID NO: 24, 60, 62, 64, 66, 68, 180 and/or 182;
m) Tryptophan decarboxylase or a non-canonical aromatic amino acid decarboxylase has at least 70% identity to the Tryptophan decarboxylase or a non-canonical aromatic amino acid decarboxylase comprised in anyone of SEQ ID NO: 26, 70, 72, 74, 76, and/or 78;
n) Chorismate mutase has at least 70% identity to the Chorismate mutase comprised in SEQ ID NO: 46;
o) Prephenate dehydrogenase has at least 70% identity to the Prephenate dehydrogenase comprised in SEQ ID NO: 48;
p) Aromatic aminotransferase has at least 70% identity to the Aromatic aminotransferase comprised in SEQ ID NO: 50;
q) Phenylalanine ammonium lyase has at least 70% identity to the Phenylalanine ammonium lyase comprised in SEQ ID NO: 52;
r) Cinnamate 4-hydroxylase has at least 70% identity to the Cinnamate 4-hydroxylase comprised in SEQ ID NO: 54;
s) cytochrome b5 has at least 70% identity to the cytochrome b5 comprised in SEQ ID NO: 254;
t) Cytochrome P450 reductase has at least 70% identity to the Cytochrome P450 reductase comprised in SEQ ID NO: 56, 102, 104, 106, 108, 110 and/or 112;
u) 4-Coumoryl-CoA ligase has at least 70% identity to the 4-Coumoryl-CoA ligase comprised in SEQ ID NO: 58.
v) Tryptamine 5-hydroxylase has at least 70% identity to the 5-hydroxylase comprised in SEQ ID NO: 96;
w) Typtamine 4-hydroxylase has at least 70% identity to the Tryptamine 4-hydroxylase comprised in SEQ ID NO 94:
x) 4-hydroxytryptamine kinase has at least 70% identity to the 4-hydroxytryptamine kinase comprised in SEQ ID NO: 160; and/or
y) Psilocybin synthase has at least 70% identity to the Psilocybin synthase comprised in SEQ ID NO: 128.

88. The host cell of claim 84 or 85, wherein the one or more expressed genes are selected from:
a) genes encoding a fructose-6-phosphate phosphoketolase said genes being at least 70% identical to the fructose-6-phosphate phosphoketolase encoding polynucleotide comprised in SEQ ID NO: 1 or genomic DNA thereof;
b) genes encoding a Phosphotransacetylase said genes being at least 70% identical to the Phosphotransacetylase encoding polynucleotide comprised in SEQ ID NO: 3 or genomic DNA thereof;
c) genes encoding a 3-deoxy-D-arabino-heptulosonate 7-phosphate synthase said genes being at least 70% identical to the 3-deoxy-D-arabino-heptulosonate 7-phosphate synthase encoding polynucleotide comprised in SEQ ID NO: 5 or genomic DNA thereof;
d) genes encoding an enzyme converting 3-deoxy-D-arabino-heptulosonate-7-phosphate to 5-enolpyruvoyl-shikimate 3-phosphate said genes being at least 70% identical to the polynucleotide encoding the enzyme converting 3-deoxy-D-arabino-heptulosonate-7-phosphate to 5-enolpyruvoyl-shikimate 3-phosphate comprised in SEQ ID NO: 7 or genomic DNA thereof;
e) genes encoding a XX said genes being at least 70% identical to the Shikimate kinase encoding polynucleotide comprised in SEQ ID NO: 9 or genomic DNA thereof;
f) genes encoding a Shikimate kinase said genes being at least 70% identical to the Chorismate synthase encoding polynucleotide comprised in SEQ ID NO: 11 or genomic DNA thereof;
g) genes encoding a Anthranilate synthase said genes being at least 70% identical to the Anthranilate synthase encoding polynucleotide comprised in SEQ ID NO: 13 or genomic DNA thereof;
h) genes encoding a Ribose-phosphate pyrophosphokinase said genes being at least 70% identical to the Ribose-phosphate pyrophosphokinase encoding polynucleotide comprised in SEQ ID NO: 15 or genomic DNA thereof;
i) genes encoding a anthranilate phosphoribosyl transferase said genes being at least 70% identical to the Anthranilate phosphoribosyl transferase encoding polynucleotide comprised in SEQ ID NO: 17 or genomic DNA thereof;
j) genes encoding a N-(5′-phosphoribosyl)-anthranilate isomerase said genes being at least 70% identical to the N-(5′-phosphoribosyl)-anthranilate isomerase encoding polynucleotide comprised in SEQ ID NO: 19 or genomic DNA thereof;
k) genes encoding a Indole-3-glycerol phosphate synthase said genes being at least 70% identical to the Indole-3-glycerol phosphate synthase encoding polynucleotide comprised in SEQ ID NO: 21 or genomic DNA thereof;
l) genes encoding a Tryptophan synthase said genes being at least 70% identical to the Tryptophan synthase encoding polynucleotide comprised in anyone of SEQ ID NO: 23, 59, 61, 63, 65, 67, 179, and/or 181 or genomic DNA thereof;
m) genes encoding a Tryptophan decarboxylase or a non-canonical aromatic amino acid decarboxylase said genes being at least 70% identical to the Tryptophan decarboxylase or a non-canonical aromatic amino acid decarboxylase encoding polynucleotide comprised in SEQ ID NO: 25, 69, 71, 73, 75, and/or 77 or genomic DNA thereof;
n) genes encoding a Chorismate mutase said genes being at least 70% identical to the Chorismate mutase encoding polynucleotide comprised in SEQ ID NO: 45 or genomic DNA thereof;
o) genes encoding a Prephenate dehydrogenase said genes being at least 70% identical to the Prephenate dehydrogenase encoding polynucleotide comprised in SEQ ID NO: 47 or genomic DNA thereof;
p) genes encoding a Aromatic aminotransferase said genes being at least 70% identical to the Aromatic aminotransferase encoding polynucleotide comprised in SEQ ID NO: 49 or genomic DNA thereof;
q) genes encoding a Phenylalanine ammonium lyase said genes being at least 70% identical to the Phenylalanine ammonium lyase encoding polynucleotide comprised in SEQ ID NO: 51 or genomic DNA thereof;
r) genes encoding a cinnamate 4-hydroxylase said genes being at least 70% identical to the Cinnamate 4-hydroxylase encoding polynucleotide comprised in SEQ ID NO: 53 or genomic DNA thereof;
s) genes encoding a cytochrome b5 said genes being at least 70% identical to the C cytochrome b5 encoding polynucleotide comprised in SEQ ID NO: 253 or genomic DNA thereof;
t) genes encoding a Cytochrome P450 reductase said genes being at least 70% identical to the Cytochrome P450 reductase encoding polynucleotide comprised in SEQ ID NO: 55, 101, 103, 105, 107, 109 and/or 111 or genomic DNA thereof;
u) genes encoding a 4-Coumoryl-CoA ligase said genes being at least 70% identical to the 4-Coumoryl-CoA ligase encoding polynucleotide comprised in SEQ ID NO: 57 or genomic DNA thereof.
v) genes encoding a Tryptamine 5-hydroxylase said genes being at least 70% identical to the Tryptamine 5-hydroxylase encoding polynucleotide comprised in SEQ ID NO: 96 or genomic DNA thereof;
w) genes encoding a Cytochrome p450 reductase said genes being at least 70% identical to the Cytochrome p450 reductase encoding polynucleotide comprised in SEQ ID NO: 111 or genomic DNA thereof.
x) genes encoding a 4-hydroxytryptamine kinase said genes being at least 70% identical to the 4-hydroxytryptamine kinase encoding polynucleotide comprised SEQ ID NO 159; and/or
y) genes encoding a psilocybin synthase said genes being at least 70% identical to the psilocybin synthase encoding polynucleotide comprised in SEQ ID NO 127.

89. The host cell of claim 86, further expressing:
a) genes encoding a Psilocybin synthase said genes which are at least 70% identical to the Psilocybin synthase encoding polynucleotide comprised in anyone of SEQ ID NO: 127 and/or 123 or genomic DNA thereof;
b) genes encoding a 4-Hydroxytryptamine kinase said genes which are at least 70% identical to the 4-Hydroxytryptamine kinase encoding polynucleotide comprised in anyone of SEQ ID NO: 159 or genomic DNA thereof;
c) genes encoding a P450 reductase said genes which are at least 70% identical to the P450 reductase (CPR) encoding polynucleotide comprised in SEQ ID NO: 105 and/or 101 or genomic DNA thereof;
d) genes encoding a P450 enzyme said genes which are at least 70% identical to the P450 enzyme encoding polynucleotide comprised in SEQ ID NO: 93 and/or 87 or genomic DNA thereof; and
e) genes encoding a Tryptophan decarboxylase said genes being at least 70% identical to the Tryptophan decarboxylase encoding polynucleotide comprised in SEQ ID NO: 77 and/or 71 or genomic DNA thereof.

90. The host cell of claims 83 or 85 to 86, wherein the sequence identity is least 90%, such as at least 95%, such as at least 99%, such as 100%.

91. The host cell of claim 88, wherein the sequence identity is at least 99%, such as 100%.

92. The host cell of claims 82 to 89, comprising at least two copies of one or more of the heterologous genes encoding the one or more enzymes, which in the presence of the indole acceptor and one or more substituent donors, transfers one or more substituents to the one or more H, OH and/or COOH of the indole acceptor, or of the pathway genes.

93. The host cell of claims 82 to 90, wherein one or more of the heterologous genes encoding the one or more enzymes, which in the presence of the indole acceptor and one or more substituent donors, transfers one or more substituents to the one or more H, OH and/or COOH of the indole acceptor, or of the pathway genes are overexpressed.

94. The cell of claims 82 to 91 further genetically modified to provide an increased amount of a substrate for at least one polypeptide of the indole acceptor pathway.

95. The host cell of claims 82 to 92, further genetically modified to exhibit increased tolerance towards one or more substrates, intermediates, or product molecules from the indole acceptor pathway.

96. The host cell claims 82 to 93, wherein the host cell is an eukaryotic, prokaryotic or archaic cell.

97. The host cell of claim 94, wherein the host cell is an eukaryote cell selected from the group consisting of mammalian, insect, plant, or fungal cells.

98. The host cell of claim 95, wherein the host cell is a fungal host cell selected from phylas consisting of Ascomycota, Basidiomycota, Neocallimastigomycota, Glomeromycota, Blastocladiomycota, Chytridiomycota, Zygomycota, Oomycota and Microsporidia.

99. The host cell of claim 96, wherein the fungal host cell is a yeast selected from the group consisting of ascosporogenous yeast (Endomycetales), basidiosporogenous yeast, and Fungi Imperfecti yeast (Blastomycetes).

100. The host cell of claim 97, wherein the yeast host cell is selected from the genera consisting of Saccharomyces, Kluveromyces, Candida, Pichia, Debaromyces, Hansenula, Yarrowia, Zygosaccharomyces, and Schizosaccharomyces.

101. The host cell of claim 98, wherein the yeast host cell is selected from the species consisting of Kluyveromyces lactis, Saccharomyces carlsbergensis, Saccharomyces cerevisiae, Saccharomyces diastaticus, Saccharomyces douglasii, Saccharomyces kluyveri, Saccharomyces norbensis, Saccharomyces oviformis, Saccharomyces boulardii and Yarrowia lipolytica.

102. The host cell of claim 96, wherein the fungal host cell is filamentous fungus.

103. The host cell of claim 100, wherein the filamentous fungal host cell is selected from the phylas consisting of Ascomycota, Eumycota and Oomycota.

104. The host cell of claim 101, wherein the filamentous fungal host cell is selected from the genera consisting of Acremonium, Aspergillus, Aureobasidium, Bjerkandera, Ceriporiopsis, Chrysosporium, Coprinus, Corio/us, Cryptococcus, Filibasidium, Fusarium, Humicola, Magnaporthe, Mucor, Myceliophthora, Neocallimastix, Neurospora, Paecilomyces, Penicillium, Phanerochaete, Phlebia, Piromyces, Pleurotus, Schizophyllum, Talaromyces, Thermoascus, Thielavia, Tolypocladium, Trametes, and Trichoderma.

105. The host cell of claim 102, wherein the filamentous fungal host cell is selected from the species consisting of Aspergillus awamori, Aspergillus foetidus, Aspergillus fumigatus, Aspergillus japonicus, Aspergillus nidulans, Aspergillus niger, Aspergillus oryzae, Bjerkandera adusta, Ceriporiopsis aneirina, Ceriporiopsis caregiea, Ceriporiopsis gilvescens, Ceriporiopsis pannocinta, Ceriporiopsis rivulosa, Ceriporiopsis subrufa, Ceriporiopsis subvermispora, Chrysosporiuminops, Chrysosporium keratinophilum, Chrysosporium lucknowense, Chrysosporium merdarium, Chrysosporium pannicola, Chrysosporium queenslandicum, Chrysosporium tropicum, Chrysosporium zonatum, Coprinus cinereus, Coriolus hirsutus, Fusarium bactridioides, Fusarium cerealis, Fusarium crookwellense, Fusarium culmorum, Fusarium graminearum, Fusarium graminum, Fusarium heterosporum, Fusarium negundi, Fusarium oxysporum, Fusarium reticulatum, Fusarium roseum, Fusarium sambucinum, Fusarium sarcochroum, Fusarium sporotrichioides, Fusarium sulphureum, Fusarium torulosum, Fusarium trichothecioides, Fusarium venenatum, Humicola insolens, Humicola lanuginosa, Mucor miehei, Myceliophthora thermophila, Neurospora crassa, Penicillium purpurogenum, Phanerochaete chrysosporium, Phlebia radiata, Pleurotus eryngii, Thielavia terrestris, Trametes villosa, Trametes versicolor, Trichoderma harzianum, Trichoderma koningii, Trichoderma longibrachiatum, Trichoderma reesei, and Trichoderma viride.

106. The host cell of claim 94, wherein the host cell is a prokaryotic cell.

107. The host cell of claim 104, wherein the prokaryotic cell is E. coli.

108. The host cell of claim 94, wherein the host cell is an archaic cell.

109. The host cell of claim 106, wherein the archaic cell is an algae.

110. The host cell of claim 82 to 93, wherein one or more native genes are attenuated, disrupted and/or deleted.

111. The host cell of claim 108 wherein the attenuated, disrupted and/or deleted gene is a phosphatase shunting psilocybin to psilocin.

112. The host cell of claim 108 wherein the host cell is a yeast strain modified by attenuating, disrupting and/or deleting one or more native genes selected from:
a) The pyruvate kinase gene comprised in anyone of SEQ ID NO: 27 or any of its paralogs or orthologs having at least 70% identity to SEQ ID NO: 27;
b) The phosphofructokinase gene comprised in anyone of SEQ ID NO: 29 or 31 or any of its paralogs or orthologs having at least 70% identity to anyone of SEQ ID NO: 29 or 31;
c) The transporters gene comprised in SEQ ID NO: 33 or any of its paralogs or orthologs having at least 70% identity to SEQ ID NO: 33;
d) The DL-glycerol-3-phosphate phosphatase gene comprised in SEQ ID NO: 34 or any of its paralogs or orthologs having at least 70% identity to SEQ ID NO: 34;
e) The tryptophan 2,3-dioxygenase gene comprised in SEQ ID NO: 35 or any of its paralogs or orthologs having at least 70% identity to SEQ ID NO: 35;
f) The cystathionine beta-synthase gene comprised in SEQ ID NO: 36 or any of its paralogs or orthologs having at least 70% identity to SEQ ID NO: 36;
g) The phenylpyruvate decarboxylase gene comprised in SEQ ID NO: 37 or any of its paralogs or orthologs having at least 70% identity to SEQ ID NO: 37;
h) The pyruvate decarboxylase gene comprised in SEQ ID NO: 38 or any of its paralogs or orthologs having at least 70% identity to SEQ ID NO: 38;
i) The histone variant H2AZ gene comprised in SEQ ID NO: 39 or any of its paralogs or orthologs having at least 70% identity to SEQ ID NO: 39;
j) The phosphatase gene comprised in SEQ ID NO: 40 or any of its paralogs or orthologs having at least 70% identity to SEQ ID NO: 40;
k) The repressible acid phosphatase gene comprised in anyone of SEQ ID NO: 41, 42, or 43 or any of its paralogs or orthologs having at least 70% identity to anyone of SEQ ID NO: 41, 42, or 43;
l) The constitutively expressed acid phosphatase gene comprised in SEQ ID NO: 44 or any of its paralogs or orthologs having at least 70% identity to SEQ ID NO: 44;
m) The sterol reductase gene comprised in SEQ ID NO: 183 or any of its paralogs or orthologs having at least 70% identity to SEQ ID NO: 183; and/or
n) The S-adenosylmethionine decarboxylase gene comprised in SEQ ID NO: 184 or any of its paralogs or orthologs having at least 70% identity to SEQ ID NO: 184.

113. The host cell of claims 108 to 110, wherein the host cell is a yeast strain modified by overexpressing one or more native genes selected from the NADH kinase gene comprised in SEQ ID NO: 185 or any of its paralogs or orthologs having at least 70% identity to SEQ ID NO: 185.

114. A cell culture, comprising host cell of claims 82 to 110 and a growth medium.

115. The method of claims 1 to 79 further comprising:
a) culturing the cell culture of claim 112 at conditions allowing the host cell to produce the tryptamine derivative (I); and
b) optionally recovering and/or isolating the tryptamine derivative (I).

116. The method of claim 113, further comprising one or more elements selected from:
a) culturing the cell culture in a nutrient growth medium;
b) culturing the cell culture under aerobic or anaerobic conditions
c) culturing the cell culture under agitation;
d) culturing the cell culture at a temperature of between 25 to 50° C.;
e) culturing the cell culture at a pH of between 3-9;
f) culturing the cell culture for between 10 hours to 30 days; and
g) culturing the cell culture under fed-batch, repeated fed-batch, continuous, or semi-continuous conditions.

117. The method of claims 113 to 114, further comprising feeding one or more exogenous indole acceptors or precursors thereof and/or substituent donors to the cell culture.

118. The method of claims 113 to 115, wherein the recovering and/or isolation step comprises separating a liquid phase of host cell or cell culture from a solid phase of host cell or cell culture to obtain a supernatant comprising the tryptamine derivative (I) by one or more steps selected from:
a) disrupting the host cell to release intracellular tryptamine derivative (I) into the supernatant;
b) separating the supernatant from the solid phase of the host cell, such as by filtration or gravity separation;
c) contacting the supernatant with one or more adsorbent resins in order to obtain at least a portion of the produced tryptamine derivative (I);
d) contacting the supernatant with one or more ion exchange or reversed-phase chromatography columns in order to obtain at least a portion of the tryptamine derivative (I);
e) extracting the tryptamine derivative (I); and
f) precipitating the tryptamine derivative (I) by crystallization or evaporating the solvent of the liquid phase; and optionally isolating the tryptamine derivative (I) by filtration or gravity separation;
thereby recovering and/or isolating the tryptamine derivative (I).

119. A fermentation liquid comprising the tryptamine derivative (I) comprised in the cell culture of claim 112.

120. The fermentation liquid of claim 117, wherein at least 50%, such as at least 75%, such as at least 95%, such as at least 99% of the host cells are disrupted.

121. The fermentation liquid of claim 117 to 118, wherein at least 50%, such as at least 75%, such as at least 95%, such as at least 99% of solid cellular material has separated from the liquid.

122. The fermentation liquid of claim 117 to 119, further comprising one or more compounds selected from:
a) precursors or products of the operative biosynthetic pathway producing the tryptamine derivative (I);
b) supplemental nutrients comprising trace metals, vitamins, salts, yeast nitrogen base, YNB, and/or amino acids; and
wherein the concentration of the tryptamine derivative (I) is at least 1 mg/I liquid.

123. A composition comprising the fermentation liquid of claims 117 to 120 and/or the tryptamine derivative (I) of claims 80 to 81 and one or more agents, additives and/or excipients.

124. The composition of claim 121, wherein the fermentation liquid and/or the tryptamine derivative (I) have been processed into in a dry solid form, optionally in form of a powder, tablet, capsule, hard chewable and or soft lozenge or a gum.

125. The composition of claim 121, wherein the composition is in a liquid form, optionally in a stabilized liquid form.

126. A genetically modified microbial host cell producing a serotonin indole acceptor expressing
a) one or more genes encoding polypeptides selected from
i. one or more enzymes converting glucose to fructose-6-phosphate;
ii. a fructose-6-phosphate phosphoketolase converting fructose-6-phosphate to Erythrose-4-phosphate and acetyl phosphate;
iii. a Phosphotransacetylase converting Acetyl phosphate to Acetyl-CoA;
iv. one or more enzymes converting Fructose-6-phosphate to Phosphoenolpyruvate;
v. a 3-deoxy-D-arabino-heptulosonate 7-phosphate synthase (DAHP synthase) converting Phosphoenolpyruvate and Erythrose-4-phosphate to 3-deoxy-D-arabino-heptulosonate-7-phosphate (DAHP);
vi. one or more enzymes converting 3-deoxy-D-arabino-heptulosonate-7-phosphate to 5-enolpyruvoyl-shikimate 3-phosphate;
vii. a Shikimate kinase converting Shikimate to Shikimate-3-phosphate;
viii. a Chorismate synthase converting 5-enolpyruvoyl-shikimate 3-phosphate to Chorismate;
ix. a Anthranilate synthase converting Chorismate to Anthranilate;
x. a Ribose-phosphate pyrophosphokinase converting Ribose-5-phosphate to Phospho-alpha-D-ribosyl-1-pyrophosphate;
xi. a Anthranilate phosphoribosyl transferase converting Anthranilate and Phospho-alpha-D-ribosyl-1-pyrophosphate to N-(5-phosphoribosyl)-anthranilate;
xii. a N-(5′-phosphoribosyl)-anthranilate isomerase converting N-(5-phosphoribosyl)-anthranilate to 1-(o-carboxyphenylamino)-1′-deoxyribulose 5′-phosphate;
xiii. an Indole-3-glycerol phosphate synthase converting 1-(o-carboxyphenylamino)-1′-deoxyribulose 5′-phosphate to (1S,2R)-1-C-(indol-3-yl)-glycerol 3-phosphate;
xiv. a Tryptophan synthase converting (1S,2R)-1-C-(indol-3-yl) glycerol 3-phosphate and Serine to L-Tryptophan; and/or
xv. a Tryptophan decarboxylase converting L-Tryptophan to Tryptamine; and
b) a heterologous Tryptamine 5-hydroxylase converting tryptamine to serotonin; and
c) a heterologous Cytochrome p450 reductase assisting the conversion of tryptamine to serotonin by a Tryptamine 5-hydroxylase.

127. The host cell of claim 124, wherein the corresponding:
a) fructose-6-phosphate phosphoketolase has at least 70% identity to the fructose-6-phosphate phosphoketolase comprised in SEQ ID NO: 2;
b) Phosphotransacetylase has at least 70% identity to the Phosphotransacetylase comprised in SEQ ID NO: 4;
c) 3-deoxy-D-arabino-heptulosonate 7-phosphate synthase has at least 70% identity to the 3-deoxy-D-arabino-heptulosonate 7-phosphate synthase comprised in SEQ ID NO: 6;
d) Enzyme converting 3-deoxy-D-arabino-heptulosonate-7-phosphate to 5-enolpyruvoyl-shikimate 3-phosphate has at least 70% identity to the Enzyme comprised in SEQ ID NO: 8;
e) Shikimate kinase has at least 70% identity to the Shikimate kinase comprised in SEQ ID NO: 10;
f) Chorismate synthase has at least 70% identity to the Chorismate synthase comprised in SEQ ID NO: 12;
g) Anthranilate synthase has at least 70% identity to the Anthranilate synthase comprised in SEQ ID NO: 14;
h) Ribose-phosphate pyrophosphokinase has at least 70% identity to the Ribose-phosphate pyrophosphokinase comprised in SEQ ID NO: 16;
i) Anthranilate phosphoribosyl transferase has at least 70% identity to the Anthranilate phosphoribosyl transferase comprised in SEQ ID NO: 18;
j) N-(5′-phosphoribosyl)-anthranilate isomerase has at least 70% identity to the N-(5′-phosphoribosyl)-anthranilate isomerase comprised in SEQ ID NO: 20;
k) Indole-3-glycerol phosphate synthase has at least 70% identity to the Indole-3-glycerol phosphate synthase comprised in SEQ ID NO: 22;
l) Tryptophan synthase has at least 70% identity to the Tryptophan synthase comprised in anyone of SEQ ID NO: 24, 60, 62, 64, 66, 68, 180 and/or 182;
m) Tryptophan decarboxylase or a non-canonical aromatic amino acid decarboxylase has at least 70% identity to the Tryptophan decarboxylase or a non-canonical aromatic amino acid decarboxylase comprised in anyone of SEQ ID NO: 26, 70, 72, 74, 76, and/or 78;
n) Tryptamine 5-hydroxylase has at least 70% identity to the Tryptamine 5-hydroxylase comprised in SEQ ID NO: 96 (OsT5H); and
o) Cytochrome p450 reductase has at least 70% identity to the Cytochrome p450 reductase comprised in SEQ ID NO: 112 (FoCPR).

128. The host cell of claim 125, wherein the one or more expressed genes are selected from:
a) genes encoding a fructose-6-phosphate phosphoketolase said genes being at least 70% identical to the fructose-6-phosphate phosphoketolase encoding polynucleotide comprised in SEQ ID NO: 1 or genomic DNA thereof;
b) genes encoding a Phosphotransacetylase said genes being at least 70% identical to the Phosphotransacetylase encoding polynucleotide comprised in SEQ ID NO: 3 or genomic DNA thereof;
c) genes encoding a 3-deoxy-D-arabino-heptulosonate 7-phosphate synthase said genes being at least 70% identical to the 3-deoxy-D-arabino-heptulosonate 7-phosphate synthase encoding polynucleotide comprised in SEQ ID NO: 5 or genomic DNA thereof;
d) genes encoding an enzyme converting 3-deoxy-D-arabino-heptulosonate-7-phosphate to 5-enolpyruvoyl-shikimate 3-phosphate said genes being at least 70% identical to the polynucleotide encoding the enzyme converting 3-deoxy-D-arabino-heptulosonate-7-phosphate to 5-enolpyruvoyl-shikimate 3-phosphate comprised in SEQ ID NO: 7 or genomic DNA thereof;
e) genes encoding a XX said genes being at least 70% identical to the Shikimate kinase encoding polynucleotide comprised in SEQ ID NO: 9 or genomic DNA thereof;
f) genes encoding a Shikimate kinase said genes being at least 70% identical to the Chorismate synthase encoding polynucleotide comprised in SEQ ID NO: 11 or genomic DNA thereof;
g) genes encoding a Anthranilate synthase said genes being at least 70% identical to the Anthranilate synthase encoding polynucleotide comprised in SEQ ID NO: 13 or genomic DNA thereof;
h) genes encoding a Ribose-phosphate pyrophosphokinase said genes being at least 70% identical to the Ribose-phosphate pyrophosphokinase encoding polynucleotide comprised in SEQ ID NO: 15 or genomic DNA thereof;
i) genes encoding a nthranilate phosphoribosyl transferase said genes being at least 70% identical to the Anthranilate phosphoribosyl transferase encoding polynucleotide comprised in SEQ ID NO: 17 or genomic DNA thereof;
j) genes encoding a N-(5′-phosphoribosyl)-anthranilate isomerase said genes being at least 70% identical to the N-(5′-phosphoribosyl)-anthranilate isomerase encoding polynucleotide comprised in SEQ ID NO: 19 or genomic DNA thereof;
k) genes encoding a Indole-3-glycerol phosphate synthase said genes being at least 70% identical to the Indole-3-glycerol phosphate synthase encoding polynucleotide comprised in SEQ ID NO: 21 or genomic DNA thereof;
l) genes encoding a Tryptophan synthase said genes being at least 70% identical to the Tryptophan synthase encoding polynucleotide comprised in anyone of SEQ ID NO: 23, 59, 61, 63, 65, 67, 179, and/or 181 or genomic DNA thereof;
m) genes encoding a Tryptophan decarboxylase, or a non-canonical aromatic amino acid decarboxylase said genes being at least 70% identical to the Tryptophan decarboxylase or a non-canonical aromatic amino acid decarboxylase encoding polynucleotide comprised in SEQ ID NO: 25, 69, 71, 73, 75, and/or 77 or genomic DNA thereof;
n) genes encoding a Tryptamine 5-hydroxylase said genes being at least 70% identical to the Tryptamine 5-hydroxylase encoding polynucleotide comprised in SEQ ID NO: 96 or genomic DNA thereof;
o) genes encoding a Cytochrome p450 reductase said genes being at least 70% identical to the Cytochrome p450 reductase encoding polynucleotide comprised in SEQ ID NO: 111 or genomic DNA thereof.

129. The host cell of claim 124 to 126 wherein the host cell is a yeast strain modified by attenuating, disrupting and/or deleting one or more native genes selected from:
a) The pyruvate kinase gene comprised in anyone of SEQ ID NO: 27 or any of its paralogs or orthologs having at least 70% identity to SEQ ID NO: 27;
b) The phosphofructokinase gene comprised in anyone of SEQ ID NO: 29 and/or 31 or any of its paralogs or orthologs having at least 70% identity to anyone of SEQ ID NO: 29 and/or 31;
c) The transporters gene comprised in SEQ ID NO: 33 or any of its paralogs or orthologs having at least 70% identity to SEQ ID NO: 33;
d) The DL-glycerol-3-phosphate phosphatase gene comprised in SEQ ID NO: 34 or any of its paralogs or orthologs having at least 70% identity to SEQ ID NO: 34; and/or
e) The tryptophan 2,3-dioxygenase gene comprised in SEQ ID NO: 35 or any of its paralogs or orthologs having at least 70% identity to SEQ ID NO: 35;
f) The cystathionine beta-synthase gene comprised in SEQ ID NO: 36 or any of its paralogs or orthologs having at least 70% identity to SEQ ID NO: 36;
g) The phenylpyruvate decarboxylase gene comprised in SEQ ID NO: 37 or any of its paralogs or orthologs having at least 70% identity to SEQ ID NO: 37;
h) The pyruvate decarboxylase gene comprised in SEQ ID NO: 38 or any of its paralogs or orthologs having at least 70% identity to SEQ ID NO: 38; and/or
i) The histone variant H2AZ gene comprised in SEQ ID NO: 39 or any of its paralogs or orthologs having at least 70% identity to SEQ ID NO: 39.

130. The host cell of claims 124 to 127, wherein the host cell is a yeast strain modified by overexpressing one or more native genes selected from the NADH kinase gene comprised in SEQ ID NO: 185 or any of its paralogs or orthologs having at least 70% identity to SEQ ID NO: 185.

131. A cell culture, comprising host cell of claims 122 to 128 and a growth medium.

132. A method of producing a serotonin indole acceptor comprising:
a) culturing the cell culture of claim 129 at conditions allowing the host cell to produce the serotonin indole acceptor; and
b) optionally recovering and/or isolating the serotonin indole acceptor.

133. The method of claim 113, further comprising one or more elements selected from:
a) culturing the cell culture in a nutrient growth medium;
b) culturing the cell culture under aerobic or anaerobic conditions
c) culturing the cell culture under agitation;
d) culturing the cell culture at a temperature of between 25 to 50° C.;
e) culturing the cell culture at a pH of between 3-9;
f) culturing the cell culture for between 10 hours to 30 days; and
g) culturing the cell culture under fed-batch, repeated fed-batch, continuous, or semi-continuous conditions.

134. The method of claims 130 to 131, further comprising feeding one or more exogenous precursors for serotonine indole acceptor to the cell culture.

135. The method of claims 130 to 132, wherein the recovering and/or isolation step comprises separating a liquid phase of host cell or cell culture from a solid phase of host cell or cell culture to obtain a supernatant comprising the serotonin indole acceptor by one or more steps selected from:
a) disrupting the host cell to release intracellular serotonin indole acceptor into the supernatant;
b) separating the supernatant from the solid phase of the host cell, such as by filtration or gravity separation;
c) contacting the supernatant with one or more adsorbent resins in order to obtain at least a portion of the produced serotonin indole acceptor;
d) contacting the supernatant with one or more ion exchange or reversed-phase chromatography columns in order to obtain at least a portion of the serotonin indole acceptor;
e) extracting the serotonin indole acceptor; and
f) precipitating the serotonin indole acceptor by crystallization or evaporating the solvent of the liquid phase; and optionally isolating the serotonin indole acceptor by filtration or gravity separation;
thereby recovering and/or isolating the serotonin indole acceptor.

136. A fermentation liquid comprising the serotonin indole acceptor comprised in the cell culture of claim 129.

137. The fermentation liquid of claim 134, wherein at least 50%, such as at least 75%, such as at least 95%, such as at least 99% of the host cells are disrupted.

138. The fermentation liquid of claim 134 to 135, wherein at least 50%, such as at least 75%, such as at least 95%, such as at least 99% of solid cellular material has separated from the liquid.

139. The fermentation liquid of claim 131 to 136, further comprising one or more compounds selected from:
a) precursors or products of the operative biosynthetic pathway producing the serotonin indole acceptor;
b) supplemental nutrients comprising trace metals, vitamins, salts, yeast nitrogen base, YNB, and/or amino acids; and
wherein the concentration of the serotonin indole acceptor is at least 1 mg/I liquid.

140. A composition comprising the fermentation liquid of claims 131 to 137 and one or more agents, additives and/or excipients.

141. The composition of claim 138, wherein the fermentation liquid and/or the serotonin indole acceptor have been processed into in a dry solid form, optionally in form of a powder, tablet, capsule, hard chewable and or soft lozenge or a gum.

142. The composition of claim 138, wherein the composition is in a liquid form, optionally in a stabilized liquid form.

143. A genetically modified microbial host cell producing a psilocybin indole acceptor expressing
a) one or more enzymes selected from
i. one or more enzymes converting glucose to fructose-6-phosphate;
ii. a fructose-6-phosphate phosphoketolase converting fructose-6-phosphate to Erythrose-4-phosphate and acetyl phosphate;
iii. a Phosphotransacetylase converting Acetyl phosphate to Acetyl-CoA;
iv. one or more enzymes converting Fructose-6-phosphate to Phosphoenolpyruvate;
v. a 3-deoxy-D-arabino-heptulosonate 7-phosphate synthase (DAHP synthase) converting Phosphoenolpyruvate and Erythrose-4-phosphate to 3-deoxy-D-arabino-heptulosonate-7-phosphate (DAHP);
vi. one or more enzymes converting 3-deoxy-D-arabino-heptulosonate-7-phosphate to 5-enolpyruvoyl-shikimate 3-phosphate;
vii. a Shikimate kinase converting Shikimate to Shikimate-3-phosphate;
viii. a Chorismate synthase converting 5-enolpyruvoyl-shikimate 3-phosphate to Chorismate;
ix. a Anthranilate synthase converting Chorismate to Anthranilate;
x. a Ribose-phosphate pyrophosphokinase converting Ribose-5-phosphate to Phospho-alpha-D-ribosyl-1-pyrophosphate;
xi. a Anthranilate phosphoribosyl transferase converting Anthranilate and Phospho-alpha-D-ribosyl-1-pyrophosphate to N-(5-phosphoribosyl)-anthranilate;
xii. a N-(5′-phosphoribosyl)-anthranilate isomerase converting N-(5-phosphoribosyl)-anthranilate to 1-(o-carboxyphenylamino)-1′-deoxyribulose 5′-phosphate;
xiii. an Indole-3-glycerol phosphate synthase converting 1-(o-carboxyphenylamino)-1′-deoxyribulose 5′-phosphate to (1S,2R)-1-C-(indol-3-yl)-glycerol 3-phosphate;
xiv. a Tryptophan synthase converting (1S,2R)-1-C-(indol-3-yl) glycerol 3-phosphate and Serine to L-Tryptophan; and/or
xv. a Tryptophan decarboxylase converting L-Tryptophan to Tryptamine; and
b) a Tryptamine 4-hydroxylase converting tryptamine to 4-hydroxytryptamine;
c) a Cytochrome p450 reductase assisting tryptamine 4-hydroxylase in converting tryptamine to 4-hydroxytryptamine;
d) a Cytochrome b5 assisting tryptamine 4-hydroxylase in converting tryptamine to 4-hydroxytryptamine;
e) 4-hydroxytryptamine kinase converting 4-hydroxytryptamine to Norbaeocystin; and
f) Psilocybin synthase converting Norbaeocystin to Psilocybin.

144. The host cell of claim 141, wherein the corresponding:
a) fructose-6-phosphate phosphoketolase has at least 70% identity to the fructose-6-phosphate phosphoketolase comprised in SEQ ID NO: 2;
b) phosphotransacetylase has at least 70% identity to the Phosphotransacetylase comprised in SEQ ID NO: 4;
c) 3-deoxy-D-arabino-heptulosonate 7-phosphate synthase has at least 70% identity to the 3-deoxy-D-arabino-heptulosonate 7-phosphate synthase comprised in SEQ ID NO: 6;
d) enzyme converting 3-deoxy-D-arabino-heptulosonate-7-phosphate to 5-enolpyruvoyl-shikimate 3-phosphate has at least 70% identity to the Enzyme comprised in SEQ ID NO: 8;
e) Shikimate kinase has at least 70% identity to the Shikimate kinase comprised in SEQ ID NO: 10;
f) chorismate synthase has at least 70% identity to the Chorismate synthase comprised in SEQ ID NO: 12;
g) anthranilate synthase has at least 70% identity to the Anthranilate synthase comprised in SEQ ID NO: 14;
h) ribose-phosphate pyrophosphokinase has at least 70% identity to the Ribose-phosphate pyrophosphokinase comprised in SEQ ID NO: 16;
i) anthranilate phosphoribosyl transferase has at least 70% identity to the Anthranilate phosphoribosyl transferase comprised in SEQ ID NO: 18;
j) N-(5′-phosphoribosyl)-anthranilate isomerase has at least 70% identity to the N-(5′-phosphoribosyl)-anthranilate isomerase comprised in SEQ ID NO: 20;
k) indole-3-glycerol phosphate synthase has at least 70% identity to the Indole-3-glycerol phosphate synthase comprised in SEQ ID NO: 22;
l) tryptophan synthase has at least 70% identity to the Tryptophan synthase comprised in anyone of SEQ ID NO: 24, 60, 62, 64, 66, 68, 180 and/or 182;
m) tryptophan decarboxylase or a non-canonical aromatic amino acid decarboxylase has at least 70% identity to the Tryptophan decarboxylase or a non-canonical aromatic amino acid decarboxylase comprised in anyone of SEQ ID NO: 26, 70, 72, 74, 76, and/or 78;
n) tryptamine 4-hydroxylase has at least 70% identity to the Tryptamine 4-hydroxylase comprised in anyone of SEQ ID NO 94 and/or 88;
o) cytochrome p450 reductase has at least 70% identity to the Cytochrome p450 reductase comprised in anyone of SEQ ID NO 106 and/or 102;
p) cytochrome b5 has at least 70% identity to the Cytochrome b5 comprised in anyone of SEQ ID NO 254;
q) 4-hydroxytryptamine kinase has at least 70% identity to the 4-hydroxytryptamine kinase comprised in anyone of SEQ ID NO 160 and/or 156; and
r) Psilocybin synthase has at least 70% identity to the psilocybin synthase comprised in anyone of SEQ ID NO 128 and/or 124.

145. The host cell of claim 142, wherein the one or more expressed genes are selected from:
a) genes encoding a fructose-6-phosphate phosphoketolase said genes being at least 70% identical to the fructose-6-phosphate phosphoketolase encoding polynucleotide comprised in SEQ ID NO: 1 or genomic DNA thereof;
b) genes encoding a Phosphotransacetylase said genes being at least 70% identical to the Phosphotransacetylase encoding polynucleotide comprised in SEQ ID NO: 3 or genomic DNA thereof;
c) genes encoding a 3-deoxy-D-arabino-heptulosonate 7-phosphate synthase said genes being at least 70% identical to the 3-deoxy-D-arabino-heptulosonate 7-phosphate synthase encoding polynucleotide comprised in SEQ ID NO: 5 or genomic DNA thereof;
d) genes encoding an enzyme converting 3-deoxy-D-arabino-heptulosonate-7-phosphate to 5-enolpyruvoyl-shikimate 3-phosphate said genes being at least 70% identical to the polynucleotide encoding the enzyme converting 3-deoxy-D-arabino-heptulosonate-7-phosphate to 5-enolpyruvoyl-shikimate 3-phosphate comprised in SEQ ID NO: 7 or genomic DNA thereof;
e) genes encoding a XX said genes being at least 70% identical to the Shikimate kinase encoding polynucleotide comprised in SEQ ID NO: 9 or genomic DNA thereof;
f) genes encoding a Shikimate kinase said genes being at least 70% identical to the Chorismate synthase encoding polynucleotide comprised in SEQ ID NO: 11 or genomic DNA thereof;
g) genes encoding a Anthranilate synthase said genes being at least 70% identical to the Anthranilate synthase encoding polynucleotide comprised in SEQ ID NO: 13 or genomic DNA thereof;
h) genes encoding a Ribose-phosphate pyrophosphokinase said genes being at least 70% identical to the Ribose-phosphate pyrophosphokinase encoding polynucleotide comprised in SEQ ID NO: 15 or genomic DNA thereof;
i) genes encoding a nthranilate phosphoribosyl transferase said genes being at least 70% identical to the Anthranilate phosphoribosyl transferase encoding polynucleotide comprised in SEQ ID NO: 17 or genomic DNA thereof;
j) genes encoding a N-(5′-phosphoribosyl)-anthranilate isomerase said genes being at least 70% identical to the N-(5′-phosphoribosyl)-anthranilate isomerase encoding polynucleotide comprised in SEQ ID NO: 19 or genomic DNA thereof;
k) genes encoding a Indole-3-glycerol phosphate synthase said genes being at least 70% identical to the Indole-3-glycerol phosphate synthase encoding polynucleotide comprised in SEQ ID NO: 21 or genomic DNA thereof;
l) genes encoding a Tryptophan synthase said genes being at least 70% identical to the Tryptophan synthase encoding polynucleotide comprised in anyone of SEQ ID NO: 23, 59, 61, 63, 65, 67, 179, and/or 181 or genomic DNA thereof;
m) genes encoding a Tryptophan decarboxylase or a non-canonical aromatic amino acid decarboxylase said genes being at least 70% identical to the Tryptophan decarboxylase or a non-canonical aromatic amino acid decarboxylase encoding polynucleotide comprised in SEQ ID NO: 25, 69, 71, 73, 75, and/or 77 or genomic DNA thereof;
n) genes encoding a tryptamine 4-hydroxylase said genes at least 70% identical to the tryptamine 4-hydroxylase encoding polynucleotide comprised in SEQ ID NO 93 and/or 87;
o) genes encoding a cytochrome p450 reductase said genes being at least 70% identical to the cytochrome p450 reductase comprised in anyone of SEQ ID NO 105 and/or 101;
p) genes encoding a cytochrome b5 said genes being at least 70% identical to the Cytochrome b5 encoding polynucleotide comprised in SEQ ID NO 253;
q) genes encoding a 4-hydroxytryptamine kinase said genes being at least 70% identical to the 4-hydroxytryptamine kinase encoding polynucleotide comprised SEQ ID NO 159 and/or 155; and/or
r) genes encoding a psilocybin synthase said genes being at least 70% identical to the psilocybin synthase encoding polynucleotide comprised in SEQ ID NO 127 and/or 123.

146. The host cell of claim 141 to 143 wherein the host cell is a yeast strain modified by attenuating, disrupting and/or deleting one or more native genes selected from:
a) The pyruvate kinase gene comprised in anyone of SEQ ID NO: 27 or any of its paralogs or orthologs having at least 70% identity to SEQ ID NO: 27;
b) the phosphofructokinase gene comprised in anyone of SEQ ID NO: 29 and/or 31 or any of its paralogs or orthologs having at least 70% identity to anyone of SEQ ID NO: 29 and/or 31;
c) the transporters gene comprised in SEQ ID NO: 33 or any of its paralogs or orthologs having at least 70% identity to SEQ ID NO: 33;
d) the DL-glycerol-3-phosphate phosphatase gene comprised in SEQ ID NO: 34 or any of its paralogs or orthologs having at least 70% identity to SEQ ID NO: 34;
e) the tryptophan 2,3-dioxygenase gene comprised in SEQ ID NO: 35 or any of its paralogs or orthologs having at least 70% identity to SEQ ID NO: 35;
f) the cystathionine beta-synthase gene comprised in SEQ ID NO: 36 or any of its paralogs or orthologs having at least 70% identity to SEQ ID NO: 36;
g) the phenylpyruvate decarboxylase gene comprised in SEQ ID NO: 37 or any of its paralogs or orthologs having at least 70% identity to SEQ ID NO: 37;
h) the pyruvate decarboxylase gene comprised in SEQ ID NO: 38 or any of its paralogs or orthologs having at least 70% identity to SEQ ID NO: 38;
i) the histone variant H2AZ gene comprised in SEQ ID NO: 39 or any of its paralogs or orthologs having at least 70% identity to SEQ ID NO: 39;
j) the phosphatase gene comprised in SEQ ID NO: 40 or any of its paralogs or orthologs having at least 70% identity to SEQ ID NO: 40;
k) the repressible acid phosphatase gene comprised in anyone of SEQ ID NO: 41, 42, or 43 and/or any of its paralogs or orthologs having at least 70% identity to anyone of SEQ ID NO: 41, 42, and/or 43; and/or
l) the constitutively expressed acid phosphatase gene comprised in SEQ ID NO: 44 and/or any of its paralogs or orthologs having at least 70% identity to SEQ ID NO: 44.

147. The host cell of claims 141 to 144, wherein the host cell is a yeast strain modified by overexpressing one or more native genes selected from the NADH kinase gene comprised in SEQ ID NO: 185 or any of its paralogs or orthologs having at least 70% identity to SEQ ID NO: 185.

148. A cell culture, comprising host cell of claims 141 to 145 and a growth medium.

149. A method of producing a psilocybin indole acceptor comprising:
a) culturing the cell culture of claim 146 at conditions allowing the host cell to produce the psilocybin indole acceptor; and
b) optionally recovering and/or isolating the psilocybin indole acceptor.

150. The method of claim 147, further comprising one or more elements selected from:
a) culturing the cell culture in a nutrient growth medium;
b) culturing the cell culture under aerobic or anaerobic conditions
c) culturing the cell culture under agitation;
d) culturing the cell culture at a temperature of between 25 to 50° C.;
e) culturing the cell culture at a pH of between 3-9;
f) culturing the cell culture for between 10 hours to 30 days; and
g) culturing the cell culture under fed-batch, repeated fed-batch, continuous, or semi-continuous conditions.

151. The method of claims 147 to 148, further comprising feeding one or more exogenous precursors for psilocybin indole acceptor to the cell culture.

152. The method of claims 147 to 149, wherein the recovering and/or isolation step comprises separating a liquid phase of host cell or cell culture from a solid phase of host cell or cell culture to obtain a supernatant comprising the psilocybin indole acceptor by one or more steps selected from:
a) disrupting the host cell to release intracellular psilocybin indole acceptor into the supernatant;
b) separating the supernatant from the solid phase of the host cell, such as by filtration or gravity separation;
c) contacting the supernatant with one or more adsorbent resins in order to obtain at least a portion of the produced psilocybin indole acceptor;
d) contacting the supernatant with one or more ion exchange or reversed-phase chromatography columns in order to obtain at least a portion of the psilocybin indole acceptor;
e) extracting the psilocybin indole acceptor; and
f) precipitating the psilocybin indole acceptor by crystallization or evaporating the solvent of the liquid phase; and optionally isolating the psilocybin indole acceptor by filtration or gravity separation;
thereby recovering and/or isolating the psilocybin indole acceptor.

153. A fermentation liquid comprising the psilocybin indole acceptor comprised in the cell culture of claim 146.

154. The fermentation liquid of claim 151, wherein at least 50%, such as at least 75%, such as at least 95%, such as at least 99% of the host cells are disrupted.

155. The fermentation liquid of claim 151 to 152, wherein at least 50%, such as at least 75%, such as at least 95%, such as at least 99% of solid cellular material has separated from the liquid.

156. The fermentation liquid of claim 151 to 153, further comprising one or more compounds selected from:
a) precursors or products of the operative biosynthetic pathway producing the psilocybin indole acceptor;
b) supplemental nutrients comprising trace metals, vitamins, salts, yeast nitrogen base, YNB, and/or amino acids; and
wherein the concentration of the psilocybin indole acceptor is at least 1 mg/I liquid.

157. A composition comprising the fermentation liquid of claims 151 to 154 and one or more agents, additives and/or excipients.

158. The composition of claim 155, wherein the fermentation liquid and/or the psilocybin indole acceptor have been processed into in a dry solid form, optionally in form of a powder, tablet, capsule, hard chewable and or soft lozenge or a gum.

159. The composition of claim 155, wherein the composition is in a liquid form, optionally in a stabilized liquid form.
